(19) United States
(12) Patent Application Publication
(10) Pub. No.: US 2023/0149507 A1
(43) Pub. Date: May 18, 2023

(54) TREATMENT OF ALT CANCERS

(71) Applicants: INSTITUTO DE MEDICINA MOLECULAR JOÃO LOBO ANTUNES, Lisboa (PT); CHILDREN'S MEDICAL RESEARCH INSTITUTE, Westmead (AU)

(72) Inventors: Hilda Amelia PICKETT, Surry Hills (AU); Claus Maria AZZALIN, Lisboa (PT); Alexander Peter SOBINOFF, Hornsby (AU)

(21) Appl. No.: 17/612,547

(22) PCT Filed: May 23, 2020
(86) PCT No.: PCT/PT2020/050021

§ 371 Date: Nov. 18, 2021

(30) Foreign Application Priority Data
May 24, 2019 (AU) 2019901766
May 28, 2019 (GB) 1907518.3

(51) Int. Cl. A61K 38/17; C12N 15/113; A61K 31/496; A61P 35/00; A61K 45/06; G01N 33/50

(52) U.S. Cl. CPC A61K 38/1709; C12N 15/113; A61K 31/496; A61P 35/00; A61K 45/06; G01N 33/5011; C12N 2310/14

(57) ABSTRACT

The present disclosure generally relates to cancer and the treatment of cancer, including the treatment of Alternative Lengthening of Telomeres (ALT) cancers. The disclosure provides methods comprising inhibiting FANCM activity or expression, such as inhibiting FANCM's interaction with RMI and/or FANCM's ATPase activity, to inhibit the growth and/or proliferation of ALT tumor cells and/or to induce death of ATL tumor cells. The methods may be practiced on patients diagnosed with an ALT tumor.

### CROSS-REFERENCE TO RELATED APPLICATIONS

This application claims priority to Australian Provisional Application No. 2019901766, filed on May 24, 2019; and U.K. (GB) Application No. 1907518.3, filed on May 28, 2019; both of which are incorporated by reference herein in their entireties.

### STATEMENT REGARDING THE SEQUENCE LISTING

The Sequence Listing associated with this application is provided in text format in lieu of a paper copy, and is hereby incorporated by reference into the specification. The name of the text file containing the Sequence Listing is PCT Sequence Listing_TREATMENT OF ALT CANCERS.txt. The text file is about 305 KB, was created on May 20, 2020, and is being submitted electronically as part of the description.

### FIELD

The present disclosure generally relates to cancer and the treatment of cancer, including the treatment of Alternative Lengthening of Telomeres (ALT) cancers.

### BACKGROUND

Genomic integrity is maintained in normal cells in part by telomeres. The progressive shortening of telomeres through successive cell divisions induces chromosomal instability. Replicative immortality is often achieved by cancer cells through overcoming telomere shortening. Telomere shortening must be counteracted in immortal cells, including the large majority of cancer cells, to avoid senescence or death¹. In the majority of cancer cells, telomere length is maintained by telomerase. Approximately 90% of human cancers have reactivated the reverse transcriptase telomerase, which adds newly synthesized telomeric repeats to the 3′ end of linear chromosomes^{2, 3}. About 10-15% of immortal cancer cells are telomerase-negative and replenish telomeres via telomerase independent strategies, which are collectively referred to as Alternative Lengthening of Telomeres (ALT) (Bryan et al., 1995 and Bryan et al., 1997) or the ALT pathway⁴. In humans, ALT was reported in tumors of mesenchymal or epithelial origin, including osteosarcomas, liposarcomas, glioblastomas, astrocytomas, and bladder carcinomas as well as in in vitro immortalized cells lines⁴⁻⁸.

Molecular features considered markers for ALT comprise: i) telomeres of heterogeneous lengths at different chromosome ends, including telomeres much longer than average telomeres in telomerase positive cells⁸; ii) elevated levels of the telomeric long noncoding RNA (lncRNA) TERRA⁹⁻¹³; iii) clustering of multiple telomeres into ALT-associated PML bodies (APBs), nuclear structures containing promyelocytic leukaemia protein (PML), telomeric factors such as TRF1, TRF2 and RAP1, TERRA, and DNA repair factors such as RAD51, RAD52, Replication Protein A (RPA), Brca1, and Bloom (BLM) and Werner helicases^{11, 14-19}; iv) abundant extrachromosomal telomeric repeats (ECTRs) comprising double-stranded (ds) circles (t-circles), partially single-stranded (ss) circles (C- and G-circles) and linear dsDNA²⁰⁻²³; v) recurrent mutations of the Alpha Thalassemia/Mental Retardation Syndrome X-Linked (ATRX) gene¹².

ALT cells are characterized by elevated levels of DNA damage compared to mortal or telomerase-positive cells, indicative of heightened telomeric replication stress in ALT cells. This is attributed to cumulative inadequacies in telomere structural integrity. Frequent or persistent replication fork stalling causes nicks and breaks in the DNA, and it has been hypothesized that the ALT mechanism emanates from stalled replication forks that deteriorate to form double stranded breaks (DSBs), that then provide the substrate for the engagement of homology-directed repair pathways, culminating in break induced telomere synthesis. ALT telomeres therefore achieve a fine balance between telomere protection and telomere damage and repair activities, and disruption of this balance has the potential to dysregulate the ALT mechanism.

Multiple DNA metabolism pathways collaborate to maintain telomeres in ALT cells. Break-induced replication (BIR) is active at ALT telomeres in the G2 phase of the cell cycle, and is stimulated by DSBs experimentally induced using the telomere-tethered DNA endonuclease TRF1-FokI 24,25. ALT BIR requires POLD3 and POLD4, two regulatory subunits of DNA polymerase delta^{24,25}. Conservative mitotic DNA synthesis (MiDAS) was also documented in human ALT cells²⁶. ALT MiDAS is stimulated by replication stress and requires RAD52²⁶. Finally, clustering of ALT telomeres within APBs is promoted by RAD51-dependent long-range movements, which are also stimulated by TRF1-FokI-induced DSBs²⁷. Telomere movements may promote efficient homology searches and telomere synthesis, although both ALT BIR and MiDAS are independent of RAD51^{25,26}.

A common notion deriving from all this work is that a sustained physiological damage must be maintained at ALT telomeres to promote telomere elongation. This is consistent with the presence of replication stress and DNA damage markers in APBs^{11,14-18}. The T triggers of this damage remain unclear, although RNA:DNA hybrids (R-loops), G-quadruplexes and oncogene expression were proposed as candidates 11, This scenario implies that telomeric damage levels be maintained within a specific threshold that is high enough to trigger DNA synthesis-based repair, yet not too high to induce cell death. Consistently, telomeric R-loops (telR-loops) formed by TERRA and telomeric DNA activate replication stress at ALT telomeres, and their levels are tightly controlled by the endoribonuclease RNaseH1^{11,28}. When RNaseH1 is depleted, excessive replication stress rapidly leads to abundant telomere free chromosome ends (TFEs) and increased C-circles. Conversely, RNaseH1 over-expression causes progressive TFE accumulation, likely due to inefficient de novo synthesis of telomeric DNA¹¹. The DNA damage signaling kinase ATM- and Rad3-Related (ATR) and the annealing helicase SWI/SNF-related matrix-associated actin-dependent regulator of chromatin subfamily A-like protein 1 (SMARCAL1) were also reported to restrict replicative stress at ALT telomeres^{29,30}.

The Fanconi anemia, complementation group M (FANCM) ATPase/translocase is a component of the Fanconi Anemia (FA) complex, where it supports efficient FANCD2 ubiquitination upon stalling of replication forks by physical impediments including DNA crosslinks³¹. Independently of the FA complex, FANCM remodels replication forks, recruits DNA repair factors at damage sites, suppresses meiotic crossovers and facilitates ATR checkpoint activation³²⁻³⁵. Moreover, the ATPase/translocase activity of FANCM resolves RNA: DNA hybrids in vitro and that R-loops accumulate genome-wide in FANCM deficient cells³⁶.

FANCM is an integral factor in the stabilization of stalled replication forks. It contains two DNA binding domains at its N- and C-termini between which are three highly conserved regions (MM1-MM3). The MM1 domain recruits the FA core complex, a multi-subunit ubiquitin ligase that is essential for DNA interstrand crosslink (ICL) repair, while the MM2 domain binds directly to the RMI1-RMI2 subcomplex of BLM-TOP3A-RMI (BTR). The BTR complex encompasses BLM helicase activity, TOP3A decatenation activity, branch migration and overall dissolvase activity and it has been suggested that FANCM and BTR may cooperate to regress, and thus stabilize, stalled forks. FANCM retention at stalled replication forks is dependent on its interaction with a functional BTR complex, but not with the FA core complex.

In ALT cells, FANCM allows efficient progression of the replication fork through the telomeric tract, and depletion of FANCM induces telomeric replication stress¹⁷. FANCM depletion leads to accumulation of BLM and Brca1 at ALT telomeres and co-depletion of FANCM with Brca1 or BLM has been shown to be lethal¹⁷.

Many cancer therapeutics act by indiscriminately damaging DNA, and cancer cells which lack robust DNA repair capacity cannot survive chemotherapeutic doses that are tolerated by healthy tissue. Whilst DNA damaging chemotherapies are initially effective, reactivation of tumour DNA repair pathways can lead to treatment failure and poor patient outcomes. The Fanconi anemia (FA) pathway is commonly activated during tumorigenesis and the reactivation or upregulation of the FA pathway has been linked to chemotherapy resistance in many cancers. Proper execution of the FA pathway requires interaction between the FANCM protein and the RMI complex, and mutations that disrupt the FANCM-RMI interaction can sensitize cells to DNA crosslinking agents. It has been hypothesized that inhibitors blocking FANCM-RMI complex formation may be useful as therapeutics to resensitize tumours that have acquired chemotherapeutic resistance. In Voter et al., 2016, a screen was developed to identify such inhibitors. However, the effect of these inhibitors on the ALT mechanism has not been investigated.

In WO 2017/146947, it was found that depletion of FANCM and at least one of BLM or BRCA1 can induce replication stress in ALT cells, which primarily takes place at the telomeres and dramatically reduces replication efficiency at ALT telomeres. However, the direct effects of FANCM depletion on ALT activity have not been fully determined.

Clearly, there is a need for new drug targets and improved treatments for cancer, including ALT cancers.

### SUMMARY

The present inventors have discovered that depletion of FANCM in cells that rely on the Alternative Lengthening of Telomeres (ALT) pathway induces cell death. The present inventors have demonstrated that inhibition of FANCM activity mediated through its interaction with RMI promotes ALT activity, and this inhibition can be applied to inhibit the growth and/or proliferation of ALT cells. In addition, the present inventors have demonstrated that inhibition of the ATPase activity of FANCM can also be applied to inhibit the growth and/or proliferation of ALT cells. On the basis of these findings, the inventors have developed new methods of inhibiting ALT cell viability and/or growth, and related methods of treating ALT cancers, comprising inhibiting or depleting FANCM, inhibiting the ATPase activity of FANCN, and/or disrupting the FANCM-RMI interaction in ALT cells. Furthermore, the inventors have found that inhibiting FANCM and BLM at the same time leads to an alleviation of the telomere dysfunction which is associated to FANCM depletion alone. Thus, in certain embodiments of any of the aspects of the invention disclosed herein, BLM and/or BRCA1 are not inhibited or depleted.

A first aspect of the invention provides a method of treating an ALT cancer in an individual in need thereof comprising reducing Fanconi anemia group M protein (FANCM) expression or activity in the individual.

A second aspect of the invention provides an agent which reduces FANCM expression or activity for use in a method of the first aspect.

A third aspect of the invention provides the use of an agent which reduces FANCM expression or activity in the manufacture of a medicament for use in a method of the first aspect.

A fourth aspect of the invention provides a method of screening for a compound that reduces viability or induces cell death in ALT cancer cells comprising determining the binding of a test compound to FANCM.

A fifth aspect of the invention provides a method of screening for a compound that reduces viability or induces cell death in ALT cancer cells comprising determining the effect of a test compound on the expression or activity of FANCM.

A sixth aspect of the invention provides a method of determining the responsiveness of a cancer in an individual to an agent which reduces FANCM expression or activity, the method comprising determining the presence of one or more ALT cancer cells in a sample of cancer cells from the individual, the presence of one or more ALT cancer cells in the sample being indicative that the cancer is responsive to said agent.

An individual with a cancer determined to be responsive to said agent may be treated by a method of the first aspect.

In a seventh aspect, the present disclosure provides a method of inhibiting ALT cell viability and/or growth, comprising disrupting the FANCM-RMI interaction. The method may be a method of treating ALT cancer in a subject.

Any of the methods disclosed herein may comprise disrupting the FANCM-RMI interaction by administering an inhibitor of the FANCM-RMI interaction.

The methods disclosed herein may comprise disrupting the binding of FANCM to RMI at the MM2 domain.

Any of the methods disclosed herein may comprise inhibiting the ATPase activity of FANCM by administering an inhibitor of FANCM ATPase activity.

Any of the methods disclosed herein may comprise disrupting the FANCM-RMI interaction by administering an inhibitor of the FANCM-RMI interaction and inhibiting the ATPase activity of FANCM by administering an inhibitor of FANCM ATPase activity.

In certain embodiments, any of the methods disclosed herein may not include inhibiting BLM and/or BRCA1.

The methods disclosed herein may further comprise the simultaneous, sequential or separate administration of a chemotherapeutic agent.

Alternatively, the methods disclosed herein may not comprise the administration of a chemotherapeutic agent. Thus, the methods disclosed herein may not comprise the simultaneous, sequential or separate administration of a chemotherapeutic agent. For example, the methods disclosed herein may not comprise the simultaneous administration of a chemotherapeutic agent.

In an eighth aspect, the present disclosure provides a method of selecting a subject for treatment with an inhibitor of the FANCM-RMI interaction, the method comprising determining whether the subject is suffering from ALT cancer, wherein the subject is selected for treatment with the inhibitor of the FANCM-RMI interaction if the subject is suffering from ALT cancer.

In a ninth aspect, the present disclosure provides a method of identifying whether a subject suffering from cancer is suitable for treatment with an inhibitor of the FANCM-RMI interaction, comprising determining whether the cancer is ALT cancer, wherein the subject is identified as suitable for treatment with the inhibitor of the FANCM-RMI interaction if the subject is suffering from ALT cancer.

In a tenth aspect, the present disclosure provides a method of determining whether a subject is responding to treatment with an inhibitor of the FANCM-RMI interaction, comprising:
determining the presence and/or extent of genomic instability at one or more telomeres in a cell taken from a subject; and/or
determining the presence and/or level of ALT activity in a cell taken from a subject.

In another aspect, the present disclosure provides a method of determining whether a subject is responding to treatment with an inhibitor of the FANCM-RMI interaction, comprising determining the growth and/or viability of ALT cells in or derived from the subject, wherein a reduction in the growth and/or viability of ALT cells after initiation of treatment compared to before initiation of treatment indicates that the subject is responding positively to the treatment. Any of the methods of identifying or detecting an ALT cell disclosed herein may be used in this regard.

In an eleventh aspect, the present disclosure provides a pharmaceutical composition comprising an inhibitor of the FANCM-RMI interaction for use in treating ALT cancer.

In a twelfth aspect, the present disclosure provides the use of an inhibitor of the FANCM-RMI interaction in the manufacture of a medicament for the treatment of ALT cancer.

The pharmaceutical composition may consist essentially of an inhibitor of the FANCM-RMI interaction. Thus, the medicament may consist essentially of an inhibitor of the FANCM-RMI interaction.

The inhibitor of the FANCM-RMI interaction may be any one or more of a genetic inhibitor, a small molecule, a peptide and a protein.

In one embodiment, the inhibitor is a genetic inhibitor. For example, the genetic inhibitor may be siRNA.

In one embodiment, the inhibitor is a small molecule. For example, the small molecule may be 4-[(1-Hydroxy-2-phenyl-1H-indol-3-yl)-pyridin-2-yl-methyl]-piperazine-1-carboxylic acid ethyl ester.

In one embodiment, the inhibitor is a peptide. For example, the peptide may be a peptide that comprises an amino acid sequence at least 90% identical to a peptide selected from the group consisting of: DLFSVTFDLGFC (SEQ ID NO: 49), DIFDCSRDLFSVTFDLGFCSPDSDDEILEHTSD (SEQ ID NO: 50) and EDIFDCSRDLFSVTFDLGFCSPDSDDEILEHTSD (SEQ ID NO: 5). In one example, the peptide is at least 90% identical to DLFSVTFDLGFC (SEQ ID NO: 49). In another example, the peptide is at least 90% identical to DIFDCSRDLFSVTFDLGFCSPDSDDEILEHTSD (SEQ ID NO: 50). In another example, the peptide is at least 90% identical to EDIFDCSRDLFSVTFDLGFCSPDSDDEILEHTSD (SEQ ID NO: 5).

In one embodiment, the inhibitor is a protein. For example, the protein may be an inactivated FANCM protein or an inactivated RMI complex. In one example, the inactivated FANCM protein may comprise a F1232A/F1236A double substitution. In another example, the protein may comprise an immunoglobulin binding domain.

In a thirteenth aspect, the present disclosure provides a pharmaceutical composition comprising an inhibitor of FANCM ATPase activity for use in treating ALT cancer.

In a fourteenth aspect, the present disclosure provides the use of an inhibitor of FANCM ATPAse activity in the manufacture of a medicament for the treatment of ALT cancer.

The pharmaceutical composition may consist essentially of an inhibitor of the FANCM ATPase activity. Thus, the medicament may consist essentially of an inhibitor of the FANCM FANCM ATPase interaction.

The inhibitor of the an inhibitor of the FANCM ATPase activity may be any one or more of a genetic inhibitor, a small molecule, a peptide and a protein.

In one embodiment, the inhibitor is a genetic inhibitor. For example, the genetic inhibitor may be siRNA.

In a fifteenth aspect, the present disclosure provides a pharmaceutical composition comprising an inhibitor of the FANCM-RMI interaction and an inhibitor of FANCM ATPase activity for use in treating ALT cancer.

In a sixteenth aspect, the present disclosure provides the use of an inhibitor of the FANCM-RMI interaction and an inhibitor of FANCM ATPAse activity in the manufacture of a medicament for the treatment of ALT cancer.

The pharmaceutical composition may consist essentially of an inhibitor of the FANCM-RMI interaction and an inhibitor of the FANCM ATPase activity. Thus, the medicament may consist essentially of an inhibitor of the FANCM-RMI interaction and an inhibitor of the FANCM FANCM ATPase interaction.

The inhibitor of the FANCM-RMI interaction and the inhibitor of the FANCM ATPase activity may be any one or more of a genetic inhibitor, a small molecule, a peptide and a protein.

Other aspects and embodiments of the invention are described in more detail below.

### BRIEF DESCRIPTION OF THE FIGURES

The following figures form part of the present specification and are included to further demonstrate certain aspects of the present disclosure. The disclosure may be better understood by reference to one or more of these figures in combination with the detailed description of specific embodiments presented herein.

FIG. 1. FANCM depletion in telomerase-positive cells.

(a) Western immunoblotting of U-2 OS, IIICF/c, GM847, Saos-2, HeLa, HeLa 1.2.11 and HCT116 cells with or without FANCM depletion (siFANCM1 or siFANCM2). (b) Quantitation of non-telomeric 53BP1 foci in U-2 OS and HeLa cells with or without FANCM depletion. Scatterplot bars represent the mean±SEM from n=150 cells from 3 experiments, **p<0.005, Mann-Whitney test. (c) Native and denaturing TRF analysis of non-digested (ND) and HinfI/RsaI digested (dig.) DNA in U-2 OS cells with or without FANCM depletion. (d) Representative dot blots and quantitation of C-circles in IIICF/c, GM847, Saos-2, HeLa, HeLa 1.2.11 and HCT116 cells with or without FANCM depletion. C-circles were normalized to a reference sample control. Error bars represent the mean±SEM from n=3 experiments, *p<0.05, **p<0.005, Student's t-test. Tukey boxplots of (e) APB frequency and (f) mean APB telomere foci intensity in IIICF/c, GM847, Saos-2, HeLa, HeLa 1.2.11 and HCT116 cells with or without FANCM depletion. Out of 3 experiments, n=150 cells scored per treatment, *p<0.05, **p<0.005, n.s.=non-significant, Mann-Whitney test.

FIG. 2. FANCM depletion results in telomere dysfunction and increased ALT activity

(a) Representative images of telomere (green) and γ-H2AX (red) colocalizations on metaphase spreads (meta-TIFs) in U-2 OS cells with or without FANCM depletion (left panel). Meta-TIFs are indicated by white arrows. Scale bars are 5 μm. Quantitation of TIFs (right panel). Scatterplot bars represent the mean±SEM. Out of 3 experiments, n≥81 metaphases scored per treatment, **p<0.005, Mann-Whitney test. (b) Native and denatured TRF analysis of U-2 OS cells with or without FANCM depletion. Colored arrows correspond to telomeric DNA species depicted in panel c. (c) Schematic of migration patterns for indicated telomeric species separated by two-dimensional gel electrophoresis (top panel). Two-dimensional TRF analysis of U-2 OS cells with or without FANCM depletion, hybridized under native and denatured conditions to detect the telomeric C-strand and G-strand (left and right panels). Colored arrows correspond to telomeric DNA species depicted in schematic. (d) Representative dot blot and quantitation of C-circle assays of U-2 OS cells with or without FANCM depletion. C-circle levels were normalized to the mean of scrambled control. Error bars represent the mean±SEM from n=3 experiments, *p<0.05, **p<0.005, Student's t-test. (e) Representative images of telomere (green) and PML (red) colocalizations (APBs) in FANCM-depleted U-2 OS cells. APBs are indicated by white arrows. Scale bars are 5 μm. Tukey box plots of (f) APB frequency and (g) mean APB telomere foci intensity per cell. Out of 3 experiments, n≥150 cells scored per treatment, *p<0.05, **p<0.005, Mann-Whitney test.

FIG. 3. FANCM depletion results in the generation of nascent telomeric DNA

(a) Representative images of TRF2 (red) and POLD3 (green) colocalizations in U-2 OS cells with or without FANCM depletion (top panel). Colocalizations are indicated by white arrows. Scale bars are 5 μm. Quantitation of colocalizations (bottom panel). Scatterplot bars represent the mean±SEM. Out of 3 experiments, n=150 cells scored treatment, *p<0.05, **p<0.005, Mann-Whitney test. (b) Representative dot blot and quantitation of nascent telomeric C-strand (top panel) and G-strand (bottom panel) DNA following BrdU incorporation and immunoprecipitation in U-2 OS cells with or without FANCM depletion. Nascent telomeric content was normalized to serial dilution of input DNA. Error bars represent mean±SEM from n=3 experiments, **p<0.005, n.s.=non-significant, Student's t-test. (c) Representative images of telomere (orange), PML (green) and EdU (violet) colocalizations (EdU-APB) in U-2 OS cells with or without FANCM depletion (top panel). Scale bars are 5 μm. Quantitation of colocalizations (bottom panel). Scatterplot bars represent the mean±SEM. Out of 3 experiments, n≥122 non-S-phase cells scored per treatment, **p<0.005, Mann-Whitney test. (d) Representative dot blot and quantitation of C-circle assays following BrdU incorporation and immunoprecipitation in U-2 OS cells with or without FANCM depletion. C-circles were normalized to the mean of scrambled control. Error bars represent mean±SEM out of n=3 experiments, **p<0.005, Student's t-test.

FIG. 4. Co-depletion of FANCM and POLD3, BLM, RAD51 or RAD52. (a) Representative Western immunoblotting of U-2 OS cells co-depleted of FANCM (siFANCM2) and either POLD3, BLM, RAD51 or RAD52. (b) Densitometry analysis of POLD3, BLM, RAD51 and RAD52 bands from n=3 experiments. Quantitation was conducted by first normalizing to the loading control (actin or vinculin), then to the scrambled control. Error bars represent the mean±SEM from n=3 experiments, *p<0.05, n.s.=non-significant, Student's t-test. (c) Native and denaturing TRF analysis of U-2 OS cells co-depleted of FANCM and either POLD3, BLM, RAD51 or RAD52.

FIG. 5. FANCM depletion results in increased break-induced telomere synthesis

(a) Representative dot blots and quantitation of C-circles in U-2 OS cells co-depleted of FANCM and either POLD3, BLM, RAD51 or RAD52. C-circles were normalized to the mean of scrambled control. Error bars represent mean±SEM from n=3 experiments, *p<0.05, n.s.=non-significant, Student's t-test. Quantitations of (b) APB frequency and (c) mean APB telomere foci intensity in U-2 OS cells co-depleted of FANCM and either POLD3, BLM, RAD51 or RAD52. Scatterplot bars represent the mean±SEM. Out of 3 experiments, n=150 cells scored per treatment, *p<0.05, **p<0.005, Mann-Whitney test. (d) Examples of telomere extension fibers (red) scored after CldU incorporation (green) (top panel). Quantitation of the number and length of telomere extension events in U-2 OS cells co-depleted of FANCM and either POLD3, BLM, RAD51 or RAD52 (left and right panels). Error bars represent mean±SEM of n≥350 fibers out of 3 experiments, *p<0.05, **p<0.005, Student's t-test.

FIG. 6. ALT activity is attenuated by the replication fork remodeling capabilities of FANCM

(a) Schematic of FANCM domain interactions, mutations or deletions. For clarity, domains have been color coded by functional role (bottom panel). (b) Quantitation of metaphase-TIFs in U-2 OS cells stably overexpressing wild-type (FANCM+) or FANCM mutants. Scatterplot bars represent the mean±SEM. Out of 3 experiments, n≥110 metaphases scored for each mutant, *p<0.05, **p<0.005, Mann-Whitney test. (c) Quantitation of fragile telomeres in U-2 OS cells stably overexpressing wild-type (FANCM+) or FANCM mutants. Scatterplot bars represent the mean±SEM. Out of 3 experiments, n>100 metaphases scored for each mutant, *p<0.05, **p<0.005, Mann-Whitney test. (d) Representative dot blots and quantitation of C-circle assays in U-2 OS cells stably overexpressing wild-type (FANCM+) or FANCM domain mutants. C-circles were normalized to the mean of vector control. Error bars represent mean±SEM from n=3 experiments, *p<0.05, **p<0.005, Student's t-test. (e) Single molecule analysis of telomeric extension events (top panel) and length of extension events (bottom panel) in U-2 OS cells stably overexpressing wild-type (FANCM+) or FANCM domain mutants. Error bars represent mean±SEM of n≥350 fibers out of 3 experiments, *p<0.05, **p<0.005, Student's t-test.

FIG. 7. Expression and analysis of FANCM mutants.

(a) Western immunoblotting of stable wild-type FANCM (FANCM+) or FANCM mutant overexpression in U-2 OS cells. NB: CV5.1 anti-FANCM mAb binds in the MM3 region and thus does not detect this variant. (b) TRF analysis of U-2 OS cells overexpressing wild-type (FANCM+) or FANCM mutants. Gels were hybridized under native and denatured conditions with radiolabelled telomeric probes to detect the C-strand and G-strand. (c) Tukey boxplots of APB frequency (top panel) and mean APB telomere intensity (bottom panel) in U-2 OS cells overexpressing wild-type (FANCM+) or FANCM mutants. Out of 3 experiments, n=150 cells scored per variant, *p<0.05, **p<0.005, Mann-Whitney test.

FIG. 8. ALT cells are hypersensitive to FANCM depletion. Cell cycle profiles of (a) U-2 OS and (b) HeLa cells stained with propidium iodide (PI). 9,800-10,000 gated events were collected. Cell cycle distribution was determined by Jett-Dean approximation. Live-cell quantitation of interphase duration (left panel) and mitotic entry (right panel) in (c) U-2 OS and (d) HeLa cells with or without FANCM depletion. Tukey boxplots represent the quantitation of n=120 pre-mitotic cells and their daughter cells monitored from 24 h to 72 h post-transfection, *p<0.05, **p<0.005, Mann-Whitney test. Live-cell quantitation of (e) mitotic outcomes and (f) mitotic duration in U-2 OS and HeLa cells with or without FANCM depletion. Tukey boxplots represent the quantitation of n=120 pre-mitotic cells from 3 experiments, *p<0.05, **p<0.005, Mann-Whitney test. \hat{ } indicates two data-points outside axis. In c-f, for each dataset, the number of cells analyzed are indicated.

FIG. 9. FANCM gene dependency scores across a panel of 517 cancer cell lines from Project Achilles. Gene dependency scores (CERES) for FANCM, determined by CRISPR-Cas9 knockout screens. The dependency score indicates the likelihood that FANCM is essential in the cell line, with a dependency score of 0 indicating that FANCM is not essential for cell viability, and a score of −1 indicating that it is essential. Histogram of FANCM dependency scores across all 517 cancer cell lines (top panel). FANCM dependency scores for each individual cell line, grouped by disease type (bottom panel). Known ALT cell lines have been colored.

FIG. 10. Characterization of the MM2-ER fusion protein. (a) Schematic showing competitive inhibition of FANCM-BTR complex formation by the MM2 peptide. (b) Flag-FANCM immunoprecipitation of the FANCM-BTR complex (TOP3A binding) diminishes with increasing concentrations (1-50 μM) of MM2-WT, but not with MM2-FF>AA mutant peptide in HEK-293 cells expressing Flag-FANCM. (c) Representative mitotic spreads showing sister-chromatid exchanges (SCEs) in U-2 OS cells expressing MM2-ER or FF>AA-ER mutant fusion proteins in the presence or absence of 40HT (left panel). Quantitation of SCEs (right panel). (d) Representative mitotic spreads showing sister-chromatid exchanges (SCEs) in HeLa cells expressing MM2-ER or FF>AA-ER mutant fusion proteins in the presence or absence of 40HT (left panel). Quantitation of SCEs (right panel). SCEs are indicated by black arrows. For c and d, scatterplot bars represent the mean±SEM number of SCEs from 15-30 mitotic spreads, *p<0.05, **p<0.005, Mann-Whitney test. NB: The frequency of double labelled mitoses in MM2-ER+4OHT samples was approximately 10% of that in the controls, likely due to significant cell cycle delays introduced by activation of the MM2-inhibitor. As thus, SCE levels are likely underestimated in this experiment.

FIG. 11. Inhibition of the FANCM-BTR complex results in loss of ALT cell viability

(a) Schematic of MM2-tamoxifen (40HT)-inducible ER fusion protein-mediated inhibition of the FANCM-BTR complex interaction. (b) Immunoprecipitation confirming the transfer of TOP3A and RMI1 from FANCM to the decoy MM2-ER fusion protein following 40HT activation in U-2 OS cells. Transfer of complex components is not seen with the FF>AA mutant MM2-ER protein (FF>AA-ER). (c) Tukey boxplots of TIFs in U-2 OS cells expressing MM2-ER or FF>AA-ER fusion proteins in the presence or absence of 40HT. Out of 3 experiments, n=150 cells scored per treatment, **p<0.005, Mann-Whitney test. (d) Representative dot blots and quantitation of C-circle assays in U-2 OS cells expressing MM2-ER or FF>AA-ER fusion proteins in the presence or absence of 40HT. C-circles were normalized to non-induced control. Error bars represent mean±SEM from n 3 experiments, *p<0.05, one-sample t-test. (e) Representative colony formation assay of GM847 and HCT116 cells expressing the MM2-ER fusion protein in the presence or absence of 40HT (top panel). Quantitation of the surviving fraction of colonies for ALT (U-2 OS, GM847 and Saos-2) and telomerase-positive (HeLa and HCT116) cell lines expressing MM2-ER or FF>AA-ER fusion proteins (bottom panel). Colony counts were normalized to non-induced controls. Error bars represent mean±SEM from n=3 experiments, **p<0.005, n.s.=non-significant, Student's t-test. (f) Representative dot blots and quantitation of C-circle assays from U-2 OS, GM847, Saos-2, HeLa and HCT116 cells treated with 0.5 μM PIP-199 or vehicle control (DMSO) for 72 h. C-circles were normalized to a reference sample control. Error bars represent mean±SEM from n=3 experiments, *p<0.05, n.s.=non-significant, Student's t-test. (g) Quantitation of the surviving fraction of colonies from U-2 OS, GM847, Saos-2, HeLa and HCT116 cells treated with PIP-199. Colony counts were normalized to DMSO controls. \hat{ }Denotes a datapoint (1.42) exceeding the visible axis. Error bars represent mean±SEM from n=3 experiments, *p<0.05, n.s.=non-significant, Student's t-test.

FIG. 12. Dose-dependent inhibition of the FANCM-BTR by PIP-199

(a) Quantitation of the surviving fraction of colonies from U-2 OS, GM847, Saos-2 (ALT), HeLa and HCT116 (telomerase-positive) cells treated with PIP-199 after 11 days. Colony counts were normalized to DMSO controls. Error bars represent mean±SEM from n=3 experiments, *p<0.05, n.s.=non-significant, Student's t-test. (b) Co-immunoprecipitation of BLM and RMI1 in U-2 OS cells confirming the disruption of FANCM-BLM and FANCM-RMI1 interaction by increasing concentrations of PIP-199 after 72 hours.

FIG. 13. Schematic of proposed model of FANCM-mediated ALT suppression

FANCM functions to reverse and remodel stalled replication forks that predominate in ALT telomeres. In the absence of FANCM, or through disruption of the FANCM-BTR complex, stalled forks deteriorate into double strand breaks, which provide the substrate for break-induced telomere synthesis events and the concomitant production of nascent ECTRs.

FIG. 14. Example of gating strategy performed for propidium-iodide cell cycle analysis. Pseudo-colored plot of SSC-A, FSC-A subset gating followed by PI-A, PI-W subset gating (left panel). PI-A, PI-W subset was then subject to Dean-Jett cell cycle analysis. Gates for G0/G1, S and G2/M phases are indicated in green, orange and cyan, respectively (middle panel) with associated statistics (right panel) and the curve of best fit indicated in violet (middle panel).

FIG. 15. FANCM supports normal cell cycle progression and proliferation of ALT cells.

(A) Western blot analysis of FANCM protein levels in ALT and Tel+ cells transfected with anti-FANCM siRNAs (siFa and siFb) or with control siRNAs (siCt). ALT cells (grey background) are: U2OS, HuO9, Saos2 and WI-38 VA13 (VA13); Tel+ cells are: HeLa, HOS, HT1080 (HT10) and SKNAS (SK). Proteins were extracted 48 hours after transfection. Lamin B1 (LMB1), Golgin 97 and KAP1 serve as loading controls. (B) Examples of FACS profiles of the indicated siRNA-transfected cells stained with propidium iodide (PI). Cell counts (y axis) are plotted against PI intensity (x axis). Cells were harvested 48 hours after transfection. (C) Quantifications of experiments as in B. The graph shows the percentage of cells in G1, S and G2/M phases from one representative experiment. (D) Examples of colony formation assays with the indicated siRNA-transfected cells. (E) Quantifications of experiments as in D. The graph shows colony numbers relative to siCt-transfected samples. Bars and error bars are means and SDs from 3 independent experiments. P values were calculated with a two-tailed Student's t-test. *P<0.05, **P<0.005, ***P<0.001. (F) Growth curves of U2OS and HeLa cells transfected with the indicated siRNAs every three days. Cell numbers are expressed relative to siCt-transfected cells. Data points and error bars are means and SDs from three independent experiments. (G) Western blot analysis of U2OS cells infected with retroviruses expressing Flag-tagged TRF1 (FL-TRF1) or with empty vector (ev) control retroviruses. Five days after infections cells were transfected with the indicated siRNAs and harvested 48 hours later. pS33: RPA32 phosphorylated at serine 33, pRPA32: phosphorylated RPA32. LMB1 and Golgin serve as loading controls. (H) Examples of FACS profiles of cells as in G. The graph on the left shows the percentage of cells in G1, S and G2/M phases from one representative experiment. Source data are provided as a Source Data file.

FIG. 16. FANCM suppresses telomeric DNA damage and localizes to telomeres in ALT cells. (A) Quantifications of numbers of TIFs per nucleus in experiments as in A performed on the indicated cell lines. ALT cells are on a grey background. Each dot represents an individual nucleus. A total of at least 196 nuclei from three independent experiments were analyzed for each sample. Bars and error bars are means and SDs. P values were calculated with a Mann-Whitney U test. **P<0.005, ****P<0.0001. (B) Dot-blot hybridization of endogenous FANCM ChIPs in the indicated cell lines using radiolabeled oligonucleotides comprising telomeric G-rich repeats or Alu repeats. A high contrasted image is shown to facilitate visualization of the telomeric signal for Tel+ cells. In: Input, Bd: only beads control, Ip: anti-FANCM immunoprecipitation. (C) Quantifications of experiments as in C. Signals are graphed as the fraction of In found in the corresponding Ip samples, after subtraction of Bd-associated signals. Bars and error bars are means and SDs from 3 independent experiments. (D) Western blot analysis of DNA damage activation in the indicated siRNA-transfected cells. Proteins were extracted 48 hours after transfection. Untransfected cells treated with camptothecin (CPT) were included to control for antibody specificity. pS824: KAP1 phosphorylated at serine 824, pS345: CHK1 phosphorylated at serine 345. Beta Actin and LMB1 serve as loading controls.

FIG. 17. FANCM depletion alters telomeric DNA in interphase U2OS cells. (A) Western blot analysis of FANCM protein levels in U2OS cells transfected and treated with RO-3306 as indicated. Cells were harvested 48 hours after transfection. POLD3, RAD51 and PML levels were also analyzed. Beta Actin and LMB1 serve as loading controls. (B) Quantifications of FACS profiles of cells as in A stained with PI. The graph shows the percentage of cells in G1, S and G2/M phases from one representative experiment. R: RO-3306. (C)) Quantifications of numbers of telomeric foci per nucleus in experiments as in A. Each dot represents an individual nucleus. A total of at least 300 nuclei from three independent experiments were analyzed for each sample. Bars and error bars are means and SDs. P values were calculated with a Mann-Whitney U test. (D) Area distribution of telomeric foci areas in experiments as in C. 3D images were sum projected and areas of individual nuclear FISH signals were measured using DAPI staining to identify nuclei (not shown). A total of at least 300 nuclei from three independent experiments were analyzed for each sample. Areas of telomeric foci (in pixels) are binned into 25 intervals of 5-pixel width (x axis; numbers indicate bin centers) and plotted against frequencies (y axis; %). S: small foci (0 to 2.5 pixels), N: normal foci (2.5 to 57.5 pixels), L: large foci (57.6 to 125.5 pixels). The distribution of large foci is represented in the right graph using a smaller y axis scale to facilitate visualization. (E) Quantification of cells with at least 5 Large (L) foci in experiments as in C. P values were calculated with a two-tailed Student's t-test. *P<0.05, **P<0.005, ***P<0.001, ****P<0.0001.

FIG. 18. FANCM depletion increases ALT features.

(A) Quantifications experiments for PML or RAD51 immunostaining combined with telomeric DNA FISH. POLD3 immunostaining combined with RAP1 immunostaining (green); and EdU detection combined with and telomeric DNA FISH. Experiments were performed on U2OS cells transfected with the indicated siRNAs and harvested 48 hours after transfection. Each dot represents an individual nucleus. A total of at least 300 nuclei from three independent experiments were analyzed for each sample. Bars and error bars are means and SDs. P values were calculated with a Mann-Whitney U test. ***P<0.001, ****P<0.0001.

FIG. 19. FANCM restricts telomeric ssDNA and ECTRs in ALT cells. (A) TRF analysis of the indicated ALT (grey backgrounds) and Tel+siRNA-transfected cells. Genomic DNA was prepared 48 hours after transfection, restriction digested and hybridized in gel in native conditions to radiolabeled oligonucleotides comprising 5 telomeric G-rich or C-rich repeats ([TTAGGG]5 and [CCCTAA]5, respectively). After signal acquisition, gels were denatured and re-hybridized to a long radiolabeled telomeric probe (Telo2). The position of the wells and the sizes in kb of a molecular weight marker are indicated on the left of the gels. (B) Dot-blot hybridizations of digested genomic DNA from cells as in A. Control transfections with siRNAs against RNaseH1 (siRH) were also included. Native or denatured DNA was first hybridized to radiolabeled telomeric oligonucleotides. After signal acquisition, membranes were denatured and re-hybridized to radiolabeled Alu repeat oligonucleotides (loading). For quantifications (table below), telomeric signals were normalized through the corresponding Alu signal and expressed relative to siCt-transfected samples. Means and SDs from 3 technical replicates are indicated. Note the accumulation of C-rich ssDNA in FANCM-depleted ALT cells (thick borders). (C) C-circle assay analysis of genomic DNA from the indicated siRNA transfected cells harvested 48 hours after transfection. Products were dot-blotted and hybridized to a radiolabeled Telo2 probe. Control reactions were performed without phi29 polymerase (Φ29). Note that Tel+ cells had no detectable signals. The graph at the bottom shows quantifications of C-circle signals relative to siCt-samples. Bars and error bars are means and SDs from 3 independent experiments. P values were calculated with a two-tailed Student's t-test. **P<0.005, ***P<0.001, ****P<0.0001. (D) 2-D gel electrophoresis of genomic DNA from siRNA-transfected U2OS cells as in A. DNA was denatured and hybridized to a radiolabeled Telo2 probe. Arrowheads point to arches corresponding to circular DNA.

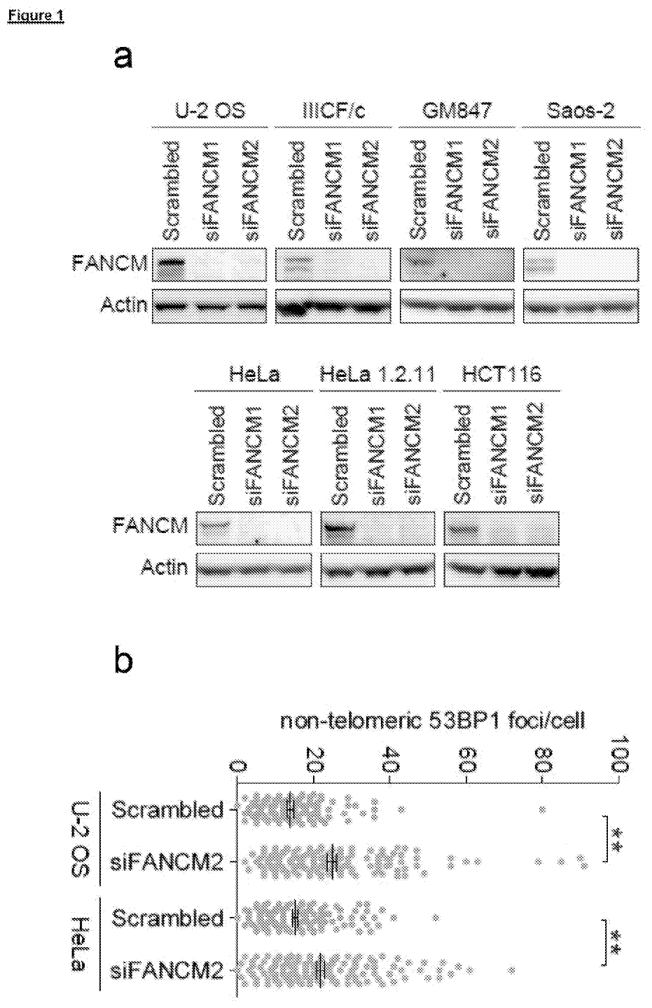

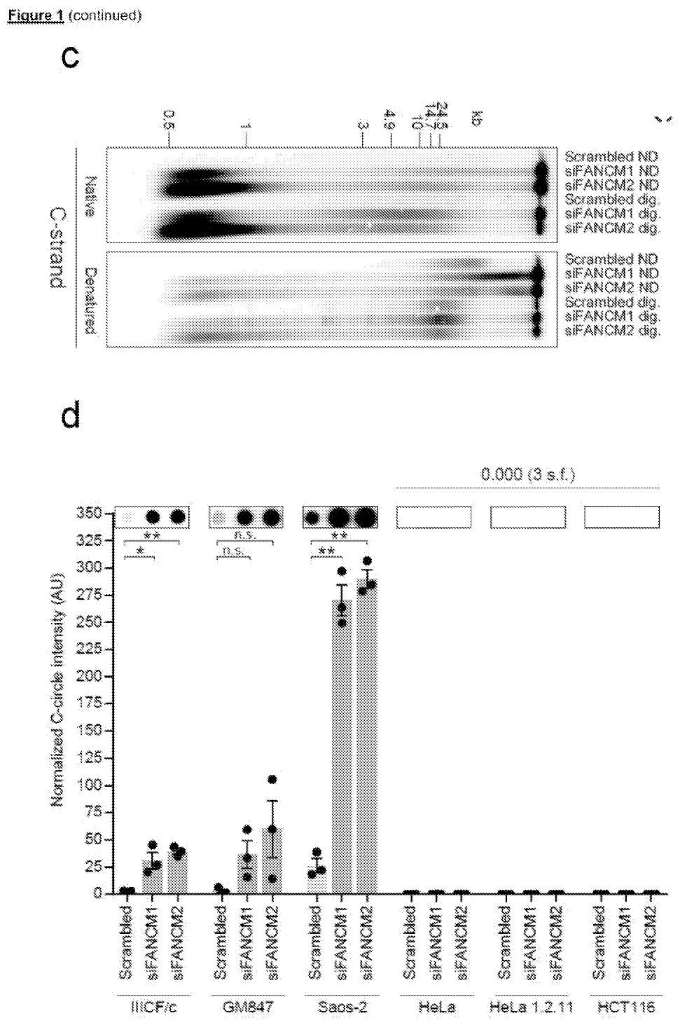

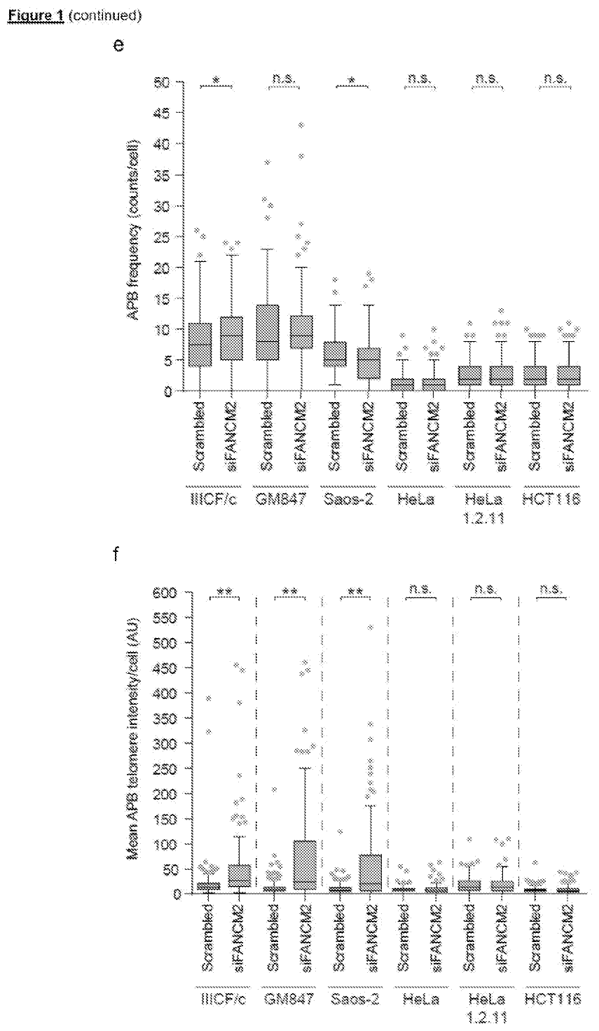

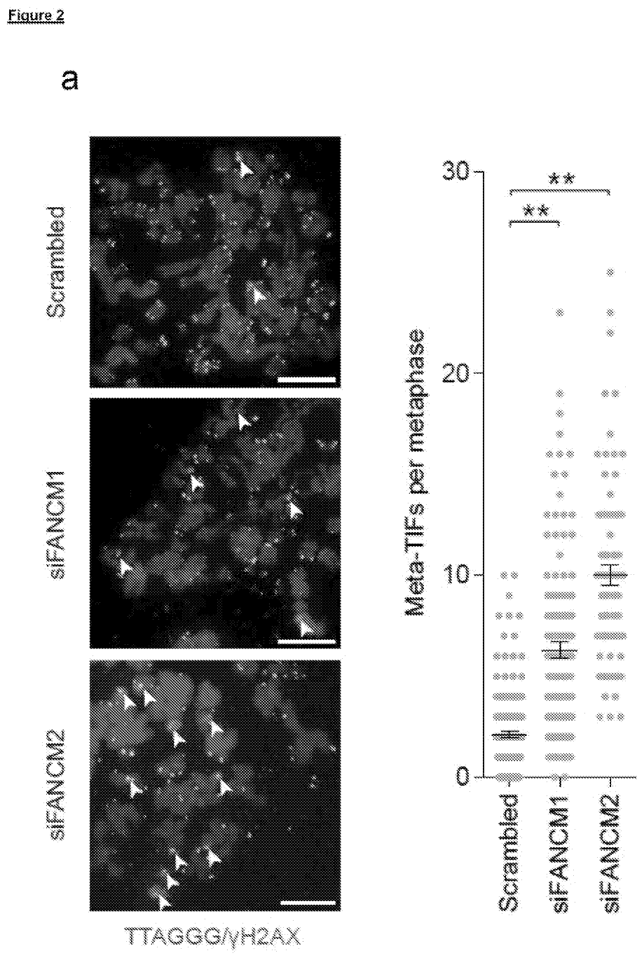

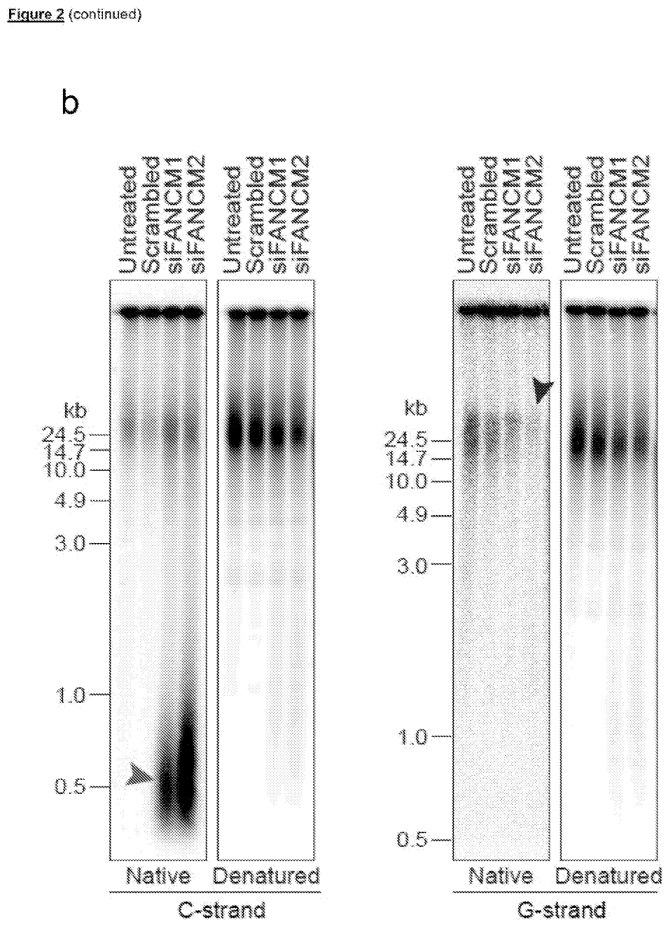

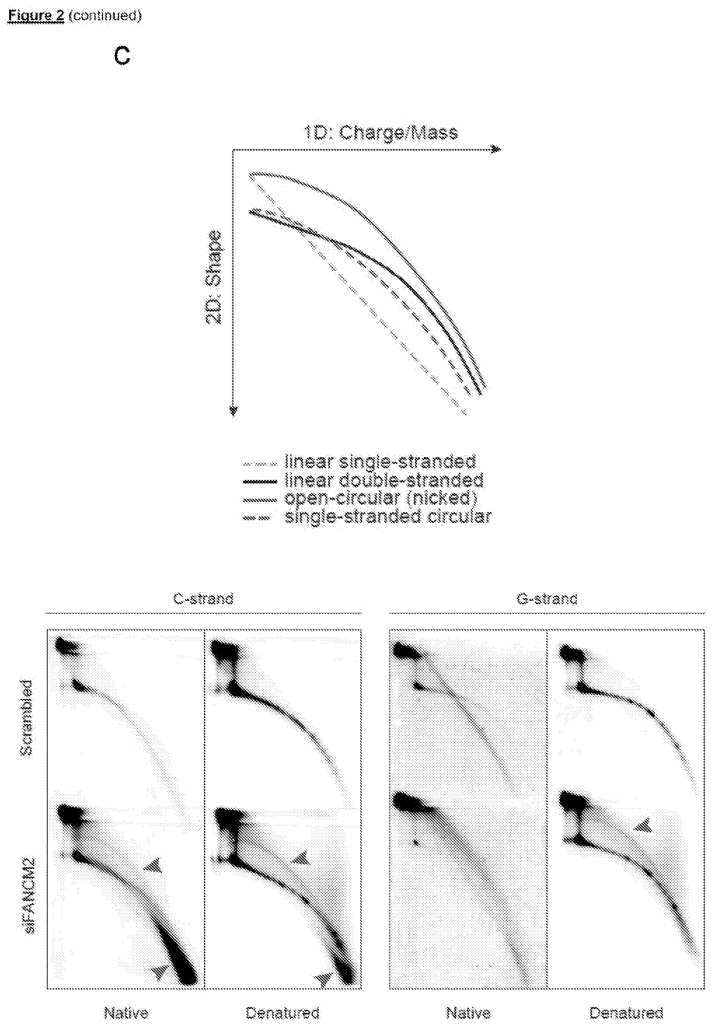

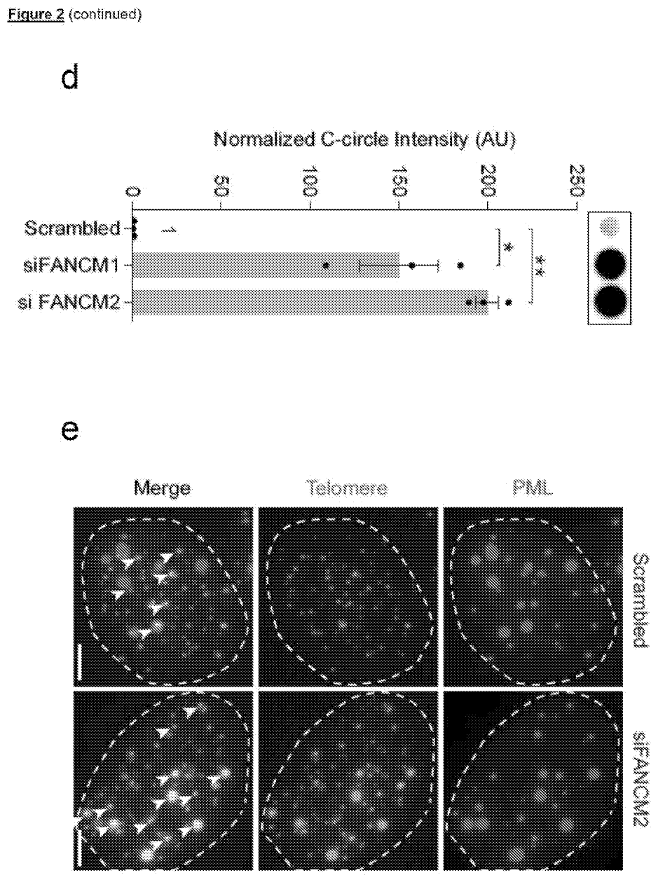

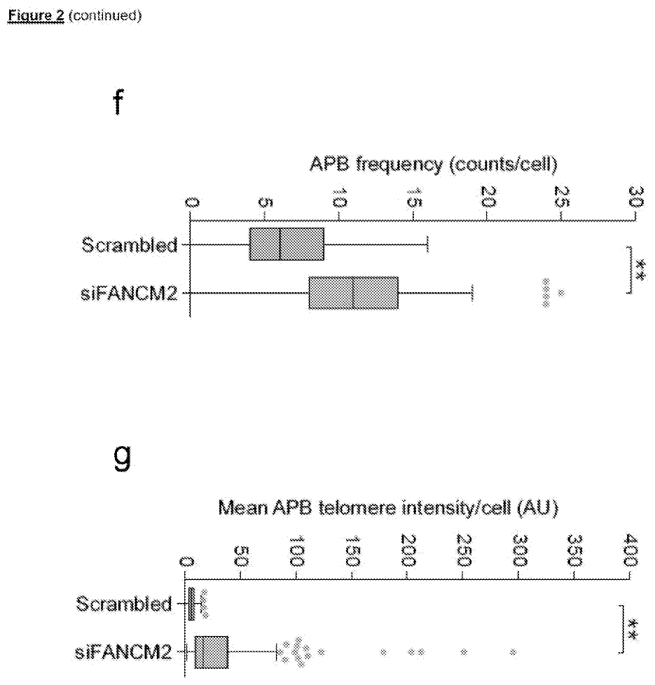

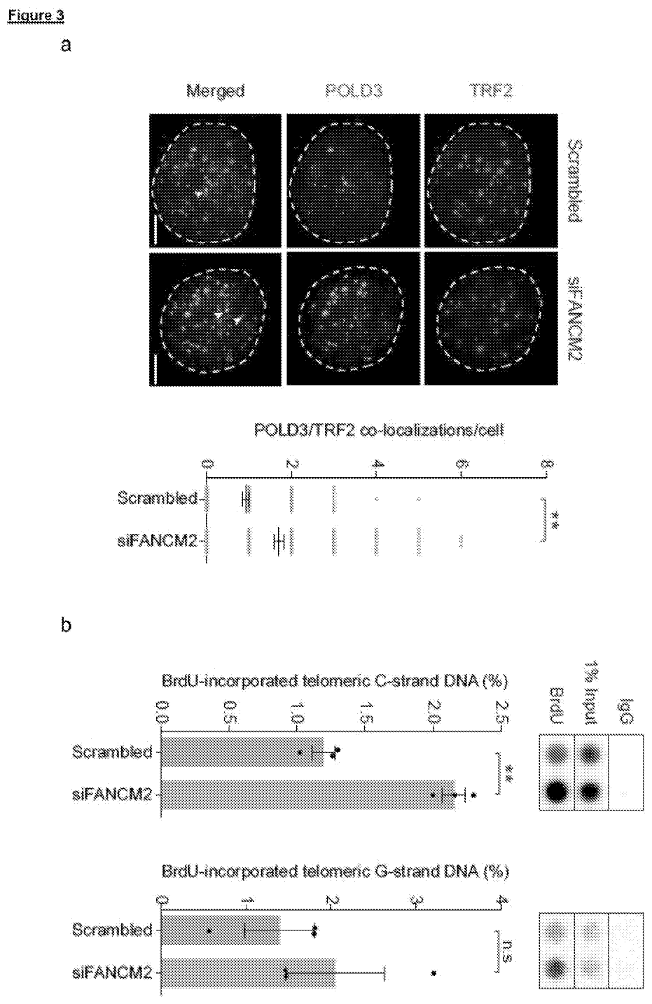

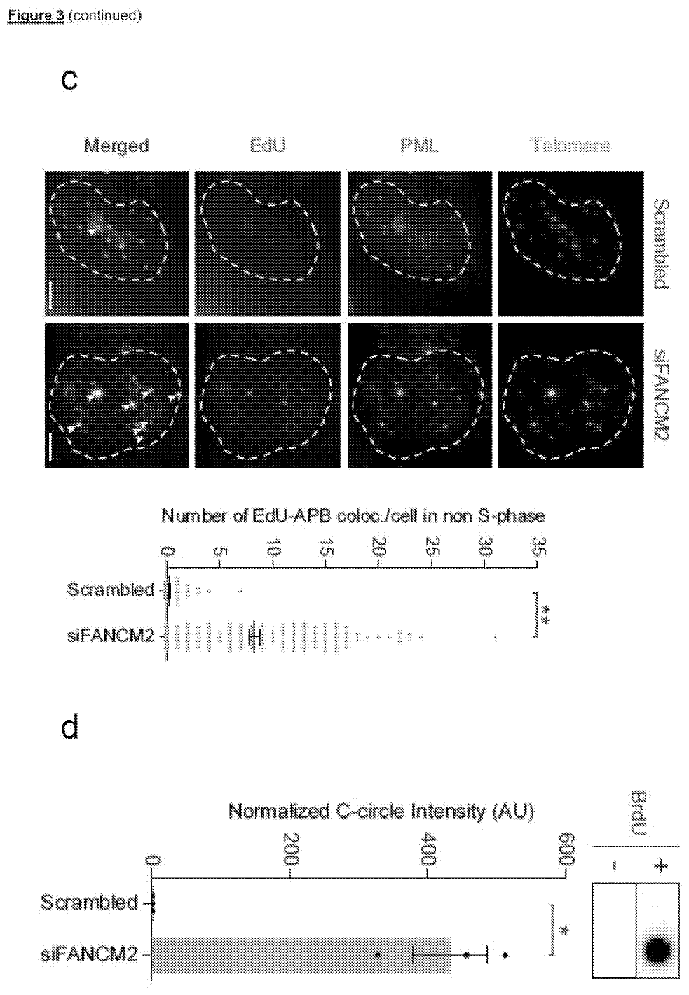

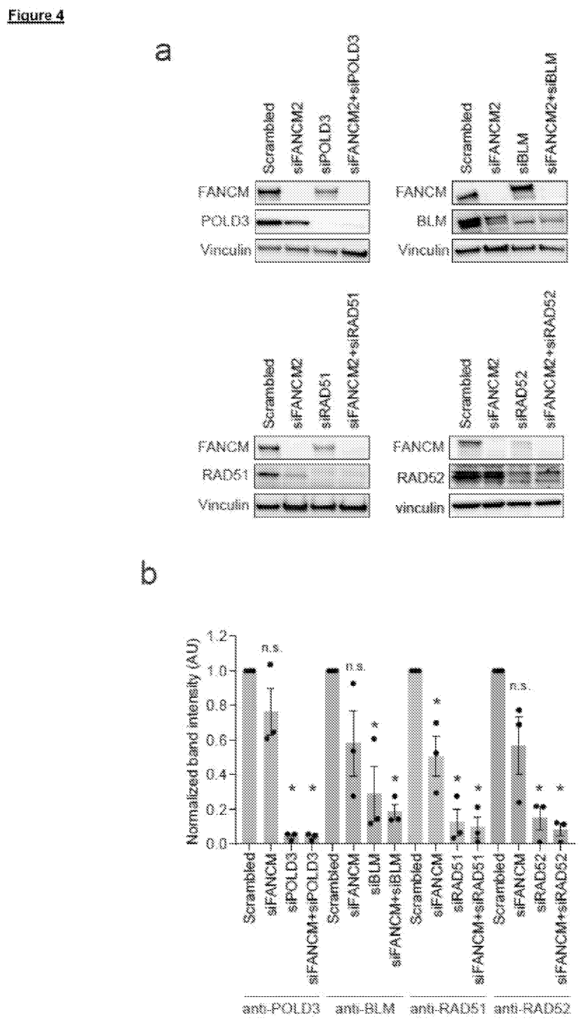

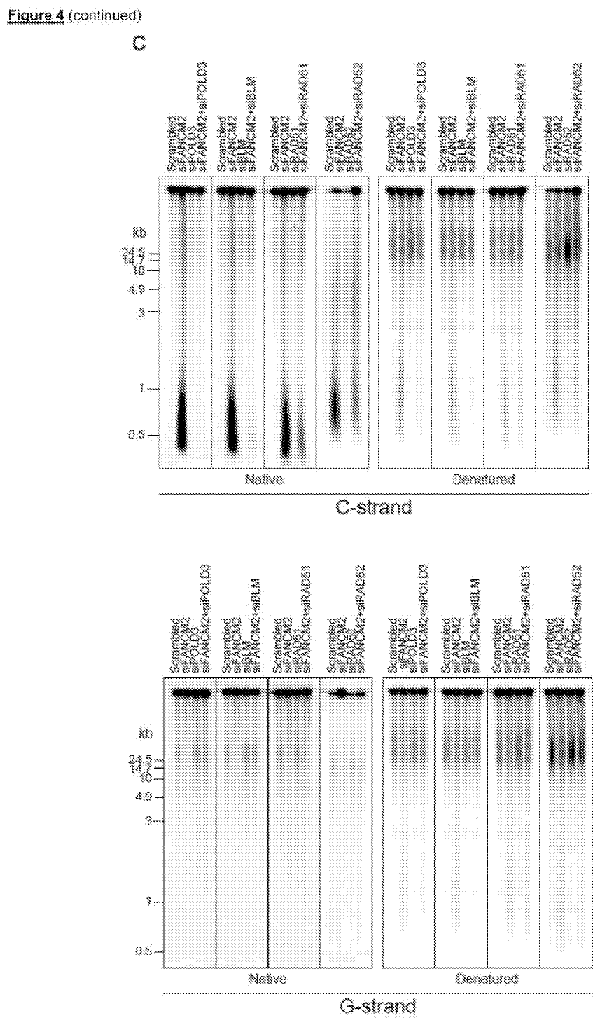

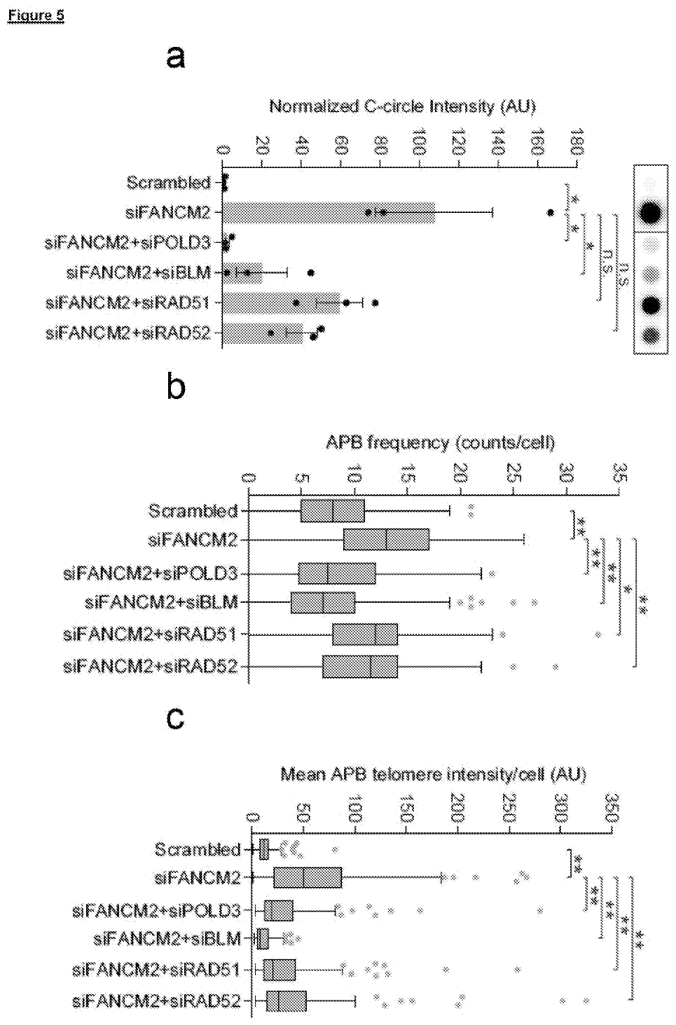

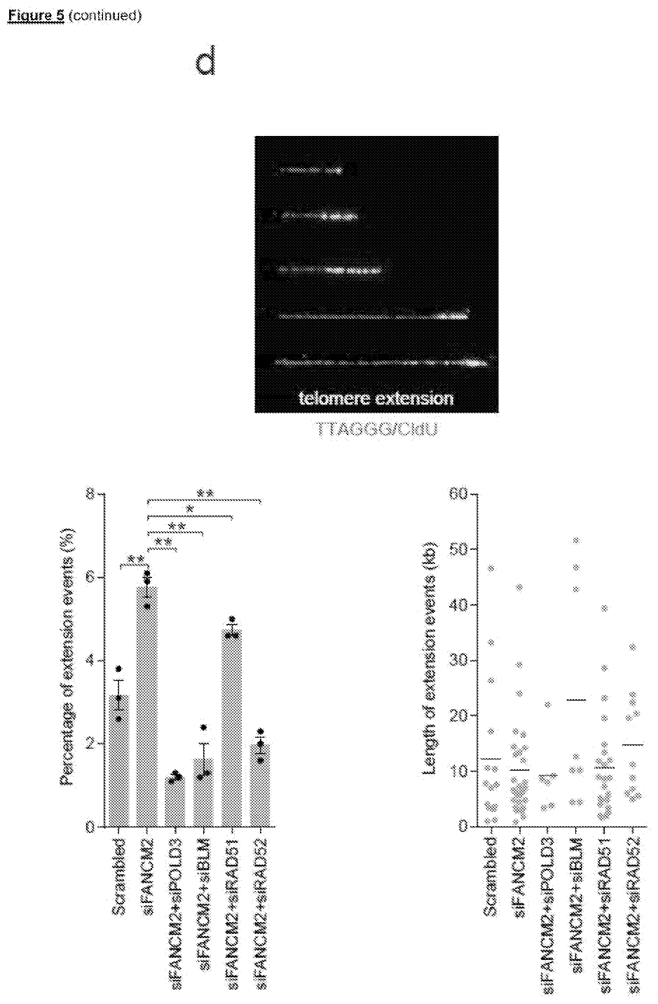

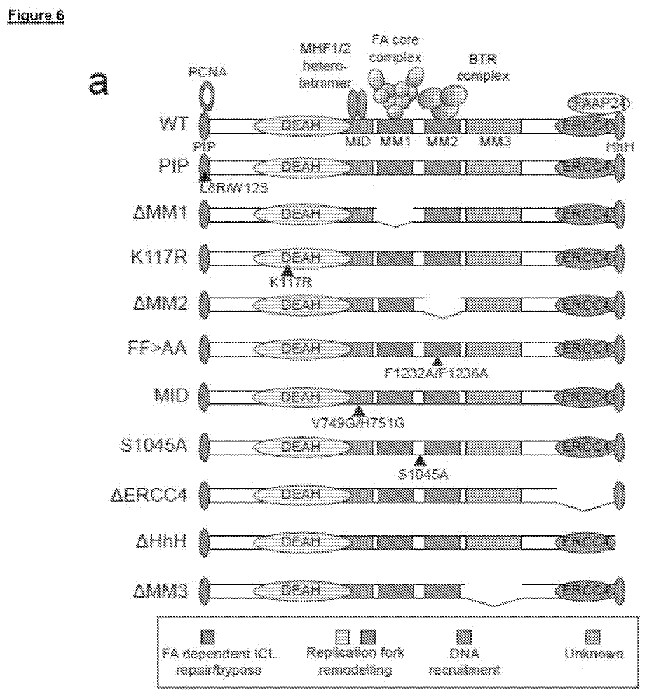

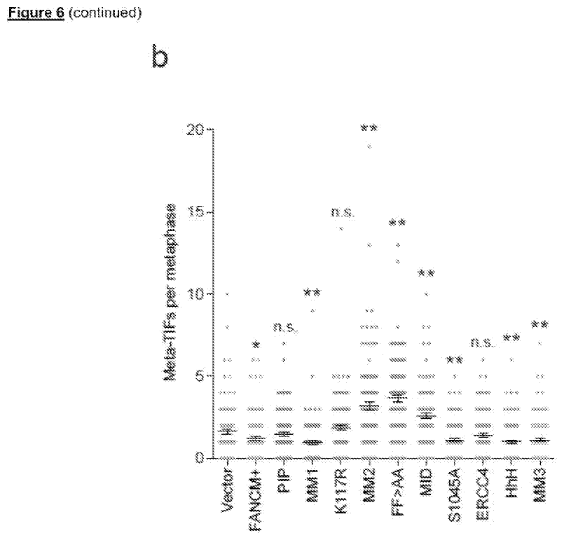

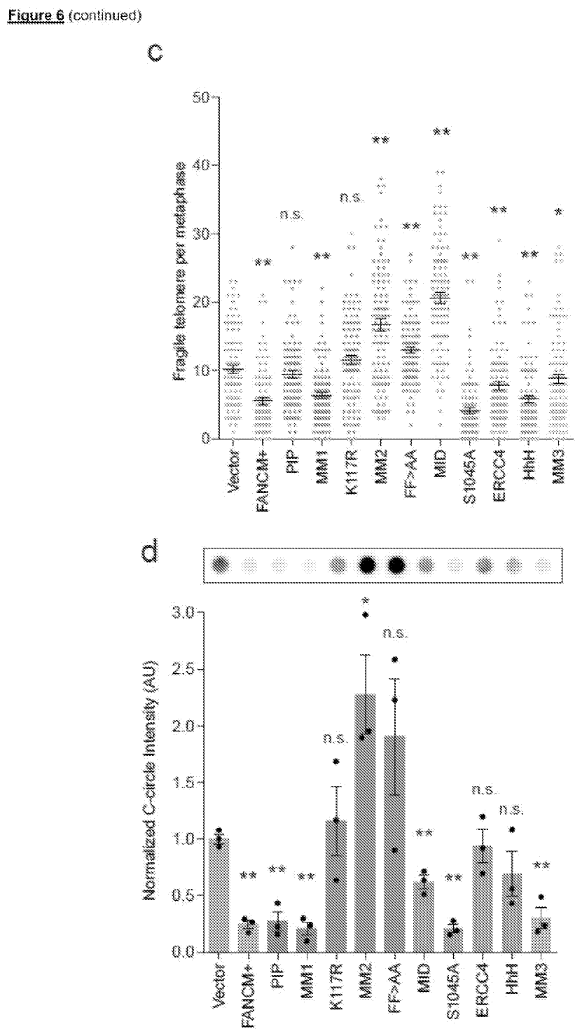

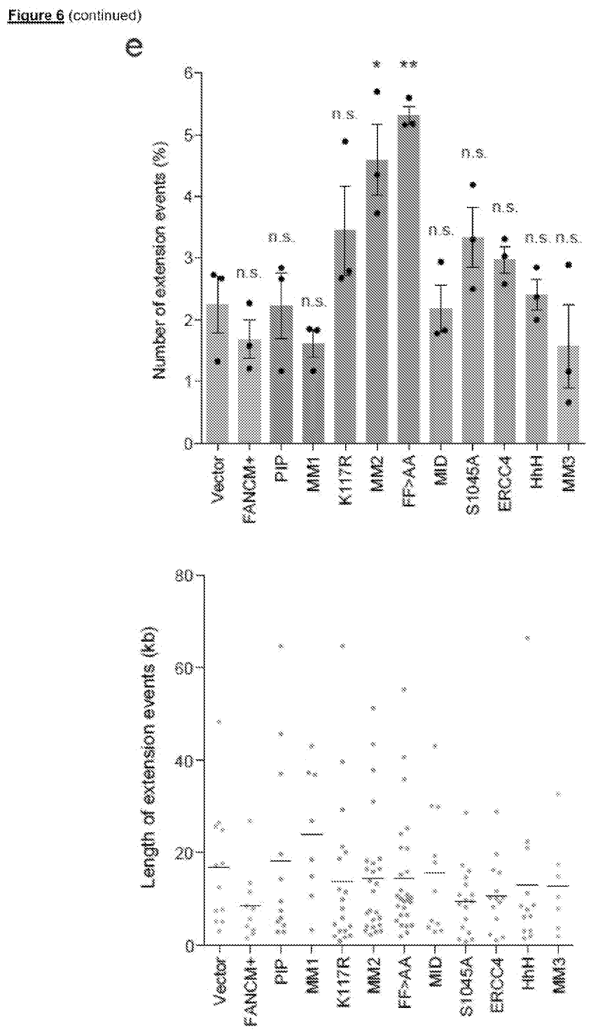

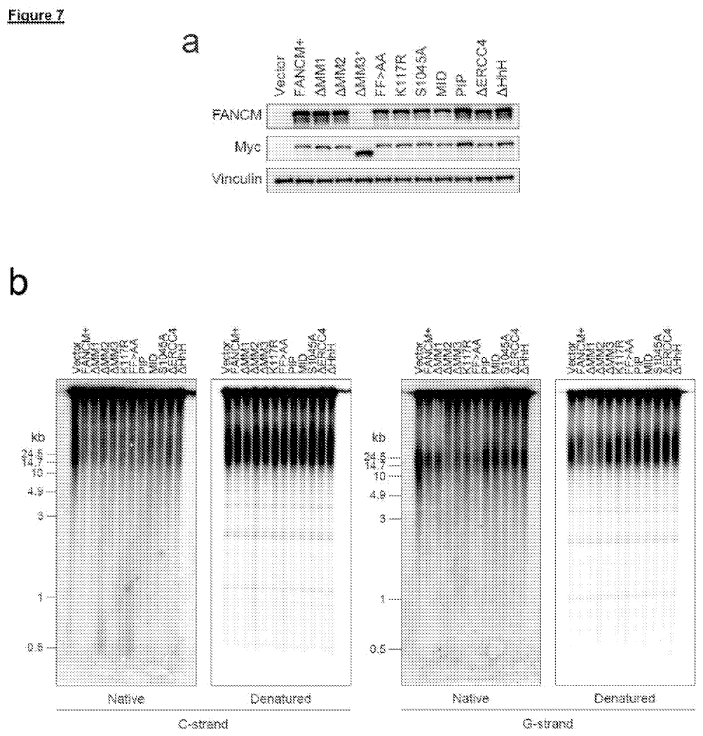

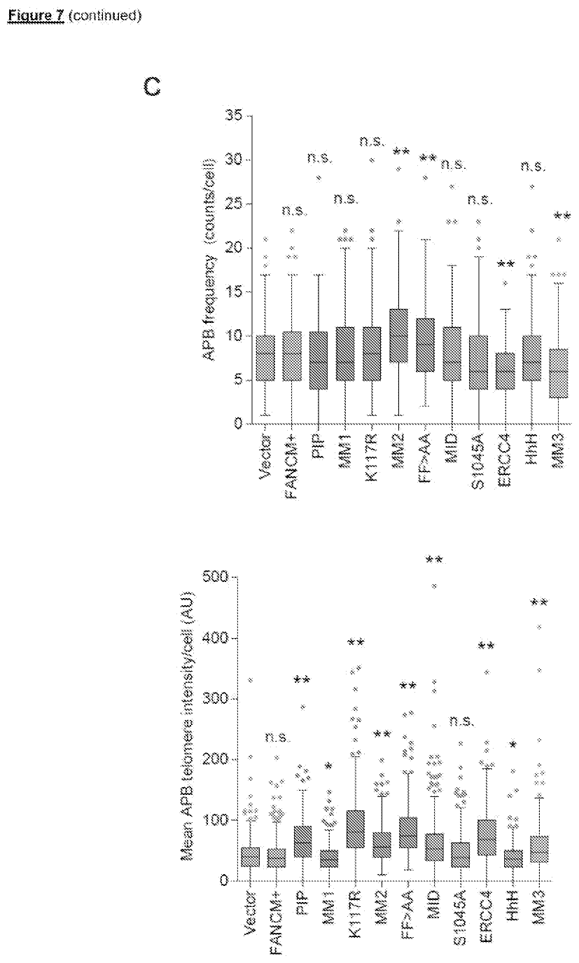

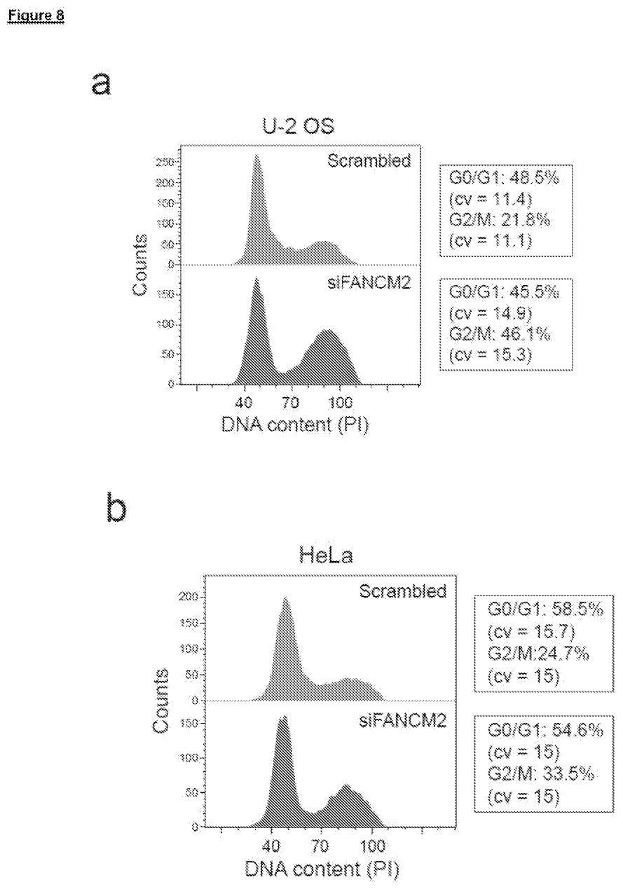

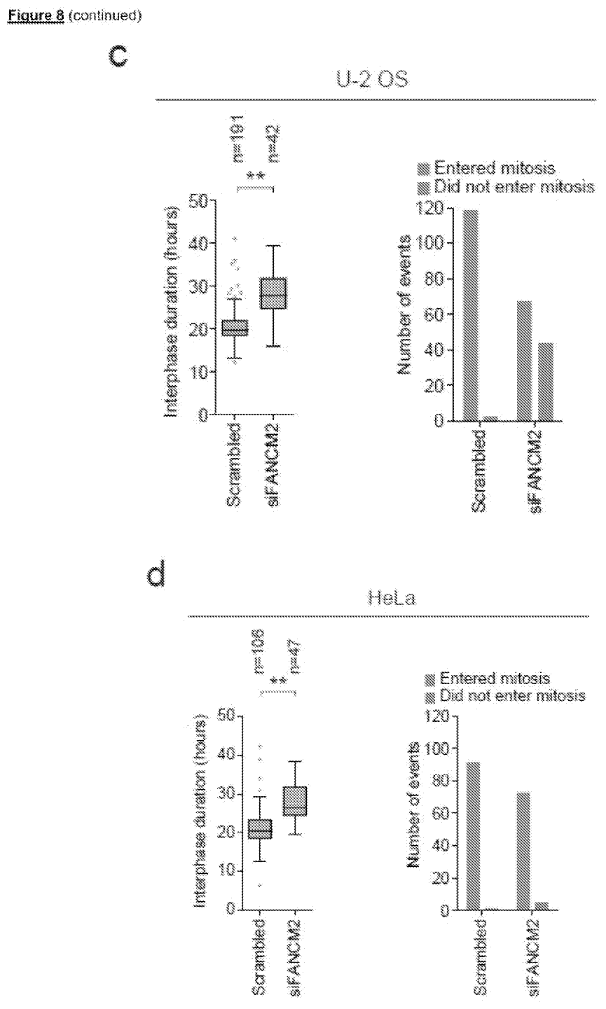

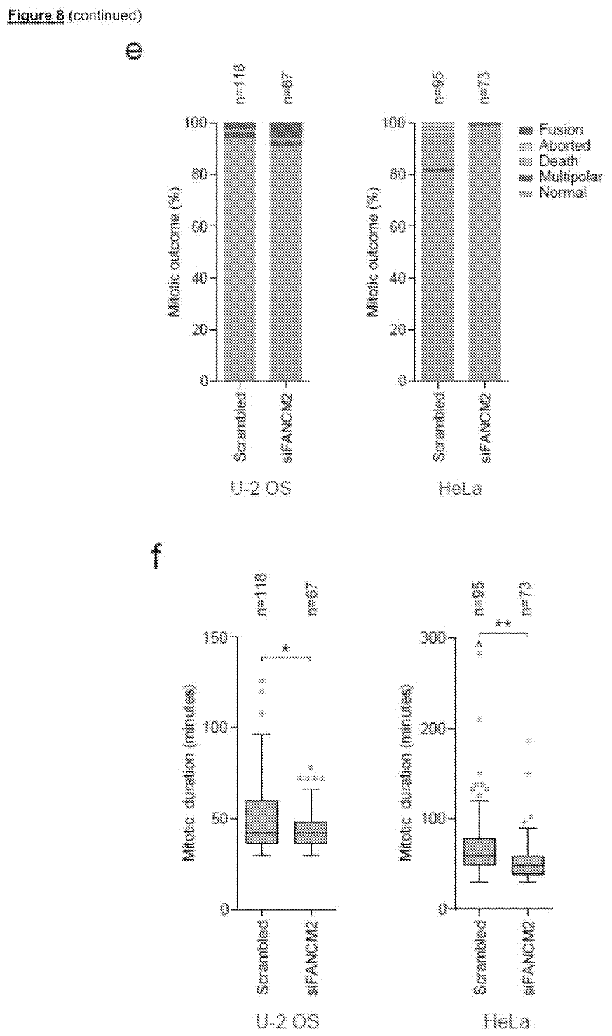

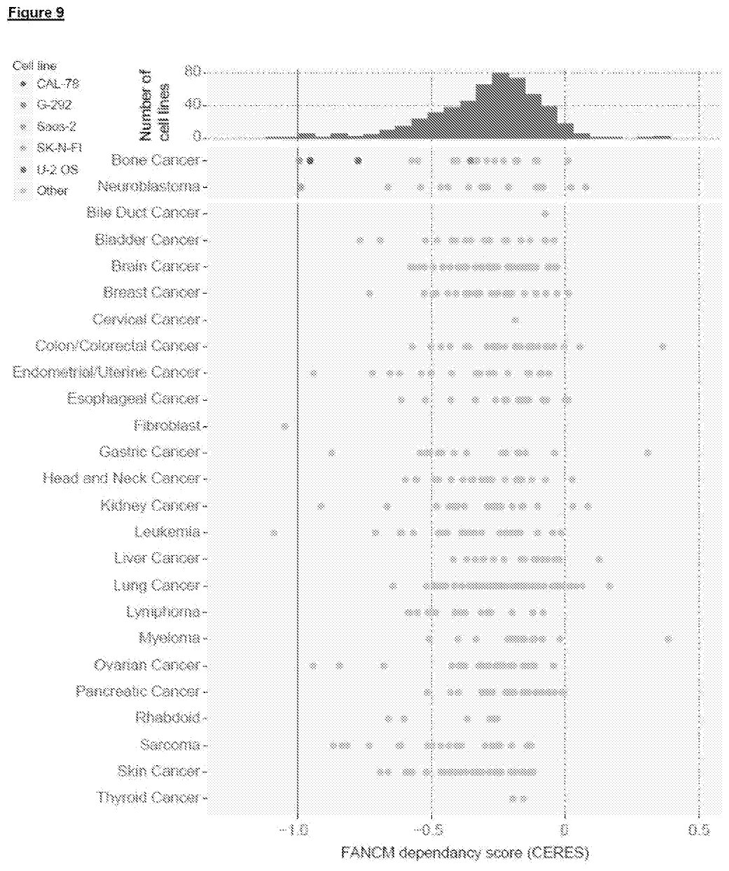

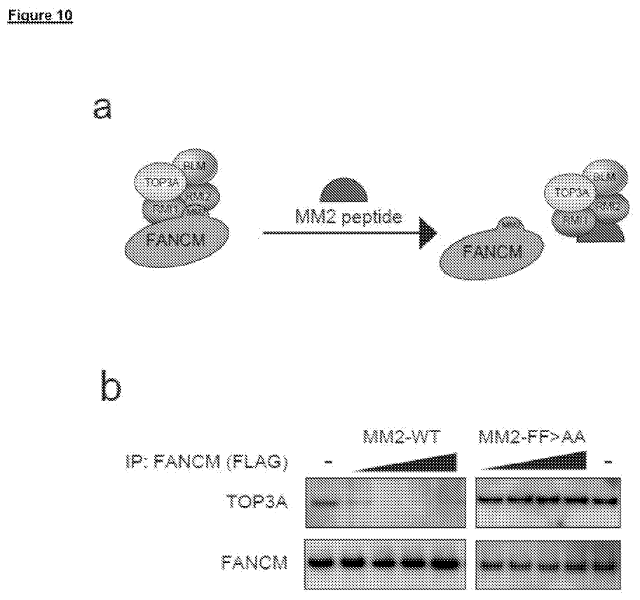

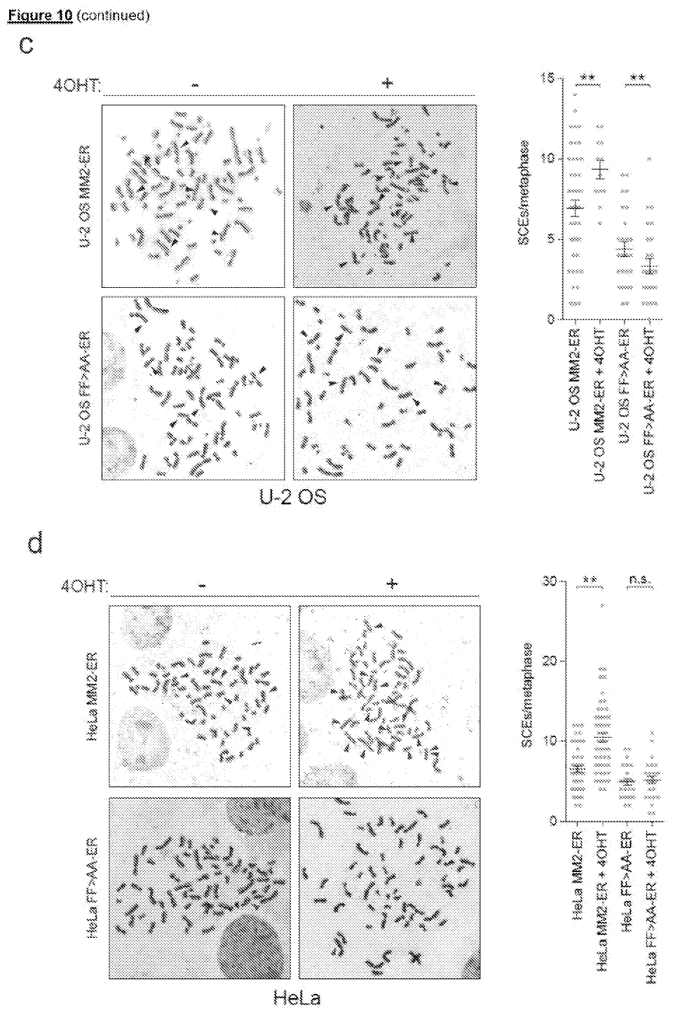

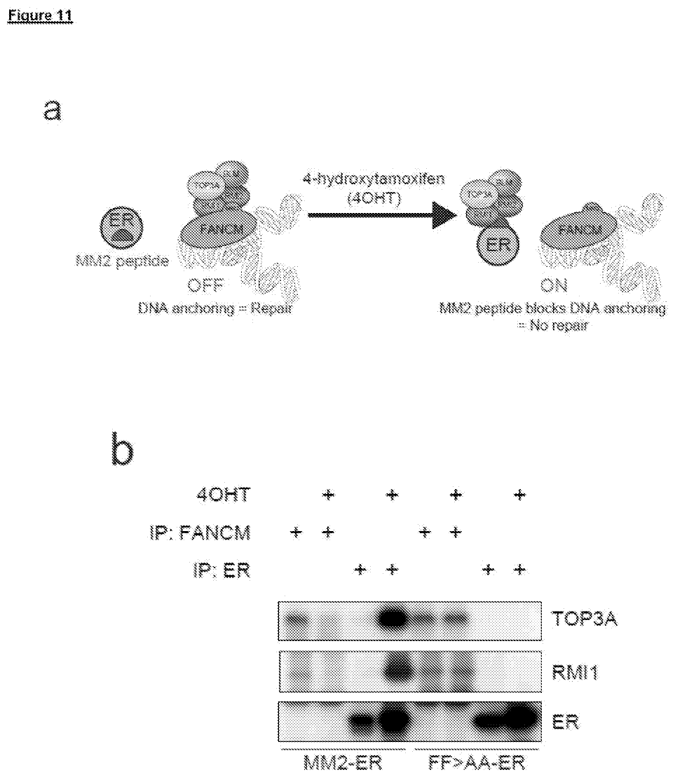

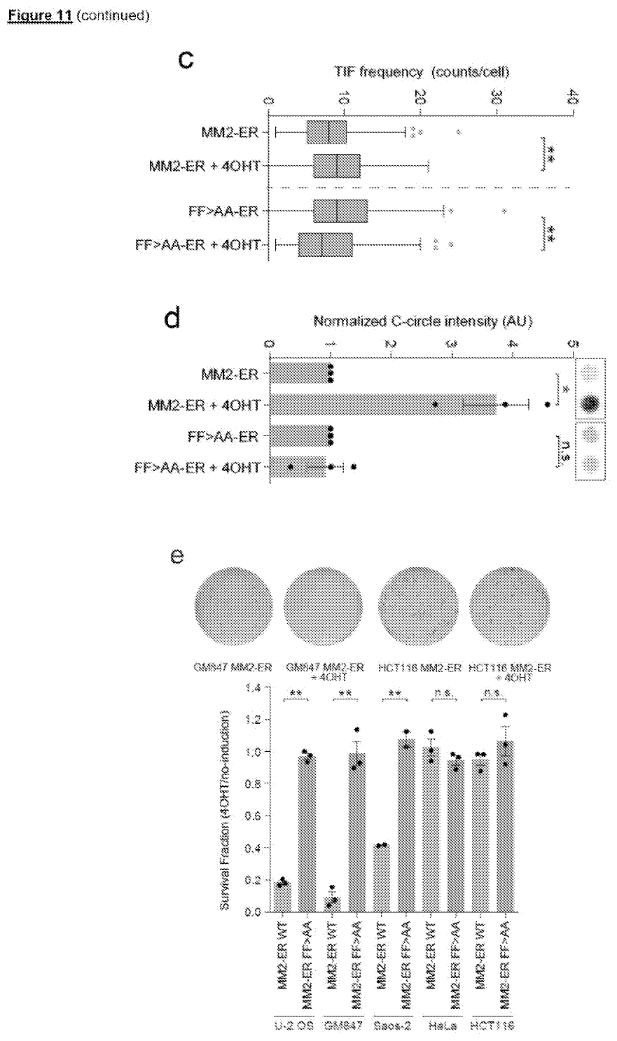

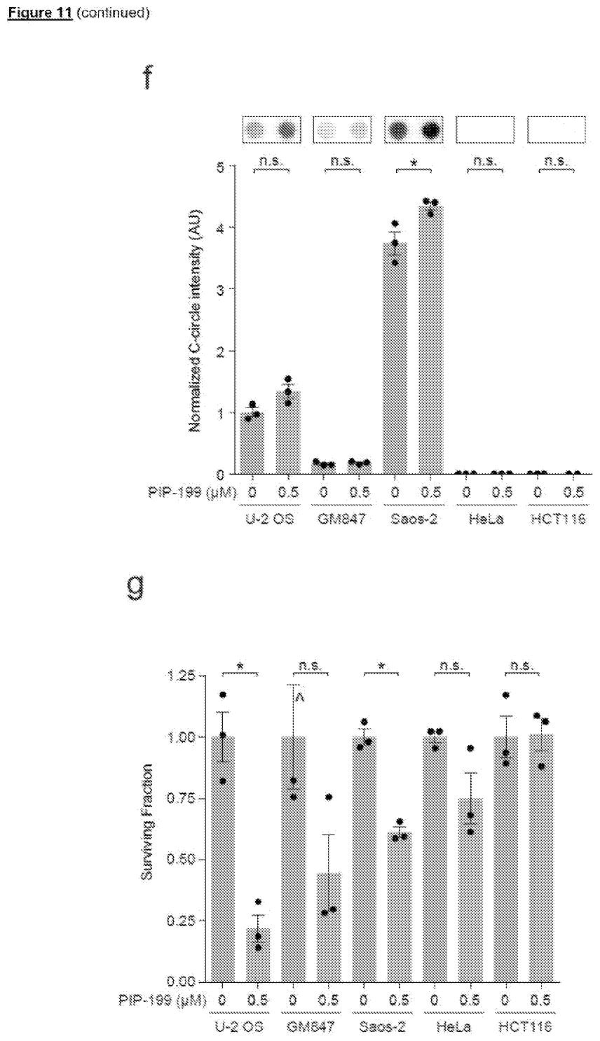

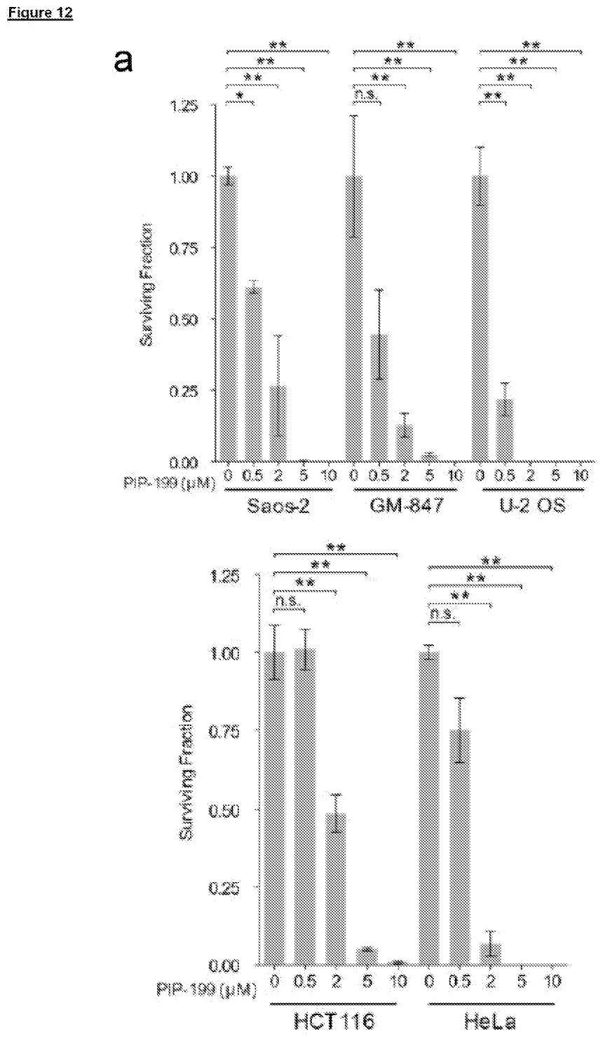

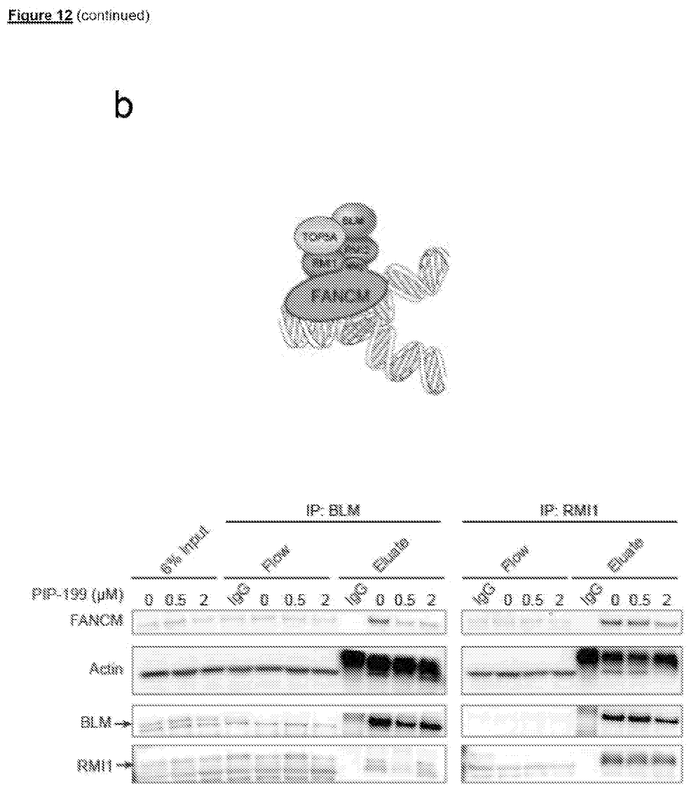

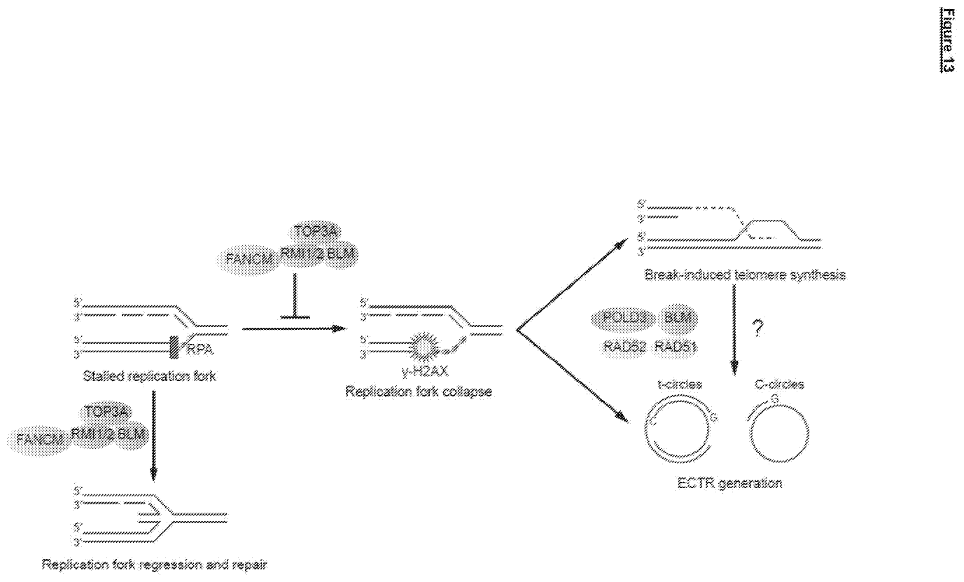

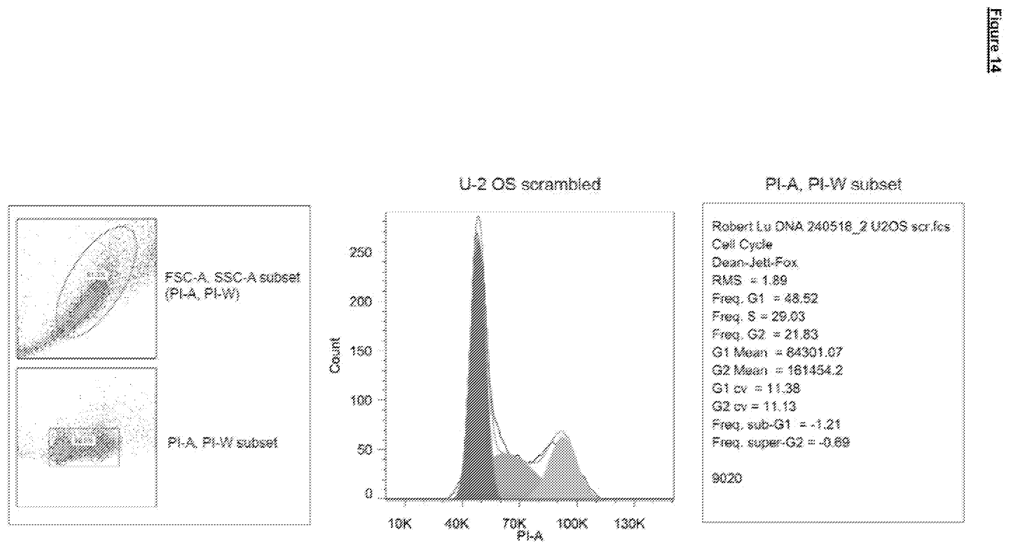

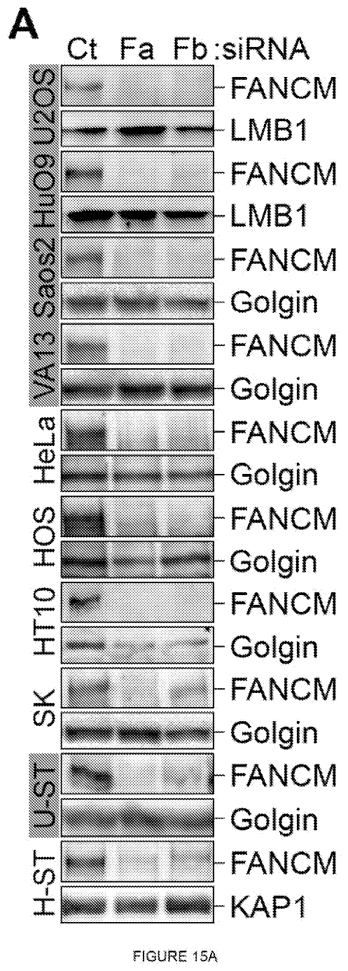

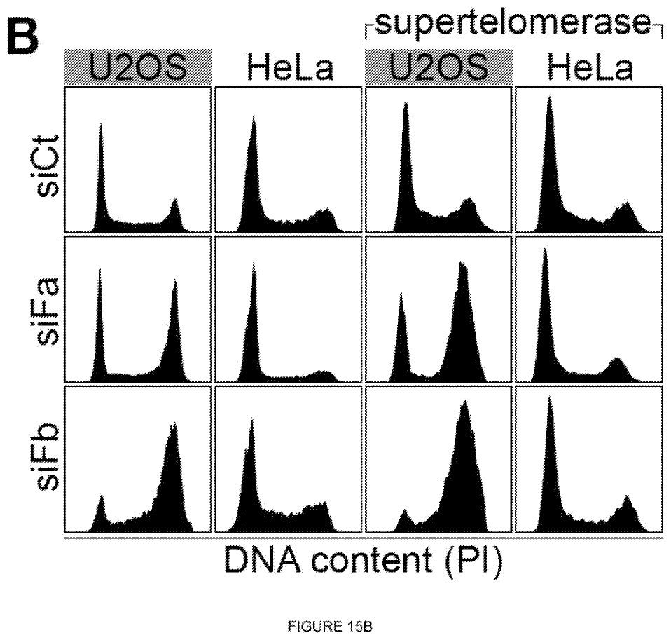

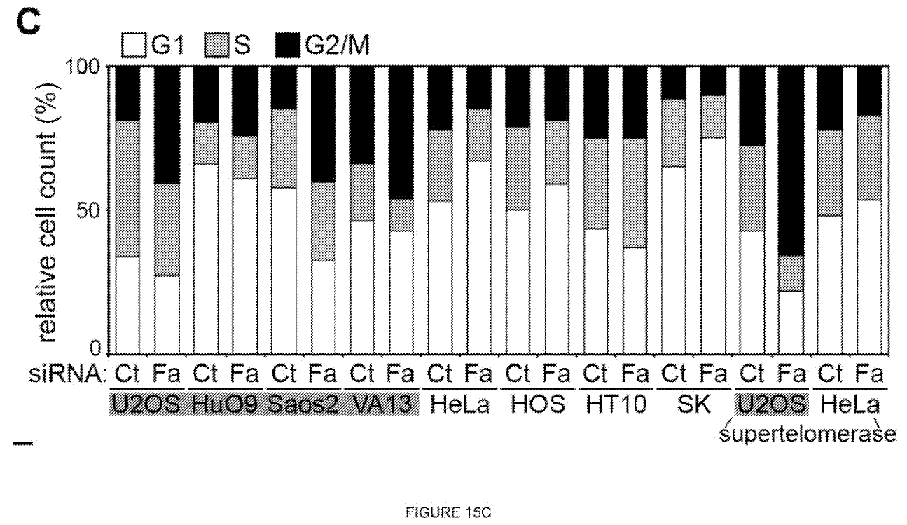

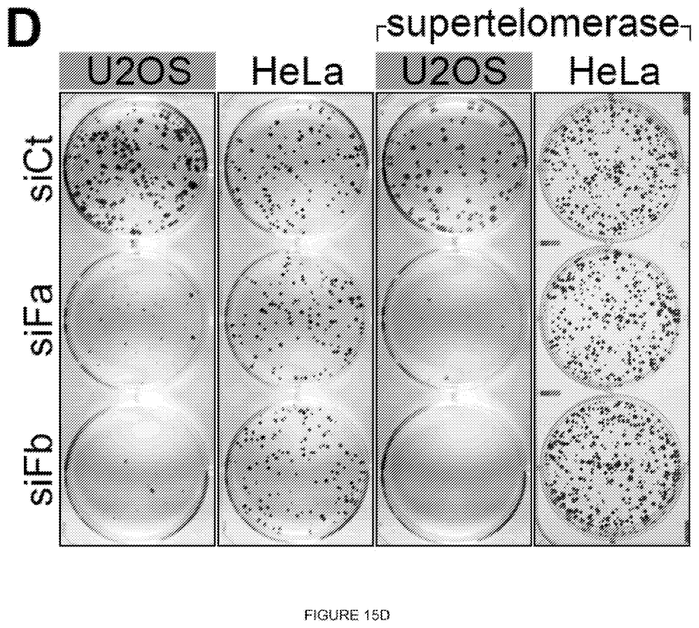

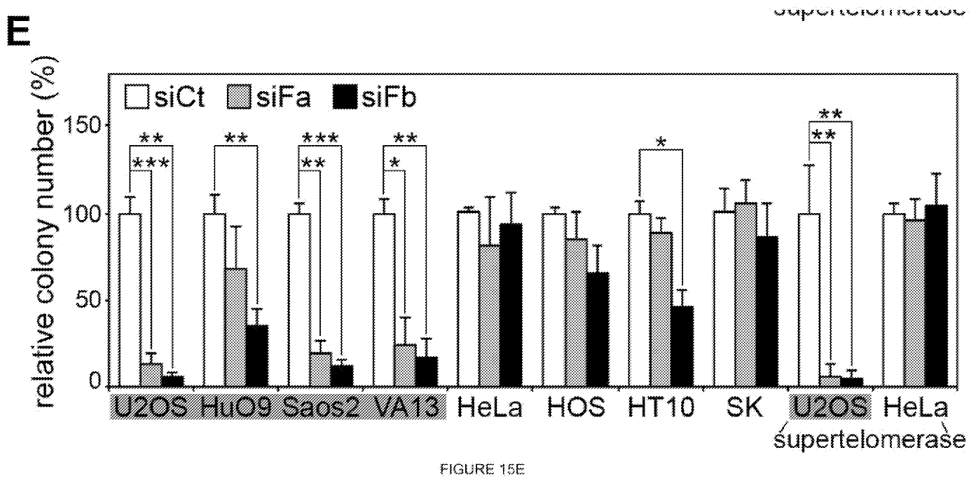

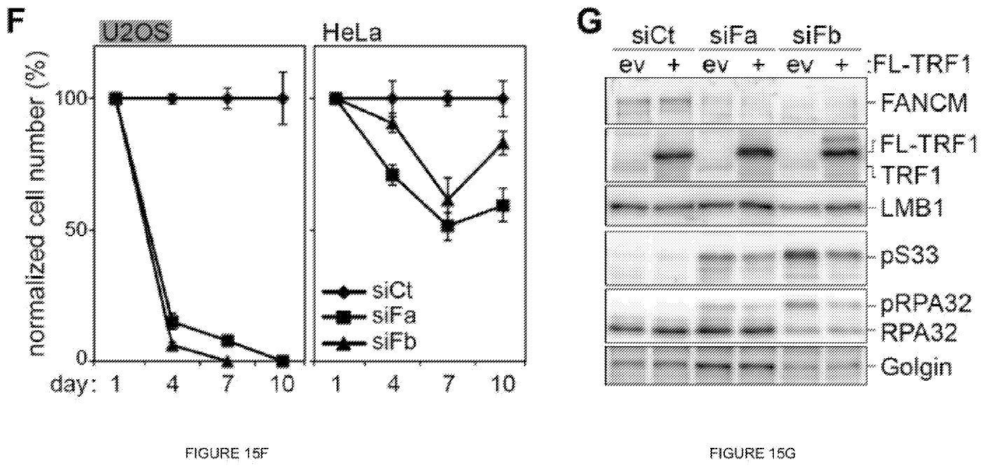

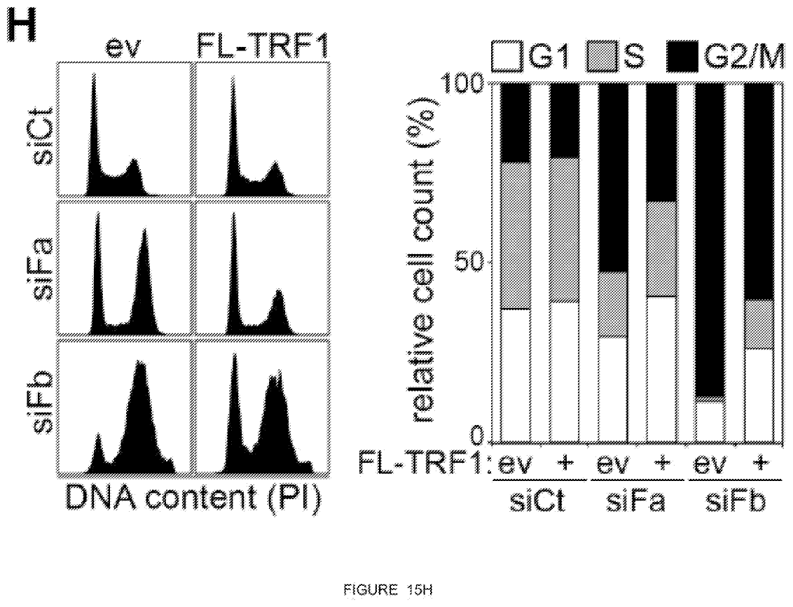

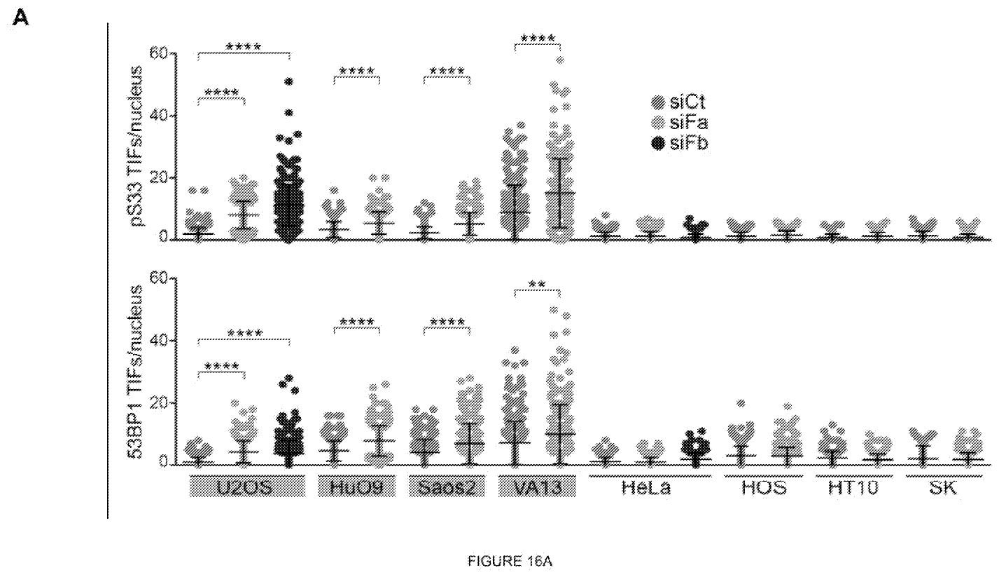

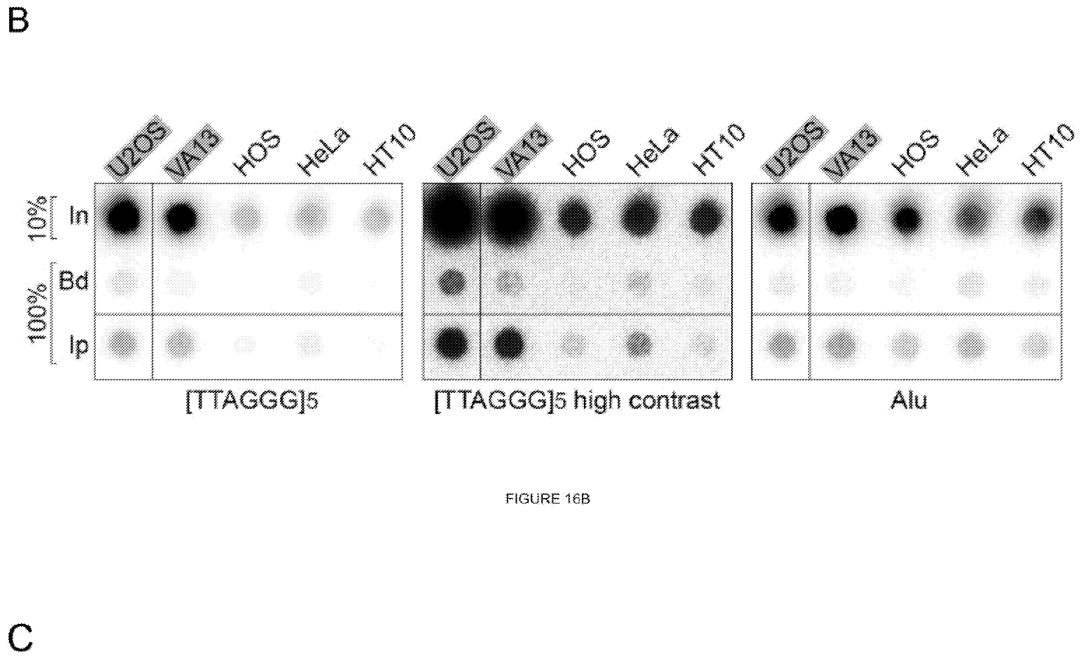

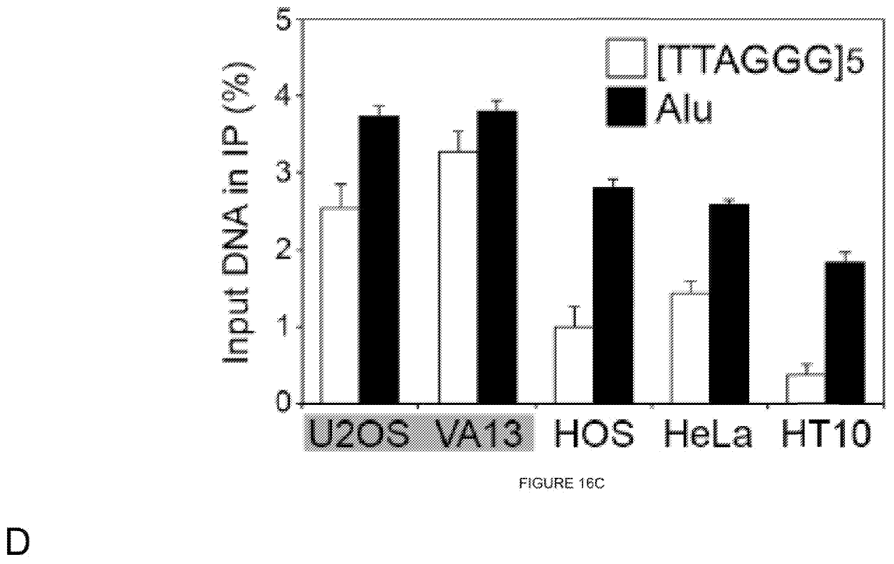

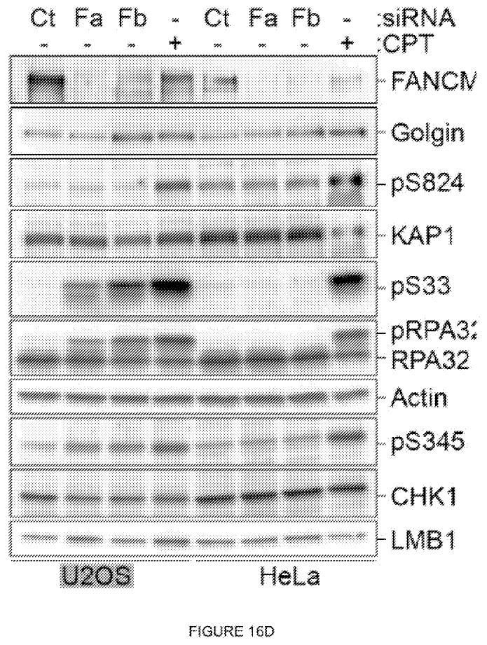

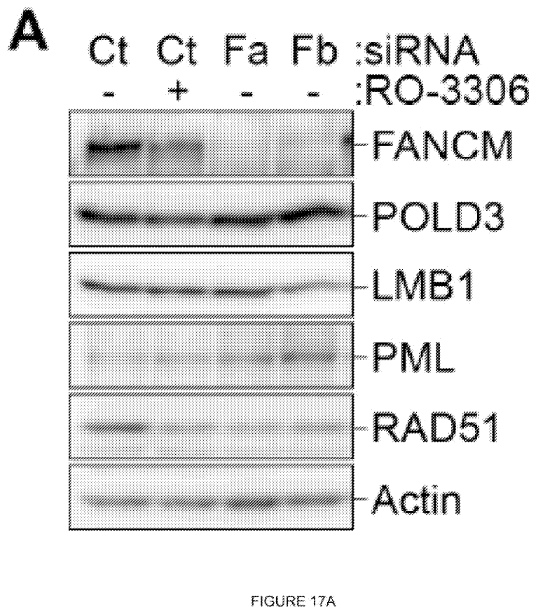

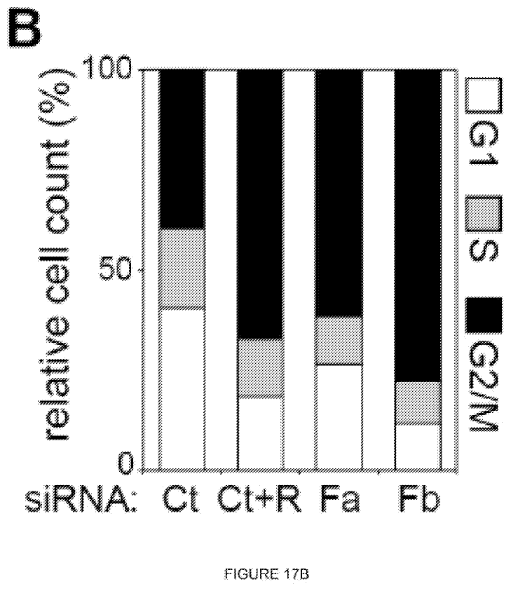

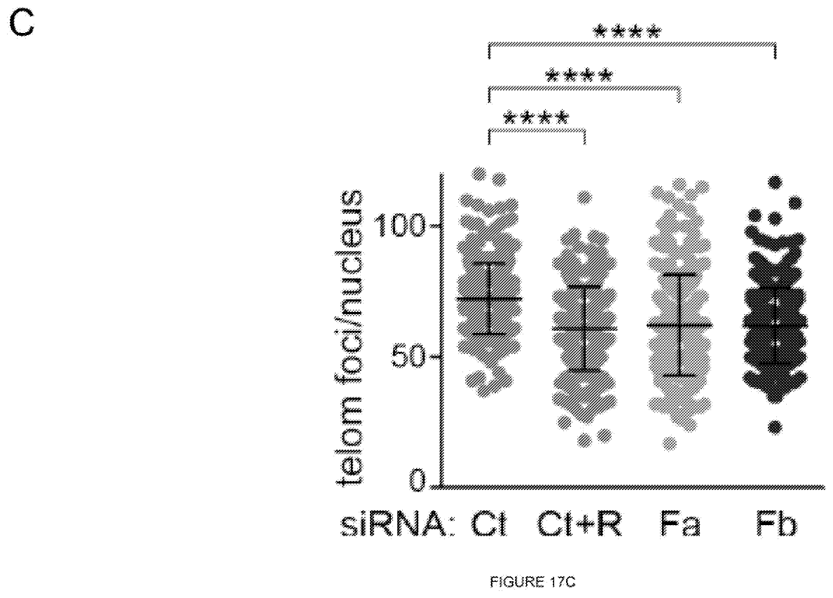

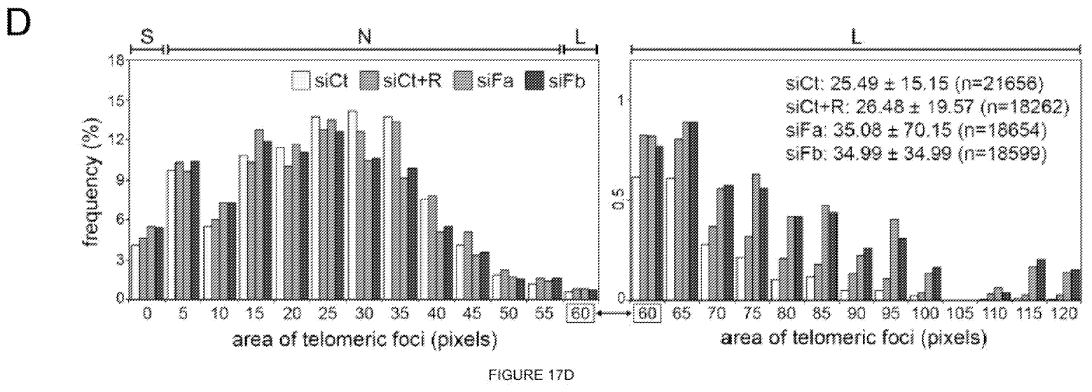

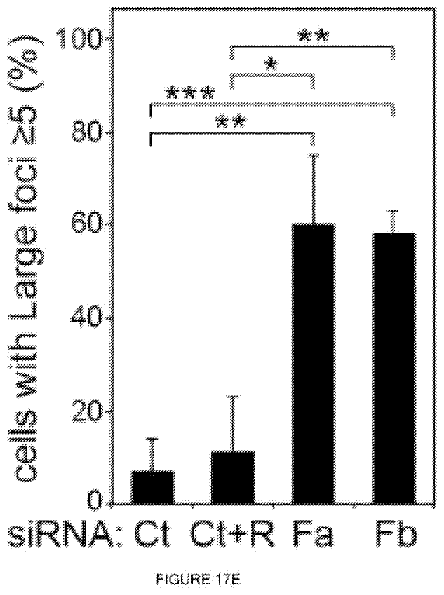

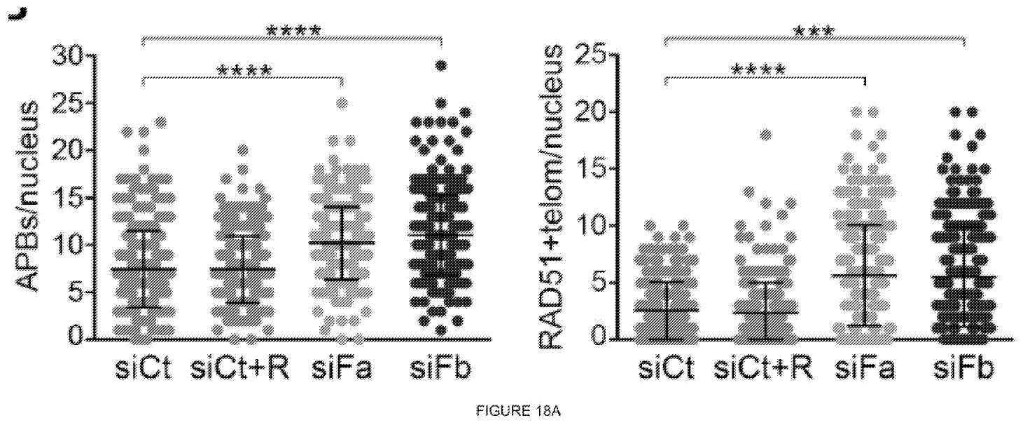

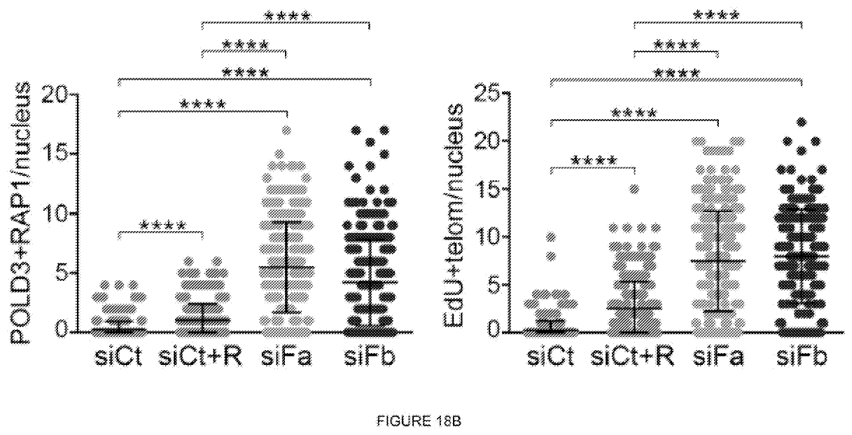

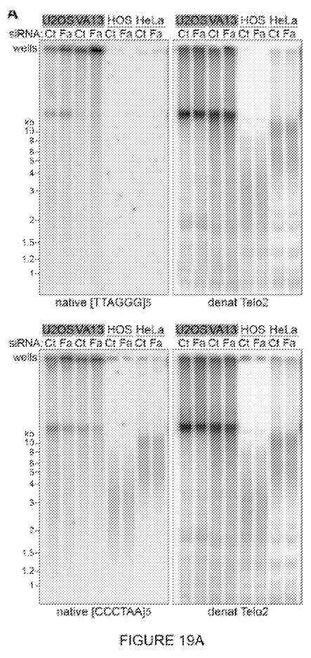

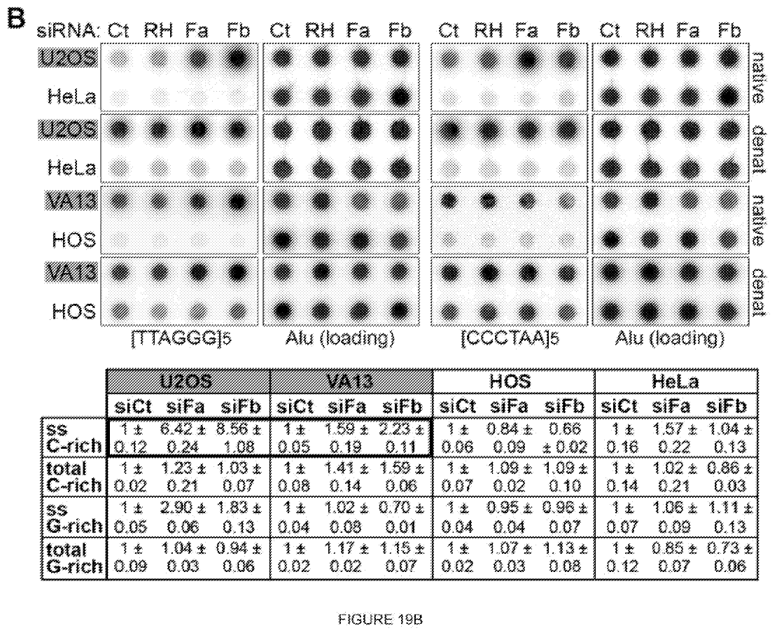

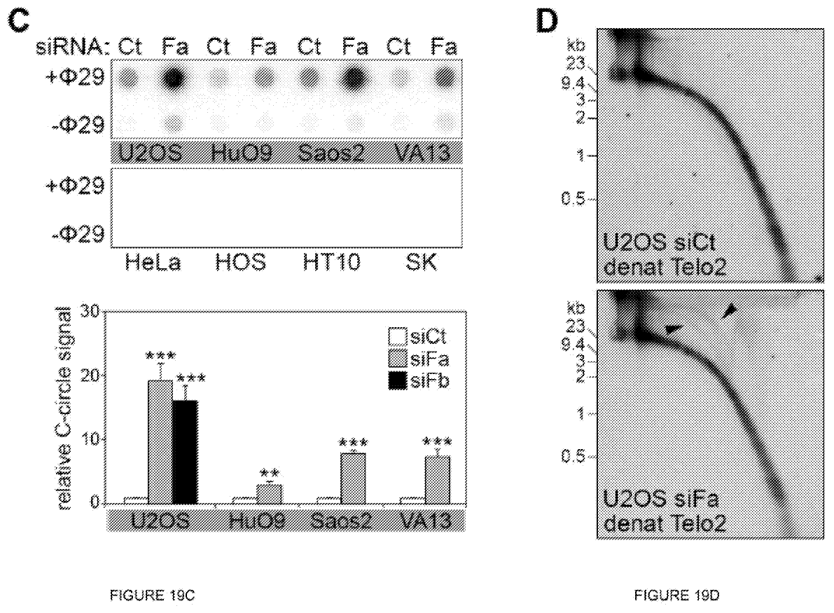

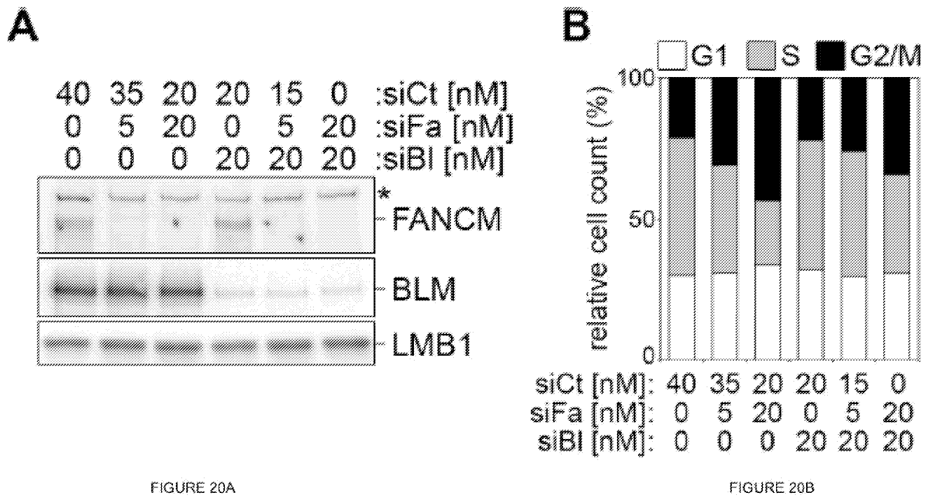

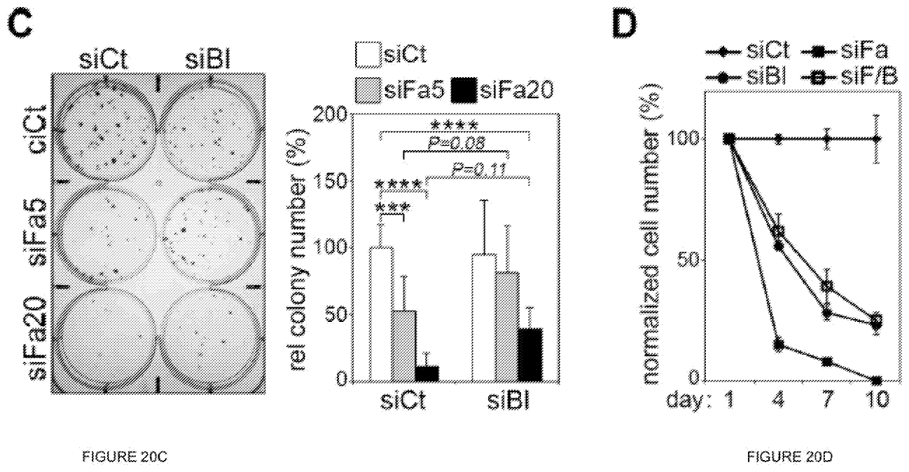

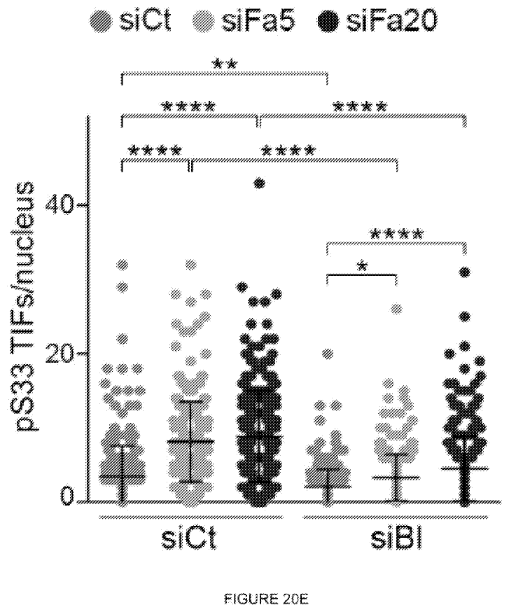

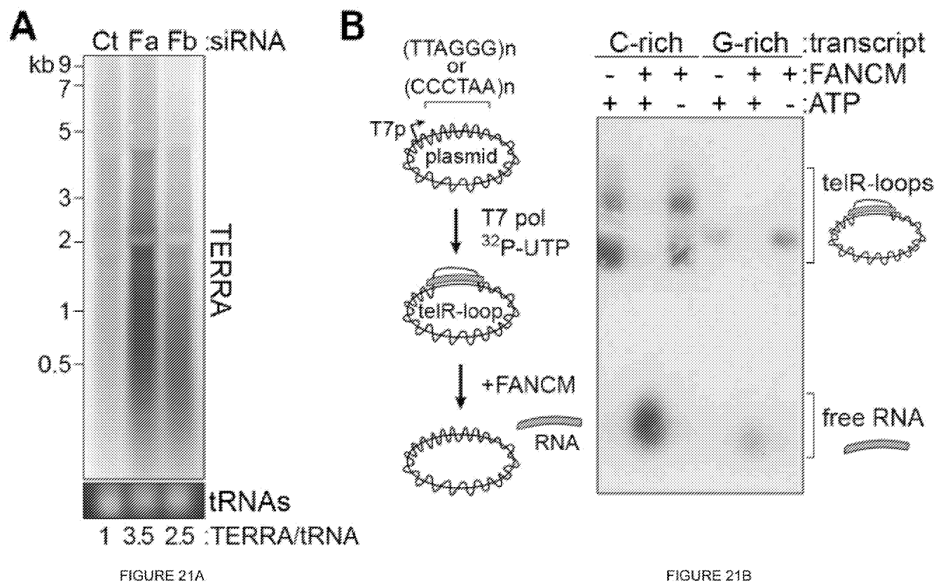

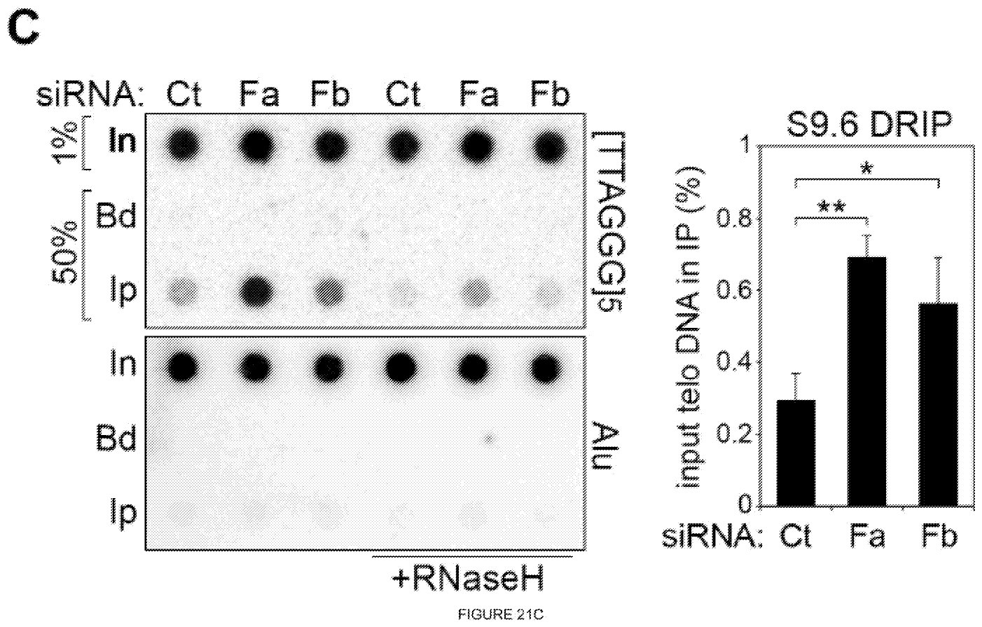

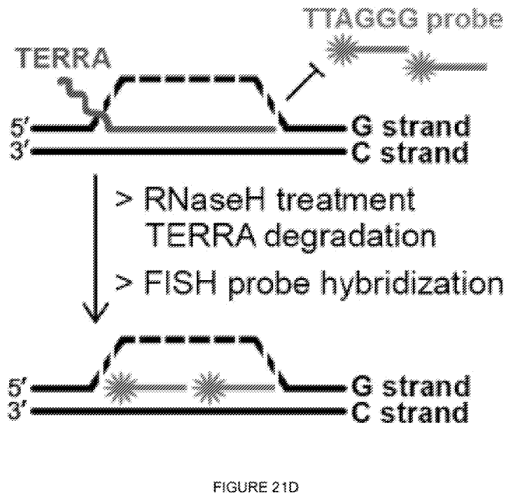

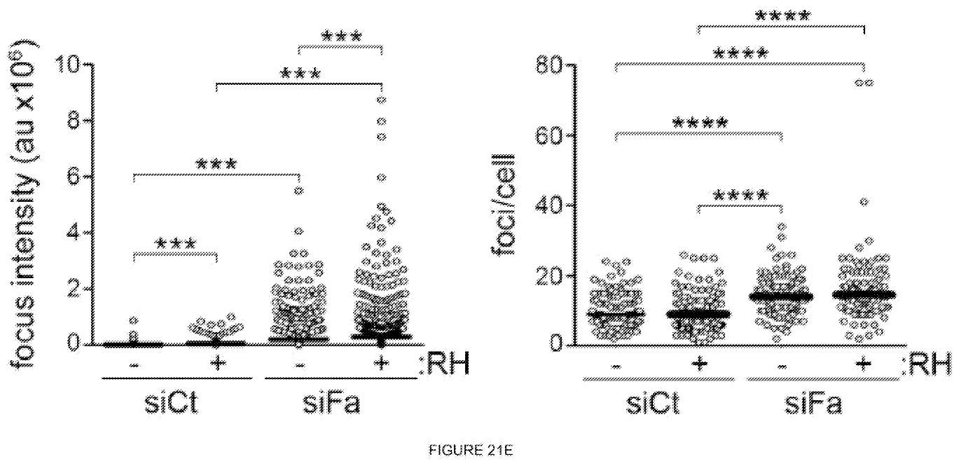

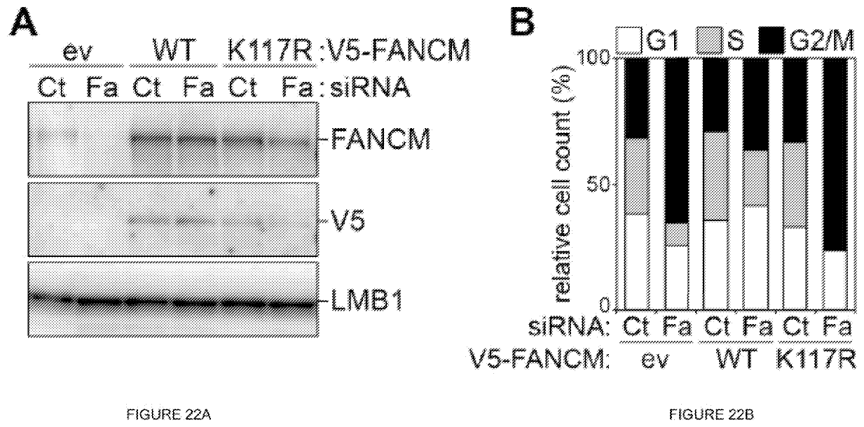

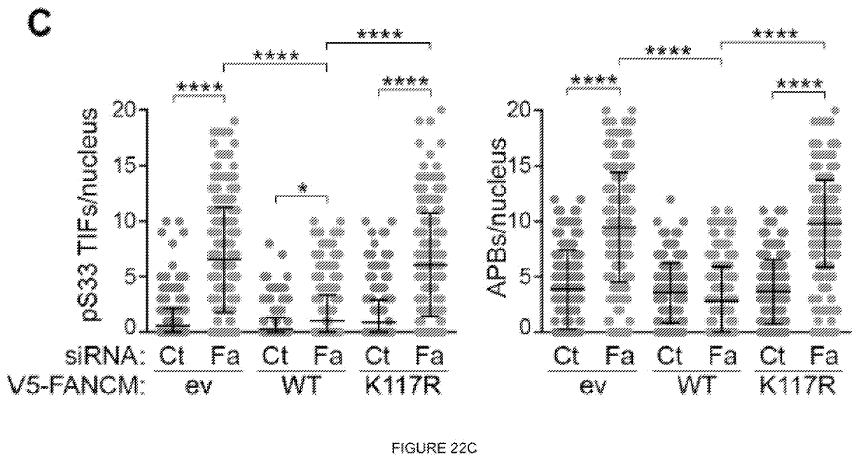

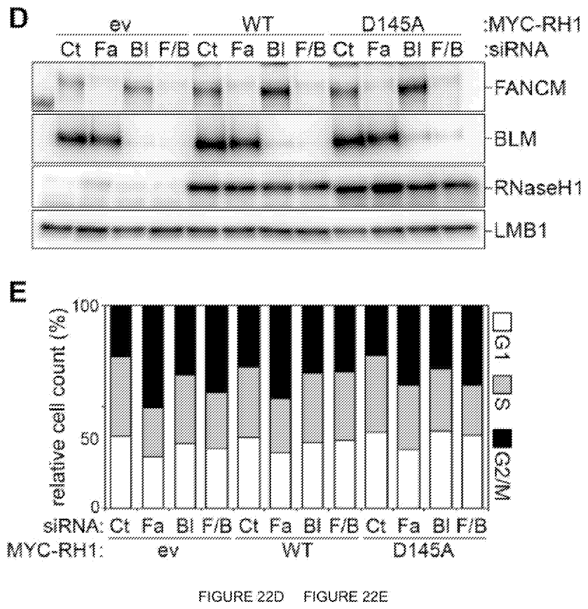

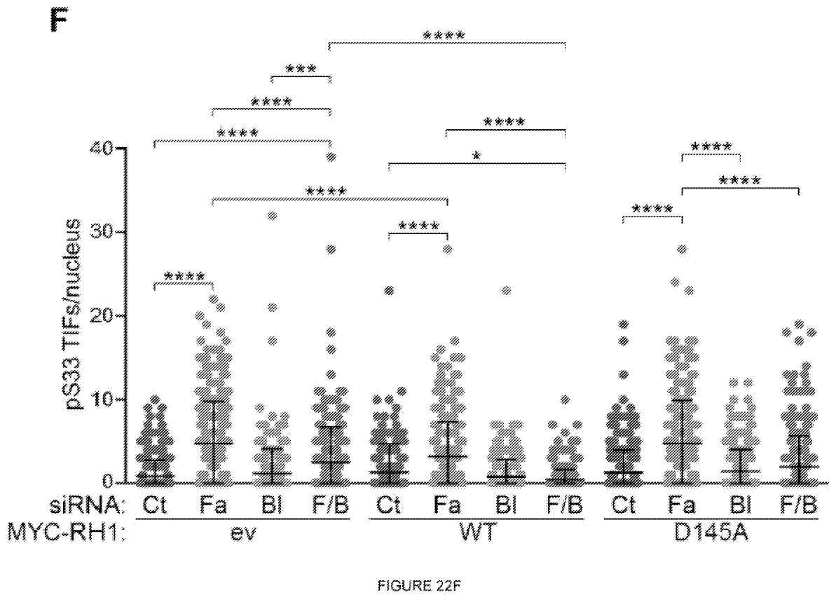

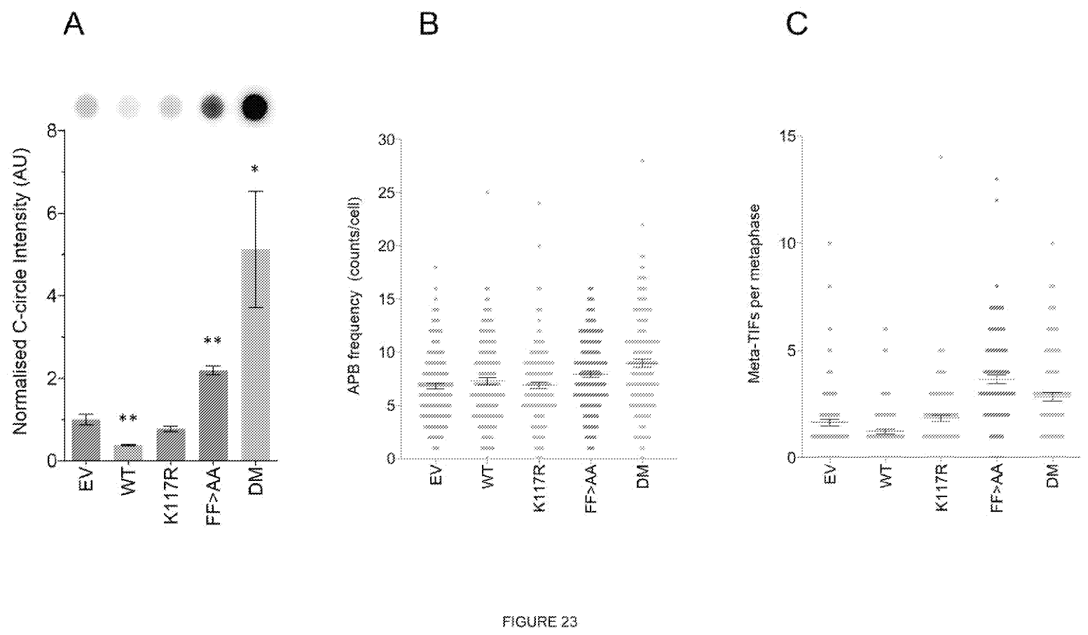

FIG. 20. BLM depletion substantially averts the phenotypes associated with FANCM depletion. (A) Western blot analysis of FANCM and BLM in U2OS cells transfected with siFa, anti-BLM siRNAs (siBl), and siCt. Two different concentrations (5 and 20 nM) of siFa were used. Cells were harvested 48 hours after transfection. LMB1 serves as loading control. The asterisk indicates a band cross-reacting with the anti-FANCM antibody. (B) Quantifications of FACS profiles of cells as in A stained with PI. The graph shows the percentage of cells in G1, S and G2/M phases from one representative experiment. (C) Example of colony formation assays using cells as in A. siFa5: 5 nM siRNA, siFa20: 20 nM siRNA. The graph on the right shows colony numbers relative to siCt-transfected samples. Bars and error bars are means and SDs from 4 independent experiments. P values were calculated with a two-way ANOVA followed by Tukey's HSD. (D) Growth curves of U2OS cells transfected with the indicated siRNAs (20 nM each) every three days. Cell numbers are expressed relative to siCt-transfected cells. Data points and error bars are means and SDs from three independent experiments. SiCt and siFa curves are the same as the ones shown in FIG. 1F. (E) Quantifications of numbers of pS33 TIFs per nucleus in cells as in A. Each dot represents an individual nucleus. A total of at least 300 nuclei from three independent experiments were analyzed for each sample. Bars and error bars are means and SDs. P values were calculated with a two-way ANOVA followed by Tukey's HSD. *P<0.05, **P<0.005, ***P<0.001, ****P<0.0001.

FIG. 21. FANCM restricts TERRA and telR-loops in ALT cells. (A) TERRA northern blot using RNA from U2OS cells harvested 48 hours after siRNA transfection. Sizes of a molecular weight marker are on the left. Numbers at the bottom are quantifications of TERRA signals normalized through the corresponding tRNA signals and expressed relative to the siCt sample. (B) Schematic representation of how telR-loops were generated and unwound in vitro. The gel on the right is an example of a telR-loop unwinding assay performed with telR-loop plasmids (1 nM) and purified recombinant FANCM-FAAP24 (2.5 nM). Note that FANCM unwinds telR-loops only when ATP is included in the reaction. (C) Dot-blot hybridization of DRIPs in U2OS cells as in A using radiolabeled G-rich telomeric oligonucleotides. After signal acquisition, membranes were denatured and re-hybridized to radiolabeled Alu repeat oligonucleotides. In: Input, Bd: only beads control, Ip: S9.6 immunoprecipitation. Signals are graphed on the right as the fraction of In found in the corresponding Ip samples, after subtraction of Bd-associated signals. Alu signals are not included in the quantification. Bars and error bars are means and SDs from 4 independent experiments. P values were calculated with a two-tailed Student's t-test. (D) Schematic representation of the protocol for native FISH. The displaced DNA strand is indicated by a dotted line because the same protocol allows detection also of C-rich DNA engaged in RNA:DNA hybrids devoid of a displacement loop. (E) Quantifications of experiments as in D. 3-D images were sum projected and integrated intensities of FISH signal were measured within individual nuclei identified by DAPI staining (not shown) and background subtracted. Each dot represents an individual nucleus. A total of 100-120 nuclei were analyzed for each sample. One representative experiment is shown. Bars are means. P values were calculated with a Mann-Whitney U test. *P<0.05, **P<0.005, ***P<0.001, ****P<0.0001.

FIG. 22. Deregulated telR-loops contribute to the replication stress arising upon FANCM depletion in ALT cells. (A) Western blot analysis of U2OS cells infected with retroviruses expressing V5 epitope-tagged FANCM variants or empty vector (ev) control retroviruses. WT: wild type, K117R: ATPase/translocase dead FANCM. Five days after infections cells were transfected with the indicated siRNAs and harvested 48 hours later. LMB1 serves as loading control. (B) Quantifications of FACS profiles of cells as in A stained with PI. The graph shows the percentage of cells in G1, S and G2/M phases from one representative experiment. (C) Quantifications of numbers of pS33 TIFs and APBs per nucleus in cells as in A. Each dot represents an individual nucleus. A total of at least 300 nuclei from three independent experiments were analyzed for each sample. Bars and error bars are means and SDs. P values were calculated with a two-way ANOVA followed by Tukey's HSD. (D) Western blot analysis of U2OS cells infected with retroviruses expressing MYC epitope-tagged RNaseH1 (RH1) variants or ev control retroviruses. D145A: endoribonuclease dead RNaseH1. Five days after infections cells were transfected with the indicated siRNAs and harvested 48 hours later. siF/B: combined siFa and siBl. LMB1 serves as loading control. (E) Quantifications of FACS profiles of cells as in D stained with PI. The graph shows the percentage of cells in G1, S and G2/M phases from one representative experiment. (F) Quantifications of numbers of pS33 TIFs per nucleus in cells as in D. Each dot represents an individual nucleus. A total of at least 300 nuclei from three independent experiments were analyzed for each sample. Bars and error bars are means and SDs. P values were calculated with a two-way ANOVA followed by Tukey's HSD. Comparisons between D145A and ev and WT are not indicated. *P<0.05, ***P<0.001, ****P<0.0001.

FIG. 23. Defects in ALT phenotype suppression are exacerbated by overexpression of a double-mutant (DM) FANCM, in which both the MM2 domain (FF>AA) and the translocase domain (K117R) of FANCM are mutated. (a) Representative dot blots and quantitation of C-circle assays in U-2 OS cells stably overexpressing wild-type (WT) or FANCM domain mutants (K117R, FF>AA; DM). C-circles were normalized to the mean of empty vector control (EV). Error bars represent mean±SEM from n=3 experiments, *p<0.05, **p<0.005, Student's t-test. (b) Quantitation of APB frequency in U-2 OS cells overexpressing wild-type (WT) or FANCM mutants. Scatterplot bars represent the mean±SEM. Out of three experiments, n=150 cells were scored for each mutant, **p<0.005, Mann-Whitney test. (c) Quantitation of metaphase-TIFs in U-2 OS cells stably overexpressing wild-type (WT) or FANCM mutants. Scatterplot bars represent the mean±SEM. Out of three experiments, n=120 metaphases scored for each mutant, *p<0.05, **p<0.005, Mann-Whitney test. All statistical comparisons are relative to EV control.

Key to the Sequence Listing
SEQ ID NO: 1 Amino acid sequence for a reference human telomerase protein (Uniprot accession no. 014746).
SEQ ID NO: 2 Amino acid sequence for a reference human FANCM protein (Uniprot accession no. Q8IYD8).
SEQ ID NO: 3 Amino acid sequence for a reference human RMI1 protein (Uniprot accession no. Q9H9A7).
SEQ ID NO: 4 Amino acid sequence for a reference human RMI2 protein (Uniprot accession no. Q96E14).
SEQ ID NO: 5 Amino acid sequence for a reference human MM2 protein domain.
SEQ ID NO: 6 Amino acid sequence for a reference human BLM protein (Uniprot accession no. P54132).
SEQ ID NO: 7 Amino acid sequence for a reference human TOP3A protein (Uniprot accession no. Q13472).
SEQ ID NO: 8 Amino acid sequence for a reference human BRCA1 protein (Uniprot accession no. P38398).
SEQ ID NO: 9 Nucleotide sequence for a reference human FANCM sequence (Genbank accession no. NM_020937.1).
SEQ ID NO: 10 Nucleotide sequence for a reference human RMI1 sequence (Genbank accession no. NM_001358291.1).
SEQ ID NO: 11 Nucleotide sequence for a reference human RMI2 sequence (Genbank accession no. NM_152308.3).
SEQ ID NO: 12 Nucleotide sequence of FANCM1 siRNA.
SEQ ID NO: 13 Nucleotide sequence of FANCM2 siRNA.
SEQ ID NO: 14 Nucleotide sequence of POLD3 siRNA.
SEQ ID NO: 15 Nucleotide sequence of BLM siRNA.
SEQ ID NO: 16 Nucleotide sequence of RAD51 siRNA.
SEQ ID NO: 17 Nucleotide sequence of RAD52 siRNA.
SEQ ID NO: 18 Nucleotide sequence of RMI1 siRNA.
SEQ ID NO: 19 Amino acid sequence for a mutant human FANCM protein (PIP).
SEQ ID NO: 20 Amino acid sequence for a mutant human FANCM protein (K117R).
SEQ ID NO: 21 Amino acid sequence for a mutant human FANCM protein (MID).
SEQ ID NO: 22 Amino acid sequence for a mutant human FANCM protein (S1045A).
SEQ ID NO: 23 Amino acid sequence for a mutant human FANCM protein (ΔMM1).
SEQ ID NO: 24 Amino acid sequence for a mutant human FANCM protein (ΔMM2).
SEQ ID NO: 25 Amino acid sequence for a mutant human FANCM protein (FF>AA).
SEQ ID NO: 26 Amino acid sequence for a mutant human FANCM protein (ΔMM3).
SEQ ID NO: 27 Amino acid sequence for a mutant human FANCM protein (ΔERCC4).
SEQ ID NO: 28 Amino acid sequence for a mutant human FANCM protein (ΔHhH).
SEQ ID NO: 29 Amino acid sequence for a wildtype human FANCM MM2 peptide.
SEQ ID NO: 30 Amino acid sequence for a mutant human FANCM MM2 peptide.
SEQ ID NO: 31 Amino acid sequence for a reference human FANCM protein.
SEQ ID NO: 32 Nucleotide sequence of a reference human FANCM coding sequence.
SEQ ID NO: 33 Nucleotide sequence an siRNA molecule for the suppression of human FANCM (siFa).
SEQ ID NO: 34 Nucleotide sequence an siRNA molecule for the suppression of human FANCM (siFb).
SEQ ID NO: 35 Amino acid sequence for a double mutant human FANCM protein (K117R and FF>AA).
SEQ ID NO: 36 Nucleotide sequence an siRNA molecule for the suppression of human FANCM (siB1).
SEQ ID NO:37 Nucleotide sequence an siRNA molecule for the suppression of human FANCM (siATRXa).
SEQ ID NO:38 Nucleotide sequence an siRNA molecule for the suppression of human FANCM (siATRXb).
SEQ ID NO:39 Nucleotide sequence an siRNA molecule for the suppression of human FANCM (siTRF1).
SEQ ID NO:40 Nucleotide sequence an siRNA molecule for the suppression of human FANCM (siRNaseH1).

### DETAILED DESCRIPTION

### General Techniques and Definitions

Unless specifically defined otherwise, all technical and scientific terms used herein shall be taken to have the same meaning as commonly understood by one of ordinary skill in the art (e.g. in genomics, immunology, molecular biology, immunohistochemistry, biochemistry, oncology, and pharmacology).

The present disclosure is performed using, unless otherwise indicated, conventional techniques of molecular biology, microbiology, recombinant DNA technology and immunology. Such procedures are described, for example in Sambrook, Fritsch & Maniatis, Molecular Cloning: A Laboratory Manual, Cold Spring Harbor Laboratories, New York, Fourth Edition (2012), whole of Vols I, II, and III; DNA Cloning: A Practical Approach, Vols. I and II (D. N. Glover, Second Edition, 1995), IRL Press, Oxford, whole of text; Oligonucleotide Synthesis: A Practical Approach (M. J. Gait, ed, 1984) IRL Press, Oxford, whole of text, and particularly the papers therein by Gait, pp 1-22; Atkinson et al, pp 35-81; Sproat et al, pp 83-115; and Wu et al, pp 135-151; 4. Nucleic Acid Hybridization: A Practical Approach (B. D. Hames & S. J. Higgins, eds., 1985) IRL Press, Oxford, whole of text; Immobilized Cells and Enzymes: A Practical Approach (1986) IRL Press, Oxford, whole of text; Perbal, B., A Practical Guide to Molecular Cloning (1984) and Methods In Enzymology (S. Colowick and N. Kaplan, eds., Academic Press, Inc.), whole of series.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations of any two or more of said steps or features.

The present disclosure is not to be limited in scope by the specific embodiments described herein, which are intended for the purpose of exemplification only. Functionally equivalent products, compositions and methods are clearly within the scope of the disclosure, as described herein.

Each feature of any particular aspect or embodiment of the present disclosure may be applied mutatis mutandis to any other aspect or embodiment of the present disclosure.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

As used herein, the singular forms of “a”, “and” and “the” include plural forms of these words, unless the context clearly dictates otherwise.

The term “and/or”, e.g., “X and/or Y” shall be understood to mean either “X and Y” or “X or Y” and shall be taken to provide explicit support for both meanings or for either meaning.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Throughout this specification, the word “comprise” or variations such as “comprises” or “comprising” will be understood to imply the inclusion of a stated element, integer or step, or group of elements, integers or steps, but not the exclusion of any other element, integer or step, or group of elements, integers or steps.

ALT Cells and ALT Cancers

The present disclosure provides methods of inhibiting ALT cell viability and/or growth. In this regard, the inventors have surprisingly shown that disrupting the FANCM-RMI interaction is selectively toxic to ALT cells. This disclosure also relates to the finding that reduction or abolition of FANCM expression or activity in ALT cancer cells induces G2/M arrest and cell death. Thus, methods of disrupting the FANCM-RMI interaction and methods of reducing or inhibiting FANCM expression or activity may be used, for example, in the treatment of ALT cancers.

In order to achieve unlimited proliferation, cancer cells must maintain their telomeres. The majority of cancers (85-90%) achieve this by reactivating telomerase (telomerase-positive cells). Telomerase is a reverse transcriptase enzyme involved in synthesizing telomeric DNA from an RNA template. The sequence of telomerase is publicly available. An exemplary sequence is set forth in SEQ ID NO: 1. The remaining 10-15% of tumour cells must stabilize their chromosome ends by alternative mechanisms to avert cessation of growth. These telomerase independent strategies are collectively known as Alternative Lengthening of Telomeres (ALT). Thus, an ALT cell as defined herein may be a cell exhibiting an active ALT mechanism. The ALT mechanism may be any mechanism of telomere stabilization that does not rely on telomerase. The ALT mechanism is not necessarily limited to any one specific mechanism by which ALT may operate in a cell to maintain telomeres. Thus, “ALT mechanism” does not necessarily refer to only one specific biochemical mechanism or pathway by which an ALT mechanism operates. There may be more than one specific pathway by which ALT operates.

Thus, an ALT cell may be any cell which does not rely on telomerase activity to maintain its telomere length. Alternatively or in addition, an ALT cell may be considered to be any cell which does not rely on telomerase activity to maintain its telomere stability. Thus, an ALT cell may be considered to be a cell which is not telomerase positive; or a telomerase negative cell.

Alternatively or in addition, an ALT cell may be considered to be a cell in which telomerase expression and/or activity is reduced compared to a telomerase positive cell. Expression and/or activity of telomerase can be determined by any means known in the art. For example, telomerase expression levels can be determined by quantifying the level of production of telomerase mRNA by any suitable method of mRNA detection (for example but without limitation: quantitative PCR, real time qPCR; next generation sequencing (NGS) methods; nanopore sequencing methods; northern blotting; and others). Alternatively or in addition, telomerase expression levels can be determined by quantifying the level of production of telomerase protein by any suitable protein detection methods. Telomerase protein levels can be detected, for example but without limitation, by western blotting; antibody detection methods (e.g., ELISA; or detection of a label such as a fluorescent label conjugated to an antibody capable of binding specifically to telomerase); and other methods. Alternatively or in addition, telomerase expression and/or activity levels can be determined through performance of a telomerase functional assay, wherein the level of telomerase activity is indicative of the level of expression and/or activity of telomerase in a cell. Telomerase activity may be detected by the Telomerase Repeat Amplification Protocol (TRAP), quantitative TRAP (qTRAP), or by a direct telomerase activity assay, such as that described in Cohen and Reddel, 2008. It will be appreciated that any of the methods of identifying an ALT cell or identifying an ALT cancer or determining whether a subject is suffering from ALT cancer disclosed herein may comprise determining whether a cell is a telomerase positive cell by any of the methods disclosed herein, wherein the cell is identified as not being an ALT cell if that cell is determined to be a telomerase positive cell.

ALT cells may be characterized by elevated levels of DNA damage compared to mortal or telomerase-positive cells, indicative of heightened telomeric replication stress in ALT cells. This heightened telomeric replication stress is attributed to cumulative inadequacies in telomere structural integrity. Frequent or persistent replication fork stalling causes nicks and breaks in the DNA, and it has been hypothesized that the ALT mechanism emanates from stalled replication forks that deteriorate to form double stranded breaks (DSBs), which then provide the substrate for the engagement of homology-directed repair pathways, culminating in break induced telomere synthesis. ALT cells therefore achieve a fine balance between telomere protection and repair activities and telomere damage, and disruption of this balance can be applied as a means of dysregulating the ALT mechanism.

An ALT cell as defined herein may be identified by detection of one or more phenotypic traits of ALT telomere repair or ALT telomeric replication stress, including any one or more of: replication fork stalling above a level that is typical of non-ALT cells (e.g., above a level that is typical of telomerase positive cells or mortal cells); DSB occurrence above a level that is typical of non-ALT cells (e.g., above a level that is typical of telomerase positive cells or mortal cells); and others. It will be appreciated that levels of telomeric replication fork stalling and/or DSBs that are typical of ALT cells can be established through identification and/or measurement of these traits in a sample of ALT cells and in a sample of non-ALT cells (e.g., telomerase positive cells or mortal cells). Suitable threshold levels can then be determined according to the particular methodology used to identify and/or measure these traits, such that a given cell can then be identified as an ALT cell or a non-ALT cell using the same or similar methodology. It will be appreciated that the precise thresholds will vary depending on the samples used to establish those threshold levels and according to the particular analytical methodology used in each instance. ALT involves recombination-dependent DNA replication (Dunham et al., 2000) and ALT may generate sudden, large increases in telomere length (Murnane et al., 1994), consistent with either a long, linear telomeric template or a rolling mechanism, such as rolling circle amplification (RCA). Cells with ALT activity also undergo rapid decreases in individual telomere lengths (Jiang et al., 2005 and Perrem et al., 2001) leading to a highly heterogeneous telomere length distribution. ALT cells often contain telomeric chromatin within promyelocytic leukemia (PML) nuclear bodies (ALT-associated promyelocytic leukemia nuclear bodies; APBs) (Yeager et al., 1999). Thus, an ALT cell as defined herein may comprise any one or more of these phenotypic traits. For example, an ALT cell may exhibit recombination-dependent DNA replication, and/or may exhibit sudden, large increases in telomere length (e.g., compared to a non-ALT cell such as a telomerase positive cell or a mortal cell), and/or may exhibit heterogeneous telomere length distribution (e.g., compared to a non-ALT cell such as a telomerase positive cell or a mortal cell), and/or may comprise APBs (e.g., a level of APBs that is greater than in a non-ALT cell, such as a telomerase positive cell or a mortal cell). Alternatively, an ALT cell may be identified by the maintenance of telomere length over one or more cell divisions, in the absence of telomerase activity and/or expression. It will be appreciated that telomeres are repetitive DNA sequences present at or near the termini of linear chromosomes. Telomeres in humans typically comprise multiple repeats of the nucleotide sequence 5′-TTAGGG-3′. Thus, the identification of telomere length may comprise determining the number of repeats of this nucleotide sequence.

The ALT cell disclosed herein may be a cancer cell. Accordingly, the ALT cell disclosed herein may be derived from a subject suffering from, suspected of suffering from, or predisposed to, a disease or condition associated with abnormal cellular proliferation. The cancer may be of any physiological origin. ALT has been identified in a wide variety of cancers, including but not limited to carcinomas arising from tissue, including tissue derived from the bladder, cervix, endometrium, esophagus, gallbladder, kidney, liver, lung, brain, bone and connective tissue. ALT has also been found in medulloblastomas, oligodendrogliomas, meningiomas, schwannomas and pediatric glioblastoma multiformes. In one example, the cancer may be any one of bladder cancer, cervical cancer, endometrial cancer, esophageal cancer, gallbladder cancer, kidney cancer, liver cancer, lung cancer, brain cancer, bone cancer or connective tissue cancer. The ALT cancer or cell may be, for example, a sarcoma, a blastoma, a carcinoma, a mesothelioma or an astrocytoma. The sarcoma may be osteosarcoma, malignant fibrous histiocytoma, liposarcoma, synovial sarcoma, fibrosarcoma, chondrosarcoma, rhabdomyosarcoma or leiomyosarcoma. The blastoma may be neuroblastoma. The carcinoma may be a non-small cell lung carcinoma such as lung adenocarcinoma or a breast carcinoma. The mesothelioma may be peritoneal mesothelioma. The astrocytoma may be low-grade astrocytoma, anaplastic astrocytoma, or glioblastoma multiforme. The ALT cancer or cell may be a medulloblastoma, oligodendroglioma, meningioma, schwannoma and/or pediatric glioblastoma multiforme. In particular embodiments, the ALT cancer is an osteosarcoma, soft tissue sarcoma (e.g. liposarcoma, undifferentiated pleomorphic sarcoma, or leiomyosarcoma), glioblastoma, astrocytoma, neuroblastoma, or bladder carcinoma. The cancer may be a primary cancer or a metastatic cancer. The metastatic cancer may be of a known or unknown origin. The methods disclosed herein may be used to treat any of these or other ALT cancers.

The ALT cell may be derived from any vertebrate, such as a mammal, and in particular, a human.

As indicated herein, methods of determining whether a cell is an ALT cell or whether a cancer is an ALT cancer are known in the art. For example, the C-circle biomarker is an ALT specific molecule which can be detected using the C-circle assay (Henson et al., 2009 and WO/2011/035375). The entire content of WO/2011/035375 is incorporated herein by reference. Briefly, the C-circle assay comprises extracting DNA from the specimen and subsequently quantifying it. For example, the C-circle can be amplified by rolling circle amplification and the products can be detected.

Any of the methods disclosed in Henson et al., 2009 and/or WO/2011/035375 can be used to identify a cell as an ALT cell in conjunction with the present disclosure. Thus, for example, the methods disclosed herein may comprise identifying a cell as an ALT cell by determining the presence and/or amount of partially double-stranded telomeric DNA circles in a cell, wherein the presence and/or amount of partially double-stranded telomeric DNA circles identifies that cell as an ALT cell. The partially double stranded telomeric DNA circles may comprise a closed circular strand and a linear strand. The circular strand may comprise a C-rich or G rich telomeric sequence. The linear strand may comprise G-rich or C-rich telomeric DNA sequence. The partially double-stranded telomeric circles may comprise repeats of the sequence (CCCTAA)ₙ on the circular strand and/or repeats of the sequence (TTAGGG)ₙ on the linear strand (wherein n is any integer greater than 1). The partially double-stranded telomeric circles may comprise repeats of the sequence (TTAGGG)ₙ on the circular strand and/or repeats of the sequence (CCCTAA)ₙ on the linear strand (wherein n is any integer greater than 1). In one example, the presence and/or amount of partially double-stranded telomeric DNA circles in a cell may be detected using rolling circle amplification.

The circular and/or linear strand may comprise variant telomeric repeat sequences, mutant telomeric repeat sequences and/or non-telomeric sequences.

The partially double-stranded telomeric circles may be detected directly or indirectly. For example, detection may be indirect following rolling circle amplification. The rolling circle amplification may use the circular strand of the partially double-stranded circles as template. In one example, the detection comprises:

(a) optionally isolating DNA from the cell;

(b) incubating the DNA in the presence of a DNA polymerase and one or more dNTPs under suitable conditions such that polymerase-mediated extension from the incomplete (linear) strand generates concatemers of single-stranded telomeric DNA; and

(c) detecting the concatemers.

The concatemers may be detected by any suitable means such as, for example, hybridisation, sequencing, PCR, molecular beacons, nucleic acid enzymes such as DNA partzymes, or by incorporating suitably labelled dNTPs in incubation step (b). In one example, the concatamers may be detected using a labelled nucleotide probe. The labelled probe may comprise the nucleotide sequence (CCCTAA)ₙ, wherein n is 1 or any integer greater than 1. The label may be any detectable label. For example, the label may be a fluorescent label.

The DNA polymerase may be, for example, φ29 DNA polymerase. Typically, wherein the partially double-stranded telomeric circles comprise repeats of the sequence (CCCTAA)ₙ on the circular strand, the dNTPs consist of dATP, dGTP and dTTP, and optionally dCTP.

The detection of the partially double-stranded telomeric circles may be detection of said circles present within the cell, or alternatively may comprise the detection of said circles in a biological sample, for example derived from a subject. The biological sample may comprise, for example, blood, urine, sputum, pleural fluid, peritoneal fluid, bronchial and bronchoalveolar lavage fluid, or a tissue section. The sample may be obtained, for example, by fine needle aspiration biopsy. The blood may be whole blood, blood serum or blood plasma.

The rolling circle amplification may be conducted with or without the provision of an exogenous primer. Advantageously, the rolling circle amplification can be conducted without an exogenous primer. Thus, the methods disclosed herein of identifying an ALT cell by performing a C-circle assay may not comprise the use of an exogenous primer.

Other suitable methods to determine ALT activity include, but are not limited to, telomerase quantitative PCR of telomeric DNA and C-circles (Lau et al., 2012); the absence of telomerase activity; the presence of very long and heterogeneous telomeres; the presence of ALT-associated promyelocytic leukemia (PML) bodies (APBs), which contain telomeric DNA and telomere binding proteins (Yeager et al., 1999); elevated telomeric sister chromatid exchange (T-SCE) events and the presence of extrachromosomal telomeric repeat (ECTR) DNA. Tumour samples may also be assessed by combined telomere-specific fluorescence in situ hybridization and immunofluorescence labelling for PML protein (Heaphy et al., 2011).

ALT cells contain a novel form of promyelocytic leukemia (PML) bodies (ALT-associated PML body, APBs) in which PML protein colocalizes with telomeric DNA and the telomere binding proteins hTRF1 and hTRF2. APBs are not found in mortal cells, strains of telomerase-positive cell lines or tumours (Yeager et al., 1999). Any method known in the art used to detect APBs may be used in conjunction with the present disclosure. For example, APBs may be detected visually. For example, APBs may be visualized by immunohistochemistry (for example, using anti-hTRF1 and/or anti-PML antibodies).

Significant differences in telomere variant repeat content have been found in tumours that use the ALT mechanism and those that do not (Lee et al., 2018). Thus, any method known in the art to determine telomere variant repeat content may be used in conjunction with the present disclosure. For example, whole genome sequencing may be used to determine telomere variant repeat content.

FANCM

Fanconi anemia group M protein (FANCM) is an ATP-dependent DNA helicase/translocase (EC 3.6.4.13) involved in homologous recombination, meiosis and DNA repair. FANCM may be human FANCM. The human gene (Gene ID: 57697) encoding the FANCM polypeptide has 25 exons and is located at 14q21.2. A reference human FANCM amino acid sequence is shown in SEQ ID NO: 31. Other reference human FANCM amino acid sequences have the database accession numbers NP 001295062.1, NP 1295063.1, and NP 065988.1. A FANCM polypeptide as described herein may comprise a reference amino acid sequence, such as SEQ ID NO: 31 or an amino acid sequence having at least 80% sequence identity, at least 85% sequence identity, at least 90% sequence identity, at least 95% identity, or at least 98% identity to a reference amino acid sequence, such as SEQ ID NO: 31.

A reference human FANCM coding nucleotide sequence is shown in SEQ ID NO: 32. Other reference human FANCM coding sequences have the database accession numbers NM_001308133.1, NM_001308134.1 and NM_020937.4. A FANCM nucleotide sequence as described herein may comprise a nucleotide sequence of a reference human FANCM coding sequence, such as SEQ ID NO: 32 or a nucleotide sequence having at least 80% sequence identity, at least 85% sequence identity, at least 90% sequence identity, at least 95% identity, or at least 98% identity to a reference human FANCM coding sequence, such as SEQ ID NO: 32.

Sequence identity is commonly defined with reference to the algorithm GAP (Wisconsin GCG package, Accelerys Inc, San Diego USA). GAP uses the Needleman and Wunsch algorithm to align two complete sequences that maximizes the number of matches and minimizes the number of gaps. Generally, default parameters are used, with a gap creation penalty=12 and gap extension penalty=4. Use of GAP may be preferred but other algorithms may be used, e.g. BLAST (which uses the method of Altschul et al. (1990) J. Mol. Biol. 215: 405-410), FASTA (which uses the method of Pearson and Lipman (1988) PNAS USA 85: 2444-2448), SSEARCH (Smith and Waterman (1981) J. Mol Biol. 147: 195-197;), HMMER3 (Johnson L S et al BMC Bioinformatics. 2010 Aug. 18; 11( ):431) or the TBLASTN program, of Altschul et al. (1990) supra, generally employing default parameters (see for example Pearson Curr Prot Bioinformatics (2013) Chapt 3 Uniy 3.1 doi:10.1002/0471250953.bi0301s42). In particular, the psi-Blast algorithm may be used (Altschul et al. Nucl. Acids Res. (1997) 25 3389-3402). Sequence identity and similarity may also be determined using Genomequest™ software (Gene-IT, Worcester Mass. USA). Sequence comparisons are preferably made over the full-length of the relevant sequence described herein.

In other embodiments, for example when the individual to be treated is a non-human mammal, FANCM may be non-human mammalian FANCM.

A reduction in FANCM expression or activity is shown herein to reduce viability and increase cell cycle arrest and death in cells that rely on the Alternative Lengthening of Telomeres (ALT) pathway to maintain telomeres (ALT cells). In contrast, a reduction in FANCM expression or activity has no effect on telomerase positive cells (which do no maintain telomeres through the ALT pathway) or primary cells. An agent which reduces FANCM expression or activity may therefore be used to induce cell death in ALT cancer cells (i.e. cancers that rely on the ALT pathway to maintain telomeres), for example in the treatment of ALT cancer.

FANCM activity may be reduced in an individual by administering an agent that reduces FANCM expression or activity, such as an FANCM antagonist. A FANCM antagonist may inhibit the activity of FANCM, for example a FANCM inhibitor, or may reduce or suppress the expression of FANCM, for example a suppressor nucleic acid or targeted nuclease.

The FANCM-RMI Complex

FANCM is an integral factor in the stabilization of stalled replication forks. It contains two DNA binding domains at its N- and C-termini, between which are three highly conserved regions (MM1-MM3). The sequence of FANCM is publicly available. An exemplary sequence is set forth in SEQ ID NO: 2. The MM1 domain recruits the FA core complex, a multi-subunit ubiquitin ligase that is essential for DNA interstrand crosslink (ICL) repair, while the MM2 domain, which has been described as a 34-amino acid motif binds directly to the RMI (RecQ-mediated genome instability) subcomplex (RMI1 and RMI2) subcomplex of BLM-TOP3A-RMI (BTR). The MM2 domain has also been described in (Deans et al., 2009). The sequence of RMI1 and RMI2 are publicly available and exemplary sequences are set forth in SEQ ID NOs: 3 and 4. An exemplary sequence comprising the MM2 domain is set forth in SEQ ID NO: 5. Thus, the MM2 domain may comprise an amino acid sequence as set forth in SEQ ID NO: 5. Alternatively, the MM2 domain as described herein may comprise an amino acid sequence that is at least 90% identical to the amino acid sequence DLFSVTFDLGFC (SEQ ID NO: 49). The MM2 domain may comprise an amino acid sequence that is at least 90%, at least 91%, at least 92%, at least 93%, at least 94%, at least 95%, at least 96%, at least 97%, at least 98%, at least 99%, or 100% identical to the amino acid sequence DLFSVTFDLGFC (SEQ ID NO: 49). This sequence has been identified as a core sequence within the MM2 domain (Hoadley et al., 2012).

The BTR complex encompasses BLM helicase activity, TOP3A decatenation activity, branch migration and overall dissolvase activity and it has been suggested that FANCM and BTR may cooperate to regress, and thus stabilize, stalled forks. The sequences of BLM and TOP3A are publicly available and exemplary sequences are set forth in SEQ ID Nos: 6 and 7. FANCM retention at stalled replication forks is dependent on its interaction with a functional BTR complex, but not with the FA core complex.

Inhibitors

In certain embodiments, FANCM inhibitors and RMI inhibitors inhibit or reduce the expression of FANCM or RMI, respectively, in a cell, e.g., an ALT cancer cell. In certain embodiments, FANCM inhibitors and RMI inhibitors inhibit or reduce one or more biological activity of FANCM or RMI, respectively, in a cell, e.g., an ALT cancer cell. In certain embodiments, FANCM inhibitors and RMI inhibitors inhibit or reduce binding of FANCM or RMI, respectively, to another protein in a cell, e.g., an ALT cancer cell. In certain embodiments, FANCM inhibitors inhibit or reduce FANCM ATPase activity in a cell, e.g., an ALT cancer cell.

It will be understood by a person skilled in the art that FANCM inhibitor or RMI inhibitor may be a direct inhibitor or an indirect inhibitor of FANCM or RMI, respectively, i.e., it may exert its inhibitory effect by directly binding to FANCM or RMI, respectively, or a nucleic acid sequence encoding FANCM or RMI, respectively, or it may exert its inhibitory effect indirectly, e.g., by inhibiting another protein required for either FANCM or RMI expression or activity. In particular embodiments, an inhibitor such as those disclosed herein may be capable of inhibiting FANCM such that one or more endogenous activity of FANCM is inhibited. In particular embodiments, an inhibitor such as those disclosed herein may be capable of inhibiting RMI such that one or more endogenous activity of RMI is inhibited. In certain embodiments, a FANCM antagonist or inhibitor inhibits or reduces DNA translocase activity or ATPase activity of FANCM. In particular embodiments, a FANCM antagonist or inhibitor inhibits or reduces ALT activity of an ALT cell, e.g., an ALT cancer cell. In particular embodiments, FANCM DNA translocase activity or ATPase activity is reduced by at least 50%, at least 60%, at least 75%, at least 80%, at least 85%, at least 90%, at least 95%, or completely, as compared to the activity present in an ALT cell not contacted with the FANCM antagonist or inhibitor. In particular embodiments, ALT activity is reduced by at least 50%, at least 60%, at least 75%, at least 80%, at least 85%, at least 90%, at least 95%, or completely, as compared to the activity present in an ALT cell not contacted with the FANCM antagonist or inhibitor.

In certain embodiments, the FANCM-RMI interaction may be disrupted by upstream or downstream effectors of FANCM or RMI. The inhibitor may be a direct inhibitor of the FANCM-RMI interaction or an indirect inhibitor of the FANCM-RMI interaction. For example, the inhibitor may bind to FANCM to inhibit its function by changing its conformation or by affecting its binding site such that it is no longer able to bind to RMI. In another example, the inhibitor may bind to RMI to inhibit its function by changing its conformation or by affecting its binding site such that it is no longer able to bind to FANCM. For example, the inhibitor may disrupt the RMI1-RMI2 subcomplex such that it is no longer able to bind to FANCM. In one example, the binding of FANCM to RMI is disrupted at the MM2 domain. Any inhibitor such as those disclosed herein may be capable of disrupting the FANCM-RMI interaction such that the endogenous function of the FANCM-RMI complex is inhibited.

FANCM activity may be measured through the C-circle assay or through any suitable means known in the art, or any of the methods disclosed herein.

FANCM inhibitors and RMI inhibitors (and other inhibitors) may be any type of molecule with inhibitory activity, for example, small chemical molecules, polypeptides (which includes peptides and proteins), nucleic acids, or molecules comprising a combination of any of these classes of molecules. The terms “FANCM inhibitor” and “RMI inhibitor” as used herein, cover pharmaceutically acceptable salts and solvates of any biological molecules or compounds disclosed herein.

In particular embodiments, an inhibitor may cause a reduction in the expression of a target protein, e.g., FANCM, or a reduction in one or more biological activities of a target protein, e.g., FANCM, in each case, e.g., a reduction of at least 50%, at least 60%, at least 70%, at least 80%, at least 85%, at least 90%, at least 95%, or about 100%. Reducing the amount of active FANCM protein to 20% of the amount in control cells or lower is shown to be sufficient to induce cell death. For example, a cell may express up to 5%, up to 10%, up to 15% or up to 20%, of the active FANCM polypeptide that is expressed by control cells. In particular embodiments, FANCM ATPAse activity is reduced, e.g., by at least 50%, at least 60%, at least 70%, at least 80%, at least 85%, at least 90%, at least 95%, or about 100% as compared to a control cell not treated with the inhibitor.

FANCM inhibitors may, for example, include biological molecules that specifically bind to FANCM. The ATPase activity of FANCM is located within the amino-terminal DEAH helicase-like domain, responsible for translocase and branch migration activities. This ATPase activity is generally found to be dispensable for core complex targeting and FANCD2 ubiquitination but is required for replication fork stability and efficient checkpoint response. In some embodiments, a biological molecule may specifically bind to the region of FANCM associated with ATPase activity. In some embodiments, a biological molecule may specifically bind to the DEAH helicase-like domain corresponding to residues 83-591 of SEQ ID NO: 31. In some embodiments, a biological molecule may specifically bind to the MM2 domain of FANCM corresponding to SEQ ID NO:5.

FANCM antagonists and FANCM inhibitors may, for example, include small chemical molecules, for example non-polymeric organic compounds having a molecular weight of 900 Daltons or less. Suitable small molecule FANCM inhibitors may, for example, inhibit ATP binding to the ATPase domain of FANCM; DNA binding to the translocase domain of FANCM and/or FANCM binding to a binding partner, such as MHF, FAAP24, BLM, RMI, Topo IIIα. Suitable techniques for the rational design of small molecule inhibitors through structural analysis of FANCM are well-known in the art.

In one example, the FANCM inhibitor is a small molecule. In accordance with one example in which the FANCM inhibitor is a small molecule, the small molecule is 4-[(1-Hydroxy-2-phenyl-1H-indol-3-yl)-pyridin-2-yl-methyl]-piperazine-1-carboxylic acid ethyl ester. Thus, the small molecule inhibitor may be the inhibitor defined in the following Formula I:

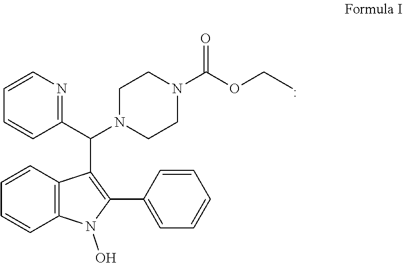 [SMILES: CCOC(=O)N1CCN(C(c2ccccn2)c2c(-c3ccccc3)n(O)c3ccccc23)CC1]

In certain embodiments, an inhibitor is a biological molecule, such as a polypeptide, e.g., a peptide or protein. Peptides may comprise or consist of from 5 to 40 amino acids, for example, from 6 to 10 amino acids and may be derived from FANCM or binding partners thereof as described herein. Polypeptide molecules may also include antibodies, antibody fragments and antibody derivatives and non-immunoglobulin binding molecules, such as aptamers, trinectins, anticalins, kunitz domains, transferrins, nurse shark antigen receptors and sea lamprey leucine-rich repeat proteins. Suitable techniques for the generation of biological molecules that specifically bind to FANCM are well known in the art.

In some embodiments, the FANCM inhibitor is a peptide. The peptide may be any peptide mimicking all or part of the MM2 domain of FANCM. For example, the peptide may be a peptide comprising or consisting of an amino acid sequence that is at least 90% identical to the amino acid sequence DLFSVTFDLGFC (SEQ ID NO:49). The peptide may be at least 90%, at least 91%, at least 92%, at least 93%, at least 94%, at least 95%, at least 96%, at least 97%, at least 98%, at least 99%, or 100% identical to the amino acid sequence DLFSVTFDLGFC (SEQ ID NO: 49). For example, the peptide may be a peptide comprising or consisting of an amino acid sequence that is at least 90% identical to the amino acid sequence DIFDCSRDLFSVTFDLGFCSPDSDDEILEHTSD (SEQ ID NO: 50). The peptide may be at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence DIFDCSRDLFSVTFDLGFCSPDSDDEILEHTSD (SEQ

In some embodiments, the peptide may be a peptide comprising or consisting of an amino acid sequence that is at least 90% identical to EDIFDCSRDLFSVTFDLGFCSPDSDDEILEHTSD (SEQ ID NO: 5). The peptide may be at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence EDIFDCSRDLFSVTFDLGFCSPDSDDEILEHTSD (SEQ ID NO: 5).

In some embodiments, the peptide or protein FANCM inhibitor may be any peptide capable of binding to FANCM and capable of occluding the MM2 binding domain. The occlusion may be such that the normal (endogenous) binding interaction between FANCM and its endogenous binding partner is disrupted. The peptide or protein may be capable of binding to the MM2 binding domain directly or indirectly. Alternatively, the peptide or protein may be any peptide of the RMI complex capable of binding to FANCM.

In some embodiments, a FANCM inhibitor may be a mutant FANCM protein that has reduced binding capability to the RMI protein or a mutant RMI protein that has reduced binding capability to the FANCM protein. Without wishing to be bound by theory, such proteins may act as decoys to the endogenous FANCM or RMI proteins, saturating the available binding sites on the endogenous proteins and thereby inhibiting their function. In one example, the mutant FANCM may be an inactivated FANCM protein comprising a F1232A/F1236A double substitution. The protein may comprise an immunoglobulin binding domain.

In some embodiments, the inhibitor may be a genetic inhibitor of FANCM or of RMI. Methods of designing suitable genetic inhibitors are known in the art. Suitable examples of genetic inhibitors include, but are not limited to, DNA (gDNA, cDNA), RNA (sense RNAs, antisense RNAs, mRNAs, tRNAs, rRNAs, small interfering RNAs (siRNAs), short hairpin RNAs (ShRNAs), micro RNAs (miRNAs), small nucleolar RNAs (SnoRNAs), small nuclear RNAs (snRNAs), ribozymes, aptamers, DNAzymes, antisense oligonucleotides, vectors, plasmids, other ribonuclease-type complexes, and mixtures thereof. The gene sequences of FANCM and RMI1 and RMI2 are publicly available and can be used to design suitable genetic inhibitors by methods known in the art. Reference nucleotide sequences of FANCM, RMI1 and RMI2 are provided in SEQ ID NOs: 9, 10 and 11 respectively.

Examples of suitable genetic inhibitors are described herein in the experimental examples. Thus, the genetic inhibitors may comprise siRNA inhibitors comprising or consisting of the nucleotide sequences disclosed in SEQ ID NO: 12 or SEQ ID NO: 13.

In certain embodiments, inhibitors for reducing or suppressing FANCM expression include suppressor nucleic acids, targetable nucleases and nucleic acids encoding such agents. Nucleic acids encoding a suppressor nucleic acid or targetable nuclease may be contained in a vector. Suitable expression vectors are well-known in the art and include viral vectors, such as retroviral, adenoviral, adeno-associated viral, lentiviral, vaccinia or herpes vectors.

The expression of active FANCM protein may be reduced by a suppressor nucleic acid or targetable nuclease compared to a control cell or may be absent, i.e. the transcription of the FANCM gene and/or translation of FANCM mRNA may be reduced or absent, such that the cell treated with the suppressor nucleic acids or targetable nuclease lacks or has a reduced amount of active FANCM protein compared to a control cell. Reducing the amount of active FANCM protein to 20% of the amount in control cells or lower is shown to be sufficient to induce cell death. For example, a cell may express up to 5%, up to 10%, up to 15% or up to 20%, of the active FANCM polypeptide that is expressed by control cells.

In some embodiments, nucleic acid suppression may be used to reduce the expression of active FANCM polypeptide. The use of nucleic acid suppression techniques such as anti-sense and RNAi suppression, to down-regulate expression of target genes is well-established in the art.

Cells may be transfected with a suppressor nucleic acid (i.e. a nucleic acid molecule which suppresses FANCM expression), such as an siRNA or shRNA, or a heterologous nucleic acid encoding the suppressor nucleic acid. The suppressor nucleic acid reduces the expression of active FANCM polypeptide by interfering with transcription and/or translation, thereby reducing FANCM activity in the cells.

RNAi involves the expression or introduction into a cell of an RNA molecule which comprises a sequence which is identical or highly similar to the FANCM coding sequence. The RNA molecule interacts with mRNA which is transcribed from the FANCM gene, resulting in the sequence specific degradation or specific post-transcriptional gene silencing (PTGS) of the mRNA. This reduces or suppresses expression of active FANCM polypeptide (Angell & Baulcombe (1997) The EMBO Journal 16, 12:3675-3684; Voinnet & Baulcombe (1997) Nature 389: pg 553).

The RNA molecule is preferably double stranded RNA (dsRNA) (Fire A. et al Nature 391, (1998)). Synthetic siRNA duplexes have been shown to specifically suppress expression of endogenous and heterologous genes in a wide range of mammalian cell lines (Elbashir S M. et al. Nature, 411, 494-498, (2001)).

Suitable RNA molecules for use in RNAi suppression include short interfering RNA (siRNA). siRNA are double stranded RNA molecules of 15 to 40 nucleotides in length, preferably 15 to 28 nucleotides or 19 to 25 nucleotides in length, for example 19, 20, 21, 22, 23, 24 or 25 nucleotides in length. For example, two unmodified 21 mer oligonucleotides may be annealed together to form a siRNA. A siRNA molecule may contain a 3′ and/or 5′ overhang on each strand having a length of about 0, 1, 2, 3, 4, or 5 nucleotides. The overhang lengths of the strands are independent, i.e., the length of the overhang on one strand is not dependent on the length of the overhang on the second strand.

Other suitable RNA molecules for use in RNAi include small hairpin RNAs (shRNAs). shRNA are single-chain RNA molecules which comprise or consist of a short (e.g. 19 to 25 nucleotides) antisense nucleotide sequence, followed by a nucleotide loop of 5 to 9 nucleotides, and the complementary sense nucleotide sequence (e.g. 19 to 25 nucleotides). Alternatively, the sense sequence may precede the nucleotide loop structure and the antisense sequence may follow. The nucleotide loop forms a hairpin turn which allows the base pairing of the complementary sense and antisense sequences to form the shRNA.

A suppressor nucleic acid, such as a siRNA or shRNA, may comprise or consist of a sequence which is identical or substantially identical (i.e. at least 90%, at least 95% or at least 98% identical) to all or part (for example, 15 to 40 nucleotides) of a reference FANCM nucleotide coding sequence, such as SEQ ID NO: 32, or its complement. Suitable reference sequences coding FANCM that may be used for the design of suppressor nucleic acids are publically available and include SEQ ID NO: 32. FANCM activity is suppressed in the cancer cells by down-regulation of the production of active FANCM polypeptide by the suppressor nucleic acid. For example, a siRNA to suppress the expression of human FANCM may comprise 18 to 22 contiguous nucleotides from SEQ ID NO: 32.

Examples of preferred siRNA molecules for the suppression of human FANCM include SEQ ID NO: 33 (siFa) and SEQ ID NO: 34 (siFb).

Suppressor nucleic acids, such as siRNAs and shRNAs, for reducing FANCM expression may be readily designed using reference FANCM coding sequences and software tools which are widely available in the art and may be produced using routine techniques. For example, a suppressor nucleic acid may be chemically synthesized; produced recombinantly in vitro or cells (Elbashir, S. M. et al., Nature 411:494-498 (2001); Elbashir, S. M., et al., Genes & Development 15:188-200 (2001)) or obtained from commercial sources (e.g. Cruachem (Glasgow, UK), Dharmacon Research (Lafayette, Colo., USA)).

In some embodiments, two or more suppressor nucleic acids may be used to suppress the expression of FANCM. For example a pool of siRNAs may be employed. Suitable siRNAs and siRNA pools may be produced using standard techniques.

Nucleic acid suppression may also be carried out using anti-sense techniques. Anti-sense oligonucleotides may be designed to hybridise to the complementary sequence of nucleic acid, pre-mRNA or mature mRNA, interfering with the production of the base excision repair pathway component so that its expression is reduced or completely or substantially completely prevented. In addition to targeting coding sequence, anti-sense techniques may be used to target control sequences of a gene, e.g. in the 5′ flanking sequence, whereby the anti-sense oligonucleotides can interfere with expression control sequences. The construction of anti-sense sequences and their use is well known in the art (Peyman and Ulman, Chemical Reviews, 90:543-584, (1990); Crooke, Ann. Rev. Pharmacol. Toxicol. 32:329-376, (1992)).

Anti-sense oligonucleotides may be generated in vitro or ex vivo for administration or anti-sense RNA may be generated in vivo within the cancer cells in which down-regulation of FANCM is desired. Thus, double-stranded DNA may be placed under the control of a promoter in a “reverse orientation” such that transcription of the anti-sense strand of the DNA yields RNA which is complementary to normal mRNA transcribed from the sense strand of the target gene. The complementary anti-sense RNA sequence is thought then to bind with mRNA to form a duplex, inhibiting translation of the endogenous mRNA from the target gene into protein.

The complete sequence corresponding to the FANCM coding sequence in reverse orientation need not be used. For example, fragments of sufficient length may be used. It is a routine matter for the person skilled in the art to screen fragments of various sizes and from various parts of the coding or flanking sequences of a gene to optimise the level of anti-sense inhibition. It may be advantageous to include the initiating methionine ATG codon, and perhaps one or more nucleotides upstream of the initiating codon. A suitable fragment may have about 14-23 nucleotides, e.g. about 15, 16 or 17.

In other embodiments, targeted mutagenesis may be used to reduce the expression of active FANCM polypeptide. The use of targeted mutagenesis techniques such as gene editing, to knock out or abolish expression of target genes is well-established in the art (see for example Gaj et al (2013) Trends Biotechnol. 31(7) 397-405).

One or more mutations, such as insertions, substitutions or deletions, may be introduced into the FANCM gene in the cancer cells. Suitable mutations include deletions of all or part of the FANCM gene, for example, one, two or more exons, frameshift mutations, or nonsense mutations introducing premature stop codons. In some preferred embodiments, one or more premature stop codons may be introduced into the FANCM coding sequence. Preferably, mutations, such as premature stop codons, are introduced into the first 400 codons of the coding sequence, to eliminate the ATPase domain of FANCM. The mutations may prevent the expression of active FANCM polypeptide, for example by impairing transcription or translation of the FANCM gene or causing an inactive polypeptide to be expressed.

Targeted mutagenesis to introduce one or more mutations may be performed by any convenient method. For example, the cancer cells may be transfected with a heterologous nucleic acid which encodes a targetable nuclease. The targetable nuclease may inactivate the FANCM gene encoding FANCM in one or more cells of the individual, for example, by introducing one or more mutations that prevent the expression of active FANCM polypeptide.

The targetable nuclease may inactivate the FANCM gene encoding FANCM selectively in cancer cells of the individual. The targetable nuclease may be targeted to specifically to cancer cells by conventional techniques, including cell targeted delivery vehicles, such as viral vectors that express a ligand for a specific cell type; direct administration of the targetable nuclease to a tumour e.g. by injection; or the expression of the targetable nuclease from heterologous nucleic acid selectively in cancer cells, for example using a tissue specific promoter.

The targetable nuclease may be site-specific (e.g. ZFN or TALEN) or may be expressed with one or more targeting sequences that target the nuclease to the FANCM gene (e.g. CRISPR/Cas).

The heterologous nucleic acid encoding the targetable nuclease may include an inducible promoter that promotes expression of the targetable nuclease and optional targeting sequence within a specific cell type, for example a tumour cell. For example, the inducible promoter could be a promoter-enhancer cassette that selectively favours expression of the targetable nuclease and the optional targeting sequence within the tumour cell over other types of host cells.

Suitable targeting nucleases include, for example, site-specific nucleases, such as zinc-finger nucleases (ZFNs), transcription activator-like effector nucleases (TALENs), and meganucleases or RNA guided nucleases, such as clustered regularly interspaced short palindromic repeat (CRISPR) nucleases.

Zinc-finger nucleases (ZFNs) comprise one or more Cys₂-His₂ zinc-finger DNA binding domains and a cleavage domain (i.e., nuclease). The DNA binding domain may be engineered to recognize and bind to any nucleic acid sequence using conventional techniques (see for example Qu et al. (2013) Nucl Ac Res 41(16):7771-7782). The use of ZFNs to introduce mutations into target genes is well-known in the art (see for example, Beerli et al Nat. Biotechnol. 2002; 20:135-141; Maeder et al Mol. Cell. 2008; 31:294-301; Gupta et al Nat. Methods. 2012; 9:588-590) and engineered ZFNs are commercially available (Sigma-Aldrich (St. Louis, Mo.).

Transcription activator-like effector nucleases (TALENs) comprise a nonspecific DNA-cleaving nuclease fused to a DNA-binding domain comprising a series of modular TALE repeats linked together to recognise a contiguous nucleotide sequence. The use of TALEN targeting nucleases is well known in the art (e.g. Joung & Sander (2013) Nat Rev Mol Cell Bio 14:49-55; Kim et al Nat Biotechnol. (2013); 31:251-258. Miller J C, et al. Nat. Biotechnol. (2011) 29:143-148. Reyon D, et al. Nat. Biotechnol. (2012); 30:460-465).

Meganucleases are endodeoxyribonucleases characterized by a large recognition site (double-stranded DNA sequences of 12 to 40 base pairs); as a result this site generally occurs only once in any given genome (see for example Silva et al. (2011) Curr Gene Ther 11(1):11-27).

CRISPR targeting nucleases (e.g. Cas9) complex with a guide RNA (gRNA) to cleave genomic DNA in a sequence-specific manner. The crRNA and tracrRNA of the guide RNA may be used separately or may be combined into a single RNA to enable site-specific mammalian genome cutting within the FANCM gene or its regulatory elements. The use of CRISPR/Cas9 systems to introduce insertions or deletions into genes as a way of decreasing transcription is well known in the art (see for example Cader et al Nat Immunol 2016 17 (9) 1046-1056, Hwang et al. (2013) Nat. Biotechnol 31:227-229; Xiao et al., (2013) Nucl Acids Res 1-11; Horvath et al., Science (2010) 327:167-170; Jinek M et al. Science (2012) 337:816-821; Cong L et al. Science (2013) 339:819-823; Jinek M et al. (2013) eLife 2:e00471; Mali P et al. (2013) Science 339:823-826; Qi L S et al. (2013) Cell 152:1173-1183; Gilbert L A et al. (2013) Cell 154:442-451; Yang H et al. (2013) Cell 154:1370-1379; and Wang H et al. (2013) Cell 153:910-918).

In some preferred embodiments, the targetable nuclease is a Cas endonuclease, preferably Cas9, which is expressed in the cancer cells in combination with a guide RNA targeting sequence that targets the Cas endonuclease to cleave genomic DNA within the FANCM gene and generate insertions or deletions that prevent expression of active FANCM polypeptide.

Nucleic acid sequences encoding a suppressor nucleic acid or targetable nuclease and optionally a guide RNA may be comprised within an expression vector. Suitable vectors can be chosen or constructed, containing appropriate regulatory sequences, including promoter sequences, terminator fragments, polyadenylation sequences, enhancer sequences, marker genes and other sequences as appropriate. Preferably, the vector contains appropriate regulatory sequences to drive the expression of the encoding nucleic acid in a host cell. Suitable regulatory sequences to drive the expression of heterologous nucleic acid coding sequences in a range of expression systems are well-known in the art and include constitutive promoters, for example viral promoters such as CMV or SV40. In some preferred embodiments, a tissue-specific or inducible promoter, such as a photoinducible promoter, may be employed to selectively express the suppressor nucleic acid or targetable nuclease and optionally guide RNA in cancer cells. A vector may also comprise sequences, such as origins of replication and selectable markers, which allow for its selection and replication and expression in bacterial hosts, such as E. coli and/or in eukaryotic cells, such as yeast, insect or mammalian cells. Vectors suitable for use in expressing a suppressor nucleic acid or targetable nuclease in mammalian cells include plasmids and viral vectors e.g. retroviruses, lentiviruses, adenoviruses, and adeno-associated viruses. Suitable techniques for expressing a suppressor nucleic acid or targetable nuclease in mammalian cells are well known in the art (see for example; Molecular Cloning: a Laboratory Manual: 3rd edition, Russell et al., 2001, Cold Spring Harbor Laboratory Press or Protocols in Molecular Biology, Second Edition, Ausubel et al. eds. John Wiley & Sons, 1992; Recombinant Gene Expression Protocols Ed R S Tuan (March 1997) Humana Press Inc).

Identification of FANCM Inhibitors

Other aspects of the invention relate to the use of FANCM to screen for compounds that increase cell death or reduce the viability of ALT cells and are potentially useful in the treatment of ALT cancer.

Screening methods may be used to identify test compounds that bind to an isolated FANCM protein. A method of screening for a compound that reduces viability or increases cell death in ALT cells may comprise determining the binding of a test compound to FANCM. For example, a test compound may be contacted with FANCM and the binding of the test compound to FANCM determined. Binding between the test compound and FANCM may be indicative that the test compound reduces viability or increases cell death in ALT cells.

The binding of a test compound to FANCM may be determined by standard techniques, such as surface plasmon resonance (SPR).

In some embodiments, the ability of a test compound to inhibit the interaction of FANCM with a binding partner may be determined. A method of screening for a compound that reduces viability or increases cell death in ALT cells may comprise determining the effect of a test compound on the binding of FANCM to a binding partner. For example, FANCM may be contacted with a binding partner in the presence and absence of a test compound. A reduction in binding between FANCM and the binding partner in the presence relative to the absence of test compound may be indicative that the test compound reduces viability or increases cell death in ALT cells.

Binding partners are proteins that naturally bind to FANCM within a cell, for example during homologous recombination, meiosis and DNA repair. FANCM binding partners include MHF, FAAP24, HCLK2, BLM, RMI, Topo IIIα.

Screening methods may be used to identify test compounds that inhibit the activity of FANCM. A method of screening for a compound that reduces viability or increases cell death in ALT cells may comprise determining the effect of a test compound on the activity of FANCM. For example, the activity of FANCM may be determined in the presence and absence of a test compound. A decrease in activity of the FANCM in the presence relative to the absence of the test compound being indicative that the compound reduces viability or increases cell death in ALT cells.

The ATP-dependent DNA helicase/translocase activity of the FANCM may be determined in the presence relative to the absence of test compound. A decrease or reduction in ATP-dependent DNA helicase/translocase activity in the presence of the test compound may be indicative that the test compound inhibits the activity of FANCM protein. For example, the test compound may be a FANCM inhibitor. Suitable methods of determining activity, including ATPase and translocase assays, are well known in the art.

The precise format of any of the screening or assay methods of the present invention may be varied by those of skill in the art using routine skill and knowledge. The skilled person is well aware of the need to employ appropriate control experiments.

FANCM for use in screening methods may be an isolated polypeptide comprising the full-length FANCM sequence, for example a FANCM reference sequence, such as SEQ ID NO:1 as set out herein, or a fragment thereof. Suitable fragments may include at least 50, at least 100 or at least 150 contiguous amino acids from a FANCM reference sequence. In some embodiments, FANCM fragments comprising the ATPase or translocase activity may be employed, for example a fragment comprising or consisting of the N terminal DEAH helicase-like domain corresponding to residues 83-591 of SEQ ID NO: 1. Isolated FANCM polypeptides may be produced using standard recombinant techniques.

A test compound may be an isolated molecule or may be comprised in a sample, mixture or extract, for example, a biological sample. Compounds which may be screened using the methods described herein may be natural or synthetic chemical compounds used in drug screening programmes and may include, for example, small organic molecules, polypeptides and nucleic acids, such as aptamers. Extracts of plants, microbes or other organisms, which contain several characterised or uncharacterised components may also be used.

Suitable test compounds for screening include compounds that inhibit similar activities to the ATP-dependent DNA helicase/translocase activity of FANCM. For example, suitable test compounds may be ATP analogues. Suitable test compounds may be produced using rational drug design to provide test candidate compounds with particular molecular shape, size and charge characteristics suitable for modulating FANCM activity.

Combinatorial library technology provides an efficient way of testing a potentially vast number of different compounds for ability to modulate FANCM activity. Such libraries and their use are known in the art, for all manner of natural products, small molecules and peptides, among others. The use of peptide libraries may be preferred in certain circumstances. In some embodiments, libraries of biological molecules, such as aptamers or antibody molecules.

The amount of test compound which may be added to an assay of the invention will normally be determined by trial and error depending upon the type of compound used. Typically, from about 0.001 nM to 1 mM or more concentrations of putative inhibitor compound may be used, for example from 0.01 nM to 100 μM, e.g. 0.1 to 50 μM, such as about 10 μM. Even a compound which has a weak effect may be a useful lead compound for further investigation and development.

Test compounds may include peptides derived from FANCM or binding partners thereof as described above. Membrane permeable peptide fragments of from 5 to 40 amino acids, for example, from 6 to 10 amino acids may be tested for their ability to bind to FANCM or inhibit its activity. The modulatory properties of a peptide above may be increased by the addition of one of the following groups to the C terminal: chloromethyl ketone, aldehyde and boronic acid. These groups are transition state analogues for serine, cysteine and threonine proteases. The N terminus of a peptide fragment may be blocked with carbobenzyl to inhibit aminopeptidases and improve stability (Proteolytic Enzymes 2nd Ed, Edited by R. Beynon and J. Bond, Oxford University Press, 2001).

Test compounds may include antibodies, antibody fragments and antibody derivatives and non-immunoglobulin binding molecules, such as aptamers, trinectins, anticalins, kunitz domains, transferrins, nurse shark antigen receptors and sea lamprey leucine-rich repeat proteins. Suitable molecules may be directed to the DEAH helicase-like domain corresponding to residues 83-591 of SEQ ID NO: 1 or another part of the FANCM protein. Candidate modulatory antibody molecules may be characterised and their binding regions determined to provide single chain antibodies and fragments thereof which are responsible for inhibiting activity or blocking interactions with binding partners. Suitable antibodies may be obtained using techniques which are standard in the art, including, for example immunising a mammal with a suitable peptide, such as a fragment of the pro-inflammatory polypeptide, or isolating a specific antibody from a recombinantly produced library of expressed immunoglobulin variable domains, e.g. using lambda bacteriophage or filamentous bacteriophage which display functional immunoglobulin binding domains on their surfaces; for instance see WO92/01047.

Aptamers directed to FANCM are also putative agents for modulating FANCM. Aptamers are nucleic acids that specifically bind to a target molecule. Typically aptamers are small nucleic acids ranging from 15-50 bases in length that fold into defined secondary and tertiary structures, such as stem-loops or G-quartets. Aptamers can bind very tightly with k_{d} for the target molecule of less than 10⁻¹² M. Aptamers may bind FANCM with a very high degree of specificity. For example, aptamers have been isolated that have greater than a 10000 fold difference in binding affinities between a target molecule and another molecule that differ at only a single position on the molecule. An aptamer may have a k_{d} with FANCM of at least 10, 100, 1000, 10,000, or 100,000 fold lower than the k_{d} with a control polypeptide. The production and use of aptamers is well known in the art (see for example Bunka et al Curr Opin Pharmacol 2010 10 (5) 557-562).

A test compound identified as inhibiting FANCM activity may be investigated further using one or more secondary screens. A secondary screen may involve testing for a biological function or activity in vitro and/or in vivo, e.g. in an animal model. For example, the ability of a test compound to reduce viability or increase cell death of ALT cells may be determined. In some embodiments, a secondary screen may involve determining the selectivity of a compound for FANCM by screening against a panel of isolated enzymes.

The effect of a test compound identified as an FANCM inhibitor may be determined in vitro on mammalian cells. For example, the effect of the test compound on an ALT cell line may be determined. Increased ALT cell death in the presence relative to the absence of the compound may be indicative that the compound displays an activity useful in the treatment of ALT cancer.

Following identification of a FANCM inhibitor that is potentially useful in the treatment of ALT cancer as described herein, a method may further comprise modifying the compound to optimise its pharmaceutical properties. Suitable methods of optimisation, for example by structural modelling, are well known in the art. Further optimisation or modification can then be carried out to arrive at one or more final compounds for in vivo or clinical testing.

A test compound identified as a FANCM inhibitor may be isolated and/or purified or alternatively, it may be synthesised using conventional techniques of recombinant expression or chemical synthesis. Furthermore, it may be manufactured and/or used in preparation, i.e. manufacture or formulation, of a composition such as a medicament, pharmaceutical composition or drug. Methods described herein may thus comprise formulating the test compound in a pharmaceutical composition with a pharmaceutically acceptable excipient, vehicle or carrier for therapeutic application.

Pharmaceutical Compositions

Whilst a therapeutic agent described herein, such as a FANCM antagonist, inhibitor, suppressor nucleic acid, targetable nuclease, nucleic acid encoding a suppressor nucleic acid or targetable nuclease, may be administered alone, the therapeutic agent will usually be administered in the form of a pharmaceutical composition, which may comprise at least one component in addition to the active agent. A therapeutic agent may be admixed with other reagents, such as buffers, carriers, diluents, preservatives and/or pharmaceutically acceptable excipients in order to produce a pharmaceutical composition for use in cancer immunotherapy. Suitable reagents are described in more detail below.

Aspects of the invention provide (i) a pharmaceutical composition comprising a therapeutic agent selected from: (a) a FANCM inhibitor (b) a FANCM suppressor nucleic acid, (c) a FANCM targetable nuclease, or (d) a nucleic acid encoding a FANCM suppressor nucleic acid or targetable nuclease, and a pharmaceutically acceptable excipient; and (ii) a method of producing a pharmaceutical composition for use in cancer immunotherapy comprising admixing a therapeutic agent as described above with a pharmaceutically acceptable excipient.

Aspects of the invention provide (i) a pharmaceutical composition comprising a therapeutic agent selected from: (a) a RMI inhibitor (b) a RMI suppressor nucleic acid, (c) a RMI targetable nuclease, or (d) a nucleic acid encoding a RMI suppressor nucleic acid or targetable nuclease, and a pharmaceutically acceptable excipient; and (ii) a method of producing a pharmaceutical composition for use in cancer immunotherapy comprising admixing a therapeutic agent as described above with a pharmaceutically acceptable excipient.

Aspects of the invention provide (i) a pharmaceutical composition comprising a therapeutic agent that inhibits binding of FANCM to RMI selected from: (a) a small molecule inhibitor (b) a polypeptide inhibitor, or (c) a nucleic acid inhibitor, and a pharmaceutically acceptable excipient and (ii) a method of producing a pharmaceutical composition for use in cancer immunotherapy comprising admixing a therapeutic agent as described above with a pharmaceutically acceptable excipient.

Aspects of the invention provide a pharmaceutical composition comprising a therapeutic agent that inhibits binding of FANCM to RMI and a therapeutic agent that inhibits FANCM ATPase activity. In particular embodiments, the therapeutic agents are selected from: (a) a small molecule inhibitor (b) a polypeptide inhibitor, or (c) a nucleic acid inhibitor, and a pharmaceutically acceptable excipient. In certain embodiments, the disclosure provides a method of producing a pharmaceutical composition for use in cancer immunotherapy comprising admixing the therapeutic agents as described above with a pharmaceutically acceptable excipient.

The term “composition” as used herein is intended to encompass a product comprising the specified ingredients in the specified amounts, as well as any product which results, directly or indirectly, from combination of the specified ingredients in the specified amounts.

The term “pharmaceutically acceptable” as used herein pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

The present disclosure also provides a pharmaceutical composition comprising an inhibitor of the FANCM-RMI interaction for use in treating ALT cancer. The present disclosure also provides the use of an inhibitor of the FANCM-RMI interaction in the manufacture of a medicament for the treatment of ALT cancer. In one example, the pharmaceutical composition or medicament consists essentially of an inhibitor of the FANCM-RMI interaction. The present disclosure also provides a pharmaceutical composition comprising an inhibitor of FANCM ATPAse activity for use in treating ALT cancer. The present disclosure also provides the use of an inhibitor of FANCM ATPAse activity in the manufacture of a medicament for the treatment of ALT cancer. The present disclosure also provides a pharmaceutical composition comprising an inhibitor of the FANCM-RMI interaction and an inhibitor of FANCM ATPAse activity for use in treating ALT cancer. The present disclosure also provides the use of an inhibitor of the FANCM-RMI interaction and an inhibitor of FANCM ATPAse activity in the manufacture of a medicament for the treatment of ALT cancer. In one example, the pharmaceutical composition or medicament consists essentially of an inhibitor of the FANCM-RMI interaction. In one example, the pharmaceutical composition or medicament consists essentially of an inhibitor of FANCM ATPAse activity. In one example, the pharmaceutical composition or medicament consists essentially of an inhibitor of FANCM ATPAse activity and an inhibitor of FANCM ATPAse activity. However, the medicament or the composition may also include excipients or agents such as solvents, diluents, dispersion media, coatings, antibacterial and antifungal agents, isotonic and absorption delaying agents and the like that are physiologically compatible and are not deleterious to the inhibitor as described herein or use thereof. The use of such carriers and agents to prepare compositions of pharmaceutically active substances is well known in the art (see, for example Remington: The Science and Practice of Pharmacy, 21^{st} Edition; Lippincott Williams & Wilkins: Philadelphia, Pa., 2005).

Pharmaceutical compositions suitable for administration (e.g. by infusion), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents. Examples of suitable isotonic vehicles for use in such formulations include Sodium Chloride Injection, Ringer's Solution, or Lactated Ringer's Injection. Suitable vehicles can be found in standard pharmaceutical texts, for example, Remington's Pharmaceutical Sciences, 18th edition, Mack Publishing Company, Easton, Pa., 1990.

The pharmaceutical composition may be diluted prior to use. Suitable diluents may be selected from, for example: Ringer's solution, Hartmann's solution, dextrose solution, saline solution and sterile water for injection.

The pharmaceutical compositions include those for oral, rectal, nasal, topical (including buccal and sub-lingual), parenteral administration (including intramuscular, intraperitoneal, sub-cutaneous and intravenous), or in a form suitable for administration by inhalation or insufflation. The inhibitor of the FANCM-RMI interaction, together with a conventional adjuvant, carrier or diluent, may be placed into the form of pharmaceutical compositions and unit dosages thereof, and in such form may be employed as solids, such as tablets or filled capsules, or liquids as solutions, suspensions, emulsions, elixirs or capsules filled with the same, all for oral use, or in the form of sterile injectable solutions for parenteral (including subcutaneous) use.

The pharmaceutical compositions for the administration of the antagonists or inhibitors of this disclosure may conveniently be presented in dosage unit form and may be prepared by any of the methods well known in the art of pharmacy.

The pharmaceutical compositions and methods disclosed herein may further comprise other therapeutically active compounds that are usually applied in the treatment of the disclosed disorders or conditions. Selection of the appropriate agents for use in combination therapy may be made by one of ordinary skill in the art, according to conventional pharmaceutical principles. The combination of therapeutic agents may act synergistically to effect the treatment or prevention of the various disorders or conditions disclosed herein. Using this approach, one may be able to achieve therapeutic efficacy with lower dosages of each agent, thus reducing the potential for adverse side effects.

When other therapeutic agents are employed in combination with those disclosed herein, they may be used for example in amounts as noted in the Physician Desk Reference (PDR) or as otherwise determined by one of ordinary skill in the art.

Methods

In certain embodiments, the disclosure provides methods for inhibiting FANCM, methods for inhibiting RMI, methods for inhibiting or disrupting the FANCM-RMI interaction, and methods for inhibiting the ATPase and/or translocase activity of FANCM, e.g., in ALT cells, e.g., ALT cancer or tumor cells. In particular embodiments of any such methods, the methods inhibit ALT cell viability and/or growth, e.g., ALT cancer or tumor cell viability or growth. In particular embodiments, the methods increase or induce death of ALT cells, e.g., ALT cancer or tumor cells. The methods may be practiced in vitro or in vivo, e.g., to treat a subject with an ALT cancer or tumor. In certain embodiments, a subject is a mammal, e.g., a human diagnosed with an ALT cancer.

With respect to “inhibiting” an activity of FANCM, such as binding to RMI, DNA translocase activity, ATPase activity and/or translocase activity, the inhibitor may be partial or complete. In certain embodiments, inhibition is a reduction of at least 20%, at least 30%, at least 40%, at least 50%, at least 60%, at least 70%, at least 75%, at least 80%, at least 85%, at least 90%, at least 95%, or about 100%, e.g., as compared to the amount in a comparable cell not contacted with the FANCM antagonist or inhibitor, or as compared to a pre-determined value.

Thus, in one embodiment, the disclosure provides a method of inhibiting ALT cell viability and/or growth, comprising contacting an ALT cell (e.g., an ALT cancer cell) with an antagonist or inhibitor of FANCM. In particular embodiments, the antagonist or inhibitor inhibits expression of FANCM in the ALT cell. In particular embodiments, the antagonist or inhibitor inhibits one or more FANCM biological activity in the ALT cell. In certain embodiments, the biological activity is FANCM's DNA translocase activity. In certain embodiments, the biological activity is FANCM's ATPase and/or translocase activity. In certain embodiments, the biological activity is mediated by FANCM's binding to the BLM-TOP3-RMI (BTR) complex. In certain embodiments, the biological activity is mediated by FANCM's ATP-dependent translocase activity (DEAH domain). In certain embodiments, the method comprising contacting an ALT cell (e.g., an ALT cancer cell) with one or more antagonist or inhibitor of FANCM, where the one or more antagonist or inhibitor of FANCM collectively inhibit both FANCM's DNA translocase activity and FANCM's ATPase and/or translocase activity). In certain embodiments, the method comprising contacting an ALT cell (e.g., an ALT cancer cell) with one or more antagonist or inhibitor of FANCM, where the one or more antagonist or inhibitor of FANCM collectively disrupts the FANCM-RMI interaction and inhibit FANCM's ATPase/translocase activity. The inhibitor that disrupts the FANCM-RMI interaction and the inhibitor that inhibits FANCM's ATPase and/or translocase activity may be the same inhibitor or different inhibitors.

In certain contexts used herein, the term “inhibit ALT cell viability and/or growth” shall be taken to mean hinder, reduce, restrain or prevent ALT cell viability and/or growth relative to an ALT cell in which the FANCM-RMI interaction is intact. Likewise, in certain contexts used herein, the term “inhibit ALT cell viability and/or growth” shall be taken to mean hinder, reduce, restrain or prevent ALT cell viability and/or growth relative to an ALT cell in which FANCM is not inhibited. In certain contexts used herein, the term “inhibit ALT cell viability and/or growth” shall be taken to mean hinder, reduce, restrain or prevent ALT cell viability and/or growth relative to an ALT cell in which RMI is not inhibited. In certain contexts used herein, the term “inhibit ALT cell viability and/or growth” shall be taken to mean hinder, reduce, restrain or prevent ALT cell viability and/or growth relative to an ALT cell in which the FANCM-RMI interaction is not disrupted.

Cell viability and/or growth may be inhibited in any measurable amount. Inhibition of cell viability may be complete or may be partial. Thus, the methods disclosed herein may comprise at least partial inhibition of ALT cell viability and/or growth. For example, cell viability and/or growth may be reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or 100% following disruption of the FANCM-RMI interaction.

Thus, in one embodiment, the disclosure provides a method of inducing ALT cell death, comprising contacting an ALT cell (e.g., an ALT cancer cell) with an antagonist or inhibitor of FANCM. In particular embodiments, the antagonist or inhibitor inhibits expression of FANCM in the ALT cell. In particular embodiments, the antagonist or inhibitor inhibits one or more FANCM biological activity in the ALT cell. In certain embodiments, the biological activity is FANCM's DNA translocase activity. In certain embodiments, the biological activity is FANCM's ATPase and/or translocase activity. In certain embodiments, the biological activity is mediated by FANCM's binding to the BLM-TOP3-RMI (BTR) complex. In certain embodiments, the biological activity is mediated by FANCM's ATP-dependent translocase activity (DEAH domain). In certain embodiments, the method comprising contacting an ALT cell (e.g., an ALT cancer cell) with one or more antagonist or inhibitor of FANCM, where the one or more antagonist or inhibitor of FANCM collectively inhibit both FANCM's DNA translocase activity and FANCM's ATPase and/or translocase activity. In certain embodiments, the method comprising contacting an ALT cell (e.g., an ALT cancer cell) with one or more antagonist or inhibitor of FANCM, where the one or more antagonist or inhibitor of FANCM collectively disrupts the FANCM-RMI interaction and inhibit FANCM's ATPase and/or translocase activity. The inhibitor that disrupts the FANCM-RMI interaction and the inhibitor that inhibits FANCM's ATPase and/or translocase activity may be the same inhibitor or different inhibitors.

In certain contexts used herein, the term “induce ALT cell death” shall be taken to mean induce, increase, cause, or promote ALT cell death relative to an ALT cell in which the FANCM-RMI interaction is intact. Likewise, in certain contexts used herein, the term “induce ALT cell death” shall be taken to mean induce, increase, cause, or promote ALT cell death relative to an ALT cell in which FANCM is not inhibited. In certain contexts used herein, the term “induce ALT cell death” shall be taken to mean induce, increase, cause, or promote ALT cell death relative to an ALT cell in which RMI is not inhibited. In certain contexts used herein, the term “induce ALT cell death” shall be taken to mean induce, increase, cause, or promote ALT cell viability and/or growth relative to an ALT cell in which the FANCM-RMI interaction is not disrupted.

Cell death may be increased in any measurable amount. Increasing cell death may be complete or may be partial. Thus, the methods disclosed herein may comprise at least partial inducement of ALT cell death. For example, ALT cell death may be increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or 100% following disruption of the FANCM-RMI interaction. Cell death may be determined in a plurality of cells, e.g., as a percentage of non-viable or dead cells following contact with an antagonist or inhibitor disclosed herein. In certain embodiments, the plurality of cells are ALT tumor cells.

Thus, in one embodiment, the disclosure provides a method to inhibit an ALT cancer in a subject, comprising contacting the subject with an antagonist or inhibitor of FANCM. In particular embodiments, the antagonist or inhibitor inhibits expression of FANCM in the ALT cell. In particular embodiments, the antagonist or inhibitor inhibits one or more FANCM biological activity in the ALT cell. In certain embodiments, the biological activity is FANCM's DNA translocase activity. In certain embodiments, the biological activity is FNCM's ATPase and/or translocase activity. In certain embodiments, the biological activity is mediated by FANCM's binding to the BLM-TOP3-RMI (BTR) complex. In certain embodiments, the biological activity is mediated by FANCM's ATP-dependent translocase activity (DEAH domain). In certain embodiments, the method comprising contacting the subject with one or more antagonist or inhibitor of FANCM, where the one or more antagonist or inhibitor of FANCM collectively inhibit both FANCM's DNA translocase activity and FANCM's ATPase and/or translocase activity. In certain embodiments, the method comprising contacting an ALT cell (e.g., an ALT cancer cell) with one or more antagonist or inhibitor of FANCM, where the one or more antagonist or inhibitor of FANCM collectively disrupts the FANCM-RMI interaction and inhibit FANCM's ATPase and/or translocase activity. The inhibitor that disrupts the FANCM-RMI interaction and the inhibitor that inhibits FANCM's ATPase and/or translocase may be the same inhibitor or different inhibitors. In particular embodiments, the ALT cancer is an osteosarcoma, soft tissue sarcoma (e.g. liposarcoma, undifferentiated pleomorphic sarcoma, or leiomyosarcoma), glioblastoma, astrocytoma, neuroblastoma, or bladder carcinoma.

In certain contexts used herein, the term “inhibit an ALT cancer” shall be taken to mean inhibit the growth or metastasis of an ALT cancer or tumor, reduce the size of an ALT cancer or tumor, reduce the growth rate of an ALT cancer or tumor relative to an ALT cell in which the FANCM-RMI interaction is intact. Likewise, in certain contexts used herein, the term “inhibit an ALT cancer” shall be taken to mean hinder, reduce, restrain or prevent ALT cell viability and/or growth relative to an ALT cell in which FANCM is not inhibited. In certain contexts used herein, the term “inhibit an ALT cancer” shall be taken to mean hinder, reduce, restrain or prevent ALT cell viability and/or growth relative to an ALT cell in which RMI is not inhibited. In certain contexts used herein, the term “inhibit an ALT cancer” shall be taken to mean hinder, reduce, restrain or prevent ALT cell viability and/or growth relative to an ALT cell in which the FANCM-RMI interaction is not disrupted.

Inhibition of an ALT cancer may occur in any measurable amount. Inhibition of an ALT cancer may be complete or may be partial. Thus, the methods disclosed herein may comprise at least partial inhibition of growth or metastasis of an ALT cancer or tumor, at least partial reduction in the size of an ALT cancer or tumor, or at least partial reduction in the growth rate of an ALT cancer or tumor.

Inhibition of FANCM or RMI may be accomplished by any suitable means in the art, including use of any of the FANCM and RMI inhibitors disclosed herein. The interaction between FANCM and RMI may be disrupted by any suitable means known in the art, including use of any of the inhibitors or disruptors of the FANCM-RMI disclosed herein. Inhibition of FANCM, RMI or disruption of the FANCM-RMI interaction may be partial or complete. FANCM activity may be measured through the C-circle assay or through any suitable means known in the art, or any of the methods disclosed herein.

In certain embodiments of any of the methods disclosed herein, the method may not comprise also inhibiting one of BLM and/or Breast cancer type 1 susceptibility protein (BRCA1). In certain embodiments of any of the methods disclosed herein, the method may not comprise also inhibiting BLM. In certain embodiments of any of the methods disclosed herein, the method may not comprise also inhibiting BRCA1. The sequence of BRCA1 is publicly available. An exemplary sequence is set forth in SEQ ID NO: 8.

Agents that reduce FANCM expression or activity are shown herein to induce G2/M arrest and cell death in ALT cancer cells and may therefore be useful in the treatment of ALT cancer.

As used herein, the terms “treating”, “treat” or “treatment” and variations thereof, refer to clinical intervention designed to alter the natural course of the individual or cell being treated during the course of clinical pathology. The term “treatment”, as used herein in the context of treating a condition, pertains generally to treatment and therapy in which some desired therapeutic effect is achieved, for example, the inhibition of the progress of the condition, and includes a reduction in the rate of progress, a halt in the rate of progress and amelioration of the condition, and cure of the condition. Desirable effects of treatment include decreasing the rate of disease progression, reducing size of the cancer, inhibiting tumour growth, inhibiting cancer progression or metastasis, ameliorating or palliating the disease state, and remission or improved prognosis.

Treatment may be any treatment and therapy, whether of a human or an animal (e.g. in veterinary applications), in which some desired therapeutic effect is achieved, for example, the inhibition or delay of the progress of the condition, and includes a reduction in the rate of progress, a halt in the rate of progress, amelioration of the condition, cure or remission (whether partial or total) of the condition, preventing, delaying, abating or arresting one or more symptoms and/or signs of the condition or prolonging survival of a subject or patient beyond that expected in the absence of treatment.

Treatment as a prophylactic measure (i.e. prophylaxis) is also included. For example, an individual susceptible to or at risk of the occurrence or re-occurrence of cancer may be treated as described herein. Such treatment may prevent or delay the occurrence or re-occurrence of cancer in the individual.

In particular, treatment may include inhibiting cancer growth, including complete cancer remission, and/or inhibiting cancer metastasis. Cancer growth generally refers to any one of a number of indices that indicate change within the cancer to a more developed form. Thus, indices for measuring an inhibition of cancer growth include a decrease in cancer cell survival, a decrease in tumor volume or morphology (for example, as determined using computed tomographic (CT), sonography, or other imaging method), a delayed tumor growth, a destruction of tumor vasculature, improved performance in delayed hypersensitivity skin test, an increase in the activity of cytolytic cancer cells, and a decrease in levels of tumor-specific antigens.

As used herein, the term “subject” refers to any animal, for example, a mammalian animal, including, but not limited to humans, non-human primates, livestock (e.g. sheep, horses, cattle, pigs, donkeys), companion animals (e.g. pets such as dogs and cats), laboratory test animals (e.g. mice, rabbits, rats, guinea pigs), performance animals (e.g. racehorses, camels, greyhounds) or captive wild animals. In one embodiment, the “subject” is a human. In particular embodiments, a subject or an individual suitable for treatment with a therapeutic agent, such as (a) a FANCM antagonist or inhibitor (b) FANCM suppressor nucleic acid, (c) FANCM targetable nuclease, or (d) nucleic acid encoding a FANCM suppressor nucleic acid or targetable nuclease, as described herein, may be a mammal, such as a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orangutan, gibbon), or a human. Typically, the terms “subject” and “patient” are used interchangeably, particularly in reference to a human subject. The subject may be receiving simultaneous, sequential or separate administration of a chemotherapeutic agent. The subject may be a subject suffering from, suspected of suffering from, or predisposed to, cancer. The cancer may be any cancer disclosed herein.

The inventors have surprisingly shown for the first time that disruption of the FANCM-RMI complex is selectively toxic to ALT cancer cells. Based on this finding, the inventors have developed and provide herein (i) methods of inhibiting ALT cell viability and/or growth, (ii) methods of treating ALT cancer, (iii) methods of selecting a subject for treatment or identifying whether a subject suffering from cancer is suitable for treatment with an inhibitor of the FANCM-RMI interaction and (iv) methods of determining whether a subject is responding to treatment with an inhibitor of the FANCM-RMI interaction.

In certain embodiments, the present disclosure provides a method of treating ALT cancer in a subject, comprising disrupting the FANCM-RMI interaction. In particular embodiments, the method comprises providing to a subject having an ALT cancer, an effective amount of an inhibitor that disrupts the FANCM-RMI interaction. In particular embodiments, the ALT cancer is any of those disclosed herein. In particular embodiments, the ALT cancer is an osteosarcoma, soft tissue sarcoma (e.g. liposarcoma, undifferentiated pleomorphic sarcoma, or leiomyosarcoma), glioblastoma, astrocytoma, neuroblastoma, or bladder carcinoma. In certain embodiments, the method comprises providing to the subject one or more antagonist or inhibitor of FANCM, where the one or more antagonist or inhibitor of FANCM collectively inhibit both FANCM's DNA translocase activity and FANCM's ATPase and/or translocase activity.

In certain embodiments, the present disclosure provides a method of treating ALT cancer in a subject, comprising inhibiting FANCM ATPAse activity. In particular embodiments, the method comprises providing to a subject having an ALT cancer, an effective amount of an FANCM ATPase inhibitor. In particular embodiments, the ALT cancer is any of those disclosed herein. In particular embodiments, the ALT cancer is an osteosarcoma, soft tissue sarcoma (e.g. liposarcoma, undifferentiated pleomorphic sarcoma, or leiomyosarcoma), glioblastoma, astrocytoma, neuroblastoma, or bladder carcinoma.

In one example, the present disclosure provides a method of treating ALT cancer comprising disrupting the FANCM-RMI interaction and/or inhibiting FANCM ATPase activity, wherein the subject is receiving simultaneous administration of a chemotherapeutic agent. In another example, the present disclosure provides a method of treating ALT cancer comprising disrupting the FANCM-RMI interaction, wherein subject is receiving sequential administration of a chemotherapeutic agent. In another example, the present disclosure provides a method of treating ALT cancer comprising disrupting the FANCM-RMI interaction and/or inhibiting FANCM ATPase activity, wherein the subject is receiving separate administration of a chemotherapeutic agent. In particular embodiments, the ALT cancer is any of those disclosed herein. In particular embodiments, the ALT cancer is an osteosarcoma, soft tissue sarcoma (e.g. liposarcoma, undifferentiated pleomorphic sarcoma, or leiomyosarcoma), glioblastoma, astrocytoma, neuroblastoma, or bladder carcinoma.

In one example, the present disclosure provides a method of treating ALT cancer comprising disrupting the FANCM-RMI interaction and/or inhibiting FANCM ATPase activity, wherein the subject is not receiving simultaneous, sequential or separate administration of a chemotherapeutic agent. Thus, the methods disclosed herein may comprise disrupting the FANCM-RMI interaction and/or inhibiting FANCM ATPase activity as the sole therapeutic modality, or the sole anti-cancer therapeutic modality. In particular embodiments, the ALT cancer is any of those disclosed herein. In particular embodiments, the ALT cancer is an osteosarcoma, soft tissue sarcoma (e.g. liposarcoma, undifferentiated pleomorphic sarcoma, or leiomyosarcoma), glioblastoma, astrocytoma, neuroblastoma, or bladder carcinoma.

Any chemotherapeutic agents approved for the treatment of cancer are suitable for optional use in combination with any of the inhibitors disclosed herein, or in combination with disrupting the FANCM-RMI interaction as disclosed herein. Examples of suitable chemotherapeutic agents include, but are not limited to, paclitaxel, doxorubicin, carboplatin, cyclophosphamide, daunorubicin, doxorubicin, epirubicin, fluorouracil, gemcitabine, eribulin, ixabepilone, methotrexate, mutamycin, mitoxantrone, vinorelbine, docetaxel, thiotepa, vincristine and capecitabine.

In certain embodiments of any of the methods disclosed herein, the method comprises treating a subject having an ALT cancer. Any of the methods described herein may also comprise identifying or diagnosing a cancer in an individual as an ALT cancer. This may be accomplished, e.g., by obtaining such a diagnosis from a physician, hospital, or diagnostic laboratory. It may also be accomplished by performing an assay on a biological sample obtained from the subject, e.g., a tumor sample, and may comprise any of the various assays disclosed herein. For example, the presence of C-circles or ALT associated PML bodies in cancer cells from the individual may be determined. Suitable methods for the identification of ALT cancers are well known in the art⁷⁵.

As discussed herein, methods for determining whether a subject is suffering from ALT cancer are known in the art. Suitable assays include, but are not limited to, the measurement of C-circles (e.g., as disclosed in Henson et al., 2009 and WO2011035375), quantitative PCR of telomeric DNA and C-circles (Lau et al., 2012), the absence of telomerase activity, the presence of very long and heterogeneous telomeres, the presence of ALT-associated PML bodies (APBs), elevated telomeric sister chromatid exchange (T-SCE) (for example, which is elevated relative to a non-ALT cell, such as a telomerase positive cell or a mortal cell) and the presence of extrachromosomal telomeric repeat (ECTR) DNA. Any of the methods disclosed herein for identifying an ALT cell may be used to determine whether a subject is suffering from ALT cancer. These methods may be performed on the subject or on a sample taken from a subject.

In one example, the present disclosure provides a method of selecting a subject for treatment with an inhibitor of the FANCM-RMI interaction, the method comprising determining whether the subject is suffering from ALT cancer, wherein the subject is selected for treatment with the inhibitor of the FANCM-RMI interaction if the subject is suffering from ALT cancer.

In one example, the present disclosure provides a method of identifying whether a subject suffering from cancer is suitable for treatment with an inhibitor of the FANCM-RMI interaction, comprising determining whether the cancer is ALT cancer, wherein the subject is identified as suitable for treatment with the inhibitor of the FANCM-RMI interaction if the subject is suffering from ALT cancer.

In one example, the present disclosure provides a method of determining whether a subject is responding to treatment with an inhibitor of the FANCM-RMI interaction and/or an inhibitor of FANCM ATPase activity, comprising determining the presence and/or extent of genomic instability at one or more telomeres in a cell taken from a subject. Alternatively, the present disclosure provides a method of determining whether a subject is responding to treatment with an inhibitor of the FANCM-RMI interaction and/or an inhibitor of FANCM ATPase activity, comprising determining the presence and/or level of ALT activity in a cell or tissue sample taken from a subject. The presence and/or level of ALT activity may be determined using any suitable method known in the art, or using any of the methods disclosed herein for identifying an ALT cell. In certain embodiments, the subject is responding if ALT activity is reduced in the cell or tissue sample, e.g., as compared to the amount previously detected in a cell or tissue sample taken from the subject before treatment, or as compared to a pre-determined value.

Additionally, the methods disclosed herein may comprise assaying a sample that may be obtained from the subject before treatment with an inhibitor of the FANCM-RMI interaction and/or an inhibitor of FANCM ATPase activity, to determine the presence/and or extent of genomic instability at one or more telomeres and/or the presence and/or level of ALT activity in a cell taken from the subject. This may be compared to a sample taken from the subject after treatment with an inhibitor of the FANCM-RMI interaction and/or an inhibitor of FANCM ATPase activity. Thus, an increase in genomic instability or level of ALT activity after treatment with an inhibitor of the FANCM-RMI interaction and/or an inhibitor of FANCM ATPase activity relative to the amount/presence of genomic instability or level of ALT activity before treatment with an inhibitor of the FANCM interaction and/or an inhibitor of FANCM ATPase activity is indicative that the subject is responding to treatment with the inhibitor of the FANCM-RMI interaction and/or the inhibitor of FANCM ATPase activity. Conversely, a lack of increase, or a decrease in genomic instability or level of ALT activity after treatment with an inhibitor of the FANCM-RMI interaction and/or an inhibitor of FANCM ATPase activity relative to the amount/presence of genomic instability or level of ALT activity before treatment with an inhibitor of the FANCM interaction and/or an inhibitor of FANCM ATPase activity is indicative that the subject is not responding to treatment with the inhibitor of the FANCM-RMI interaction and/or an inhibitor of FANCM ATPase activity.

It will be understood by the person skilled in the art that assays which are used to determine whether a cell is an ALT cell or a cancer is ALT cancer may also be used to determine whether a subject is responding to treatment with an inhibitor of the FANCM-RMI interaction.

For example, any of the methods disclosed in Henson et al., 2009 and/or WO/2011/035375 can be used to determine whether a subject is responding to the treatment methods of present disclosure. Thus, for example, the methods disclosed herein may comprise assaying a sample obtained from a subject treated with an inhibitor of the FANCM-RMI interaction for the presence and/or amount of partially double-stranded telomeric circles, wherein the partially double-stranded telomeric circles are detected following rolling circle amplification using the partially double-stranded circular telomeric DNA as template, wherein the presence and/or amount of said circles in indicative of whether the subject is responding to treatment. The methods may be performed with or without an exogenous primer. For example, the methods may be performed without an exogenous primer. A sample may be obtained from the subject prior to beginning treatment with an inhibitor of the FANCM-RMI interaction and after treatment with an inhibitor of the FANCM-RMI interaction. The presence and/or amount of C-circles in the samples may be compared to determine whether the subject is responding to treatment. For example, an increase in the presence and/or amount of C-circles in a subject's sample is indicative that the subject is responding to treatment with the inhibitor of the FANCM-RMI interaction.

In certain embodiments, the present disclosure provides methods for treating an ALT cancer in a subject in need thereof, comprising providing to the subject an effective amount of a therapeutic agent, such as (a) a FANCM inhibitor (b) FANCM suppressor nucleic acid, (c) a FANCM targetable nuclease, or (d) nucleic acid encoding a FANCM suppressor nucleic acid or targetable nuclease, each of which, as described herein, may be useful in therapy. For example, a therapeutic agent that reduces FANCM expression or activity may be administered to an individual for the treatment of ALT cancer. In particular embodiments, the ALT cancer is any of those disclosed herein. In particular embodiments, the ALT cancer is an osteosarcoma, soft tissue sarcoma (e.g. liposarcoma, undifferentiated pleomorphic sarcoma, or leiomyosarcoma), glioblastoma, astrocytoma, neuroblastoma, or bladder carcinoma.

Reduction of FANCM expression or activity is shown herein to have a strong and specific effect on ALT cancer cells. Thus, in some embodiments, a therapeutic agent, such as (a) a FANCM inhibitor (b) FANCM suppressor nucleic acid, (c) FANCM targetable nuclease, or (d) nucleic acid encoding a FANCM suppressor nucleic acid or targetable nuclease as described herein may therefore be administered to the individual without other concomitant cancer therapy, such as cytotoxic chemotherapy or radiotherapy i.e., the therapeutic agent may be administered alone.

In other embodiments of any of the methods disclosed herein, a therapeutic agent, such as, e.g., (a) a FANCM inhibitor (b) FANCM suppressor nucleic acid, (c) FANCM targetable nuclease, or (d) nucleic acid encoding a FANCM suppressor nucleic acid or targetable nuclease as described herein, may be administered in combination with one or more other therapies, such as cytotoxic chemotherapy or radiotherapy. This may be useful for example in treating cancers that comprise both ALT cancer cells and telomerase positive cancer cells or cancers where the ALT/telomerase status is not determined. In some embodiments, a suitable cytotoxic chemotherapy for use in combination with an ATM antagonist may not be a DNA damaging agent.

When the inhibitors or therapeutic agents are used in combination with additional therapeutic agents, the compounds may be administered either sequentially or simultaneously by any convenient route. When a therapeutic agent is used in combination with an additional therapeutic agent active against the same disease, the dose of each agent in the combination may differ from that when the therapeutic agents are used alone. Appropriate doses will be readily appreciated by those skilled in the art.

The data herein shows that depletion of FANCM alone increases ALT cell death and reduces the viability of ALT cells, whereas simultaneous depletion of both BLM and FANCM does not have this effect. In some preferred embodiments, a FANCM antagonist, such as a FANCM inhibitor, FANCM suppressor nucleic acids, FANCM targetable nuclease, nucleic acids encoding a FANCM suppressor nucleic acid or targetable nuclease, or an agent that disrupts the FANCM-RMI interaction and/or an inhibitor of FANCM ATPase activity may be administered without reducing the expression or activity of BLM and/or BRCA1. For example, the FANCM antagonist and/or an inhibitor of FANCM ATPase activity may be administered to an individual without concomitant administration of a BRCA1 antagonist and/or a BLM antagonist to the individual. Thus, embodiments of any of the methods disclosed herein do not include also providing or administering to the subject a BRCA1 antagonist or a BLM antagonist.

Administration of inhibitors or therapeutic agents, such as: (a) FANCM inhibitors (b) FANCM suppressor nucleic acids (c) FANCM targetable nucleases, (d) nucleic acids encoding FANCM suppressor nucleic acids or targetable nucleases, (e) an agent that disrupts the FANCM-RMI interaction, or (f) an inhibitor of FANCM ATPase activity, as described herein, can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

In some preferred embodiments, the subject or individual is a human. In other preferred embodiments, non-human mammals, especially mammals that are conventionally used as models for demonstrating therapeutic efficacy in humans (e.g. murine, primate, porcine, canine, or rabbit animals) may be employed.

In some embodiments, the individual may have minimal residual disease (MRD) after an initial cancer treatment.

An individual with an ALT cancer may display at least one identifiable sign, symptom, or laboratory finding that is sufficient to make a diagnosis of ALT cancer in accordance with clinical standards known in the art. Examples of such clinical standards can be found in textbooks of medicine such as Harrison's Principles of Internal Medicine, 15th Ed., Fauci A S et al., eds., McGraw-Hill, New York, 2001. In some instances, a diagnosis of an ALT cancer in an individual may include identification of a particular cell type (e.g. an ALT cancer cell) in a sample of a body fluid or tissue obtained from the individual. Methods disclosed herein may be used for diagnosis of an ALT cancer.

Other aspects of the invention relate to the identification of individuals with cancer who are suitable for treatment with a FANCM antagonist, such as (a) a FANCM antagonist or inhibitor (b) FANCM suppressor nucleic acid, (c) FANCM targetable nuclease, or (d) nucleic acid encoding a FANCM suppressor nucleic acid or targetable nuclease, as described herein. For example, an individual with cancer may be assessed using a method described herein to determine whether treatment with a FANCM antagonist would be likely to be beneficial to the individual i.e. whether the individual is suitable for treatment by a method of the first aspect of the invention. A method of predicting, determining or assessing the responsiveness of a cancer in an individual to an agent which reduces FANCM expression or activity may comprise determining the presence of one or more ALT cancer cells in a sample of cancer cells from the individual, the presence of one or more ALT cancer cells in the sample being indicative that the cancer is responsive to said agent.

A sample of cancer cells may be obtained from an individual using conventional techniques. The presence of ALT cancer cells in the sample may be determined by determining the presence of a cancer cells with one or more characteristic features of an ALT cancer cell, for example one or more of features (i) to (v) as set out above. Suitable methods for identifying the presence of characteristic features of an ALT cancer cell, such as the presence of C-circles or ALT associated PML bodies, may be determined using standard techniques.

An individual with a cancer identified as being responsive to an FANCM antagonist may be treated as described herein, for example using a method of the first aspect of the invention.

A therapeutic agent or the pharmaceutical composition comprising the therapeutic agent as described herein may be administered to a subject by any convenient route of administration, whether systemically/peripherally or at the site of desired action, including but not limited to; parenteral, for example, by infusion, including intravenous infusion, in particular intravenous bolus infusion. Suitable infusion techniques are known in the art and commonly used in therapy (see, e.g., Rosenberg et al., New Eng. J. of Med., 319:1676, 1988).

It will be appreciated that appropriate dosages of the therapeutic agent, and compositions comprising the therapeutic agent, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular cells, the route of administration, the time of administration, the rate of loss or inactivation of the cells, the duration of the treatment, other drugs, compounds, and/or materials used in combination, and the age, sex, weight, condition, general health, and prior medical history of the patient. Thus, in certain embodiments, the specific dose level and frequency of dosage for any particular subject may be varied and will depend upon a variety of factors including the activity of the specific compound employed, the metabolic stability and length of action of that compound, the age, body weight, general health, gender, diet, mode and time of administration, rate of excretion, drug combination, the severity of the particular condition, and the host undergoing therapy. The amount of cells and the route of administration will ultimately be at the discretion of the physician, although generally the dosage will be to achieve local concentrations at the site of action which achieve the desired effect without causing substantial harmful or deleterious side-effects.

In some embodiments, a typical oral dosage of a small molecule inhibitor is in the range of from about 0.05 to about 1000 mg, preferably from about 0.1 to about 500 mg, and more preferred from about 1.0 mg to about 200 mg administered in one or more dosages such as 1 to 3 dosages. The exact dosage will depend upon the frequency and mode of administration, the sex, age, weight and general condition of the subject treated, the nature and severity of the condition treated and any concomitant diseases to be treated and other factors evident to those skilled in the art. For parenteral routes such as intravenous, intrathecal, intramuscular and similar administration, typically doses are in the order of about half the dose employed for oral administration.

Other aspects and embodiments of the invention provide the aspects and embodiments described herein with the term “comprising” replaced by the term “consisting of” and the aspects and embodiments described above with the term “comprising” replaced by the term “consisting essentially of”.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to the skilled person on reading this disclosure, and as such, these are within the scope of the present invention.

All documents and sequence database entries mentioned in this specification are incorporated herein by reference in their entirety for all purposes.

### EXAMPLES

### Example 1. Cell Culture and Cell Lines

The cell lines U-2 OS (ALT), IIICF/c (ALT), HeLa (Telomerase-positive), HeLa 1.2.11 (Telomerase-positive), HCT116 (Telomerase-positive), GM847 (ALT), Saos-2 (ALT) and HEK-293 (Telomerase-positive) were cultured in Dulbecco's modified Eagle's medium (DMEM) supplemented with 10% (v/v) fetal bovine serum (FBS) in a humidified incubator at 37° C. with 10% CO₂. Cell lines were authenticated by 16-locus short-tandem-repeat profiling and tested for mycoplasma contamination by CellBank Australia (Children's Medical Research Institute).

### Example 2. RNA Interference

The following Silencer Select siRNAs were designed and synthesized by Life Technologies: FANCM1 (s33619) (SEQ ID NO: 12) and FANCM2 (s33621) (SEQ ID NO: 13), POLD3 (s21045) (SEQ ID NO: 14), BLM (s1998) (SEQ ID NO: 15), RAD51 (s11735) (SEQ ID NO:16), RAD52 (s11747) (SEQ ID NO: 17) and the Silencer Select RNAi siRNA Negative Control #2 (#4390847). Cell suspensions were transfected at 20-50% confluency with Lipofectamine RNAiMAX (Life Technologies) at a final siRNA concentration of 30 nM. Culture media was changed after 48 h and cells harvested for analysis 72 h post-transfection. Knockdown efficiency was validated by western blot analysis.

### Example 3. Vectors

Empty vector and wild-type FANCM (pCMV6-Myc-DDK) constructs were obtained from Origene Technologies. Wild type FANCM (SEQ ID NO: 2) and FANCM mutants (SEQ ID NOs: 19 to 28) were cloned into the pLenti-C-Myc-DDK-IRES-Neo backbone (Origene Technologies) by restriction enzyme subcloning of gene blocks synthesized by Integrated DNA Technologies (IDT). Lentivirus was produced by the Vector and Genome Engineering Facility (Children's Medical Research Institute). For stable overexpression, cells were transduced with lentivirus, allowed to recover for 24 h, and subject to G418 selection. Cells were maintained in G418 to ensure sustained overexpression.

### Example 4. Genomic DNA Extraction and Purification

Cells were harvested by trypsinization, washed in PBS, and lysed in DNA extraction buffer (100 mM Tris-HCl pH 7.6, 100 mM NaCl, 10 mM EDTA, 1% (w/v) N-lauroylsarcosine). Lysates were digested with 50 μg/ml RNase A for 20 min at room temperature, followed by digestion with 100 μg/ml proteinase K overnight at 55° C. DNA was extracted using three rounds of phenol/chloroform/isoamyl alcohol (25:24:1) solution (Sigma Aldrich) in MaXtract High Density tubes (Qiagen). DNA from the aqueous phase was precipitated with 0.1 volume of 3 M sodium acetate pH 5.2 and 2.5 volumes of cold 100% ethanol. DNA was washed with 70% ethanol, dried, and dissolved in 10 mM Tris-HCl pH 8.0, 1 mM EDTA.

### Example 5. C-Circle Assay

C-circles were amplified with Phi29 polymerase using dATP, dTTP and dGTP overnight. Products were dot blotted onto Biodyne B membranes (Pall) and pre-hybridised in PerfectHyb Plus (Sigma) for at least 30 min. Radiolabeled telomeric C-probe was then added and blots were hybridized overnight at 37° C. (Henson et al., 2009). Blots were washed with 0.5×SSC, 0.1% SDS three times for 5 min each then exposed to phosphor screen. Imaging was performed on the Typhoon FLA 7000 system (GE Healthcare) with a PMT of 750 V.

### Example 6. Terminal Restriction Fragment (TRF) Analysis

Genomic DNA was digested with 4 U/μg of HinfI and RsaI overnight at 37° C. Digested DNA was precipitated with 0.1 volume of 3 M sodium acetate pH 5.2 and 2.5 volumes of 100% ethanol. DNA was washed with 70% ethanol, dried, and dissolved in 10 mM Tris-HCl pH 7.6, 1 mM EDTA. For one-dimensional gel electrophoresis, digested DNA (2 μg) was loaded on 1% (w/v) pulse-field certified agarose (Bio-Rad) gels and separated at 6 V/cm for 12 h with an initial switch time of 1 s and a final switch time of 6 s. For two dimensional gel electrophoresis, digested DNA (20 μg) was separated by standard gel electrophoresis in the first dimension in a 0.6% (w/v) agarose gel in 0.5×TBE at 1 V/cm for 13.5 h. Lanes were excised and run in the second dimension in a 1.1% (w/v) agarose gel containing 300 ng/ml ethidium bromide at 6 V/cm for 4 h. Gels were dried for 150 min at 50° C. and rehydrated in 2×SSC for 30 min. Gels were prehybridized in Church buffer (250 mM sodium phosphate buffer pH 7.2, 7% (w/v) SDS, 1% (w/v) BSA fraction V grade (Roche), 1 mM EDTA) for 2 h at 50° C. Native gels were hybridized overnight with γ-[32P]-ATP-labelled (GGGTTA)₄ or (CCCTAA)₄ oligonucleotide probes. Gels were washed three times in 0.2×SSC for 15 min at room temperature, and exposed to a PhosphorImager screen for three days. Gels were then denatured in 0.5 M NaOH, 1.5 M NaCl at 65° C. for 40 min, followed by two washes in 2×SSC. Gels were pre-hybridized for 1 h and then hybridized in Church buffer at 50° C. with γ-[32P]-ATP-labelled (GGGTTA)₄ or (CCCTAA)₄ oligonucleotide probes overnight. Denatured gels were washed and exposed to a PhosphorImager screen overnight. Imaging was performed on the Typhoon FLA 7000 system (GE Healthcare) with a PMT of 750 V.

### Example 7. Immunoblotting

Cells were collected and lysed in RIPA buffer (50 mM Tris-HCl pH 7.6, 150 mM NaCl, 1% Nonidet P-40, 0.5% sodium deoxycholate, 0.1% SDS, 4 mM EDTA) supplemented with complete Mini EDTA-free protease inhibitor cocktail (Roche). Proteins were resolved on either 3-8% Tris-Acetate or 4-12% Bis-Tris gels (Life Technologies). Proteins were transferred to Immobilon P PVDF membranes (Merck). Membranes were optionally stained with Ponceau S (Sigma Aldrich) then destained with PBST. Membranes were then blocked with either 5% skim milk or bovine serum albumin (BSA) in PBST. Blots were incubated with primary antibody (for list of antibodies, see Table 1) at either 4° C. overnight or room temperature for 2 h. Membranes were then incubated with corresponding HRP-conjugated secondary antibodies (Dako) for 1 h at room temperature, and bands visualized using PICO, PICO PLUS or FEMTO enhanced chemiluminescence reagents (Thermo Scientific).

TABLE 1
List of antibodies used:

| | | | Western | Immuno- | Immuno- |
|---|---|---|---|---|---|
| | | | Blot | fluorescence | precipitation |
| Target | Species | Source, Cat # | Dilution | Dilution | Amount |
| Actin | Rabbit | Sigma Aldrich, | 1:5000 | — | — |
| | | A2066 | | | |
| Vinculin | Mouse | Sigma Aldrich, | 1 μg/ml | — | — |
| | | V9131 | | | |
| FANCM | Mouse | CV5.1 (Vuono | 1:1000 | — | — |
| | | et al., 2016) | | | |
| | | Novus, | | | |
| | | NBP2-50418 | | | |
| FANCM | Rabbit | Abcam, ab35620 | — | — | 1 μg |
| TRF2 | Rabbit | Novus, | — | 1:200 | — |
| | | NB110-57130 | | | |
| Myc-tag | Mouse | Cell Signalling | 1:1000 | — | — |
| | | Technologies, | | | |
| | | 2276 | | | |
| FLAG | Mouse | Aviva Systems | — | — | 400 ng |
| (DDK) | | Biology, | | | |
| | | OAEA00002 | | | |
| PML | Goat | Santa Cruz | — | 1:400 | — |
| γ-H2AX | Mouse | Merck Millipore | — | 1:500 | — |
| BLM | Rabbit | Bethyl, | 1:1000 | — | — |
| | | A300-110A | | | |
| POLD3 | Mouse | Novus, | 1:1000 | 1:200 | — |
| | | H00010714- | | | |
| | | M01 | | | |
| RAD51 | Mouse | Abcam, ab213 | 1:1000 | — | — |
| RAD52 | Mouse | Santa Cruz, | 1:1000 | — | — |
| | | SC-365341 | | | |
| RMI1 | Mouse | Abnova, | 1:1000 | — | — |
| | | H00080010- | | | |
| | | B02P | | | |
| ERα | Rabbit | Santa Cruz, | 1:1000 | — | 400 ng |
| (MC-20) | | sc-542 | | | |
| TOP3A | Rabbit | D6 (Wu et al., | 1:1000 | — | — |
| | | 2000) | | | |
| Anti-FLAG | Mouse | Sigma Aldrich, | 1:2500 | — | — |
| M2 Peroxi- | | A8592 | | | |
| dase HRP) | | | | | |
| Anti-BrdU | Rat | Bio Rad, | — | 1:25 | — |
| (cross-reacts | | OBT0030 | | | |
| with CldU) | | | | | |
| Anti-BrdU | Mouse | BD Biosciences, | — | — | 2 μg |
| | | 347580 | | | |

### Example 8. Co-Immunoprecipitation (Co-IP)

For co-IP experiments involved the ER-inducible system, cells were lysed in IP buffer (20 mM Tris-HCl pH 7.5, 100 mM NaCl, 10% (v/v) glycerol, 0.5 mM EDTA, 1 mM DTT) supplemented with 1× mammalian protease inhibitor cocktail (Sigma Aldrich) and 50 U/ml Benzonase (Novagen) for 2 h at 4° C., with or without MM2 peptide variants (Mimotopes). Following lysis, NaCl concentration was increased to 250 mM, then the lysate cleared at 16,000×g for 15 mins. Supernatant was then mixed with 1 μg of α-FANCM (Abcam) or α-ER (Santa Cruz Biotechnology) and 20 μl protein G sepharose (GE Healthcare), or using α-Flag M2 agarose (Sigma Aldrich). Following 3 h of mixing at 4° C., beads were washed 4× with IP buffer, and 1× with 50 mM NH₄(CO₃)₂, 0.5 mM EDTA, then eluted with 500 mM NH₄OH (pH 11.0), 0.5 mM EDTA. Samples were then lyophilized and resuspended in 1×LDS loading buffer (Life Technologies) prior to immunoblotting.

For co-IP experiments involving PIP-199, HeLa cells treated with PIP-199 or DMSO for 72 h were lysed in 20 mM HEPES-KOH pH 7.9, 200 mM NaCl, 2 mM MgCl₂, 10% glycerol, 0.1% Triton X-100 supplemented with 1 mM PMSF, 1 mM DTT and 1× Complete protease inhibitor (Roche) for 1 h at 4° C. Lysate was cleared at 13,000 rpm at 4° C. for 40 mins. Supernatant was then incubated overnight at 4° C. with 30 μl of protein G Dynabeads at 4° C. and 2.5 μg of anti-BLM (Bethyl: #A300-110A), 0.5 μg of anti-RMI1 (Proteintech: 14630-1-AP) or 2.5 μg of Normal Rabbit IgG control (Cell Signalling: 2729S). Protein was eluted in 30 μl of LDS buffer (Life Technologies) at 70° C. prior to analysis via immunoblotting.

### Example 9. Immunofluorescence (IF) and Fluorescence In-Situ Hybridization (FISH)

Indirect IF and telomere FISH were performed on both interphase nuclei and metaphase spreads. For interphase IF experiments, cells were grown on coverslips or LabTek chamber slides (Thermo Scientific). Slides were prepared as described in Sobinoff et al., 2017. Cells on coverslips were washed twice with PBS, permeabilized with KCM buffer (120 mM KCl, 20 mM NaCl, 10 mM Tris pH 7.5, 0.1% Triton), washed again with PBST and PBS, then fixed with ice-cold 4% formaldehyde PBS solution at room temperature for 10 min. Coverslips were blocked with antibody-dilution buffer (20 mM Tris-HCl, pH 7.5, 2% (w/v) BSA, 0.2% (v/v) fish gelatin, 150 mM NaCl, 0.1% (v/v) Triton X-100 and 0.1% (w/v) sodium azide) and 0.1 mg/ml RNaseA for 30 min at 37° C. Cells were incubated with primary antibodies (Supplementary Table 1) for 1 h at 37° C. or 2 h at room temperature, then incubated with 1:1,000 dilution of appropriate Alexa Fluor conjugated secondary antibodies (Thermo Scientific). Coverslips were rinsed with PBS then fixed with 4% (v/v) formaldehyde at room temperature prior to telomere FISH. Coverslips were subjected to a graded ethanol series (75% for 2 min, 85% for 2 min, and 100% for 2 min) and allowed to air-dry. Dehydrated coverslips were overlaid with 0.3 μg/ml FAM-OO-(CCCTAA)₃ telomeric PNA probe (Panagene) in PNA hybridization solution (70% deionized formamide, 0.25% (v/v) NEN blocking reagent (PerkinElmer), 10 mM Tris-HCl, pH 7.5, 4 mM Na2HPO4, 0.5 mM citric acid, and 1.25 mM MgCl2), denatured at 80° C. for 5 min, and hybridized at room temperature overnight. Coverslips were washed twice with PNA wash A (70% formamide, 10 mM Trish pH 7.5) and then PNA wash B (50 mM Tris pH 7.5, 150 mM NaCl, 0.8% Tween-20) for 5 min each. DAPI was added at 50 ng/ml to the second PNA wash B. Finally, coverslips were rinsed briefly in deionized water, air dried and mounted in DABCO (2.3% 1,4 Diazabicyclo (2.2.2) octane, 90% glycerol, 50 mM Tris pH 8.0). Microscopy images were acquired on a Zeiss Axio Imager microscope with appropriate filter sets.

### Example 10. EdU Detection

Cells were pulsed with 10 μM EdU for 2 h. Cells were permeabilized, then fixed with 4% formaldehyde PBS solution. The Click-iT® Alexa Fluor 647 azide reaction was then performed according to the manufacturer's instructions, before blocking with antibody-dilution buffer and RNaseA. Telomeres were visualized by FISH using a TAMRA-OO-(CCCTAA)₃ telomeric PNA probe (Panagene), and PML was visualized by IF.

### Example 11. Single-Molecule Analysis of Telomeric DNA (SMAT)

Cells were labelled with 100 μM CIdU for 5 h prior to harvesting by trypsinization. Cells were embedded in low-melting agarose plugs then subjected to proteinase K digestion overnight. Plugs were dissolved with agarase (Thermo Scientific) according to the manufacturer's instructions. Molecular combing was performed using the Molecular Combing System (Genomic Vision S.A.) with a constant stretch factor of 2 kb/μm using vinyl silane coverslips (20×20 mm; Genomic Vision S.A.), according to the manufacturer's instructions. After combing, coverslips were dried for 4 h at 60° C. Quality and integrity of combed DNA fibers were checked using the YoYo-1 counterstain (Molecular Probes). Coverslips were denatured for 25 min in alkali-denaturing buffer (0.2 M NaOH, 0.1% b-mercaptoethanol in 70% ethanol) and fixed by addition of 0.5% glutaraldehyde for 5 min. Telomeric DNA was visualized by hybridization with a TAMRA-OO-KKK(TTAGGG)₃ PNA probe (Panagene). Halogenated nucleotides were detected with a rat anti-CldU monoclonal antibody (Accurate) and Alexa Fluor 488-conjugated goat anti-rat antibody (Molecular Probes). Telomere fibers were detected on a Zeiss Axio Imager microscope with ApoTome module and analyzed with Zen software (Zeiss).

### Example 12. Nascent Telomere Analysis

BrdU pulldown was performed as described in Verma et al., 2018, with some adaptations. Cells were harvested after pulsing with 100 μM BrdU (Sigma) for 5 h. Genomic DNA (gDNA) was extracted and resuspended in 100 μl of EB buffer (Qiagen). gDNA was sheared into 100-1,000 bp fragments using a Covaris M220 sonicator. 4 μg of sheared gDNA was denatured for 10 min at 95° C., then chilled immediately. Denatured gDNA was incubated with 2 μg control mouse IgG (Millipore) or anti-BrdU antibody (BD Biosciences) in 250 μl immunoprecipitation buffer (0.0625% (v/v) Triton X-100 in PBS), rotating overnight at 4° C. Samples were then incubated with 60 μl BSA-blocked (nuclease-free) Protein G agarose beads (Roche) at 4° C. overnight. The next day, beads were collected via centrifugation for 60 s at 13,000 rpm and washed twice with 1 ml of buffer A (20 mM HEPES-KOH, pH 8.0, 2 mM MgCl₂, 300 mM KCl, 1 mM EDTA, 10% (v/v) glycerol, and 48 0.1% (v/v) Triton X-100) and then 1 ml of TE (10 mM Tris-HCl, 1 mM EDTA, pH 8.0). Immunoprecipitated DNA was eluted from the Protein G agarose beads in 100 μl of elution buffer (50 mM NaHCO₃, and 1% (v/v) SDS) then purified with a QIAquick PCR Purification Kit (Qiagen) and eluted in 140 μl TE. Samples and inputs were diluted with 200 μl of 0.46 M NaOH, denatured at 95° C. for 5 min and cooled on ice, then dot blotted onto Hybond XL (GE Healthcare Life Sciences). Membranes were cross-linked with 2×240 mJ in a Stratalinker (Stratagene) at 254 nm, prehybridized in PerfectHyb Plus hybridization buffer (Sigma) for 54 min at 37° C., and hybridized overnight with γ-[32P]-ATP-labelled (TTAGGG)₄ telomere probe to detect the C-strand. To detect the G-strand, blots were stripped with five washes of near boiling 0.1% SDS for 20 min and reprobed with γ-[32P]-ATP-labelled (CCCTAA)₄ telomere probe. Membranes were washed three times for 15 min in 2×SSC at room temperature, then exposed to a PhosphorImager screen. Imaging was performed with the array analysis function using ImageQuant software (GE Healthcare Life Sciences).

Alternatively, gDNA was extracted and digested with HinfI and RsaI prior to BrdU immunoprecipitation. Immunoprecipitated DNA was then eluted from the Protein G agarose beads in 100 μl of elution buffer (50 mM NaHCO₃, and 1% 61 (v/v) SDS), purified with a QIAquick PCR Purification Kit (Qiagen), and eluted in 25 μL TE. C-circle amplification and detection was carried out as described in Henson et al., 2009.

### Example 13. Automated Image Analysis

ZEN microscopy images (.czi) were processed into extended projections of z-stacks using ZEN desk 2011 software (Zeiss) and imported into Cellprofiler v2.1.1 (Carpenter et al., 2006) for analysis. The DAPI channel was used to mask individual nuclei as primary objects. Foci within each segmented nucleus were identified using an intensity-threshold based mask. Any given object was considered to be overlapping another object when at least 20% of the first object's area was enclosed within the area of a second object.

### Example 14. Sister Chromatid Exchange (SCE) Assays

SCE assays were performed as described in Bayani, 2005, with cells cultured in 100 μM BrdU (Sigma Aldrich), and with or without 200 nM 4-hydroxy-tamoxifen (Sigma Aldrich) for two cell cycles followed by 1 h in 0.2 μg/ml colcemid (Life Technologies). Images of mitotic spreads were captured using a Zeiss Axioplan 2 microscope, and SCEs were manually scored and blind verified. Exchanges that were obviously due to “flipping” at the centrosome were omitted from quantitation. Mitotic abnormalities were scored as described in Caldon et al., 2013 and Bayani, 2005.

### Example 15. Clonogenic Assay

Clonogenic assays were performed as described in Franken et al., 2006, with cells plated at 1,000 cells/10 cm dish with or without 200 nM 4-hydroxytamoxifen (Sigma Aldrich), which was replenished on days 4 and 7. For cells treated with PIP-199 (Aobious #AOB33732, CAS No: 622795-76-0), cells were plated at 300 cells/well in 6-well plates, and treated with 0-10 μM of drug in dimethyl sulfoxide (DMSO). Cells were left to grow for 11 days. Plates were washed with 1×PBS, then fixed and stained with 0.5% (w/v) crystal violet in 1 part acetic acid, 7 parts methanol for 2 h at room temperature. Plates were then washed with tap water and left to air dry. Colonies on each plate were counted and the plating efficiency and surviving fraction calculated.

### Example 16. Live Cell Imaging

Cells were seeded in Alcian-Blue coated 12-well 1.5 mm glass-bottom wells (MatTek) 24 h prior to imaging. 120 cells and subsequent daughter cells were monitored for 48 h at 6 min intervals. The number of nuclei, interphase duration, mitotic duration (defined by duration between initial rounding up of cell to completion of cytokinesis), and mitotic outcome (normal, death during mitosis, aborted, multipolar division, and cell fusion leading to multinuclear cells), were recorded.

### Example 17. Flow Cytometry

Ethanol-fixed single-cell suspensions (approximately 1×10⁶ cells) were stained for DNA analysis with 2 mg/ml RNase A and 0.1 mg/ml propidium iodide (PI) in 0.25 ml PBS. Cells were incubated for 30 min at 37° C. and equilibrated at room temperature in the dark for at least 10 min. Cells were analyzed by BD FACSCanto Flow Cytometry (BD Biosciences) using an air-cooled 488 nm argon laser to excite PI. A total of 9,800-10,000 stopping gate events were collected at an approximate flow rate of 200 events/s. The forward scatter (FSC, size) and side scatter (SSC, internal granularity) of each cell were recorded. To discriminate and eliminate cell debris and doublets, the pulse area (PI-A) was plotted against the pulse width (PI-W). Doublets identified as cells with 4N DNA content and increasing pulse width were eliminated. Cell cycle population analysis was conducted with FlowJo v5 software (FlowJo). Cell phase (G0/G1 and G2/M) gating was performed using the Dean-Jett algorithm. The percentage of cells in S-phase was calculated as the remaining percentage after G0/G1 and G2/M gating.

### Example 18. Statistical Analysis

Details regarding quantitation and statistical analysis are provided in the figure legends. The two-sided Student's t-test was performed on data assumed to be normally distributed, while the two-sided Mann-Whitney test was performed on data assumed to be non-normally distributed. Statistical analysis was not performed on telomere extension lengths due to the substantial difference in events between treatments. Data analysis was performed using Microsoft Excel and GraphPad Prism.

### Example 19. FANCM Depletion Induces Telomere Dysfunction and ALT Markers

ALT telomeres are characterized by an elevated DNA damage response (DDR) compared to mortal and telomerase-positive cells (Cesare et al., 2009). Such damage is observed as telomere dysfunction-induced foci (TIFs) marked by colocalization of the DNA damage marker γ-H2AX with telomeric DNA. Knockdown of FANCM, using siFANCM1 and siFANCM2 (FIG. 1a), resulted in a significant increase in metaphase-TIFs (meta-TIFs) compared to the scrambled control in U-2 OS ALT cells (FIG. 2a). In contrast to the ALT-specific induction of telomere dysfunction, FANCM depletion induced comparable levels of global DDR signaling in both U-2 OS (ALT) and HeLa (telomerase-positive) cell lines (FIG. 1b).

ALT cells characteristically have long and heterogeneous telomere lengths and abundant extrachromosomal telomere repeat (ECTR) DNA, including t-circles and C-circles (Henson et al., 2009). One-dimensional gel electrophoresis of isolated terminal restriction fragments (TRFs) followed by hybridization under native conditions revealed both a decrease in the G-rich overhang and a striking increase in low molecular weight single-stranded (ss) C-rich telomeric DNA with FANCM depletion (FIG. 2b; black arrow and red arrow, respectively). This low molecular weight species of DNA was also detected following separation of undigested genomic DNA (FIG. 1c), indicative of it being extrachromosomal in origin. Despite these observations, no change in mean telomere length was observed (FIG. 2b). These effects were observed following FANCM depletion using both siRNAs, with the most marked effects being seen with siFANCM2. Consequently, siFANCM2 was used for the subsequent experiments. Separation of TRFs by two-dimensional gel electrophoresis identified an increase in extrachromosomal t-circles, which characteristically resolve as an arc above the linear telomeric DNA arc, following FANCM depletion (FIG. 2c; blue arrows). The low molecular weight ss C-rich telomeric DNA ran as a distinct and separate arc below both the t-circle and linear telomeric DNA arcs in FANCM depleted cells (FIG. 2c; red arrows).

FANCM depletion resulted in a striking increase in C-circles, detected by rolling circle amplification (FIG. 1d, FIG. 2d). This increase in amplified C-circles corresponded with the low molecular weight ss ECTR C-rich telomeric DNA identified by one- and two-dimensional gel electrophoresis (FIGS. 2b and 2c), consistent with this species of DNA being C-circles. This is the first reported visualization of C-circles by gel electrophoresis, demonstrating that t-circles and C-circles resolve as distinct DNA species. Quantitation of ALT-associated PML bodies (APBs), in which telomeric DNA and telomere-associated proteins colocalize with PML protein, revealed a significant increase in the number of APBs following FANCM depletion in U-2 OS and IIICF/c cells, but no change in GM847 and Saos-2 cells (FIGS. 1e, 2e and 2f).

Interestingly, a significant increase in the intensity of the telomeric signal within APBs was also observed in all ALT cell lines analyzed (FIGS. 1f, 2e and 2g), indicative of increased telomeric DNA accumulation or telomere clustering in APBs in response to FANCM depletion. Together, these data demonstrate a striking induction of ALT phenotypes, but no overall telomere lengthening, following transient loss of FANCM. These observations were specific to ALT cell lines (U-2 OS, IIICF/c, GM847 and Saos-2) and independent of p53 status, while no induction of ALT characteristics was observed in response to FANCM depletion in telomerase-positive cell lines (HeLa, HeLa 1.2.11 and HCT116) (FIG. 1), indicative of the induction of ALT phenotypes being attributable to ALT-specific telomere dysfunction.

### Example 20. FANCM Depletion Promotes Break-Induced Telomere Synthesis

ALT involves a mechanism of conservative break-induced telomere synthesis that is analogous to break induced replication (BIR) in yeast, and is dependent on the POLD3 subunit of Polδ. A significant increase in POLD3 recruitment to telomeres following FANCM depletion (FIG. 3a) was identified. This coincided with an increase in total nascent telomere repeat generation (FIG. 3b). Nascent telomeric DNA localized with APBs (FIG. 3c), and contributed to the dramatic induction of C-circles generated in response to FANCM depletion (FIG. 3d).

It has previously been shown that break-induced telomere synthesis is dependent on BLM, and that both RAD51-dependent and -independent pathways of telomere extension can exist (Sobinoff et al., 2017, Dilley et al., 2016, Cho et al., 2014 and Verma et al., 2016). These pathways are reminiscent of Type I (Rad51-dependent) and Type II (Rad51-independent) telomerase-null Saccharomyces cerevisiae survivors, which require Rad52 and BLM homolog Sgs1. To characterize the involvement of these proteins in the context of FANCM knockdown, the inventors co-depleted FANCM and either POLD3, BLM, RAD51 and RAD52 (Supplementary FIG. 4a). Interestingly, FANCM depletion resulted in a concomitant decrease in protein levels of POLD3, BLM, and RAD51 (FIGS. 4a and 4b), which was significant for RAD51, suggesting that FANCM coregulates these proteins. It is unlikely that this coregulation contributes to the exacerbated ALT phenotype observed, as independent depletion of POLD3, BLM or RAD51 causes subtle or antagonistic effects to ALT activity, compared to that seen with FANCM depletion. Co-depletion experiments showed that the elevated levels of C-circles detected by both the C-circle assay and by TRF analysis following FANCM depletion were dependent on POLD3 and BLM, and partially dependent on RAD51 and RAD52 (FIGS. 4c and 5a). Similarly, the increased number and intensity of APBs in response to FANCM depletion was dependent on POLD3 and BLM, and partially dependent on RAD51 and RAD52 (FIGS. 5b and 5c).

To directly measure the frequency and length of telomere synthesis events, single molecule analysis of telomeres (SMAT) on CldU-incorporated DNA fibers was used. FANCM-depletion resulted in a significant increase in the number of telomere extension events, while the length of the extension products remained unchanged (FIG. 5d). This increase was predominantly dependent on POLD3, BLM and RAD52 (FIG. 5d). Overall, these data demonstrate that FANCM depletion results in an increase in POLD3- and BLM-mediated break-induced telomere synthesis at ALT telomeres, and coincides with a rapid induction of nascent ECTRs that are predominantly ss and C-rich. These data indicate a stronger reliance on RAD52 than RAD51, but support a role for both proteins in break-induced telomere synthesis.

### Example 21. The MM2 Domain of FANCM is Required to Attenuate ALT Activity

The multiple functional domains of FANCM have been well characterized. Specifically, the PIP domain interacts with proliferating cell nuclear antigen (PCNA) via a conserved PIP-box sequence (Rohleder et al., 2016). The conserved DEAH domain has ATP-dependent DNA-remodeling translocase activity, and binds to replication forks and DNA repair intermediates to promote displacement and annealing of nascent and parental DNA strands (Xue et al., 2008 and Gari et al., 2008). The Major Histone Fold 1 and 2 (MHF1/2) heterotetramer binds to the MHF1/2 interacting domain (MID), and is an obligate cofactor that targets FANCM to DNA branch points to facilitate replication fork traversal (Yan et al., 2010, Huang et al., 2013, Fox et al., 2014 and Zhao et al., 2014). The MM1 domain recruits the FA core complex to ICL sites; the MM2 domain interacts with the BTR complex to coordinate the stabilization of replication forks; and the MM3 domain has no known function (Singh et al., 2013). A key site at 51045 is phosphorylated upon genotoxic stress and is required for efficient ATR-CHK1 checkpoint activation. The ERCC4 endonuclease domain and the Helix-hairpin-Helix (HhH) motif recognize ss DNA gaps and lesions in DNA present at ICL sites, and facilitate heterodimerization with FAAP24, another obligate FANCM cofactor (Xue et al., 2008, Blackford et al., 2012, Kim et al., 2008 and Huang et al., 2010).

It has been shown elsewhere that FANCM directly associates with telomeric DNA (Pan et al., 2017 and Pentz et al., 2019). To determine the specific functional activity of FANCM required to suppress the ALT phenotype, a panel of ten mutant lentiviral constructs (SEQ ID NOs: 19-28), in which each identified domain was disrupted (FIG. 6a) was established. The PIP mutant (SEQ ID NO: 19) is substituted at L8R and W12S. The K117R mutant (SEQ ID NO: 20) is substituted at K117R. The MID domain mutant (SEQ ID NO: 21) is substituted at V749G/H751G to disrupt interaction with the MHF1/2 heterotetramer interface. The S1045A mutant (SEQ ID NO: 22) is substituted at S1045A. The MM1 domain mutant (SEQ ID NO: 23) has a deletion at amino acid residues 943-1004. The MM2 domain mutant (SEQ ID NO: 24) has a deletion at amino acid residues 1219-1251. The FF>AA mutant (SED ID NO: 25) has a double substitution of phenylalanine for alanine (FANCMF1232A/F1236A) within the MM2 domain, and has previously been characterized to substantially disrupt BTR binding (Deans et al., 2009). The MM3 domain mutant (SEQ ID NO: 26) has a deletion at amino acid residues 1337-1707. The ERCC4 domain mutant (SEQ ID NO: 27) has a deletion at amino acid residues 1815-1922. The HhH domain mutant (SEQ ID NO: 28) has a deletion at amino acid residues 1971-2048. These constructs, including wild-type FANCM, were stably transduced into U-2 OS cells, and exogenous expression of the FANCM mutants confirmed by western blot analysis (FIG. 7a). Stable overexpression of FANCM resulted in a significant decrease in both telomere dysfunction (FIG. 6b) and the number of fragile telomeres (FIG. 6c). Most of the mutants also suppressed the telomere DDR and telomere fragility (FIGS. 6b and 6c). The exceptions were the MM2 (SEQ ID NO: 24), FF>AA and MID domain mutants that significantly increased both telomere dysfunction and telomere fragility, and the K117R mutant (SEQ ID NO: 20) in which the DEAH domain is disrupted, that failed to suppress the phenotypes (FIGS. 6b and 6c). The effects of FANCM mutant overexpression on ECTR generation and ALT activity were examined. Consistent with stable overexpression of wild-type FANCM, overexpression of most FANCM mutants resulted in a significant decrease in C-circle generation (FIG. 6d). The exceptions were the K117R (SEQ ID NO: 20), ERCC4 (SEQ ID NO: 27) and HhH domain (SEQ ID NO: 28) mutants that did not cause a change in C-circle levels, and the MM2 (SEQ ID NO: 24) and FF>AA (SEQ ID NO: 25) domain mutants that caused an approximate two-fold increase in C-circles compared to vector control (FIG. 6d). No changes in mean telomere length were observed following wild-type or mutant FANCM overexpression; however, the previously identified smear of low molecular weight ss C-rich telomeric DNA was observed in cells overexpressing the K117R, MM2 and FF>AA domain mutants (FIG. 7b). These changes coincided with a significant increase in both the number of APBs and the intensity of telomeric signal within APBs following overexpression of the K117R, MM2 and FF>AA mutants (FIG. 7c). More subtle differences in the number and intensity of APBs were observed following overexpression of some of the other mutants, indicative of the underlying complexity of APB formation and function.

The number and length of telomere extension events to determine whether overexpression of FANCM mutants had a direct impact on telomere synthesis was measured. A significant increase in the number of extension events was observed in response to overexpression of the MM2 and FF>AA domain mutants (FIG. 6e), while the length of extension events remained relatively stable following overexpression of wild-type FANCM and the other mutants. These data show that wild-type FANCM suppresses telomere replication stress, but does not directly impact the length of telomere extension events. Overall, the functional significance of the replication fork remodeling and restart activities of FANCM, provided by its ATP-dependent translocase activity (DEAH domain) and its BTR binding capability (MM2 domain), in regulating ALT activity was demonstrated. The data also indicate dominant-negative effects following overexpression of the MM2 domain mutants.

Further studies were performed using a double mutant (DM) lentiviral construct that included both the K117R substitution and the FF>AA double substitution (SEQ ID NO:35). The effects of the DM, as compared to the K117R mutant and the FF>AA mutant, on telomere dysfunction, APB frequency and C-circle levels were examined. As shown in FIG. 23A-C, the DM caused a significantly greater increase in C-circles as compared to either the K117R mutant or the FF>AA mutant. The DM mutant also showed enhanced telomere dysfunction, as compared to either empty vector or wild type. These changes were accompanied by a greater increase in the number of APBs as compared to either the K117R mutant or the FF>AA mutant. Thus, the inventors have identified two independent targets on FANCM (the MM2 interaction domain and the ATPase domain). Given the data showing that the ATPase domain of FANCM is necessary to resolve telomeric R-loops, which are main triggers of replication stress at telomeres, these studies suggest that FANCM ATPase activity and RMI interaction are independently supporting telomere stability in ALT cells. These studies suggest that ALT cancers may also be treated using an inhibitor of the FANCM-RMI interaction alone or in combination with an inhibitor of FANCM's ATPase and/or translocase activity.

Defects in ALT phenotype suppression are exacerbated by overexpression of a double-mutant (DM) FANCM, in which both the MM2 domain (FF>AA) and the translocase domain (K117R) of FANCM are mutated.

(a) Representative dot blots and quantitation of C-circle assays in U-2 OS cells stably overexpressing wild-type (WT) or FANCM domain mutants (K117R, FF>AA, DM). C-circles were normalized to the mean of empty vector control (EV). Error bars represent mean±SEM from n=3 experiments, *p<0.05, **p<0.005, Student's t-test. (b) Quantitation of APB frequency in U-2 OS cells overexpressing wild-type (WT) or FANCM mutants. Scatterplot bars represent the mean±SEM. Out of three experiments, n=150 cells were scored for each mutant, **p<0.005, Mann-Whitney test. (c) Quantitation of metaphase-TIFs in U-2 OS cells stably overexpressing wild-type (WT) or FANCM mutants. Scatterplot bars represent the mean±SEM. Out of three experiments, n=120 metaphases scored for each mutant, *p<0.05, **p<0.005, Mann-Whitney test. All statistical comparisons are relative to EV control.

### Example 22. Disruption of the FANCM-BTR Complex Inhibits ALT Cell Viability

It has previously been shown that FANCM depletion induced replication stress at ALT telomeres, but did not affect cell viability (Pan et al., 2017). In contrast, throughout the course of this study, a paucity of mitotic cells following FANCM depletion was consistently observed. To determine the effects of FANCM depletion on cell cycle progression, cell cycle analysis and live cell imaging was used. An accumulation of cells in G2/M was observed in FANCM depleted U-2 OS cells compared to FANCM depleted HeLa cells (FIGS. 8a and 8b). Both U-2 OS and HeLa cells showed delayed cycling speed following FANCM depletion, as evident by an increase in interphase duration (FIGS. 8c and 8d). U-2 OS cells depleted of FANCM exhibited cell cycle attenuation, with more than a third of cells failing to enter mitosis over the 48 h observation window, while the effect of FANCM depletion on mitotic entry in HeLa cells was considerably less severe (FIGS. 8c and 8d). No changes in mitotic outcome (aberrant mitosis or mitotic death) were apparent following FANCM depletion, consistent with cells arresting prior to mitosis (FIGS. 8e and 8f). It is likely that cells capable of progressing through to mitosis had evaded critical levels of FANCM knockdown. These data demonstrate ALT cells are hypersensitive to replication stress caused by FANCM depletion.

To further evaluate the effect of FANCM perturbation on cell survival, Project Achilles, an initiative to identify and catalogue gene essentiality across cancer cell lines was utilized (Meyers et al., 2017). The gene dependency scores for FANCM following CRISPR-Cas9-mediated knockout was compared. Gene dependency scores were calculated using CERES 43 and indicate the likelihood that FANCM is essential in the cell line. A lower gene dependency score is indicative of a higher likelihood that the gene is essential, with −1 representing the median score for all pan-cancer essential genes. Overall, across the panel of cell lines, FANCM did not appear to be essential for cell viability (FIG. 9). However, for the subset of ALT cell lines that were identified, the majority (4/5) clustered around a gene dependency score of −1 (FIG. 9). These data support an essential role for FANCM in ALT cancer cell viability.

To explore the possibility of specifically targeting ALT cell viability through FANCM, two approaches to disrupt the FANCM-BTR complex were adopted. First, a chemically synthesized peptide (SEQ ID NO: 29) corresponding to the highly conserved 28 amino acids of the MM2 domain, that has been shown to have similar binding affinity to the BTR complex as full length FANCM was used (Deans et al., 2009). Addition of the MM2 peptide to cellular lysates resulted in dose-dependent inhibition of FANCM-BTR complex formation, whilst addition of an MM2 peptide containing the FF>AA mutant (SEQ ID NO: 30) that does not bind to BTR was unable to inhibit complex formation (FIGS. 10a and 10b). To investigate the effects of FANCM-BTR complex disruption in ALT cells, the MM2 peptide was rendered functionally dependent on tamoxifen by fusion to the C-terminus of the estrogen receptor (MM2-ER) (FIG. 11a). The ability of the MM2-ER fusion protein to inhibit FANCM-BTR complex formation was tested by immunoprecipitation with either FANCM or ER. In the absence of tamoxifen, TOP3A and RMI1 components of the BTR complex were immunoprecipitated by FANCM, but not by ER (FIG. 11b). Addition of tamoxifen resulted in activation of the MM2-ER fusion protein, detected by immunoprecipitation of TOP3A and RMI1 with ER, but not with FANCM. Activation of the control FF>AA mutant MM2-ER fusion protein was unable to sequester the BTR complex away from FANCM (FIG. 11b).

To confirm the genomic efficacy of FANCM-BTR complex disruption, sister chromatid exchanges (SCEs) were quantified as it has previously been shown that FANCM depletion results in increased SCE formation (Deans et al., 2009). An increase in the frequency of SCEs in response to activation of the MM2-ER fusion protein was identified, but not the FF>AA mutant (FIGS. 10c and 10d), consistent with effective disruption of the FANCM-BTR complex in both ALT and telomerase-positive cell lines. Activation of the MM2-ER fusion protein resulted in a significant increase in TIFs in U-2 OS cells, while a small but significant decrease in TIFs was observed following activation of the FF>AA mutant fusion protein (FIG. 11c). Activation of the MM2-ER fusion protein, but not the FF>AA mutant, also induced C-circles (FIG. 11d), indicative of enhanced replication stress at ALT telomeres leading to increased ALT activity. Clonogenic survival assays revealed a 10-20-fold decrease in the survival of U-2 OS, GM847 and Saos-2 ALT cells following activation of the MM2-ER fusion protein, while activation of the FF>AA mutant MM2-ER fusion protein did not affect cell survival (FIG. 11e). No impact on cell survival was observed with activation of either the wild-type MM2-ER or FF>AA mutant MM2-ER fusion proteins in the telomerase positive cell lines HeLa and HCT116 (FIG. 11e).

The second approach involved treating cells with increasing concentrations of PIP-199, a small molecule inhibitor of the MM2-RMI interaction within FANCM-BTR. An increase in C-circles in the U-2 OS, GM847 and Saos-2 ALT cell lines (FIG. 11f) was observed, consistent with PIP-199-mediated disruption of the FANCM-BTR complex. Clonogenic assays identified hypersensitivity of ALT cells to PIP-199 compared to telomerase-positive cells (FIG. 11g and FIG. 12a). Co-immunoprecipitation experiments of BLM and RMI1 in U-2 OS cells confirmed the disruption of FANCM-BLM and FANCM-RMI1 interaction by increasing concentrations of PIP-199 after 72 hours (FIG. 12b).

These experiments employ two independent approaches to disrupt the critical binding interaction between FANCM and the BTR complex, and demonstrate that inhibition of the FANCM-BTR complex causes replication stress and elevated C-circles.

In summary, the inventors' study has defined the mechanism by which FANCM regulates ALT activity. Specifically, FANCM functions in a BTR-dependent, FA-core complex-independent manner, to resolve replication stress that arises spontaneously within telomeres. ALT telomeres possess inherent structural aberrations that predispose them to replication defects, and are therefore hypersensitive to FANCM depletion. In the absence of FANCM, or through synthetic inhibition of the FANCM-BTR complex, stalled forks deteriorate to form DSBs. This causes induction of break-induced telomere synthesis events to repair dysfunctional telomeres, and coincides with the production of nascent ECTR DNA species. ECTR accumulation may further exacerbate the DDR through the exhaustion of cellular RPA reserves. The consequences of DSB formation and the ensuing DDR include excessive ALT activity and loss of cell viability through G2/M stalling. Importantly, the inventors have found that FANCM-BTR complex inhibition can be used to selectively suppress the growth and viability of ALT cancer cells and that targeting the FANCM-RMI interaction is a potent strategy for killing ALT cancer cells.

### Example 23. FANCM Regulates BML in ALT Cells

Methods

Cell Lines and Culture Conditions

HeLa cervical cancer, HT1080 fibrosarcoma, and HEK293 embryonic kidney cells were purchased from ATCC. U2OS osteosarcoma cells were a kind gift from M. Lopes (IMCR, Zurich, Switzerland). HuO9, Saos2 and HOS osteosarcoma cells were a kind gift from B. Fuchs (Balgrist University Hospital, Zurich, Switzerland). WI-38 VA13 in vitro SV40-transformed lung fibroblasts were a kind gift from A. Londoño-Vallejo (CNRS, Paris, France). SKNAS neuroblastoma cells were a kind gift from O. Shakhova (University Hospital Zurich, Switzerland). HeLa, HT1080, HEK293, U2OS, and WI-38 VA13 cells were cultured in high glucose DMEM, GlutaMAX (Thermo Fisher Scientific) supplemented with 10% tetracycline-free fetal bovine serum (Pan BioTech) and 100 U/ml penicillin-streptomycin (Thermo Fisher Scientific). HuO9, Saos2, HOS and SKNAS cells were cultured in high glucose DMEM/F12, GlutaMAX (Thermo Fisher Scientific), supplemented with 10% tetracycline-free fetal bovine serum (Pan BioTech), 100 U/ml penicillin-streptomycin (Thermo Fisher Scientific) and non-essential amino acids (Thermo Fisher Scientific). Mycoplasma contaminations were tested using the VenorGeM Mycoplasma PCR Detection Kit (Minerva Biolabs) according to the manufacturer's instructions. When indicated, cells were incubated with 1 μM camptothecin (Sigma-Aldrich) for 3 hours, 0.2 mM hydroxyurea (Sigma-Aldrich) for 16 h, 20 μM BIBR 1532 (Merck Millipore) for 7 days, and 10 μM RO-3306 (Selleckchem) for 18 h.

Ectopic Protein Expression

For FANCM complementation experiments, siFa- and siFb-resistant cDNAs coding for N-terminally V5-tagged FANCM variants were synthesized at GenScript and cloned into the into the lentiviral vector pLVX-TetOne-Puro (Clontech). The obtained plasmids, pLVX-VSFANCM and pLVX-VSFANCMK117R, were used to produce lentiviruses and infect U2OS cells, followed by selection in medium containing 1 μg/ml puromycin (Merck Millipore). Experiments were performed in medium containing 1 μg/ml doxycycline (Sigma-Aldrich). For RNaseH1 overexpression, U2OS cells were infected with retroviruses produced using the pLHCX-MYC-RH1WT and pLHCX-MYC-RH1D145A plasmids¹¹, followed by selection in medium containing 200 μg/ml hygromycin B (VWR). For TRF1 overexpression, U2OS cells were infected with retroviruses produced using the pLPC-NFLAG-TRF1 (a kind gift from T. de Lange, Addgene plasmid #16058), followed by puromycin selection. Transgene expression was validated by western blotting. For telomerase overexpression, U2OS and HeLa cells were infected with retroviruses produced using the pBABEpuroUThTERT+U3-hTR-500 plasmid (Addgene plasmid #27665), followed by puromycin selection. Viruses were produced in HEK293 cells according to standard procedures. FANCM, RNaseH1 and TRF1 ectopic expression was validated by western blotting (see below). hTERT and hTR expression was validated by quantitative RT-PCR on total RNA using the following oligonucleotides:

hTERTfor,
(SEQ ID NO: 41)
5′-agagtgtctggagcaagttgc-3′;
hTERTrev,
(SEQ ID NO: 42)
5′-cgtagtccatgttcacaatcg-3′;
hTRfor,
(SEQ ID NO: 43)
5′-gtggtggccattttttgtctaac-3′;
hTRrev,
(SEQ ID NO: 44)
5′-tgctctagaatgaacggtggaa-3′;
ActinB1for,
(SEQ ID NO: 45)
5′-tccctggagaagagctacga-3′;
Actin B1rev,
(SEQ ID NO: 46)
5′-agcactgtgttggcgtacag-3′.
Actin B1 was used as a normalizer.

siRNA-mediated protein depletion DsiRNAs (Integrated DNA Technologies) were transfected using the Lipofectamine RNAiMAX reagent (Invitrogen) according to the manufacturer instructions. DsiRNAs were used at a final concentration of 20 nM unless otherwise indicated. Medium was changed 5 hours after transfection and samples collected 48 h after transfection unless otherwise indicated. The following mRNA target sequences were used:

siFa:
(SEQ ID NO: 33)
5′-GGATGTTTAGGAGAACAAAGAGCTA-3′;
siFb:
(SEQ ID NO: 34)
5′-CCCATCAAATGAAGATATGCAGAAT-3′;
siBl:
(SEQ ID NO: 36)
5′-GCTAGGAGTCTGCGTGCGAGGATTA-3′;
siATRXa:
(SEQ ID NO: 37)
5′-GAGGAAACCUUCAAUUGUAACAAAGUA-3′;
siATRXb:
(SEQ ID NO: 38)
5′-UGCAAGCUCUAUCAGUACUACUUAGAU-3′;
siTRF1:
(SEQ ID NO: 39)
5′-CUUUCUUUCUUAUUAAGGUCUUGUUGC-3′;
siRNaseH1:
(SEQ ID NO: 40)
5′-UUGUCUAAUGCCUACAUUUAAAGGAUG-3′.
NCI Negative Control (51-01-14-03)
was used as siCt.

Cell Proliferation and Viability Assays

For colony forming assays, cells were transfected with siRNAs and 24 hours later 300 to 500 cells were plated in 3 cm dishes and grown until visible colonies were formed. Cells were stained in 1% Crystal violet, 1% formaldehyde, 1% MeOH (Sigma-Aldrich) for 20 min at room temperature, followed by washes in tap water. Plates were air-dried and photographed with a FluorChem HD2 imaging apparatus (Alpha Innotech). Colonies were counted using ImageJ software. For growth curves, cells were transfected with siRNAs and 24 h later 1×10⁵ cells were seeded in 6 cm dishes and passaged for 10 days. Cells were re-transfected with siRNAs and counted every 3 days. For fluorescence-activated cell sorting, cells were trypsinized and pelleted by centrifugation at 500 g at 4° C. for 5 min. Cell pellets were either left untreated for viability assays or fixed in 70% ethanol at −20° C. for 30 min and treated with 25 μg/ml RNaseA (Sigma-Aldrich) in 1×PBS at 37° C. for 20 min. Cells were then washed in 1×PBS and stained with 20 μg/ml propidium iodide (Sigma-Aldrich) in 1×PBS at 4° C. for 10 min. Flow cytometry was performed on a BD FACSCalibur or a BD Accuri C6 (BD Biosciences). Data were analyzed using FlowJo software.

Western Blotting

Cells were trypsinized and pelleted by centrifugation at 500 g at 4° C. for 5 min. Pellets were resuspended in 2× lysis buffer (4% SDS, 20% Glycerol, 120 mM Tris-HCl pH 6.8), boiled at 95° C. for 5 min and centrifuged at 1600 g at 4° C. for 10 min. Supernatant were recovered and protein concentrations were determined by Lowry assay using bovine serum albumin (BSA; Sigma-Aldrich) as standard. 20-40 μg of proteins were supplemented with 0.004% Bromophenol blue and 1% β-Mercaptoethanol (Sigma-Aldrich), incubated at 95° C. for 5 min, separated in 6 or 10% polyacrylamide gels, and transferred to nitrocellulose membranes (Maine Manufacturing, LLC) using a Trans-Blot SD Semi-Dry Transfer Cell apparatus (Bio-Rad). The following primary antibodies were also used: mouse monoclonal anti-FANCM (CV5.1⁷², 1:1000 dilution); mouse monoclonal anti-Golgin 97 (Molecular Probes, A-21270, 1:5000 dilution); rabbit polyclonal anti-KAP1 (Bethyl Laboratories, A300-274A, 1:2000 dilution); rabbit polyclonal anti-pKAP1 (Ser 824) (Bethyl Laboratories, A300-767A, 1:2000 dilution); mouse monoclonal anti-CHK1 (Santa Cruz Biotechnology, sc-8408, 1:1000 dilution); rabbit monoclonal anti-pCHK1 Ser 345 (Cell Signaling, 2348, 1:500 dilution); rabbit polyclonal anti-RPA32 (Bethyl Laboratories, A300-244A, 1:3000 dilution); rabbit polyclonal anti-pRPA32 Ser 33 (Bethyl Laboratories, A300-246A, 1:1000 dilution); rabbit polyclonal anti-Lamin B1 (GeneTex, GTX103292, 1:1000 dilution); rabbit polyclonal anti-RNaseH1 (GeneTex, GTX117624, 1:500 dilution); mouse monoclonal anti-beta Actin (Abcam, ab8224, 1:5000 dilution); rabbit polyclonal anti-BLM (Bethyl Laboratories, A300-110A, 1:3000 dilution); rabbit polyclonal anti-PML (1:1000 dilution); rabbit polyclonal anti-PARP1 (Cell Signaling, 9542, 1:1000 dilution); mouse monoclonal anti-POLD3 (Novus Biologicals, H00010714-M01, 1:500 dilution); rabbit polyclonal anti-ATRX (Bethyl Laboratories, A301-045A-T, 1:1000 dilution); sheep polyclonal anti-TRF1 (R&D Systems, AF5300, 1:1000 dilution). Secondary antibodies were HRP-conjugated goat anti-mouse and goat anti-rabbit IgGs (Bethyl Laboratories, A90-116P and A120-101P, 1:2000 dilution) and HRP-conjugated donkey anti-sheep IgG (Novus Bio, NBP1-75437, 1:3000 dilution). Signal detection was performed using the ECL detection reagents (GE Healthcare) and a FluorChem HD2 imaging apparatus (Alpha Innotech).

Fluorescence In Situ Hybridization (FISH)

Metaphase spreads were prepared by incubating cells with 200 ng/ml Colchicine (Sigma-Aldrich) for 2-6 h, mitotic cells were harvested by shake-off and incubated in 0.075 M KCl at 37° C. for 10 min. Chromosomes were fixed in ice-cold methanol/acetic acid (3:1) and spread on glass slides. Slides were treated with 20 μg/ml RNase A (Sigma-Aldrich), in 1×PBS at 37° C. for 1 h, fixed in 4% formaldehyde (Sigma-Aldrich) in 1×PBS for 2 min, and then treated with 70 μg/ml pepsin (Sigma-Aldrich) in 2 mM glycine, pH 2 (Sigma-Aldrich) at 37° C. for 5 min. Slides were fixed again with 4% formaldehyde in 1×PBS for 2 min, incubated subsequently in 70%, 90% and 100% ethanol for 5 min each, and air-dried.

A Cy3-conjugated C-rich telomeric PNA probe (TelC-Cy3; 5′-Cy3-OO-CCCTAACCCTAACCCTAA-3′ [SEQ ID NO:47]; Panagene) diluted in hybridization solution (10 mM Tris-HCl pH 7.2, 70% formamide, 0.5% blocking solution (Roche) was applied onto the slides followed by one incubation at 80° C. for 5 min and one at room temperature for 2 h. Slides were washed twice in 10 mM Tris-HCl pH 7.2, 70% formamide, 0.1% BSA and three times in 0.1 M Tris-HCl pH 7.2, 0.15 M NaCl, 0.08% Tween-20 at room temperature for 10 min each. For native FISH experiments on interphase nuclei, cells grown on coverslips were incubated in CSK buffer (100 mM NaCl, 300 mM sucrose, 3 mM MgCl₂, 10 mM PIPES pH 6.8, 0.5% Triton-X) for 7 min on ice. Cells were then fixed in 4% formaldehyde in 1×PBS for 10 min and permeabilized with CSK buffer for 5 min at room temperature. RNaseH treatments were performed by incubating slides with 30 U of RNaseH (Takara) in 1× RNaseH buffer or only with buffer at 37° C. for 2 h. Hybridizations and washes were performed as above but using a TYE 563-conjugated G-rich telomeric LNA probe (TelG-TYE 563; 5′-TYE563-T*TAGGGT*TAGGGT*TAGGG-3′, asterisks indicate LNA nucleotides; Exiqon). DNA was counterstained with 100 ng/ml DAPI (Sigma-Aldrich) in 1×PBS and slides were mounted in Vectashield (Vectorlabs). Images were acquired with an Olympus IX 81 microscope equipped with a Hamamatsu ORCA-ER camera and a 60×/1.42NA oil PlanApo N objective, or a Zeiss Cell Observer equipped with a cooled Axiocam 506 m camera and a 63×/1.4NA oil DIC M27 PlanApo N objective. Image analysis was performed using ImageJ and Photoshop software.

Combined FISH and EdU Incorporation/Detection

24 h after siRNA transfection cells were incubated in 10 μM RO-3306 (Selleckchem). 21.5 h later 10 μM EdU (Thermo Fisher Scientific) was added to the culture medium, followed by a 2.5 h incubation. Cells were first stained as for DNA FISH using the TelC-Cy3 probe and then washed twice with 1×PBS followed by EdU detection using the Click-iT EdU Alexa Fluor 488 Imaging Kit (Thermo Fisher Scientific) according to the manufacturer's instructions. DNA was counterstained with 100 ng/ml DAPI in 1×PBS and coverslips were mounted on slides in Vectashield. Image acquisition and analysis were as for DNA FISH.

Indirect Immunofluorescence (IF)

Cells grown on coverslips were incubated in CSK buffer for 7 min on ice. All subsequent treatments were performed at room temperature. Cells were fixed with 4% formaldehyde (Sigma-Aldrich) in 1×PBS for 10 min, permeabilized with CSK buffer for 5 min, and incubated in blocking solution (0.5% BSA, 0.1% Tween-20 in 1×PBS) for 1 h. Coverslips were incubated in blocking solution containing primary antibodies for 1 h, washed three times with 0.1% Tween-20 in 1×PBS for 10 min each, and incubated with secondary antibodies diluted in blocking solution for 50 min. DNA was counterstained with 100 ng/ml DAPI in 1×PBS. For combined IF and DNA FISH, cells were again fixed with 4% formaldehyde in 1×PBS for 10 min, washed three times with 1×PBS, incubated in 10 mM Tris-HCl pH 7.2 for 5 min and then denatured and hybridized with TelC-Cy3 probes as described above. DNA was counterstained with 100 ng/ml DAPI in 0.1 M Tris-HCl pH 7.2, 0.15 M NaCl, 0.08% Tween-20 and coverslips were mounted on slides in Vectashield. The following primary antibodies were used: rabbit polyclonal anti-pRPA32 pSer 33 (Bethyl Laboratories, A300-246A, 1:1000 dilution); rabbit polyclonal anti-53BP1 (Abcam, ab21083, 1:1000 dilution); mouse monoclonal anti-TRF2 (Millipore, 05-521, 1:500 dilution); rabbit polyclonal anti-BLM (Bethyl, A300-110A, 1:5000 dilution); rabbit polyclonal anti-PML (a kind gift from M. Carmo-Fonseca, iMM, Lisbon, Portugal, 1:500 dilution); mouse monoclonal anti-RAD51 (Abcam, ab213, 1:100 dilution); mouse monoclonal anti-POLD3 (Novus Biologicals, H00010714-M01, 1:100 dilution); rabbit polyclonal anti-RAP1 (Bethyl, A300-306A, 1:500 dilution). Secondary antibodies were Alexa Fluor 568-conjugated donkey anti-rabbit IgGs (Thermo Fisher Scientific, A10042) and Alexa Fluor 488-conjugated donkey anti-mouse IgGs (Thermo Fisher Scientific, A21202). Image acquisition and analysis were as for DNA FISH.

Genomic DNA Analysis

Genomic DNA was isolated by phenol:chloroform extraction and treatment with 40 μg/ml RNaseA, followed by ethanol precipitation. Reconstituted DNA was digested with HinfI and RsaI (New England Biolabs) and again purified by phenol:chloroform extraction. For TRF analysis, 2 μg of digested DNA were separated on 0.6% agarose gels, which were vacuum-dried at 50° C. for 50 min. Gels were hybridized at 50° C. overnight with telomeric oligonucleotide probes (5′-(TTAGGG)5-3′ or 5′-(CCCTAA)5-3′), 5′-end labeled with T4 polynucleotide kinase (New England Biolabs) and [α-³²P]ATP. Post-hybridization washes were twice in 2×SSC, 0.2% SDS for 20 min and once in 0.5×SSC, 0.2% SDS for 30 min at 50° C. After radioactive signal acquisition, gels were incubated in denaturing solution (1.5 M NaCl, 0.5 M NaOH) at room temperature for 20 min and then hybridized at 55° C. overnight with a double-stranded telomeric probe (Telo2 probe), radioactively labeled using Klenow fragment (New England Biolabs) and [α-³²P]dCTP. Post-hybridization washes were twice in 2×SSC, 0.2% SDS for 20 min and once in 0.2×SSC, 0.2% SDS for 30 min at 55° C. For dot-blot hybridizations, 1 μg of genomic DNA digested as above was denatured for of 5 min at 98° C. or left untreated and dot-blotted on nylon membranes. Membranes were first hybridized with telomeric oligonucleotide probes as above. After radioactive signal acquisition, gels were incubated in denaturing solution as above and then re-hybridized overnight to radiolabeled Alu-repeat oligonucleotides (5′-GTGATCCGCCCGCCTCGGCCTCCCAAAGTG-3′ [SEQ ID NO:48]) at 50° C. Post-hybridization washes were twice in 2×SSC, 0.2% SDS for 20 min and once in 0.5×SSC, 0.2% SDS for 30 min at 50° C. For C-circle assays, 150-500 ng of digested DNA were incubated with 7.5 U phi29 DNA polymerase (New England Biolabs) in presence of dATP, dTTP and dGTP (1 mM each) at 30° C. for 8 h, followed by heat-inactivation at 65° C. for 20 min. Amplification products were dot-blotted onto nylon membranes (GE Healthcare) and hybridized to a radiolabeled Telo2 probe as above. For two-dimensional gel electrophoresis 10 μg of digested DNA were separated on 0.6% agarose gels (pulsed field certified agarose; Biorad) at 30 V for 7 h, followed by excision of the lane and separation of the DNA in the second dimension on 1.1% agarose gels (UltraPure agarose; Life Technologies) at 100 V for 3 h. DNA was then transferred onto nylon membranes, denatured and hybridized to a radiolabeled Telo2 probe as above. Radioactive signals were detected using a Typhoon FLA 9000 imager (GE Healthcare) and quantified using ImageJ software.

Northern Blotting

Total RNA was isolated using the TRIzol reagent (Invitrogen) and treated three times with DNaseI (New England Biolabs). 15 μg of RNA were separated on 1.2% agarose gels containing 0.7% formaldehyde. RNA was then transferred onto nylon membranes and hybridized to a radiolabeled Telo2 probe as above. Ethidium bromide (Sigma-Aldrich) stained tRNAs were used to control for loading. Radioactive signals were detected using a Typhoon FLA 9000 imager (GE Healthcare) and quantified using ImageJ software.

Chromatin Immunoprecipitation (ChIP)

10⁶ cells were harvested by scraping and resuspended in 1 ml of 1% formaldehyde at room temperature for 15 min. After quenching with 125 mM glycine, cells were washed 3 times in 1×PBS by centrifuging at 800 g for 5 min. Cell pellets were resuspended in 500 μl of lysis buffer (1% SDS, 50 mM Tris-HCl pH 8, 10 mM EDTA pH 8) supplemented with complete Protease Inhibitor Cocktail (Roche) and sonicated twice using a Bioruptor apparatus (Diagenode) at 4° C. (settings: 30 seconds “ON”/30 seconds “OFF”; power: “High”; time: 15 min). Cellular debris were pelleted by centrifugation at 1600 g at 4° C. for 10 min and 100 μl of supernatant were mixed with 1.1 ml of IP buffer (1% Triton X-100, 20 mM Tris-HCl pH 8, 2 mM EDTA pH 8, 150 mM NaCl). Diluted extracts were pre-cleared by incubation with 50 μl of protein A/G-sepharose beads (GE Healthcare) blocked with sheared E. coli genomic DNA and BSA at 4° C. for 30 min on a rotating wheel, followed by centrifugation at 800 g at 4° C. for 5 min. Cleared extracts were incubated with 1 μg of anti-FANCM mouse monoclonal antibody (CE56.1⁷²) at 4° C. for 4 hours on a rotating wheel. Immunocomplexes were isolated by incubation with blocked protein A/G beads at 4° C. overnight on a rotating wheel. Beads were washed 4 times with wash buffer 1 (0.1% SDS, 1% Triton X-100, 2 mM EDTA pH 8, 150 mM NaCl, 20 mM Tris-HCl pH 8) and once with wash buffer 2 (0.1% SDS, 1% Triton X-100, 2 mM EDTA pH 8, 500 mM NaCl, 20 mM Tris-HCl pH 8) by centrifuging at 800 g at 4° C. for 5 min. Beads were then incubated in 100 μl of elution buffer (1% Triton X-100, 20 mM Tris-HCl pH 8, 2 mM EDTA pH 8, 150 mM NaCl) containing 40 μg/ml RNaseA at 37° C. for 1 h, followed by incubation at 65° C. overnight to reverse crosslinks. DNA was purified using the Wizard SV Gel and PCR Clean-up kit (Promega), dot-blotted onto nylon membranes and hybridized overnight to a radiolabeled Telo2 probe as for TRF analysis. After signal detection membranes were stripped and re-hybridized overnight to radiolabeled Alu-repeat oligonucleotides as above. Radioactive signals were detected using a Typhoon FLA 9000 imager (GE Healthcare) and quantified using ImageJ software.

DNA:RNA Immunoprecipitation (DRIP)

Cells were harvested by scraping and lysed in 1 ml of RA1 buffer (Macherey-Nagel) containing 1% v/v β-mercaptoethanol and 100 mM NaCl. Nucleic acids were extracted with phenol/chloroform/isoamyl alcohol (25:24:1 saturated with 10 mM Tris-Cl pH 7.0, 1 mM EDTA) and precipitated with isopropanol followed by centrifugation at 15000 g at 4° C. for 10 min. Pellets were washed in 70% ethanol, resuspended in 200 μl Tris-EDTA, 100 mM NaCl and sonicated using a Bioruptor apparatus (Diagenode) at 4° C. (settings: 30 seconds “ON”/30 seconds “OFF”; power: “High”; time: 5 min). 5 μg of nucleic acids were incubated with 1 μg of S9.6 antibody (a kind gift from B. Luke, IMB, Mainz, Germany) in IP buffer (0.1% SDS, 1% Triton X-100, 10 mM HEPES pH 7.2, 0.1% sodium deoxycholate, 275 mM NaCl) at 4° C. for 5 h on a rotating wheel. For RNaseH control experiments, nucleic acids were incubated with 60 U of RNaseH in 1× RNaseH buffer or only with buffer at 37° C. for 3 h prior to incubation with the S9.6 antibody. Immunocomplexes were isolated by incubation with protein G Sepharose beads (GE Healthcare) blocked with sheared E. coli DNA and BSA. Beads were washed four times inIP buffer by centrifuging at 800 g, and incubated in elution buffer (50 mM Tris-Cl pH 8, 10 mM EDTA, 0.5% SDS) containing 10 μg/ml proteinase K (Sigma-Aldrich) and 40 μg/ml RNase A at 50° C. for 30 min. Beads were centrifuged as above and supernatants recovered. Isopropanol-precipitated DNA was dot-blotted onto nylon membranes and hybridized to radiolabeled 5′-(TTAGGG)₅-3′ oligonucleotides as for TRF analysis. After signal detection membranes were stripped and re-hybridized to radiolabeled Alu-repeat oligonucleotides as for ChIP analysis. Radioactive signals were detected using a Typhoon FLA 9000 imager (GE Healthcare) and quantified using ImageJ software.

In Vitro R-Loop Resolution Assays

Flag-FANCM-8HIS:FAAP24 complex was purified using a baculovirus expression system in Sf9 cells. Cells were pelleted at 500×g and lysed on ice in 0.5 M NaCl, 0.02 M Triethanolamine pH 7.5, 1 mM DTT, 10% glycerol plus mammalian protease inhibitors (Sigma-Aldrich) and sonicated on ice 5×10 sec bursts. Clarified lysates were incubated with equilibrated Flag M2 resin (Sigma-Aldrich) for 1 hr and 4° C. Flag resin was subjected to 5× batch washes and eluted with 100 μg/ml Flag peptide. Pooled FANCM-FAAP24 containing elutions were diluted to a final concentration of 100 mM NaCl, 20 mM TEA pH 7.5, 10% glycerol, 1 mM DTT (Buffer B) and bound to 400 μl ssDNA affinity resin (Sigma-Aldrich). The resin was washed with 10 CV of buffer B. FANCM-FAAP24 complexes were eluted with buffer B containing 0.5 M NaCl. 2 μg of pcDNA6-Telo or pcDNA6-TeloR plasmids containing a ˜1 kb fragment of human telomeric repeats cloned downstream of a T7 promoter were in vitro transcribed using T7 polymerase (New England Biolabs) in presence of CTP, GTP, ATP (2.25 mM each) 825 nM [α³²P]UTP (3000 Ci/mmol; Perkin Elmer). Reactions were stopped by heating to 65° C. for 20 min followed by RNase A (EpiCentre) treatment in 330 mM NaCl. R-loop-containing plasmids where purified by two phenol:chloroform extractions. Unincorporated nucleotides were removed by passing nucleic acids twice through S-400 columns (GE Healthcare). R-loop unwinding reactions (10 μl final volume) contained 1 nM R-loop plasmids, 1 mM ATP, 2.5 nM FANCM-FAAP24 in R-loop buffer (6.6 mM Tris pH 7.5, 3% glycerol, 0.1 mM EDTA 1 mM DTT, 0.5 mM MgCl2). Reactions were performed at 37° C. for 10 min and then stopped by adding 2 μl of stop buffer (10 mg/ml proteinase K (New England Biolabs), 1% SDS) and incubating at 37° C. for 15 min. Samples were run on 0.8% agarose TAE gels in TAE buffer (40 mM Tris, 20 mM acetic acid, 1 mM EDTA) at 100V for 60-90 min, followed by gel drying and autoradiography.

Statistical Analysis

For direct comparison of two groups, we employed a paired two-tailed student's t-test using Microsoft Excel or a nonparametric two-tailed Mann-Whitney U test using GraphPad Prism. For comparison of two or more factors for each group and their interaction, we used a two-way analysis of variance (ANOVA) followed by Tukey's HSD for the pairwise comparisons. The analysis was carried out using the aov and TukeyHSD functions of R version 3.3.2. The significance levels are from the Tukey's HSD adjusted P values. P values are indicated as: *P<0.05, **P<0.005, ***P<0.001, ****P<0.0001.

Results

FANCM Supports Viability of ALT Cells

We depleted FANCM in several ALT (U2OS, HuO9, Saos2 and WI-38 VA13) and telomerase positive (Tel+; HeLa, HOS, HT1080 and SKNAS) cells using short interference RNAs (siRNAs) against two sequences from FANCM coding region (siFa and siFb). Non-targeting siRNAs were used as controls (siCt). Two days after transfection, nearly complete depletion of FANCM protein was detected by western blot in Fa- and Fb-transfected cells, with the exception of siFb-transfected SKNAS cells, where about 10% of the protein remained (FIG. 15A). Fluorescence-activated cell sorting (FACS) of ethanol-fixed, propidium iodide (PI)-stained cells revealed that FANCM-depleted ALT cells, but not Tel+, accumulated in G2/M phase (FIGS. 15B and 15C). The clonogenic potential of ALT cells was largely abolished upon transfection of FANCM siRNAs, while the one of Tel+ cells remained essentially unaffected (FIGS. 15D and 15E). For colony formation experiments, cells were transfected only once with siRNAs before seeding and colonies were counted at least 8 days later. Hence, the anti-proliferative effects exerted by FANCM depletion on ALT cells are fast and irreversible. Cell growth analysis upon prolonged siRNA treatment showed that FANCM-depleted U2OS cells were quickly eliminated from the population, while HeLa cells continued to grow although at lower rates (FIG. 15F). Finally, FANCM-depleted U2OS cells, but not HeLa cells, started to be permeable to PI already after 3 days of siRNA-treatment denoting cell death. No major changes in PARP1 cleavage were detected in the same cells. Thus, FANCM depletion causes aberrant accumulation of ALT cells in G2/M phase, followed by PARP1-independent cell death.

Our data indicate that FANCM is essential for cell cycle progression and viability in ALT cells. This is different to what was observed previously″. It is possible that less efficient protein depletion obtained by Pan and colleagues or retained expression of crucial FANCM splice variants (possibly including cell-type specific ones that are not reported in public databases), left residual amounts of FANCM protein sufficient to sustain cell proliferation. Less sensitive cell viability assays might also have underestimated the effects of FANCM depletion in the previous study.

Telomeric Replication Stress Sensitizes ALT Cells to FANCM Depletion

Several features of ALT cells could explain their sensitivity to FANCM depletion: absence of telomerase activity, very long telomeres, ATRX inactivation, and sustained telomeric replication stress. We ectopically expressed the catalytic (hTERT) and RNA (hTR) subunits of telomerase in U2OS and HeLa cells to generate supertelomerase cells³⁷. Over-expression of hTERT and hTR was confirmed by quantitative RT-PCR. As expected, HeLa supertelomerase cells had much longer telomeres than HeLa control cells; U2OS supertelomerase cells had reduced TFE frequencies, while the incidence of under-replicated, fragile telomeres (TFs) remained unchanged^{11, 37}, FANCM depletion inhibited cell proliferation and led to G2/M accumulation in U2OS supertelomerase cells, but not in HeLa supertelomerase cells (FIG. 15B-15E). Moreover, HeLa cells treated with the telomerase inhibitor BIBR 1532³⁸ did not accumulated in G2/M when depleted for FANCM. We then co-depleted FANCM and ATRX in HeLa cells and did not observe accumulation of G2/M cells. Thus, the presence of ultra-long telomeres or the absence of active telomerase or ATRX alone do not explain the sensitivity of ALT cells to FANCM depletion.

We then over-expressed the shelterin factor TRF1 in U2OS cells by retroviral infection, as this treatment halves the incidence of FTs³⁹. FANCM depletion in TRF1 over-expressing cells still led to G2/M accumulation, yet less severely than in cells infected with empty vector (ev) retroviruses (FIGS. 15G and 15H). However, HeLa cells depleted for TRF1 using an siRNA previously shown to induce telomere fragility³⁹ did not accumulate in G2/M when co-depleted for FANCM. Similarly, FANCM-depleted HeLa cells did not show an altered cell cycle distribution when treated with the replication stress inducer hydroxyurea (HU) followed by block release. Hence, telomeric replication stress contributes to the sensitivity of ALT cells to FANCM depletion; nevertheless, telomeric or generalized replication stress alone are not sufficient to sensitize non-ALT cells to FANCM depletion.

FANCM Suppresses Telomeric Replication Stress in ALT Cells

To test the involvement of FANCM in telomere stability, we performed indirect immunofluorescence (IF) using antibodies against TRF2 combined with antibodies against RPA32 phosphorylated at Serine 33 (pS33) or p53 binding protein 1 (53BP1). RPA32 is phosphorylated at serine 33 during S phase by ATR upon replication fork stalling⁴⁰; 53BP1 forms foci at dysfunctional telomeres that have activated either ATR, or the other DNA damage signaling kinase ataxia-telangiectasia mutated (ATM), or both^{41, 42}. Within 48 hours of transfection, pS33 and 53BP1 accumulated at telomeres in FANCM-depleted ALT cells (FIG. 16A) forming the so-called telomere dysfunction-induced foci (TIFs)⁴¹. FANCM depletion did not induce TIF formation in Tel+ cells (FIG. 16A). Accumulation of pSer33 and 53BP1 outside of telomeres was negligible in all FANCM-depleted cell lines (FIG. 16A). FANCM-mediated suppression of telomere instability is likely to be direct, because the protein associated with telomeric DNA in chromatin immunoprecipitation (ChIP) experiments (FIGS. 16B and 16C). FANCM also immunoprecipitated with the abundant, genomewide-spread Alu repeat DNA (FIGS. 16B and 16C), indicating that the protein is not exclusively associated with telomeres. This is consistent with the reported localization of FANCM to cellular chromatin fractions⁴³.

Western blot analysis confirmed that FANCM depletion causes pS33 accumulation and revealed phosphorylation of the other ATR target checkpoint kinase 1 (CHK1) in U2OS but not HeLa cells (FIG. 16D). The ATM target KRAB domain-associated protein 1 (KAP1) was not phosphorylated in any of the tested cell lines (FIG. 16D). Moreover, pS33 accumulation was weakened in FANCM-depleted U2OS cells over-expressing TRF1 (FIG. 15G), while telomerase inhibition, ATRX or TRF1 depletion and HU treatment did not promote pS33 accumulation in FANCM-depleted HeLa cells. Actually, pS33 failed to accumulate efficiently in HeLa cells depleted for FANCM and treated with HU, consistent with a role for FANCM in supporting activation of the canonical ATR-dependent intra S-phase checkpoint³². We propose that FANCM deficiency in ALT cells activates a specific ATR-dependent signaling cascade, which is not fully identical to the one triggered by generalized replication stress and stems at least partly from excessive telomeric replication stress. Such ATR response likely provokes the observed G2/M arrest and cell death.

FANCM Suppresses ALT Features

In our IF images, TRF2 foci in FANCM-depleted ALT cells are both larger and brighter than in control cells. To confirm that this was not simply due to increased TRF2 at telomeres, we subjected siRNA-transfected U2OS interphase cells to DNA fluorescence in situ hybridization (FISH) using telomeric probes, and measured the number and area of telomeric foci. We controlled for possible secondary effects related to cell-cycle stage by arresting siCt-transfected cells at the G2/M border with the cyclin dependent kinase 1 (CDK1) inhibitor RO-3306⁴⁴ (FIGS. 17A and 17B). The overall number of telomeric foci decreased upon FANCM depletion (FIGS. 17C and 17D), while their area distribution was broader, with slightly increased frequencies of very small foci (S in FIGS. 17C and 17E) and substantially increased frequencies of very large foci (L in FIGS. 17C and 17E). RO-3306-treated cells also had less telomeric foci than control cells (FIG. 17D), likely due to clustering of ALT telomeres in G2^{19, 45}. However, the increase in very small and very large foci was more pronounced upon FANCM depletion than RO-3306 treatment (FIGS. 17C and 17E). Approximately 60% of FANCM-depleted cells had at least 5 large foci, versus approximately 10% and 15% of untreated or RO-3306-treated siCt-transfected cells, respectively (FIG. 17F).

We then analyzed the localization of PML, RAD51 and POLD3 at telomeres by combining PML and RAD51 IF with telomere FISH, and double IF for POLD3 and RAPT. We observed increased telomeric localization of all three factors in FANCM-depleted U2OS cells, without obvious increase in PML, POLD3 and RAD51 total protein levels (FIG. 17A; FIG. 18). RO-3306 treatment did not substantially affect the number of telomeric PML and RAD51 foci, while it increased the one of telomeric POLD3 foci yet less importantly than FANCM depletion (FIG. 18). Moreover, we incubated cells treated as above with the thymidine analogue 5-Ethynyl-2′-deoxyuridine (EdU) for 2.5 hours, and performed telomere FISH combined with EdU detection to visualize newly synthesized telomeric DNA. To exclude S phase cells, we only scored cells showing a punctuate EdU staining and with not more than 25 EdU foci. FANCM depletion increased the incidence of telomeric EdU foci, as it did RO-3306 treatment albeit to lower extents (FIG. 18).

We conclude that FANCM depletion exacerbates ALT activity as shown by robust telomere clustering within large APBs containing PML, RAD51 and POLD3, and increased synthesis of telomeric DNA outside of S phase. FANCM depletion also generates short telomeric species, possibly representing ECTRs (see below). G2/M arrest alone cannot explain the aberrantly elevated ALT features observed in FANCM-depleted cells.

FANCM Suppresses Telomeric ssDNA and ECTRs in ALT Cells

We performed in gel telomere restriction fragment (TRF) analysis of genomic DNA from ALT (U2OS and WI-38 VA13) and Tel+(HOS and HeLa) cells harvested 48 hours after siRNA transfection. Blots were hybridized with telomeric oligonucleotides of either 5′-TTAGGG-3′ or 5′-CCCTAA-3′ repeats. When hybridization was performed under native (non-denatured) conditions, we observed increased C-rich telomeric ssDNA of very diverse lengths in FANCM-depleted ALT cells (FIG. 19A, upper panel). Conversely, a decrease of G-rich ssDNA was observed in correspondence of the bulk of telomeres, likely due to shortening of the G-overhang (FIG. 19A, lower panels). For both probes, a fraction of the signal was in the gel wells, possibly corresponding to ssDNA exposed from molecules with significant secondary structures (FIG. 19A). We did not observe alteration of telomeric ssDNA in Tel+ cells (FIG. 19A). Hybridization of the same gels in denatured conditions using a long telomeric probe (Telo2 probe) revealed no appreciable alteration of telomere length in FANCM-depleted cells (FIG. 19A).

We then dot-blotted genomic DNA from cells as above and hybridized it under native conditions to telomeric oligonucleotides, followed by denaturation and hybridization with an Alu repeat, as a control for total DNA loaded. This experiment confirmed that FANCM-depleted ALT cells contain more telomeric C-rich ssDNA than siCt-transfected cells (FIG. 19B). As previously reported¹¹, depletion of RNaseH1 in U2OS cells also increased telomeric C-rich ssDNA, albeit at lower levels than FANCM depletion (FIG. 19B). No major difference in total telomeric DNA was detected using dot-blot hybridization of denatured DNA for U2OS, HOS and HeLa cells (FIG. 19B). An increase in total C-rich telomeric DNA was observed in FANCM-depleted WI-38 VA13 cells (FIG. 19B).

We then performed phi-29-mediated C-circle assays⁴⁶ using DNA from ALT and Tel+ cells and found a remarkable increase in C-circles in ALT cells depleted for FANCM (FIG. 19C). Accumulation of ECTRs, likely to correspond partly but not exclusively to C-circles, was also detected in FANCM-depleted U2OS cells using 2-dimensional gel electrophoresis (FIG. 19D). Metaphase chromosome FISH of FANCM-depleted U2OS cells showed abundant extrachromosomal telomeric signals, probably corresponding to ECTRs, and DNA threads extending from the termini of single chromosomes, or bridging two independent chromosome ends. C-rich ssDNA-containing ECTRs and DNA threads may explain the well-retained DNA molecules observed in our TRF analysis and the increased telomeric ssDNA observed in our dot-blot analysis (FIGS. 19A and 19B). We did not observe an increase in the incidence of TFEs in FANCM depleted U2OS cells.

FANCM Regulates BLM in ALT Cells

FANCM and BLM were reported to collaborate in maintaining ALT telomeres17. We depleted FANCM in U2OS and HeLa cells and performed indirect IF using anti-BLM and anti-TRF2 antibodies. Because FANCM is necessary for BLM recruitment to damage sites induced by stalled replication³⁴, we included cells treated with the topoisomerase I inhibitor Camptothecin (CPT). CPT induced robust formation of nuclear (non-telomeric) BLM foci in siCt-transfected U2OS and HeLa cells, but not in siFa-transfected cells. On the other hand, BLM TIFs were already abundant in siCt-transfected U2OS and only rarely observed in siCt-transfected HeLa cells, and FANCM depletion increased the number of BLM TIFs in U2OS cells. CPT treatment marginally affected TIF frequencies in all samples. BLM nuclear re-localization occurred without major changes in total protein levels. Hence, we confirm that FANCM depletion causes BLM accumulation at ALT telomeres¹⁷, while it prevents it at non-telomeric sites of damage both in ALT and Tel+ cells³⁴. The telomeric accumulation of BLM upon FANCM depletion might involve reported interactions with TRF1 and TRF2⁴⁷

We then depleted FANCM and BLM simultaneously in U2OS cells (FIG. 20A). BLM depletion alone did not alter cell cycle distribution and number of colonies formed, while it decreased proliferation rates and only minimally augmented the fraction of PI-permeable cells (FIG. 20B-20D). Unexpectedly, FANCM and BLM co-depletion resulted in a partial rescue of the aberrant cell cycle distribution and cell proliferation and viability deriving from depleting FANCM (FIG. 20B-20D). Moreover, BLM depletion halved the incidence of pS33 TIFs in cells depleted for FANCM (FIG. 20E). These results establish that BLM depletion alleviates the adverse effects exerted by FANCM deficiency on ALT cells.

FANCM Suppresses TERRA and telR-Loops in ALT Cells

To test whether FANCM suppresses telomere replication stress in ALT cells by regulating TERRA and/or telR-loops, we first performed TERRA northern blot and found that the levels of this lncRNA were 3.5 and 2.5 folds higher in siFa- and siFb-transfected cells, respectively, than in siCt-transfected ones. TERRA species up to ˜2 kb in length were the most affected (FIG. 21A). We then performed in vitro R-loop resolution assays using telR-loop-containing plasmids generated by T7 transcription of a telomeric tract of approximately 1 kb¹¹. We used two plasmids with different insert orientations as to produce transcripts containing TERRA-like, G-rich RNA repeats, or complementary C-rich transcripts (FIG. 21B). As expected¹¹, G-rich transcripts were less efficiently produced than C-rich ones (FIG. 21B). TelR-loop plasmids were incubated with recombinant FANCM in heterodimer with its stabilization partner FAAP24, with or without ATP and then resolved in agarose gels. FANCM promoted complete release of both G-rich and C-rich transcripts from R-loop-plasmids without RNA degradation and in an ATP dependent-manner (FIG. 21B). Thus, FANCM efficiently unwinds the RNA moiety of telR-loops in vitro. To examine telR-loops in FANCM-depleted U2OS cells, we performed DNA: RNA immunoprecipitations (DRIP) using the monoclonal antibody 59.6⁴⁸. Dot-blot hybridization detected telomeric DNA in immunoprecipitated material from all samples, with a ˜3-fold increase in siFa and siFb samples (FIG. 21C). Treatment of nucleic acids with recombinant RNaseH prior to antibody incubation largely abolished hybridization signals, confirming that they emanated from DNA:RNA hybrids (FIG. 21C). We also performed native DNA FISH using G-rich telomeric probes on interphase nuclei treated or not with RNaseH¹¹. A punctate staining corresponding to C-rich telomeric DNA was already visible in untreated siCt-transfected cells, and its intensity was higher in RNaseH treated-cells likely due to degradation of TERRA transcripts within telR-loops and consequent increased binding sites for the probe (FIGS. 21D and 21E). In untreated siFa-transfected cells, the C-rich ssDNA signal was more prominent than in control cells and it was further augmented by RNaseH treatment (FIGS. 21D and 21E). The total number of foci per cell was higher in FANCM-depleted cells but was not affected by RNaseH treatment (FIGS. 21D and 21E). We conclude that FANCM suppresses TERRA and TERRA-containing telR-loops in ALT cells. Considering the ability of FANCM to resolve telR-loops in vitro (FIG. 21B) and the localization of FANCM to telomeres (FIGS. 16C and 16D), we propose that FANCM directly resolves telR-loops on telomeric chromatin. The more prominent C-rich ssDNA signal already present in FANCM-depleted cells not treated with RNaseH (FIGS. 21D and 21E) might originate from gaps in DNA replication or cellular degradation of the RNA moiety of telR-loops. Also, although we refer to the telomeric RNA:DNA hybrid structures arising upon FANCM depletion as telR-loops, our experiments do not distinguish between conventional R-loops, three-stranded nucleic acids comprising an RNA:DNA hybrid and a displaced ssDNA, and ds RNA:DNA hybrids devoid of a displacement loop.

FANCM Averts telR-Loop-Induced Telomeric Replication Stress

We speculated that FANCM suppresses telomeric replication stress by dismantling telR-loops. We depleted FANCM in U2OS cells infected with retroviruses expressing an siRNA-resistant, V5 epitope-tagged FANCM variant (V5-FANCM WT) or an ATPase/translocase inactive counterpart unable to resolve R-loops (V5-FANCM K117R³⁶). Both variants were expressed at higher levels than endogenous FANCM (FIG. 22A). Confirming the specificity of our siRNAs, V5-FANCM WT largely averted G2/M arrest and accumulation of ps33 TIFs and APBs in siFa-transfected cells (FIGS. 22B and 22C). On the contrary, cell cycle distribution and incidence of pS33 TIFs and APBs were similar in V5-FANCM K117R and control cells transfected with siFa (FIGS. 22B and 22C). We then exploited the ability of over-expressed RNaseH1 to suppress telR-loops in cells^{11, 39, 49}. We depleted FANCM alone or in combination with BLM in U2OS cells infected with retroviruses driving over-expression of MYC epitope-tagged RNaseH1 (MYC-RH1 WT) or a catalytically dead counterpart (MYC-RH1 D145A), or with ev control retroviruses (FIG. 22D). MYC-RH1 WT further enhanced the rescue of G2/M arrest defect in cells co-depleted for FANCM and BLM (FIG. 22E). Decreased frequencies of FANCM depletion-induced pS33 TIFs were measured in cells expressing MYC-RH1 WT as compared to ev-infected cells (FIG. 22F). In cells co-depleted for FANCM and BLM and over-expressing MYC-RH1 WT, pS33 TIFs were restored to levels similar to ev control cells (FIG. 22F). In all experiments, MYC-RH1 D145A failed to function as its catalytically active counterpart (FIGS. 22E and 22F). These results indicate that the telomeric replication stress arising upon FANCM depletion is suppressed by FANCM enzymatic activity and stems from unresolved telR-loops and uncontrolled BLM.

We demonstrate here that, in absence of FANCM, ALT cells experience severe telomeric replication stress and activate an ATR-mediated DNA damage signaling, which is likely the trigger of the observed G2/M arrest and cell death⁵⁰. The fact that over-expression of TRF1 renders ALT cells less sensitive to FANCM depletion (FIGS. 15G and 15H) and the lack of accumulation of non-telomeric pSer33 and 53BP1 foci in FANCM-depleted ALT cells (FIG. 16A) confirm the centrality of the signal emanating from damaged telomeres in promoting G2/M arrest. However, FANCM may serve essential functions in ALT cells also outside telomeres, as suggested by its physical interaction with Alu repeat DNA.

We also confirm that FANCM is not essential in all cells, as proliferation and viability of Tel+ cells was not majorly affected by FANCM depletion. Consistently, adult humans carrying biallelic loss of function FANCM mutations were reported^{51, 52}. Moreover, Tel+ human colorectal carcinoma cells, mouse embryonic fibroblasts and chicken lymphoblasts knocked-out for FANCM were successfully generated and proliferated normally unless challenged with DNA damage⁵³⁻⁵⁵. As such, FANCM represents an attractive target in ALT cancer therapy. While it is true that FANCM deficiency is associated with higher risk of breast and liver cancer^{51, 55}, the irreversible lesions rapidly inflicted by FANCM depletion on ALT cells indicate that short-term inhibition of FANCM may efficiently eradicate ALT tumors in absence of secondary effects. On the other side, the previously proposed therapy based on co-inhibition of FANCM and BLM should be avoided″.

We also show that FANCM suppresses ALT-associated features, including clustering of telomeres in PML-, POLD3- and RAD51-containing APBs, and production of ECTRs which include C-circles but possibly other forms (FIG. 18A; FIGS. 19C and 19D). Because the same features were not evident when we depleted FANCM in Tel+ cells, FANCM deficiency alone is not sufficient to initiate ALT de novo. Further in line with FANCM limiting ALT, its depletion increased synthesis of telomeric DNA outside of S phase (FIGS. 18A and 18B) and led to appearance of DNA threads, which may represent intermediates of intermolecular recombination events. We propose that FANCM suppresses POLD3-dependent telomeric BIR in G2 and possibly MiDAS²⁴⁻²⁶. Consistently, deletion of the yeast helicase Mph1, the Saccharomyces cerevisiae FANCM ortholog⁵⁶, directed repair of an HO endonuclease-induced DSB towards BIR57. Moreover, Mph1 over-expression inhibited BIR at intrachromosomal DSBs⁵⁸, while it did not prevent insurgence of telomerase-deficient type II survivors, which are ALT yeasts that maintain their telomeres though BIR⁵⁸⁻⁶². Finally, MphI localizes to short telomeres in an R-loop-dependent manner⁶³. FANCM proteins appear to play specific roles at ALT and uncapped telomeres that are different from the ones that they exert at intrachromosomal damage sites, and that are mediated by R-loops. TelR-loops may directly promote recruitment and/or stabilization and in turn activation of FANCM at telomeres in human ALT cells, thus regulating POLD3-dependent telomeric BIR. The relevance of RAD51 accumulation in APBs when FANCM is depleted remains unclear (FIG. 18A). RAD51 could mediate the telomere clustering observed in FANCM-depleted cells or other molecular events which we did not investigate, such as sister telomere exchanges.

Despite increasing ALT activity, FANCM depletion did not elicit major gain of total (G-rich plus C-rich) telomeric DNA (FIG. 19B). It is possible that in our experiments the amount of newly produced telomeric DNA was below the detection limit. Additionally, de novo synthesis of telomeric DNA in FANCM-depleted cells is likely counterbalanced by incomplete semiconservative replication of telomeric DNA in S phase¹⁷ and by elimination of ECTRs from cells. The exact mechanism by which circular ECTRs are generated remains to be elucidated, nevertheless they are associated with features that increase in FANCM-depleted ALT cells, including replication stress, activated ATR, C-rich telomeric ssDNA and telR-loops^{11,64-66} One observation of our study is that FANCM depletion does not alter TFE frequencies. This suggests that the observed ECTRs do not derive from excision of entire telomeric tracts.

The replication stress arising at ALT telomeres upon FANCM depletion mainly originates from two sources, deregulated BLM and telR-loops. Given the increased BLM recruitment to ALT telomeres when FANCM is depleted, FANCM could directly displace BLM from telomeres. Alternatively, FANCM could suppress the triggers provoking BLM recruitment such as arrested telomeric replication forks or R- and D-loop intermediates. BLM activity promotes ALT by supporting telomeric recombination and BIR-based synthesis of telomeric DNA possibly by resolving recombination intermediates formed during BIR-associated strand invasion as part of the BLM-TOP3A-RMI (BTR) dissolvase complex^{15, 67, 68}. Consistently, FANCM depletion increases telomere synthesis outside of S-phase (FIG. 18A). Moreover, because BLM mediates long-range resection of DNA ends^{69,70}, hyperactive BLM is likely to directly contribute to the production telomeric ssDNA when FANCM is depleted. This is consistent with the low levels of pS33 TIFs detected in cells double depleted for FANCM and BLM (FIG. 22F). As FANCD2 has also been shown to suppress BLM toxicity in ALT cells⁶⁸, one could speculate that this may be a general role for the FA pathway. Nevertheless, the ATPase/translocase activity of FANCM is dispensable for FANCD2 monoubiquitination³¹, and over-expression in U2OS cells of a variant of FANCM unable to recruit the FA complex to chromatin did not suppress any ALT-associated feature (see above). It seems therefore unlikely that the entire FA complex functions to maintain ALT.

As for the nature of telR-loops in FANCM-depleted cells, our data suggest that they accumulate because they are not properly dismantled at telomeric chromatin by the ATPase/translocase activity of FANCM (FIG. 21B-21E). TelR-loops could be generated co-transcriptionally, as telomeric DNA is a difficult substrate for RNA polymerases^{11, 49}. The increased short TERRA species observed in FANCM-depleted ALT cells (FIG. 21A) might indeed derive from premature termination of telomere transcription due to improper telR-loop resolution. Also, the accumulation of pS33 indicates that FANCM most likely resolves telR-loops during S-phase. Thus, improper telR-loop resolution can at least in part explain the diminished efficiency of replication fork progression through the telomeric tract in FANCM-depleted cells¹⁷. Moreover, some FANCM-depletion-associated features of replication-stress, accumulation of C-rich telomeric ssDNA and C-circles (FIGS. 19A, 19B and 19E), are more evident in U2OS cells than in other ALT cells. This might be explained by the fact that TERRA and telR-loops are particularly abundant in U2OS cells¹¹.

Due to the fast and dramatic response of ALT cells to FANCM inactivation (FIG. 15B-15F), our studies had to be performed using siRNAs rather than CRISPR/Cas9-based gene inactivation, obfuscating the analysis of genetic interactions. Nevertheless, the synergism between BLM depletion and RNaseH1 over-expression in suppressing replicative stress in FANCM-depleted cells suggests that BLM activity and telR-loops may be functionally related. Although BLM suppresses R-loops genome-wide,⁷¹ it could promote telR-loop formation specifically in ALT cells, for example by generating C-rich ssDNA followed by TERRA annealing. Conversely, telR-loops could recruit BLM to telomeres by stalling telomeric replication forks or by forming D-loop mimicking structures. Future studies, possibly utilizing conditional knockout cells for FANCM and BLM, should refine this intriguing cellular scenario and open the way to novel avenues for curing ALT cancers.

### REFERENCES

1. Shay, J. W. et al Telomeres and telomerase: three decades of progress. Nat Rev Genet (2019).
2. Shay, J. W. & Bacchetti, S. Eur J Cancer 33, 787-91 (1997).
3. Kim, N. W. et al. Science 266, 2011-5 (1994).
4. Apte, M. S. & Cooper, J. P. Crit Rev Biochem Mol Biol 52, 57-73 (2017).
5. Dunham, M. A., Neumann, A. A., Fasching, C. L. & Reddel, R. R.

Nat Genet 26, 447-50 (2000).
6. Sobinoff, A. P. & Pickett, H. A. Trends Genet 33, 921-932 (2017).
7. Bryan, T. M., et al. Nat Med 3, 1271-4 (1997).
8. Bryan, T. M., Englezou, A., Gupta, J., Bacchetti, S. & Reddel, R. R. EMBO J 14, 4240-8 (1995).
9. Azzalin, C. M. & Lingner, J. Trends Cell Biol 25, 29-36 (2015).
10. Azzalin, C. M., et al J. Science 318, 798-801 (2007).
11. Arora, R. et al. Nat Commun 5, 5220 (2014).
12. Lovejoy, C. A. et al. PLoS Genet 8, e1002772 (2012).
13. Episkopou, H. et al. Nucleic Acids Res 42, 4391-405 (2014).
14. Yeager, T. R. et al. Cancer Res 59, 4175-9 (1999).
15. Stavropoulos, D. J. et al. Hum Mol Genet 11, 3135-44 (2002).
16. Johnson, F. B. et al. EMBO J 20, 905-13 (2001).
17. Pan, X. et al. Proc Natl Acad Sci USA 114, E5940-E5949 (2017).
18. Acharya, S. et al. PLoS One 9, e103819 (2014).
19. Wu, G., Lee, W. H. & Chen, P. L. J Biol Chem 275, 30618-22 (2000).
20. Tokutake, Y. et al. Biochem Biophys Res Commun 247, 765-72 (1998).
21. Ogino, H. et al. Biochem Biophys Res Commun 248, 223-7 (1998).
22. Cesare, A. J. & Griffith, J. D. Mol Cell Biol 24, 9948-57
23. Wang, R. C., Smogorzewska, A. & de Lange, Cell 119, 355-68 (2004).
24. Roumelioti, F. M. et al. EMBO Rep 17, 1731-1737 (2016).
25. Dilley, R. L. et al. Nature 539, 54-58 (2016).
26. Min, J., Wright, W. E. & Shay, J. W. Mol Cell Biol 37(2017).
27. Cho, N. W et al Cell 159, 108-21 (2014). Arora, R. & Azzalin, C. M. RNA Biol, 0 (2015).
29. Flynn, R. L. et al. Science 347, 273-7 (2015). K. E., Marechal, A. & Flynn, R. L. Cell Rep 14, 1032-1040 (2016).
31. Xue, Y., Li, Y., Guo, R., Ling, C. & Wang, W. Hum Mol Genet 17, 1641-52 (2008).
32. Collis, S. J. et al. Mol Cell 32, 313-24 (2008).
33. Crismani, W. et al. Science 336, 1588-90 (2012).
34. Deans, A. J. & West, Mol Cell 36, 943-53 (2009).
35. Gari, K. Proc Natl Acad Sci USA 105, 16107-12 (2008).
36. Schwab, R. A. et al. Mol Cell 60, 351-61 (2015).
37. Cristofari, G. & Lingner, J. EMBO J 25, 565-74 (2006).
38. Damm, K. et al. EMBO J 20, 6958-68 (2001).
39. Lee, Y. W., Arora, R., Wischnewski, H. & Azzalin, C. M. Nat Struct Mol Biol 25, 147-153 (2018).
40. Olson, E., Nievera, C. J., Klimovich, V., Fanning, E. & Wu, X. J Biol Chem 281, 39517-33 (2006).
41. Takai, H., Smogorzewska, A. & de Lange, T. Current Biology 13, 1549-1556 (2003).
42. Denchi, E. L. & de Lange, T. Nature 448, 1068-71 (2007).
43. Kim, J. M., Kee, Y., Gurtan, A. & D'Andrea, A. D. Blood 111, 5215-22 (2008).
44. Vassilev, L. T. et al. Proc Natl Acad Sci USA 103, 10660-5 (2006).
45. Grobelny, J. V., Godwin, A. K. & Broccoli, D. J Cell Sci 113 Pt 24, 4577-85 (2000).
46. Henson, J. D. et al. Methods 114, 74-84 (2017).
47. Lillard-Wetherell, K. et al. Hum Mol Genet 13, 1919-32 (2004).
48. Boguslawski, S. J. et al. J Immunol Methods 89, 123-30 (1986).
49. Sagie, S. et al. Nat Commun 8, 14015 (2017).
50. Saldivar, J. C., Cortez, D. & Cimprich, K. A. Nat Rev Mol Cell Biol 18, 622-636 (2017).
51. Catucci, I. et al. Genet Med 20, 452-457 (2018).
52. Meetei, A. R. et al. Nat Genet 37, 958-63 (2005).
53. Wang, Y. et al. Mol Cell 49, 997-1009 (2013).
54. Mosedale, G. et al. Nat Struct Mol Biol 12, 763-71 (2005).
55. Bakker, S. T. et al. Hum Mol Genet 18, 3484-95 (2009).
56. Xue, X., Sung, P. & Zhao, X. Genes Dev 29, 1777-88 (2015).
57. Mehta, A., Beach, A. & Haber, J. E. Mol Cell 65, 515-526 e3 (2017).
58. Luke-Glaser, S. & Luke, B. PLoS One 7, e42028 (2012).
59. Teng, S. C. & Zakian, V. A. Mol Cell Biol 19, 8083-93 (1999).
60. Lundblad, V. & Blackburn, E. H. Cell 73, 347-60 (1993).
61. Le, S., Moore, J. K., Haber, J. E. & Greider, C. W. Genetics 152, 143-52 (1999).
62. Lydeard, J. R., Jain, S., Yamaguchi, M. & Haber, J. E. Nature 448, 820-3 (2007).
63. Lafuente-Barquero, J. et al. PLoS Genet 13, e1007136 (2017).
64. O'Sullivan, R. J. et al Nat Struct Mol Biol 21, 167-74 (2014).
65. Plantinga, M. J. et al. Mol Cancer Res 11, 557-67 (2013).
66. Oganesian, L. & Karlseder, J. DNA Repair (Amst) 12, 238-45 (2013).
67. Sobinoff, A. P. et al. EMBO J 36, 2907-2919 (2017).
68. Root, H. et al. Hum Mol Genet 25, 3255-3268 (2016).
69. Sturzenegger, A. et al. J Biol Chem 289, 27314-26 (2014).
70. Nimonkar, A. V. et al. Genes Dev 25, 350-62 (2011).
71. Chang, E. Y. et al J Cell Biol 216, 3991-4005 (2017).
72. Vuono, E. A. et al. Sci Rep 6, 36439 (2016).
73. Bryan T M et al. EMBO J. 1995; 14: 4240-4248
74. Bryan T M et al Nat Med. 1997; 3: 1271-1274.
75. Cesare A J et al (2010) Nat Rev Genet 11(5) 319-330.
76. Bayani J, Squire J A. Curr. Protoc. Cell Biol. Chapter 22, Unit 22 27 (2005).
77. Blackford A N, et al., Hum. Mol. Genet. 21, 2005-2016 (2012).
78. Bryan, T M, et al., EMBO J. 14, 4240-4248 (1995).
79. Bryan, T M, et al., Nat. Med. 3, 1271-1274 (1997).
80. Caldon C E, et al., Cell Cycle 12, 606-617 (2013).
81. Cesare A J, et al., Nat. Struct. Mol. Biol. 16, 1244-1251 (2009).
82. Cho N W, et al., Cell 159, 108-121 (2014).
83. Cohen S B and Reddel R R, Nat. Methods. 5, 355-360.
84. Deans A J and West S C. Mol. Cell 36, 943-953 (2009).
85. Dilley R L, et al., Nature 539, 54-58 (2016).
86. Dunham, M A, et al., Telomere maintenance by recombination in human cells. Nat. Genet. 26, 447-450 (2000).
87. Franken N A et al., Nat. Protoc. 1, 2315-2319 (2006).
88. Fox D, 3rd, et al., Cell Res. 24, 560-575 (2014).
89. Gari K, et al., Mol. Cell 29, 141-148 (2008).
90. Hoadley K A, et al., Proc. Natl. Acad. Sci. U.S.A., 109, 4437-4442 (2012).
91. Heaphy C M et al., Am J Pathol. 179(4):1608-1615 (2011).
92. Henson J D, et al., Nat. Biotechnol. 27, 1181-1185 (2009).
93. Huang M et al., Mol. Cell 39, 259-268 (2010).
94. Huang J, et al., Mol. Cell 52, 434-446 (2013).
95. Jiang, W Q, et al., Mol. Cell. Biol. 25, 2708-2721 (2005).
96. Kim J M, et al., Blood 111, 5215-5222 (2008).
97. Lau M S et al., Nucleic Acids Res. 41, e34 (2013).
98. Lee M et al., Nucleic Acids Res. 146(10):4903-4918. (2018).
99. Meyers R M, et al., Nat. Genet. 49, 1779-1784 (2017).
100. Murnane, J P, et al., EMBO J. 13, 4953-4962 (1994).
101. Pan X et al., Proc. Natl. Acad. Sci. U.S.A. 114, E5940-E5949 (2017).
102. Pentz R, et al., Nat. Commun. (2019).
103. Perrem, K, et al., Mol. Cell. Biol. 21, 3862-3875 (2001).
104. Rohleder F, et al., Nucleic Acids Res. 44, 3219-3232 (2016).
105. Singh T R, et al., Cancer Res. 73, 4300-4310 (2013).
106. Sobinoff A P, et al., EMBO J. 36, 2907-2919 (2017).
107. Verma P and Greenberg R A, Genes Dev. 30, 1138-1154 (2016).
108. Verma P, et al., Methods Enzymol. 600, 107-134 (2018).
109. Voter A F, et al., J. Biomol. Screen. 21, 626-633 (2016).
110. Xue Y et al., Hum. Mol. Genet. 17, 1641-1652 (2008).
111. Yan Z, et al., Mol. Cell 37, 865-878 (2010).
112. Yeager, T R, et al., Cancer Res. 59, 4175-4179 (1999).
113. Zhao Q, et al., Nat. Commun. 5, 2987 (2014).

1. A method of inhibiting Alternative Lengthening of Telomeres (ALT) cell viability and/or growth of an ALT cell, comprising reducing Fanconi anemia, complementation group M (FANCM) expression or activity in the ALT cell.

2. The method according to claim 1, wherein FANCM expression or activity is reduced by administering a FANCM antagonist to the individual.

3. The method according to claim 2, wherein the FANCM antagonist inhibits one or more activity of FANCM.

4. The method according to claim 3, wherein the FANCM antagonist is an organic compound having a molecular weight of 900 Da or less.

5. The method according to claim 3, wherein the FANCM antagonist is an antibody molecule or aptamer that specifically binds to FANCM.

6. The method according to claim 2, wherein the FANCM antagonist reduces the expression of FANCM.

7. The method according to claim 6, wherein the FANCM antagonist is a suppressor nucleic acid.

8. The method according to claim 7, wherein the suppressor nucleic acid is a siRNA or shRNA.

9. The method according to claim 8, wherein the suppressor nucleic acid comprises a nucleotide sequence at least 95% identical to a contiguous sequence of 15 to 40 nucleotides of SEQ ID NO: 32.

10. The method according to claim 9, wherein the suppressor nucleic acid comprises the nucleotide sequence of SEQ ID NO: 3 or SEQ ID NO: 4.

11. The method according to claim 7, wherein the suppressor nucleic acid is an antisense oligonucleotide.

12. The method according to claim 6, wherein the FANCM antagonist is a targeted nuclease that reduces expression of FANCM.

13. The method according to claim 12, wherein the targeted nuclease is a ZFN, TALEN or meganuclease that recognises a target sequence within the FANCM gene.

14. The method according to claim 12 wherein the targeted nuclease is a CRISPR associated nuclease, said CRISPR associated nuclease being administered in combination with a guide RNA that recognises a target sequence within the FANCM gene.

15. The method according to claim 12 or claim 13 wherein the targeted nuclease cleaves genomic DNA at the target sequence of the FANCM gene, thereby causing a deletion or insertion which reduces or prevents expression of active FANCM polypeptide.

16. The method according to any one of the preceding claims, wherein activity or expression of BLM and/or BRCA1 is not reduced in the ALT cell.

17. The method according to any one of the preceding claims, wherein the ALT cell is a mesenchymal or epithelial cancer cell.

18. The method according to claim 17, wherein the ALT cell is an osteosarcoma, liposarcoma, glioblastoma, astrocytoma, or bladder carcinoma cell.

19. The method according to claim 1, wherein the method comprises disrupting the FANCM-RMI interaction and/or inhibiting FANCM ATPase activity.

20. The method according to claim 19, wherein disrupting the FANCM-RMI interaction comprises administering an inhibitor of the FANCM-RMI interaction, and wherein inhibiting FANCM ATPase activity comprises administering an inhibitor of FANCM ATPase activity.

21. The method according to claim 19 or claim 20, comprising disrupting the binding of FANCM to RMI at the MM2 domain.

22. The method according to any one of claims 19-21, wherein the inhibitor is any one or more of a genetic inhibitor, a small molecule, a peptide and a protein.

23. The method according to claim 22, wherein the genetic inhibitor is siRNA.

24. The method according to claim 22, wherein the small molecule is 4-[(1-Hydroxy-2-phenyl-1H-indol-3-yl)-pyridin-2-yl-methyl]-piperazine-1-carboxylic acid ethyl ester.

25. The method according to claim 22, wherein the peptide is a peptide that comprises an amino acid sequence at least 90% identical to a peptide selected from the group consisting of:
(SEQ ID NO: 49)
DLFSVTFDLGFC,
(SEQ ID NO: 50)
DIFDCSRDLFSVTFDLGFCSPDSDDEILEHTSD,
and
(SEQ ID NO: 5)
IFDCSRDLFSVTFDLGFCSPDSDDEILEHTSD.

26. The method according to claim 22, wherein the protein is an inactivated FANCM protein.

27. The method according to claim 26, wherein the inactivated FANCM protein comprises a F1232A/F1236A double substitution.

28. The method according to claim 22, wherein the protein comprises an immunoglobulin binding domain.

29. The method of claim 19, which is a method of treating ALT cancer in a subject.

30. A method of treating an Alternative Lengthening of Telomeres (ALT) cancer in an individual in need thereof, comprising reducing Fanconi anemia, complementation group M (FANCM) expression or activity in the individual.

31. The method according to claim 30, wherein FANCM expression or activity is reduced by administering an FANCM antagonist to the individual.

32. The method according to claim 31, wherein the FANCM antagonist inhibits an activity of FANCM.

33. The method according to claim 32, wherein the FANCM antagonist is an organic compound having a molecular weight of 900 Da or less.

34. The method according to claim 32, wherein the FANCM antagonist is an antibody molecule or aptamer that specifically binds to FANCM.

35. The method according to claim 31, wherein the FANCM antagonist reduces the expression of FANCM.

36. The method according to claim 35, wherein the FANCM antagonist is a suppressor nucleic acid.

37. The method according to claim 36, wherein the suppressor nucleic acid is a siRNA or shRNA.

38. The method according to claim 37, wherein the suppressor nucleic acid comprises a nucleotide sequence at least 95% identical to a contiguous sequence of 15 to 40 nucleotides of SEQ ID NO: 32.

39. The method according to claim 38, wherein the suppressor nucleic acid comprises the nucleotide sequence of SEQ ID NO: 3 or SEQ ID NO: 4.

40. The method according to claim 36, wherein the suppressor nucleic acid is an antisense oligonucleotide.

41. The method according to claim 35, wherein the FANCM antagonist is a targeted nuclease that reduces expression of FANCM.

42. The method according to claim 41, wherein the targeted nuclease is a ZFN, TALEN or meganuclease that recognises a target sequence within the FANCM gene.

43. The method according to claim 41 wherein the targeted nuclease is a CRISPR associated nuclease, said CRISPR associated nuclease being administered in combination with a guide RNA that recognises a target sequence within the FANCM gene.

44. The method according to claim 41 or claim 42 wherein the targeted nuclease cleaves genomic DNA at the target sequence of the FANCM gene, thereby causing a deletion or insertion which reduces or prevents expression of active FANCM polypeptide.

45. The method according to any one of claims 30-44, wherein activity or expression of BLM and/or BRCA1 is not reduced in the ALT cell.

46. The method according to any one of claims 30-45, wherein the ALT cancer is a mesenchymal or epithelial cancer.

47. The method according to claim 46, wherein the ALT cancer is an osteosarcoma, soft tissue sarcoma (e.g., liposarcoma, undifferentiated pleomorphic sarcoma, or leiomyosarcoma), glioblastoma, astrocytoma, neuroblastoma, or bladder carcinoma.

48. An agent that reduces the expression or activity of FANCM for use in a method of treatment according to any one of claims 30 to 47.

49. Use of an FANCM antagonist in the manufacture of a medicament for use in a method of treatment according to any one of claims 30 to 47.

50. The method according to claim 30, wherein the method comprises disrupting the FANCM-RMI interaction and/or inhibiting the ATPase activity of FANCM.

51. The method according to claim 50, wherein disrupting the FANCM-RMI interaction comprises administering an inhibitor of the FANCM-RMI interaction and/or an inhibitor of the ATPase activity of FANCM and/or administering an agent that inhibits the ATPase activity of FANCM.

52. The method according to claim 50 or claim 51, comprising disrupting the binding of FANCM to RMI at the MM2 domain.

53. The method according to any one of claims 50-52, wherein the inhibitor is any one or more of a genetic inhibitor, a small molecule, a peptide and a protein.

54. The method according to claim 53, wherein the genetic inhibitor is siRNA.

55. The method according to claim 53, wherein the small molecule is 4-[(1-Hydroxy-2-phenyl-1H-indol-3-yl)-pyridin-2-yl-methyl]-piperazine-1-carboxylic acid ethyl ester.

56. The method according to claim 53, wherein the peptide is a peptide that comprises an amino acid sequence at least 90% identical to a peptide selected from the group consisting of:
(SEQ ID NO: 49)
DLFSVTFDLGFC,
(SEQ ID NO: 50)
DIFDCSRDLFSVTFDLGFCSPDSDDEILEHTSD,
and
(SEQ ID NO: 5)
EDIFDCSRDLFSVTFDLGFCSPDSDDEILEHTSD.

57. The method according to claim 53, wherein the protein is an inactivated FANCM protein.

58. The method according to claim 57, wherein the inactivated FANCM protein comprises a F1232A/F1236A double substitution and/or a K117R substitution.

59. The method according to claim 53, wherein the protein comprises an immunoglobulin binding domain.

60. The method according to claim 51 or claim 52, further comprising the simultaneous, sequential or separate administration of a chemotherapeutic agent.

61. The method according to claim 51 or claim 52, wherein the method does not comprise the simultaneous, sequential or separate administration of a chemotherapeutic agent.

62. A pharmaceutical composition comprising an inhibitor of the FANCM-RMI interaction and/or an inhibitor of the ATPase activity of FANCM, for use in treating an Alternative Lengthening of Telomeres (ALT) cancer.

63. Use of an inhibitor of the FANCM-RMI interaction and/or an inhibitor of the ATPase activity of FANCM, in the manufacture of a medicament for the treatment of an Alternative Lengthening of Telomeres (ALT) cancer.

64. The pharmaceutical composition according to claim 62, wherein the pharmaceutical composition consists essentially of an inhibitor of the FANCM-RMI interaction and/or an inhibitor of the ATPase activity of FANCM; or the use according to claim 19, wherein the medicament consists essentially of an inhibitor of the FANCM-RMI interaction and/or an inhibitor of the ATPase activity of FANCM.

65. The pharmaceutical composition according to claim 62 or claim 64, or the use according to claim 19 or claim 20, wherein the inhibitor of the FANCM-RMI interaction and/or the inhibitor of the ATPase activity of FANCM, is any one or more of a genetic inhibitor, a small molecule, a peptide and a protein.

66. The pharmaceutical composition or the use of claim 65, wherein the genetic inhibitor is siRNA.

67. The pharmaceutical composition or the use of claim 65, wherein the small molecule is 4-[(1-Hydroxy-2-phenyl-1H-indol-3-yl)-pyridin-2-yl-methyl]-piperazine-1-carboxylic acid ethyl ester.

68. The pharmaceutical composition or the use of claim 65, wherein the peptide comprises an amino acid sequence at least 90% identical to a peptide selected from the group consisting of:
(SEQ ID NO: 49)
DLFSVTFDLGFC,
(SEQ ID NO: 50)
DIFDCSRDLFSVTFDLGFCSPDSDDEILEHTSD
and
(SEQ ID NO: 5)
EDIFDCSRDLFSVTFDLGFCSPDSDDEILEHTSD.

69. The pharmaceutical composition or the use of claim 65, wherein the protein is an inactivated FANCM protein.

70. The pharmaceutical composition or the use of claim 69, wherein the inactivated FANCM protein comprises a F1232A/F1236A double substitution and/or a K117R substitution.

71. The pharmaceutical composition or the use of claim 65, wherein the protein comprises an immunoglobulin binding domain.

72. A method of determining the responsiveness of a cancer in an individual to treatment with a FANCM antagonist, comprising determining the presence of one or more ALT cancer cells in a sample of cancer cells from the individual, the presence of one or more ALT cancer cells in the sample being indicative that the cancer is responsive to treatment with the FANCM antagonist.

73. The method according to claim 72, wherein the presence of ALT cancer cells is determined by assessing the presence of C-circles or ALT associated PML bodies in one or more cancer cells in the sample, the presence of C-circles or ALT associated PML bodies in the one or more cancer cells being indicative that the cancer cells are ALT cancer cells.

74. The method according to claim 72 or claim 73, comprising identifying the cancer in the individual as responsive to a FANCM antagonist.

75. A method of selecting a subject for treatment with an inhibitor of the FANCM-RMI interaction, the method comprising determining whether the subject is suffering from ALT cancer, wherein the subject is selected for treatment with the inhibitor of the FANCM-RMI interaction if the subject is suffering from ALT cancer.

76. A method of identifying whether a subject suffering from cancer is suitable for treatment with an inhibitor of the FANCM-RMI interaction, comprising determining whether the cancer is ALT cancer, wherein the subject is identified as suitable for treatment with the inhibitor of the FANCM-RMI interaction if the subject is suffering from ALT cancer.

77. A method of determining whether a subject is responding to treatment with an inhibitor of the FANCM-RMI interaction, comprising:
determining the presence and/or extent of genomic instability at one or more telomeres in a cell taken from a subject; and/or
determining the presence and/or level of ALT activity in a cell taken from a subject.

78. The method according to claim 77, wherein the cell is an ALT cancer cell.

79. The method according to any one of claims 75-78, wherein the inhibitor is any one or more of a genetic inhibitor, a small molecule, a peptide and a protein.

80. The method according to claim 79, wherein the genetic inhibitor is siRNA.

81. The method according to claim 79, wherein the small molecule is 4-[(1-Hydroxy-2-phenyl-1H-indol-3-yl)-pyridin-2-yl-methyl]-piperazine-1-carboxylic acid ethyl ester.

82. The method according to claim 79, wherein the peptide is a peptide that comprises an amino acid sequence at least 90% identical to a peptide selected from the group consisting of:
(SEQ ID NO: 49)
DLFSVTFDLGFC,
(SEQ ID NO: 50)
DIFDCSRDLFSVTFDLGFCSPDSDDEILEHTSD
and
(SEQ ID NO: 5)
EDIFDCSRDLFSVTFDLGFCSPDSDDEILEHTSD.

83. The method according to claim 79, wherein the protein is an inactivated FANCM protein.

84. The method according to claim 83, wherein the inactivated FANCM protein comprises a F1232A/F1236A double substitution and/or a K117R substitution.

85. The method according to claim 79, wherein the protein comprises an immunoglobulin binding domain.

86. A method of screening for a compound that induces the death of ALT cancer cells comprising:
determining the binding of a test compound to FANCM,
wherein binding to FANCM is indicative that the compound induces cell death in ALT cells.

87. A method of screening for a compound that induces the death of ALT cancer cells, comprising determining the effect of a test compound on the expression or activity of FANCM,
wherein a reduction in expression or activity of FANCM is indicative that the compound induces cell death in ALT cells.

88. A method according to claim 87 or claim 88, comprising identifying the test compound as a compound which reduces the express or activity of FANCM.

89. A method according to claim 88, comprising isolating or purifying the identified compound.


SEQUENCE LISTING (50 sequences)

SEQ ID NO: 1 - protein, 1132 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 2 - protein, 2048 aa, Homo sapiens
MSGRQRTLFQTWGSSISRSSGTPGCSSGTERPQSPGSSKAPLPAAAEAQLESDDDVLLVAAYEAERQLCLENGGFCTSAGALWIYPTNCPVRDYQLHISRAALFCNTLVCLPTGLGKTFIAAVVMYNFYRWFPSGKVVFMAPTKPLVTQQIEACYQVMGIPQSHMAEMTGSTQASTRKEIWCSKRVLFLTPQVMVNDLSRGACPAAEIKCLVIDEAHKALGNYAYCQVVRELVKYTNHFRILALSATPGSDIKAVQQVITNLLIGQIELRSEDSPDILTYSHERKVEKLIVPLGEELAAIQKTYIQILESFARSLIQRNVLMRRDIPNLTKYQIILARDQFRKNPSPNIVGIQQGIIEGEFAICISLYHGYELLQQMGMRSLYFFLCGIMDGTKGMTRSKNELGRNEDFMKLYNHLECMFARTRSTSANGISAIQQGDKNKKFVYSHPKLKKLEEVVIEHFKSWNAENTTEKKRDETRVMIFSSFRDSVQEIAEMLSQHQPIIRVMTFVGHASGKSTKGFTQKEQLEVVKQFRDGGYNTLVSTCVGEEGLDIGEVDLIICFDSQKSPIRLVQRMGRTGRKRQGRIVIILSEGREERIYNQSQSNKRSIYKAISSNRQVLHFYQRSPRMVPDGINPKLHKMFITHGVYEPEKPSRNLQRKSSIFSYRDGMRQSSLKKDWFLSEEEFKLWNRLYRLRDSDEIKEITLPQVQFSSLQNEENKPAQESTTGIHQLSLSEWRLWQDHPLPTHQVDHSDRCRHFIGLMQMIEGMRHEEGECSYELEVESYLQMEDVTSTFIAPRNESNNLASDTFITHKKSSFIKNINQGSSSSVIESDEECAEIVKQTHIKPTKIVSLKKKVSKEIKKDQLKKENNHGIIDSVDNDRNSTVENIFQEDLPNDKRTSDTDEIAATCTINENVIKEPCVLLTECQFTNKSTSSLAGNVLDSGYNSFNDEKSVSSNLFLPFEEELYIVRTDDQFYNCHSLTKEVLANVERFLSYSPPPLSGLSDLEYEIAKGTALENLLFLPCAEHLRSDKCTCLLSHSAVNSQQNLELNSLKCINYPSEKSCLYDIPNDNISDEPSLCDCDVHKHNQNENLVPNNRVQIHRSPAQNLVGENNHDVDNSDLPVLSTDQDESLLLFEDVNTEFDDVSLSPLNSKSESLPVSDKTAISETPLVSQFLISDELLLDNNSELQDQITRDANSFKSRDQRGVQEEKVKNHEDIFDCSRDLFSVTFDLGFCSPDSDDEILEHTSDSNRPLDDLYGRYLEIKEISDANYVSNQALIPRDHSKNFTSGTVIIPSNEDMQNPNYVHLPLSAAKNEELLSPGYSQFSLPVQKKVMSTPLSKSNTLNSFSKIRKEILKTPDSSKEKVNLQRFKEALNSTFDYSEFSLEKSKSSGPMYLHKSCHSVEDGQLLTSNESEDDEIFRRKVKRAKGNVLNSPEDQKNSEVDSPLHAVKKRRFPINRSELSSSDESENFPKPCSQLEDFKVCNGNARRGIKVPKRQSHLKHVARKFLDDEAELSEEDAEYVSSDENDESENEQDSSLLDFLNDETQLSQAINDSEMRAIYMKSLRSPMMNNKYKMIHKTHKNINIFSQIPEQDETYLEDSFCVDEEESCKGQSSEEEVCVDFNLITDDCFANSKKYKTRRAVMLKEMMEQNCAHSKKKLSRIILPDDSSEEENNVNDKRESNIAVNPSTVKKNKQQDHCLNSVPSGSSAQSKVRSTPRVNPLAKQSKQTSLNLKDTISEVSDFKPQNHNEVQSTTPPFTTVDSQKDCRKFPVPQKDGSALEDSSTSGASCSKSRPHLAGTHTSLRLPQEGKGTCILVGGHEITSGLEVISSLRAIHGLQVEVCPLNGCDYIVSNRMVVERRSQSEMLNSVNKNKFIEQIQHLQSMFERICVIVEKDREKTGDTSRMFRRTKSYDSLLTTLIGAGIRILFSSCQEETADLLKELSLVEQRKNVGIHVPTVVNSNKSEALQFYLSIPNISYITALNMCHQFSSVKRMANSSLQEISMYAQVTHQKAEEIYRYIHYVFDIQMLPNDLNQDRLKSDI

SEQ ID NO: 3 - protein, 625 aa, Homo sapiens
MNVTSIALRAETWLLAAWHVKVPPMWLEACINWIQEENNNVNLSQAQMNKQVFEQWLLTDLRDLEHPLLPDGILEIPKGELNGFYALQINSLVDVSQPAYSQIQKLRGKNTTNDLVTAEAQVTPKPWEAKPSRMLMLQLTDGIVQIQGMEYQPIPILHSDLPPGTKILIYGNISFRLGVLLLKPENVKVLGGEVDALLEEYAQEKVLARLIGEPDLVVSVIPNNSNENIPRVTDVLDPALGPSDEELLASLDENDELTANNDTSSERCFTTGSSSNTIPTRQSSFEPEFVISPRPKEEPSNLSIHVMDGELDDFSLEEALLLEETVQKEQMETKELQPLTFNRNADRSIERFSHNPNTTNNFSLTCKNGNNNWSEKNVSEQMTNEDKSFGCPSVRDQNRSIFSVHCNVPLAHDFTNKEKNLETDNKIKQTSSSDSHSLNNKILNREVVNYVQKRNSQISNENDCNLQSCSLRSSENSINLSIAMDLYSPPFVYLSVLMASKPKEVTTVKVKAFIVTLTGNLSSSGGIWSITAKVSDGTAYLDVDFVDEILTSLIGFSVPEMKQSKKDPLQYQKFLEGLQKCQRDLIDLCCLMTISFNPSLSKAMVLALQDVNMEHLENLKKRLNK

SEQ ID NO: 4 - protein, 147 aa, Homo sapiens
MAAAADSFSGGPAGVRLPRSPPLKVLAEQLRRDAEGGPGAWRLSRAAAGRGPLDLAAVWMQGRVVMADRGEARLRDPSGDFSVRGLERVPRGRPCLVPGKYVMVMGVVQACSPEPCLQAVKMTDLSDNPIHESMWELEVEDLHRNIP

SEQ ID NO: 5 - protein, 34 aa, Homo sapiens
EDIFDCSRDLFSVTFDLGFCSPDSDDEILEHTSD

SEQ ID NO: 6 - protein, 1417 aa, Homo sapiens
MAAVPQNNLQEQLERHSARTLNNKLSLSKPKFSGFTFKKKTSSDNNVSVTNVSVAKTPVLRNKDVNVTEDFSFSEPLPNTTNQQRVKDFFKNAPAGQETQRGGSKSLLPDFLQTPKEVVCTTQNTPTVKKSRDTALKKLEFSSSPDSLSTINDWDDMDDFDTSETSKSFVTPPQSHFVRVSTAQKSKKGKRNFFKAQLYTTNTVKTDLPPPSSESEQIDLTEEQKDDSEWLSSDVICIDDGPIAEVHINEDAQESDSLKTHLEDERDNSEKKKNLEEAELHSTEKVPCIEFDDDDYDTDFVPPSPEEIISASSSSSKCLSTLKDLDTSDRKEDVLSTSKDLLSKPEKMSMQELNPETSTDCDARQISLQQQLIHVMEHICKLIDTIPDDKLKLLDCGNELLQQRNIRRKLLTEVDFNKSDASLLGSLWRYRPDSLDGPMEGDSCPTGNSMKELNFSHLPSNSVSPGDCLLTTTLGKTGFSATRKNLFERPLFNTHLQKSFVSSNWAETPRLGKKNESSYFPGNVLTSTAVKDQNKHTASINDLERETQPSYDIDNFDIDDFDDDDDWEDIMHNLAASKSSTAAYQPIKEGRPIKSVSERLSSAKTDCLPVSSTAQNINFSESIQNYTDKSAQNLASRNLKHERFQSLSFPHTKEMMKIFHKKFGLHNFRTNQLEAINAALLGEDCFILMPTGGGKSLCYQLPACVSPGVTVVISPLRSLIVDQVQKLTSLDIPATYLTGDKTDSEATNIYLQLSKKDPIIKLLYVTPEKICASNRLISTLENLYERKLLARFVIDEAHCVSQWGHDFRQDYKRMNMLRQKFPSVPVMALTATANPRVQKDILTQLKILRPQVFSMSFNRHNLKYYVLPKKPKKVAFDCLEWIRKHHPYDSGIIYCLSRRECDTMADTLQRDGLAALAYHAGLSDSARDEVQQKWINQDGCQVICATIAFGMGIDKPDVRFVIHASLPKSVEGYYQESGRAGRDGEISHCLLFYTYHDVTRLKRLIMMEKDGNHHTRETHFNNLYSMVHYCENITECRRIQLLAYFGENGFNPDFCKKHPDVSCDNCCKTKDYKTRDVTDDVKSIVRFVQEHSSSQGMRNIKHVGPSGRFTMNMLVDIFLGSKSAKIQSGIFGKGSAYSRHNAERLFKKLILDKILDEDLYINANDQAIAYVMLGNKAQTVLNGNLKVDFMETENSSSVKKQKALVAKVSQREEMVKKCLGELTEVCKSLGKVFGVHYFNIFNTVTLKKLAESLSSDPEVLLQIDGVTEDKLEKYGAEVISVLQKYSEWTSPAEDSSPGISLSSSRGPGRSAAEELDEEIPVSSHYFASKTRNERKRKKMPASQRSKRRKTASSGSKAKGGSATCRKISSKTKSSSIIGSSSASHTSQATSGANSKLGIMAPPKPINRPFLKPSYAFS

SEQ ID NO: 7 - protein, 1001 aa, Homo sapiens
MIFPVARYALRWLRRPEDRAFSRAAMEMALRGVRKVLCVAEKNDAAKGIADLLSNGRMRRREGLSKFNKIYEFDYHLYGQNVTMVMTSVSGHLLAHDFQMQFRKWQSCNPLVLFEAEIEKYCPENFVDIKKTLERETRQCQALVIWTDCDREGENIGFEIIHVCKAVKPNLQVLRARFSEITPHAVRTACENLTEPDQRVSDAVDVRQELDLRIGAAFTRFQTLRLQRIFPEVLAEQLISYGSCQFPTLGFVVERFKAIQAFVPEIFHRIKVTHDHKDGIVEFNWKRHRLFNHTACLVLYQLCVEDPMATVVEVRSKPKSKWRPQALDTVELEKLASRKLRINAKETMRIAEKLYTQGYISYPRTETNIFPRDLNLTVLVEQQTPDPRWGAFAQSILERGGPTPRNGNKSDQAHPPIHPTKYTNNLQGDEQRLYEFIVRHFLACCSQDAQGQETTVEIDIAQERFVAHGLMILARNYLDVYPYDHWSDKILPVYEQGSHFQPSTVEMVDGETSPPKLLTEADLIALMEKHGIGTDATHAEHIETIKARMYVGLTPDKRFLPGHLGMGLVEGYDSMGYEMSKPDLRAELEADLKLICDGKKDKFVVLRQQVQKYKQVFIEAVAKAKKLDEALAQYFGNGTELAQQEDIYPAMPEPIRKCPQCNKDMVLKTKKNGGFYLSCMGFPECRSAVWLPDSVLEASRDSSVCPVCQPHPVYRLKLKFKRGSLPPTMPLEFVCCIGGCDDTLREILDLRFSGGPPRASQPSGRLQANQSLNRMDNSQHPQPADSRQTGSSKALAQTLPPPTAAGESNSVTCNCGQEAVLLTVRKEGPNRGRQFFKCNGGSCNFFLWADSPNPGAGGPPALAYRPLGASLGCPPGPGIHLGGFGNPGDGSGSGTSCLCSQPSVTRTVQKDGPNKGRQFHTCAKPREQQCGFFQWVDENTAPGTSGAPSWTGDRGRTLESEARSKRPRASSSDMGSTAKKPRKCSLCHQPGHTRPFCPQNR

SEQ ID NO: 8 - protein, 1863 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 9 - DNA, 6147 bp, Homo sapiens
atgagcggacggcaaagaacgctttttcagacgtggggctcaagtatctcccgatcatctgggactccgggttgcagctccggaactgagcgacctcagagccctggcagctccaaggcgcctttgccagcagcagcggaggctcagctggagtcggacgatgatgtgttgcttgtcgcggcgtacgaggctgagcggcagttgtgtctagagaatggcgggttctgcacctccgcgggcgccctgtggatttaccctaccaattgcccagtgcgggactaccagctgcacatttcccgggctgctctgttttgcaatacgctggtgtgtctgcctaccggactgggaaagacctttattgccgccgtggtcatgtacaatttctaccgctggttcccttcaggaaaggtggtcttcatggccccaacgaaacccttggtgacacagcagatcgaggcttgctaccaggtgatgggtatcccgcaatcccacatggccgaaatgacagggtctacacaagcttccaccaggaaggaaatatggtgcagtaagagagtgctttttcttacacctcaggtcatggtaaatgacctttctagaggagcttgtcccgctgctgaaataaagtgtttagttattgatgaagctcataaagctctcggaaactatgcttattgccaggttgtaagagaactagtcaaatatacaaatcactttagaatcttggctctaagtgccacaccaggtagtgatataaaggctgtgcaacaagttattactaacctgctaattgggcagatagagcttcgttctgaagattctccagatattttgacatattctcatgaaagaaaagttgaaaagcttattgttccgcttggtgaagaacttgcagccatccaaaagacctatatccagattttggaatcatttgctcgttctttgattcagaggaatgttttgatgagaagggatatcccaaatctaacaaaatatcagataattctggcaagagatcagtttaggaaaaacccatctccgaatattgtgggaatacaacaaggcataatcgagggagagtttgctatttgtattagtttatatcatggttatgaattattgcagcaaatgggaatgagatcattatatttcttcctttgtggaattatggatggaactaaagggatgacacggtcaaaaaatgaacttggccgaaatgaagacttcatgaaactctataatcatctagagtgtatgtttgcacgtacacgtagtacttcagcaaatggtatttctgctatccaacaaggagataaaaataaaaaatttgtttatagtcatccaaagttaaagaaattagaagaagttgtaattgaacacttcaagtcatggaatgctgaaaacactactgaaaagaaacgtgatgagacccgagttatgatcttctcttcatttcgagatagtgttcaagaaattgcagaaatgctttcacagcatcagccaattattagagtaatgacttttgtcggccatgcctcagggaaaagcacgaagggttttacccagaaggagcaactggaggtagtgaaacagtttcgtgacggtggttacaacacgctggtttctacctgtgtgggtgaagaaggtttggatataggagaagttgatcttataatatgttttgattcccagaagagcccaattcgtcttgtacaacgaatgggtagaactggccgtaaacgtcaaggcaggatagttattatcctttctgaaggacgagaggaacgtatttataatcagagtcagtccaacaaaagaagtatatataaagctatttcaagtaacaggcaggtccttcatttttaccaaagaagtccacgaatggttcctgatggaatcaacccaaaattacacaaaatgttcatcacacatggtgtctatgaaccagagaagccttctcggaacttgcagcgaaagtcatctatcttttcctatagggatggaatgaggcaaagtagcctaaagaaagattggttcttatcagaagaagaatttaaattatggaacagactttatagattaagggacagtgatgaaattaaagagataacattgcctcaagttcagttttcttctttacaaaatgaggaaaacaaaccagctcaagaatcaaccactggaattcatcaactctctctctctgaatggagactgtggcaagatcatcctttgcctacacatcaagttgatcactcagatcgatgccgccattttataggccttatgcaaatgatagagggaatgagacacgaagagggagaatgcagctatgaattggaagttgaatcttatttacaaatggaagatgttacctcaacatttattgctcccaggaatgaatctaataatcttgccagtgacacctttatcactcacaagaaatcgtcatttataaagaacataaatcaaggcagttcatcctcagtgatagaatctgatgaagaatgtgctgaaattgttaaacaaactcatatcaaacctactaaaattgtttctttaaagaaaaaagtgtctaaagaaataaaaaaagatcagcttaaaaaagaaaataatcacggtattatagattctgtagataatgacagaaattccactgttgaaaatatttttcaagaagacctaccaaatgataaaaggacatcagatacagatgaaattgctgccacatgtactattaatgaaaatgttattaaagaaccgtgtgtgttattaacagagtgtcagtttacaaataaatccactagttcacttgctggaaatgttttagattctggttataacagtttcaatgatgaaaaatctgtttcatctaacttatttcttccattcgaagaagagctttatattgttagaacagatgaccaattttataattgtcactcattgacaaaagaggtactagctaatgtagagagatttttatcttattctcctccgcctctcagtggactctcagacttggaatatgaaattgctaagggtactgcacttgagaatttgcttttcttaccctgtgcagagcatttacgaagtgataaatgcacctgtttgctgtcacattcagctgtgaattctcaacagaatttagaattgaattcacttaaatgtataaattatccatctgaaaaaagttgcctttatgatatacctaatgataatatttctgatgagccaagtctctgtgactgtgatgtacataaacataatcaaaatgaaaatttagtacctaacaatcgtgttcaaatacacagaagccctgcacagaatttagttggagagaacaatcatgatgttgataacagtgacctcccagtattgtccactgatcaagatgaaagtttgctgttatttgaagatgttaatacagagttcgacgatgtgagtctttcacccttgaacagtaaaagcgaatctttacctgtgtcagacaaaactgctattagtgaaacgcctctggtctctcagttcttaatttctgatgaacttttgttggacaataattctgaactccaagatcaaatcacccgtgatgctaatagttttaaatctcgtgatcagagaggtgtacaggaagaaaaagtgaagaatcatgaggatatttttgattgctctagggatttattttctgttacctttgatttaggattctgtagtccagattctgatgatgaaatattggaacatacatcagatagcaatagacctctagatgatctatatggaaggtatttggaaattaaggagataagtgatgcaaattatgtttcgaatcaagcactaataccaagagatcatagtaaaaattttactagtggaactgttattatcccatcaaatgaagatatgcagaatccaaattatgtacatttgccactgagtgcagcaaaaaatgaagaattgttatctcctggttattctcagttttctttaccagtgcaaaaaaaagttatgagtacaccactctctaaatcaaacacattgaactcattttctaagataagaaaggaaatacttaagacaccagattctagtaaggaaaaagtaaacctacaaagattcaaagaagcattgaattcaacttttgattattcagaattttctctagaaaagtctaaaagcagtggtccaatgtatctgcataaatcctgtcattctgttgaagatggacaattattaacaagtaacgaaagtgaagatgacgagattttccgaagaaaagttaaaagagcaaaaggaaatgttttaaactctcctgaggatcagaaaaatagtgaagttgattctccacttcatgctgtcaaaaagcgcagatttcctataaacagatcagaattatcatctagtgatgagagtgagaattttcccaaaccatgttcacaattagaagacttcaaggtttgtaacgggaatgccagaagaggcatcaaagtcccaaagagacagagtcacttaaagcatgtagctaggaagtttttagatgatgaagcagaactttctgaagaagatgcagaatatgtttcatcagatgaaaatgatgagtcagaaaatgaacaagattcctcattacttgactttttaaatgatgaaactcaactttcacaggctataaatgattctgaaatgagagctatttacatgaaatctttgcgtagtccaatgatgaacaataagtacaaaatgattcataagacacataaaaacataaacattttctcgcagattcctgaacaagatgaaacctatttagaggatagtttttgtgttgatgaagaggagtcttgcaaaggccaatcaagtgaagaagaagtttgtgttgattttaacttaataactgatgattgctttgcaaatagtaaaaagtataaaactcgacgtgcagtaatgctaaaagaaatgatggaacaaaattgtgcacattcaaaaaagaaattatccagaattattttaccagatgattcaagtgaggaggagaacaatgtaaatgataaaagagaatctaatattgcggttaacccaagcactgttaagaagaacaaacaacaggaccattgtttaaattcagtgccttctggatcttctgcgcagtccaaggtgcgttctactccaagagttaatccattagcaaagcagagcaaacagacatcgctgaatttaaaggatacaatttccgaagtctcagacttcaaacctcagaatcataatgaagtccagtctaccacaccacccttcactactgttgattcacagaaagactgtagaaaatttccagttccacagaaggatggtagtgctttggaggattctagcacttcaggggcatcctgttccaagtcaagaccacatttagctgggacacatacttctcttagacttccgcaggaaggaaaaggaacctgtattcttgtaggtggtcatgaaatcacttctggattagaagtaatttcttccctaagagcaattcatgggttgcaagtagaagtttgtcctcttaatggctgtgattacatcgtgagtaatcgcatggtggtggaaaggaggtctcaatctgagatgttaaatagtgtcaataagaacaagttcattgagcagatccagcacctgcagagtatgtttgaaagaatatgtgtgattgtggaaaaggacagagaaaaaacaggagacacatcaaggatgtttaggagaacaaagagctatgacagcctgctgactaccttaattggcgctggaatccgaattcttttcagttcctgccaagaagaaaccgcagatttgctaaaggaactgtctttagtggaacaaagaaagaatgttggtattcatgttccaacagtggtgaatagtaataaaagtgaggcactccagttttatttaagtattcccaatataagttatataactgcattaaatatgtgtcaccagttttcatctgtgaaaaggatggctaacagctcacttcaagaaatctccatgtatgcacaagtaactcatcagaaggctgaggagatctatagatatattcactatgtatttgacatacaaatgttaccaaatgatcttaaccaagatagactgaaatctgatatataa

SEQ ID NO: 10 - DNA, 3395 bp, Homo sapiens
gttttccagccccgaaggagtcctttggggttggaggaggggcgcgagcgcgcggcaaatcgggcgggaagaggtaaaaaaggaactgggagcgcgccgcggcggttcctgtccttacagttgcgctgcccagggaccgatgttgcgcgaggaaaatgcgggacgcccagggccatttctgagcagtggaggtttcaagtaatccactaacaaccagttccaaattctgtcatcaaatcctgtgctgctgttcctcgtggtaatagatgcatattatttcttttatttaaaagaaatgaatgtgactagtattgcattaagagctgaaacttggcttttagctgcatggcatgttaaagtacctccgatgtggctggaagcttgtattaactggattcaagaagaaaataataatgttaacttgagtcaggcccaaatgaataaacaagtgtttgagcagtggctccttactgatctgagggatttggagcatcctcttttacccgatggcattttagaaattccaaaaggagaattaaatggattttatgctctgcagattaattccttggttgatgtaagtcagcctgcatactcccagatacagaagttgagaggaaagaatacaacaaatgatctagttacagctgaagcacaagtaaccccaaaaccttgggaagcaaagccttcacgaatgttgatgctgcagctaactgatggaatcgtacaaatacagggaatggaatatcagcctattccaattcttcatagtgatcttcctccaggtacaaaaattttgatttatggaaatatatctttccgtcttggtgttctcttattgaaaccagaaaacgtgaaagtgttaggaggtgaagtagatgctcttttagaagaatatgcccaagaaaaagtacttgcaagattaataggggaacctgatcttgtagtttcagtcataccaaacaattctaacgaaaacattcccagagttacagatgttctagatcctgcattaggtccttctgatgaagaactcttggcaagtcttgatgaaaatgatgagcttacagcaaataatgacacttcctcagaacgatgtttcaccacaggtagttcctcaaataccattcccacaagacagtcaagttttgagccagaatttgttatttctccaagaccaaaagaggaaccatcaaacctatctatacatgtaatggatggagaattagatgacttttcactggaggaggccttgcttttagaagaaactgtccagaaagaacagatggaaactaaggaattgcaaccattgacttttaacagaaatgccgatcgaagtatagagagattttcacataatcctaatactacgaataacttttctttgacttgcaaaaatggaaacaataattggagtgaaaaaaatgtatctgaacaaatgactaatgaagacaaatcatttggttgtccatctgttagagaccaaaacaggagtattttttcagttcattgtaatgtacccttagcccatgattttacaaataaagaaaagaacttagagacagataataaaataaaacaaaccagcagttcagatagccattccttaaataataaaatattaaatagagaggtggtcaactatgtacagaaaaggaattcacaaatttctaatgaaaatgattgtaatttacagagttgttctttaagatcatcagagaatagcattaatctttctattgccatggatttgtattctccaccctttgtctatttgtctgttctaatggccagcaaaccaaaggaagttacaacagtgaaagtgaaagcatttattgtaaccttaactggaaatctctcaagttctggtggtatttggagtataactgcaaaggtgtctgatggtactgcatatctagatgtagactttgtggatgaaatacttactagcttgatagggttctcagtaccagaaatgaaacagtcaaaaaaggatcctcttcaataccaaaagttcctggaagggttgcagaaatgtcaaagagatctaatagatttgtgctgtctaatgactatttcatttaatccttccttgtctaaagcaatggtactggcattacaagatgttaatatggaacaccttgagaatctaaagaagcggttaaataaataattaaactaaaatagtattaggaacaattaaaaacaacaaggaaatatttagaatttgttcacaattttacttatgatactttgtgtaaaaaggaaaaatgaaatttcatagcttatttcagtttaattttaaagtgtttaatcatgtttgttatatgtggagcttttgaaaataagttaatctttttttttttttttttaatgtcagggtattgctctgttgcccaggctagagtgcagtggcatgatcataggttattgcatcaacctcttgagctcaagcgatcctcctgccttagcctcttgagttgctgggactataggcatgtaccaccatgcccagctaatttataattactattattgttttgtagggacagagttttgctatgttgcccagattggtctcaaactcctggcctctcactcctgccgtcatggcctccaaagtttactttttttaaaagtaactgttagatggaggaatatggtgacccactattgttgagaaatttgttttccacttgtcacgtcacataagatgattgctaaaatattgtactggtgttcggtggtgagtcatatttttgaaagttaataattataaataggtaatttccttctaatatgttggtactgtctatggccataccaccctgaacatgcctgagcttgtcataatatgttgagtacccaaaagatttgtttatattgttaatcttagggaaaaaaaattaaaatccagtagatcagaacatcaggctttcagatacaaattgatttactggtttttattttgctgattataatatttggtatatttaaggtaatctagttaactagatgctatttcatagattatattgaatgatttaaaactttattttcaaggatagtttattttaaatggcatattgaaaacatcattattaagatccagtaggtaggacatttattggattaaaatgaagcatttatctatgtctttaggtgtcattgttccctttctgaattagctgtacatataagccttcctttggttttaagtactgatttttttttaaaaaaaagagggactgtttaccattcttccactgtgctgttataaagttgtatttgaaaggtaatgttgtttttattaatcttttgtcttaaaataatttaaagtgctttgaattttaaaacattaaacaaatccttaaataacaaaaaaaaaaaaaaaa

SEQ ID NO: 11 - DNA, 1427 bp, Homo sapiens
gcggcgaggcggaatggcggcggctgcggactcgttctcaggcggccccgcgggggtgcggcttccgaggtcgccgccactcaaggtgctggcggagcagctgcggcgcgacgcggagggcggcccgggcgcgtggcggctgtcacgggcggcggcgggccgcgggccgctggacctggcggccgtgtggatgcagggcagggtagtgatggcggaccgcggcgaggctcggctgagggacccgagcggggacttctcggtccgcggcctggagcgggtgccgcgcgggcggccctgtctagtcccaggaaagtatgtgatggtgatgggagtggttcaggcctgcagccctgagccctgcctgcaggctgtgaagatgacagacctttctgataatcccatccatgaaagtatgtgggaactggaggtagaagatttacacaggaatattccttagagtatgttggaactgtcgttaaaaacaaccaaaatcccgaaactatttagaagcttataatgatgtggatttcatggacacttttcaatgcgtatttttcaaatgcttctcagagagccttgctttggttgaccaaggagtccggatgtaggaatgtttaaatcctcggatacttcagtgacacagcctctgctgccccttgctttgcctgtgtttgctgatgaaaagcagatgcttgtgtttcattttccttcctggtttgtgtgtgttaattctctctctctctctcagacacagaagtctcatgttgcattttccaaattttatgagtgatgatactttttccattactgctgcgtccctgttttacaatgcaaaatttaagtacggtcgttgcccatggtgattaaagtgtggttatgggcaggaagacagactgtgtaaaaaaggaatgacatcctggctcctcatcttcttcatcagcaactaccataaccagtttgcgagtcaaatggcatttcctaacggcaggcatggcggcccctgaaagacaacagctccctttctgcttcggacaccactcaaacatttagacgcagctctatcccttttcctagctagagaaggtgatgccttcttccattactcagagatgttgagacgttttcagaatttcttgttgaaatgaaaaacatcaagataaaggacgcctttcaggcattagctaaacttccacttcataactttcggcgagacatggtgagcctcctggtgtagagttcttttgtctttgtatggaatgactttttgctgtgatggttttgaatgttgggtttctgctgtctgcttagtacccatgcctgaattttttgagattgtaaatatcaaaggagttagattgtgttctgacatggttgtagactttcacctggattattgatattctacctctaataaatttttaataggc

SEQ ID NO: 12 - RNA, 19 nt, Artificial Sequence
  siFANCM1
gcucguucuuugauucaga

SEQ ID NO: 13 - RNA, 19 nt, Artificial Sequence
  siFANCM2
gaacauacaucagauagca

SEQ ID NO: 14 - RNA, 19 nt, Artificial Sequence
  siPOLD3
gaugcuguaugauuauguu

SEQ ID NO: 15 - RNA, 19 nt, Artificial Sequence
  siBLM
ggauguucuuagcacauca

SEQ ID NO: 16 - RNA, 19 nt, Artificial Sequence
  siRAD51
cagugguaaucacuaauca

SEQ ID NO: 17 - RNA, 19 nt, Artificial Sequence
  siRAD52
gacuaccugagaucacuaa

SEQ ID NO: 18 - RNA, 19 nt, Artificial Sequence
  siRMI1
gaguuguucuuuaagauca

SEQ ID NO: 19 - protein, 2048 aa, Artificial Sequence
  Mutant FANCM protein (PIP)
MSGRQRTRFQTSGSSISRSSGTPGCSSGTERPQSPGSSKAPLPAAAEAQLESDDDVLLVAAYEAERQLCLENGGFCTSAGALWIYPTNCPVRDYQLHISRAALFCNTLVCLPTGLGKTFIAAVVMYNFYRWFPSGKVVFMAPTKPLVTQQIEACYQVMGIPQSHMAEMTGSTQASTRKEIWCSKRVLFLTPQVMVNDLSRGACPAAEIKCLVIDEAHKALGNYAYCQVVRELVKYTNHFRILALSATPGSDIKAVQQVITNLLIGQIELRSEDSPDILTYSHERKVEKLIVPLGEELAAIQKTYIQILESFARSLIQRNVLMRRDIPNLTKYQIILARDQFRKNPSPNIVGIQQGIIEGEFAICISLYHGYELLQQMGMRSLYFFLCGIMDGTKGMTRSKNELGRNEDFMKLYNHLECMFARTRSTSANGISAIQQGDKNKKFVYSHPKLKKLEEVVIEHFKSWNAENTTEKKRDETRVMIFSSFRDSVQEIAEMLSQHQPIIRVMTFVGHASGKSTKGFTQKEQLEVVKQFRDGGYNTLVSTCVGEEGLDIGEVDLIICFDSQKSPIRLVQRMGRTGRKRQGRIVIILSEGREERIYNQSQSNKRSIYKAISSNRQVLHFYQRSPRMVPDGINPKLHKMFITHGVYEPEKPSRNLQRKSSIFSYRDGMRQSSLKKDWFLSEEEFKLWNRLYRLRDSDEIKEITLPQVQFSSLQNEENKPAQESTTGIHQLSLSEWRLWQDHPLPTHQVDHSDRCRHFIGLMQMIEGMRHEEGECSYELEVESYLQMEDVTSTFIAPRNESNNLASDTFITHKKSSFIKNINQGSSSSVIESDEECAEIVKQTHIKPTKIVSLKKKVSKEIKKDQLKKENNHGIIDSVDNDRNSTVENIFQEDLPNDKRTSDTDEIAATCTINENVIKEPCVLLTECQFTNKSTSSLAGNVLDSGYNSFNDEKSVSSNLFLPFEEELYIVRTDDQFYNCHSLTKEVLANVERFLSYSPPPLSGLSDLEYEIAKGTALENLLFLPCAEHLRSDKCTCLLSHSAVNSQQNLELNSLKCINYPSEKSCLYDIPNDNISDEPSLCDCDVHKHNQNENLVPNNRVQIHRSPAQNLVGENNHDVDNSDLPVLSTDQDESLLLFEDVNTEFDDVSLSPLNSKSESLPVSDKTAISETPLVSQFLISDELLLDNNSELQDQITRDANSFKSRDQRGVQEEKVKNHEDIFDCSRDLFSVTFDLGFCSPDSDDEILEHTSDSNRPLDDLYGRYLEIKEISDANYVSNQALIPRDHSKNFTSGTVIIPSNEDMQNPNYVHLPLSAAKNEELLSPGYSQFSLPVQKKVMSTPLSKSNTLNSFSKIRKEILKTPDSSKEKVNLQRFKEALNSTFDYSEFSLEKSKSSGPMYLHKSCHSVEDGQLLTSNESEDDEIFRRKVKRAKGNVLNSPEDQKNSEVDSPLHAVKKRRFPINRSELSSSDESENFPKPCSQLEDFKVCNGNARRGIKVPKRQSHLKHVARKFLDDEAELSEEDAEYVSSDENDESENEQDSSLLDFLNDETQLSQAINDSEMRAIYMKSLRSPMMNNKYKMIHKTHKNINIFSQIPEQDETYLEDSFCVDEEESCKGQSSEEEVCVDFNLITDDCFANSKKYKTRRAVMLKEMMEQNCAHSKKKLSRIILPDDSSEEENNVNDKRESNIAVNPSTVKKNKQQDHCLNSVPSGSSAQSKVRSTPRVNPLAKQSKQTSLNLKDTISEVSDFKPQNHNEVQSTTPPFTTVDSQKDCRKFPVPQKDGSALEDSSTSGASCSKSRPHLAGTHTSLRLPQEGKGTCILVGGHEITSGLEVISSLRAIHGLQVEVCPLNGCDYIVSNRMVVERRSQSEMLNSVNKNKFIEQIQHLQSMFERICVIVEKDREKTGDTSRMFRRTKSYDSLLTTLIGAGIRILFSSCQEETADLLKELSLVEQRKNVGIHVPTVVNSNKSEALQFYLSIPNISYITALNMCHQFSSVKRMANSSLQEISMYAQVTHQKAEEIYRYIHYVFDIQMLPNDLNQDRLKSDI

SEQ ID NO: 20 - protein, 2048 aa, Artificial Sequence
  mutant FANCM protein (K117R)
MSGRQRTLFQTWGSSISRSSGTPGCSSGTERPQSPGSSKAPLPAAAEAQLESDDDVLLVAAYEAERQLCLENGGFCTSAGALWIYPTNCPVRDYQLHISRAALFCNTLVCLPTGLGRTFIAAVVMYNFYRWFPSGKVVFMAPTKPLVTQQIEACYQVMGIPQSHMAEMTGSTQASTRKEIWCSKRVLFLTPQVMVNDLSRGACPAAEIKCLVIDEAHKALGNYAYCQVVRELVKYTNHFRILALSATPGSDIKAVQQVITNLLIGQIELRSEDSPDILTYSHERKVEKLIVPLGEELAAIQKTYIQILESFARSLIQRNVLMRRDIPNLTKYQIILARDQFRKNPSPNIVGIQQGIIEGEFAICISLYHGYELLQQMGMRSLYFFLCGIMDGTKGMTRSKNELGRNEDFMKLYNHLECMFARTRSTSANGISAIQQGDKNKKFVYSHPKLKKLEEVVIEHFKSWNAENTTEKKRDETRVMIFSSFRDSVQEIAEMLSQHQPIIRVMTFVGHASGKSTKGFTQKEQLEVVKQFRDGGYNTLVSTCVGEEGLDIGEVDLIICFDSQKSPIRLVQRMGRTGRKRQGRIVIILSEGREERIYNQSQSNKRSIYKAISSNRQVLHFYQRSPRMVPDGINPKLHKMFITHGVYEPEKPSRNLQRKSSIFSYRDGMRQSSLKKDWFLSEEEFKLWNRLYRLRDSDEIKEITLPQVQFSSLQNEENKPAQESTTGIHQLSLSEWRLWQDHPLPTHQVDHSDRCRHFIGLMQMIEGMRHEEGECSYELEVESYLQMEDVTSTFIAPRNESNNLASDTFITHKKSSFIKNINQGSSSSVIESDEECAEIVKQTHIKPTKIVSLKKKVSKEIKKDQLKKENNHGIIDSVDNDRNSTVENIFQEDLPNDKRTSDTDEIAATCTINENVIKEPCVLLTECQFTNKSTSSLAGNVLDSGYNSFNDEKSVSSNLFLPFEEELYIVRTDDQFYNCHSLTKEVLANVERFLSYSPPPLSGLSDLEYEIAKGTALENLLFLPCAEHLRSDKCTCLLSHSAVNSQQNLELNSLKCINYPSEKSCLYDIPNDNISDEPSLCDCDVHKHNQNENLVPNNRVQIHRSPAQNLVGENNHDVDNSDLPVLSTDQDESLLLFEDVNTEFDDVSLSPLNSKSESLPVSDKTAISETPLVSQFLISDELLLDNNSELQDQITRDANSFKSRDQRGVQEEKVKNHEDIFDCSRDLFSVTFDLGFCSPDSDDEILEHTSDSNRPLDDLYGRYLEIKEISDANYVSNQALIPRDHSKNFTSGTVIIPSNEDMQNPNYVHLPLSAAKNEELLSPGYSQFSLPVQKKVMSTPLSKSNTLNSFSKIRKEILKTPDSSKEKVNLQRFKEALNSTFDYSEFSLEKSKSSGPMYLHKSCHSVEDGQLLTSNESEDDEIFRRKVKRAKGNVLNSPEDQKNSEVDSPLHAVKKRRFPINRSELSSSDESENFPKPCSQLEDFKVCNGNARRGIKVPKRQSHLKHVARKFLDDEAELSEEDAEYVSSDENDESENEQDSSLLDFLNDETQLSQAINDSEMRAIYMKSLRSPMMNNKYKMIHKTHKNINIFSQIPEQDETYLEDSFCVDEEESCKGQSSEEEVCVDFNLITDDCFANSKKYKTRRAVMLKEMMEQNCAHSKKKLSRIILPDDSSEEENNVNDKRESNIAVNPSTVKKNKQQDHCLNSVPSGSSAQSKVRSTPRVNPLAKQSKQTSLNLKDTISEVSDFKPQNHNEVQSTTPPFTTVDSQKDCRKFPVPQKDGSALEDSSTSGASCSKSRPHLAGTHTSLRLPQEGKGTCILVGGHEITSGLEVISSLRAIHGLQVEVCPLNGCDYIVSNRMVVERRSQSEMLNSVNKNKFIEQIQHLQSMFERICVIVEKDREKTGDTSRMFRRTKSYDSLLTTLIGAGIRILFSSCQEETADLLKELSLVEQRKNVGIHVPTVVNSNKSEALQFYLSIPNISYITALNMCHQFSSVKRMANSSLQEISMYAQVTHQKAEEIYRYIHYVFDIQMLPNDLNQDRLKSDI

SEQ ID NO: 21 - protein, 2048 aa, Artificial Sequence
  mutant FANCM protein (MID)
MSGRQRTLFQTWGSSISRSSGTPGCSSGTERPQSPGSSKAPLPAAAEAQLESDDDVLLVAAYEAERQLCLENGGFCTSAGALWIYPTNCPVRDYQLHISRAALFCNTLVCLPTGLGKTFIAAVVMYNFYRWFPSGKVVFMAPTKPLVTQQIEACYQVMGIPQSHMAEMTGSTQASTRKEIWCSKRVLFLTPQVMVNDLSRGACPAAEIKCLVIDEAHKALGNYAYCQVVRELVKYTNHFRILALSATPGSDIKAVQQVITNLLIGQIELRSEDSPDILTYSHERKVEKLIVPLGEELAAIQKTYIQILESFARSLIQRNVLMRRDIPNLTKYQIILARDQFRKNPSPNIVGIQQGIIEGEFAICISLYHGYELLQQMGMRSLYFFLCGIMDGTKGMTRSKNELGRNEDFMKLYNHLECMFARTRSTSANGISAIQQGDKNKKFVYSHPKLKKLEEVVIEHFKSWNAENTTEKKRDETRVMIFSSFRDSVQEIAEMLSQHQPIIRVMTFVGHASGKSTKGFTQKEQLEVVKQFRDGGYNTLVSTCVGEEGLDIGEVDLIICFDSQKSPIRLVQRMGRTGRKRQGRIVIILSEGREERIYNQSQSNKRSIYKAISSNRQVLHFYQRSPRMVPDGINPKLHKMFITHGVYEPEKPSRNLQRKSSIFSYRDGMRQSSLKKDWFLSEEEFKLWNRLYRLRDSDEIKEITLPQVQFSSLQNEENKPAQESTTGIHQLSLSEWRLWQDHPLPTHQGDGSDRCRHFIGLMQMIEGMRHEEGECSYELEVESYLQMEDVTSTFIAPRNESNNLASDTFITHKKSSFIKNINQGSSSSVIESDEECAEIVKQTHIKPTKIVSLKKKVSKEIKKDQLKKENNHGIIDSVDNDRNSTVENIFQEDLPNDKRTSDTDEIAATCTINENVIKEPCVLLTECQFTNKSTSSLAGNVLDSGYNSFNDEKSVSSNLFLPFEEELYIVRTDDQFYNCHSLTKEVLANVERFLSYSPPPLSGLSDLEYEIAKGTALENLLFLPCAEHLRSDKCTCLLSHSAVNSQQNLELNSLKCINYPSEKSCLYDIPNDNISDEPSLCDCDVHKHNQNENLVPNNRVQIHRSPAQNLVGENNHDVDNSDLPVLSTDQDESLLLFEDVNTEFDDVSLSPLNSKSESLPVSDKTAISETPLVSQFLISDELLLDNNSELQDQITRDANSFKSRDQRGVQEEKVKNHEDIFDCSRDLFSVTFDLGFCSPDSDDEILEHTSDSNRPLDDLYGRYLEIKEISDANYVSNQALIPRDHSKNFTSGTVIIPSNEDMQNPNYVHLPLSAAKNEELLSPGYSQFSLPVQKKVMSTPLSKSNTLNSFSKIRKEILKTPDSSKEKVNLQRFKEALNSTFDYSEFSLEKSKSSGPMYLHKSCHSVEDGQLLTSNESEDDEIFRRKVKRAKGNVLNSPEDQKNSEVDSPLHAVKKRRFPINRSELSSSDESENFPKPCSQLEDFKVCNGNARRGIKVPKRQSHLKHVARKFLDDEAELSEEDAEYVSSDENDESENEQDSSLLDFLNDETQLSQAINDSEMRAIYMKSLRSPMMNNKYKMIHKTHKNINIFSQIPEQDETYLEDSFCVDEEESCKGQSSEEEVCVDFNLITDDCFANSKKYKTRRAVMLKEMMEQNCAHSKKKLSRIILPDDSSEEENNVNDKRESNIAVNPSTVKKNKQQDHCLNSVPSGSSAQSKVRSTPRVNPLAKQSKQTSLNLKDTISEVSDFKPQNHNEVQSTTPPFTTVDSQKDCRKFPVPQKDGSALEDSSTSGASCSKSRPHLAGTHTSLRLPQEGKGTCILVGGHEITSGLEVISSLRAIHGLQVEVCPLNGCDYIVSNRMVVERRSQSEMLNSVNKNKFIEQIQHLQSMFERICVIVEKDREKTGDTSRMFRRTKSYDSLLTTLIGAGIRILFSSCQEETADLLKELSLVEQRKNVGIHVPTVVNSNKSEALQFYLSIPNISYITALNMCHQFSSVKRMANSSLQEISMYAQVTHQKAEEIYRYIHYVFDIQMLPNDLNQDRLKSDI

SEQ ID NO: 22 - protein, 2048 aa, Artificial Sequence
  mutant FANCM protein (S1045A)
MSGRQRTLFQTWGSSISRSSGTPGCSSGTERPQSPGSSKAPLPAAAEAQLESDDDVLLVAAYEAERQLCLENGGFCTSAGALWIYPTNCPVRDYQLHISRAALFCNTLVCLPTGLGKTFIAAVVMYNFYRWFPSGKVVFMAPTKPLVTQQIEACYQVMGIPQSHMAEMTGSTQASTRKEIWCSKRVLFLTPQVMVNDLSRGACPAAEIKCLVIDEAHKALGNYAYCQVVRELVKYTNHFRILALSATPGSDIKAVQQVITNLLIGQIELRSEDSPDILTYSHERKVEKLIVPLGEELAAIQKTYIQILESFARSLIQRNVLMRRDIPNLTKYQIILARDQFRKNPSPNIVGIQQGIIEGEFAICISLYHGYELLQQMGMRSLYFFLCGIMDGTKGMTRSKNELGRNEDFMKLYNHLECMFARTRSTSANGISAIQQGDKNKKFVYSHPKLKKLEEVVIEHFKSWNAENTTEKKRDETRVMIFSSFRDSVQEIAEMLSQHQPIIRVMTFVGHASGKSTKGFTQKEQLEVVKQFRDGGYNTLVSTCVGEEGLDIGEVDLIICFDSQKSPIRLVQRMGRTGRKRQGRIVIILSEGREERIYNQSQSNKRSIYKAISSNRQVLHFYQRSPRMVPDGINPKLHKMFITHGVYEPEKPSRNLQRKSSIFSYRDGMRQSSLKKDWFLSEEEFKLWNRLYRLRDSDEIKEITLPQVQFSSLQNEENKPAQESTTGIHQLSLSEWRLWQDHPLPTHQVDHSDRCRHFIGLMQMIEGMRHEEGECSYELEVESYLQMEDVTSTFIAPRNESNNLASDTFITHKKSSFIKNINQGSSSSVIESDEECAEIVKQTHIKPTKIVSLKKKVSKEIKKDQLKKENNHGIIDSVDNDRNSTVENIFQEDLPNDKRTSDTDEIAATCTINENVIKEPCVLLTECQFTNKSTSSLAGNVLDSGYNSFNDEKSVSSNLFLPFEEELYIVRTDDQFYNCHSLTKEVLANVERFLSYSPPPLSGLSDLEYEIAKGTALENLLFLPCAEHLRSDKCTCLLSHSAVNAQQNLELNSLKCINYPSEKSCLYDIPNDNISDEPSLCDCDVHKHNQNENLVPNNRVQIHRSPAQNLVGENNHDVDNSDLPVLSTDQDESLLLFEDVNTEFDDVSLSPLNSKSESLPVSDKTAISETPLVSQFLISDELLLDNNSELQDQITRDANSFKSRDQRGVQEEKVKNHEDIFDCSRDLFSVTFDLGFCSPDSDDEILEHTSDSNRPLDDLYGRYLEIKEISDANYVSNQALIPRDHSKNFTSGTVIIPSNEDMQNPNYVHLPLSAAKNEELLSPGYSQFSLPVQKKVMSTPLSKSNTLNSFSKIRKEILKTPDSSKEKVNLQRFKEALNSTFDYSEFSLEKSKSSGPMYLHKSCHSVEDGQLLTSNESEDDEIFRRKVKRAKGNVLNSPEDQKNSEVDSPLHAVKKRRFPINRSELSSSDESENFPKPCSQLEDFKVCNGNARRGIKVPKRQSHLKHVARKFLDDEAELSEEDAEYVSSDENDESENEQDSSLLDFLNDETQLSQAINDSEMRAIYMKSLRSPMMNNKYKMIHKTHKNINIFSQIPEQDETYLEDSFCVDEEESCKGQSSEEEVCVDFNLITDDCFANSKKYKTRRAVMLKEMMEQNCAHSKKKLSRIILPDDSSEEENNVNDKRESNIAVNPSTVKKNKQQDHCLNSVPSGSSAQSKVRSTPRVNPLAKQSKQTSLNLKDTISEVSDFKPQNHNEVQSTTPPFTTVDSQKDCRKFPVPQKDGSALEDSSTSGASCSKSRPHLAGTHTSLRLPQEGKGTCILVGGHEITSGLEVISSLRAIHGLQVEVCPLNGCDYIVSNRMVVERRSQSEMLNSVNKNKFIEQIQHLQSMFERICVIVEKDREKTGDTSRMFRRTKSYDSLLTTLIGAGIRILFSSCQEETADLLKELSLVEQRKNVGIHVPTVVNSNKSEALQFYLSIPNISYITALNMCHQFSSVKRMANSSLQEISMYAQVTHQKAEEIYRYIHYVFDIQMLPNDLNQDRLKSDI

SEQ ID NO: 23 - protein, 1986 aa, Artificial Sequence
  mutant FANCM protein delta MM1
MSGRQRTLFQTWGSSISRSSGTPGCSSGTERPQSPGSSKAPLPAAAEAQLESDDDVLLVAAYEAERQLCLENGGFCTSAGALWIYPTNCPVRDYQLHISRAALFCNTLVCLPTGLGKTFIAAVVMYNFYRWFPSGKVVFMAPTKPLVTQQIEACYQVMGIPQSHMAEMTGSTQASTRKEIWCSKRVLFLTPQVMVNDLSRGACPAAEIKCLVIDEAHKALGNYAYCQVVRELVKYTNHFRILALSATPGSDIKAVQQVITNLLIGQIELRSEDSPDILTYSHERKVEKLIVPLGEELAAIQKTYIQILESFARSLIQRNVLMRRDIPNLTKYQIILARDQFRKNPSPNIVGIQQGIIEGEFAICISLYHGYELLQQMGMRSLYFFLCGIMDGTKGMTRSKNELGRNEDFMKLYNHLECMFARTRSTSANGISAIQQGDKNKKFVYSHPKLKKLEEVVIEHFKSWNAENTTEKKRDETRVMIFSSFRDSVQEIAEMLSQHQPIIRVMTFVGHASGKSTKGFTQKEQLEVVKQFRDGGYNTLVSTCVGEEGLDIGEVDLIICFDSQKSPIRLVQRMGRTGRKRQGRIVIILSEGREERIYNQSQSNKRSIYKAISSNRQVLHFYQRSPRMVPDGINPKLHKMFITHGVYEPEKPSRNLQRKSSIFSYRDGMRQSSLKKDWFLSEEEFKLWNRLYRLRDSDEIKEITLPQVQFSSLQNEENKPAQESTTGIHQLSLSEWRLWQDHPLPTHQVDHSDRCRHFIGLMQMIEGMRHEEGECSYELEVESYLQMEDVTSTFIAPRNESNNLASDTFITHKKSSFIKNINQGSSSSVIESDEECAEIVKQTHIKPTKIVSLKKKVSKEIKKDQLKKENNHGIIDSVDNDRNSTVENIFQEDLPNDKRTSDTDEIAATCTINENVIKEPCVLLTECQFTNKSTSSLAGNVLSDLEYEIAKGTALENLLFLPCAEHLRSDKCTCLLSHSAVNSQQNLELNSLKCINYPSEKSCLYDIPNDNISDEPSLCDCDVHKHNQNENLVPNNRVQIHRSPAQNLVGENNHDVDNSDLPVLSTDQDESLLLFEDVNTEFDDVSLSPLNSKSESLPVSDKTAISETPLVSQFLISDELLLDNNSELQDQITRDANSFKSRDQRGVQEEKVKNHEDIFDCSRDLFSVTFDLGFCSPDSDDEILEHTSDSNRPLDDLYGRYLEIKEISDANYVSNQALIPRDHSKNFTSGTVIIPSNEDMQNPNYVHLPLSAAKNEELLSPGYSQFSLPVQKKVMSTPLSKSNTLNSFSKIRKEILKTPDSSKEKVNLQRFKEALNSTFDYSEFSLEKSKSSGPMYLHKSCHSVEDGQLLTSNESEDDEIFRRKVKRAKGNVLNSPEDQKNSEVDSPLHAVKKRRFPINRSELSSSDESENFPKPCSQLEDFKVCNGNARRGIKVPKRQSHLKHVARKFLDDEAELSEEDAEYVSSDENDESENEQDSSLLDFLNDETQLSQAINDSEMRAIYMKSLRSPMMNNKYKMIHKTHKNINIFSQIPEQDETYLEDSFCVDEEESCKGQSSEEEVCVDFNLITDDCFANSKKYKTRRAVMLKEMMEQNCAHSKKKLSRIILPDDSSEEENNVNDKRESNIAVNPSTVKKNKQQDHCLNSVPSGSSAQSKVRSTPRVNPLAKQSKQTSLNLKDTISEVSDFKPQNHNEVQSTTPPFTTVDSQKDCRKFPVPQKDGSALEDSSTSGASCSKSRPHLAGTHTSLRLPQEGKGTCILVGGHEITSGLEVISSLRAIHGLQVEVCPLNGCDYIVSNRMVVERRSQSEMLNSVNKNKFIEQIQHLQSMFERICVIVEKDREKTGDTSRMFRRTKSYDSLLTTLIGAGIRILFSSCQEETADLLKELSLVEQRKNVGIHVPTVVNSNKSEALQFYLSIPNISYITALNMCHQFSSVKRMANSSLQEISMYAQVTHQKAEEIYRYIHYVFDIQMLPNDLNQDRLKSDI

SEQ ID NO: 24 - protein, 2015 aa, Artificial Sequence
  mutant FANCM protein delta MM2
MSGRQRTLFQTWGSSISRSSGTPGCSSGTERPQSPGSSKAPLPAAAEAQLESDDDVLLVAAYEAERQLCLENGGFCTSAGALWIYPTNCPVRDYQLHISRAALFCNTLVCLPTGLGKTFIAAVVMYNFYRWFPSGKVVFMAPTKPLVTQQIEACYQVMGIPQSHMAEMTGSTQASTRKEIWCSKRVLFLTPQVMVNDLSRGACPAAEIKCLVIDEAHKALGNYAYCQVVRELVKYTNHFRILALSATPGSDIKAVQQVITNLLIGQIELRSEDSPDILTYSHERKVEKLIVPLGEELAAIQKTYIQILESFARSLIQRNVLMRRDIPNLTKYQIILARDQFRKNPSPNIVGIQQGIIEGEFAICISLYHGYELLQQMGMRSLYFFLCGIMDGTKGMTRSKNELGRNEDFMKLYNHLECMFARTRSTSANGISAIQQGDKNKKFVYSHPKLKKLEEVVIEHFKSWNAENTTEKKRDETRVMIFSSFRDSVQEIAEMLSQHQPIIRVMTFVGHASGKSTKGFTQKEQLEVVKQFRDGGYNTLVSTCVGEEGLDIGEVDLIICFDSQKSPIRLVQRMGRTGRKRQGRIVIILSEGREERIYNQSQSNKRSIYKAISSNRQVLHFYQRSPRMVPDGINPKLHKMFITHGVYEPEKPSRNLQRKSSIFSYRDGMRQSSLKKDWFLSEEEFKLWNRLYRLRDSDEIKEITLPQVQFSSLQNEENKPAQESTTGIHQLSLSEWRLWQDHPLPTHQVDHSDRCRHFIGLMQMIEGMRHEEGECSYELEVESYLQMEDVTSTFIAPRNESNNLASDTFITHKKSSFIKNINQGSSSSVIESDEECAEIVKQTHIKPTKIVSLKKKVSKEIKKDQLKKENNHGIIDSVDNDRNSTVENIFQEDLPNDKRTSDTDEIAATCTINENVIKEPCVLLTECQFTNKSTSSLAGNVLDSGYNSFNDEKSVSSNLFLPFEEELYIVRTDDQFYNCHSLTKEVLANVERFLSYSPPPLSGLSDLEYEIAKGTALENLLFLPCAEHLRSDKCTCLLSHSAVNSQQNLELNSLKCINYPSEKSCLYDIPNDNISDEPSLCDCDVHKHNQNENLVPNNRVQIHRSPAQNLVGENNHDVDNSDLPVLSTDQDESLLLFEDVNTEFDDVSLSPLNSKSESLPVSDKTAISETPLVSQFLISDELLLDNNSELQDQITRDANSFKSRDQRGVQEEKVKNHESNRPLDDLYGRYLEIKEISDANYVSNQALIPRDHSKNFTSGTVIIPSNEDMQNPNYVHLPLSAAKNEELLSPGYSQFSLPVQKKVMSTPLSKSNTLNSFSKIRKEILKTPDSSKEKVNLQRFKEALNSTFDYSEFSLEKSKSSGPMYLHKSCHSVEDGQLLTSNESEDDEIFRRKVKRAKGNVLNSPEDQKNSEVDSPLHAVKKRRFPINRSELSSSDESENFPKPCSQLEDFKVCNGNARRGIKVPKRQSHLKHVARKFLDDEAELSEEDAEYVSSDENDESENEQDSSLLDFLNDETQLSQAINDSEMRAIYMKSLRSPMMNNKYKMIHKTHKNINIFSQIPEQDETYLEDSFCVDEEESCKGQSSEEEVCVDFNLITDDCFANSKKYKTRRAVMLKEMMEQNCAHSKKKLSRIILPDDSSEEENNVNDKRESNIAVNPSTVKKNKQQDHCLNSVPSGSSAQSKVRSTPRVNPLAKQSKQTSLNLKDTISEVSDFKPQNHNEVQSTTPPFTTVDSQKDCRKFPVPQKDGSALEDSSTSGASCSKSRPHLAGTHTSLRLPQEGKGTCILVGGHEITSGLEVISSLRAIHGLQVEVCPLNGCDYIVSNRMVVERRSQSEMLNSVNKNKFIEQIQHLQSMFERICVIVEKDREKTGDTSRMFRRTKSYDSLLTTLIGAGIRILFSSCQEETADLLKELSLVEQRKNVGIHVPTVVNSNKSEALQFYLSIPNISYITALNMCHQFSSVKRMANSSLQEISMYAQVTHQKAEEIYRYIHYVFDIQMLPNDLNQDRLKSDI

SEQ ID NO: 25 - protein, 2048 aa, Artificial Sequence
  mutant FANCM FF>AA
MSGRQRTLFQTWGSSISRSSGTPGCSSGTERPQSPGSSKAPLPAAAEAQLESDDDVLLVAAYEAERQLCLENGGFCTSAGALWIYPTNCPVRDYQLHISRAALFCNTLVCLPTGLGKTFIAAVVMYNFYRWFPSGKVVFMAPTKPLVTQQIEACYQVMGIPQSHMAEMTGSTQASTRKEIWCSKRVLFLTPQVMVNDLSRGACPAAEIKCLVIDEAHKALGNYAYCQVVRELVKYTNHFRILALSATPGSDIKAVQQVITNLLIGQIELRSEDSPDILTYSHERKVEKLIVPLGEELAAIQKTYIQILESFARSLIQRNVLMRRDIPNLTKYQIILARDQFRKNPSPNIVGIQQGIIEGEFAICISLYHGYELLQQMGMRSLYFFLCGIMDGTKGMTRSKNELGRNEDFMKLYNHLECMFARTRSTSANGISAIQQGDKNKKFVYSHPKLKKLEEVVIEHFKSWNAENTTEKKRDETRVMIFSSFRDSVQEIAEMLSQHQPIIRVMTFVGHASGKSTKGFTQKEQLEVVKQFRDGGYNTLVSTCVGEEGLDIGEVDLIICFDSQKSPIRLVQRMGRTGRKRQGRIVIILSEGREERIYNQSQSNKRSIYKAISSNRQVLHFYQRSPRMVPDGINPKLHKMFITHGVYEPEKPSRNLQRKSSIFSYRDGMRQSSLKKDWFLSEEEFKLWNRLYRLRDSDEIKEITLPQVQFSSLQNEENKPAQESTTGIHQLSLSEWRLWQDHPLPTHQVDHSDRCRHFIGLMQMIEGMRHEEGECSYELEVESYLQMEDVTSTFIAPRNESNNLASDTFITHKKSSFIKNINQGSSSSVIESDEECAEIVKQTHIKPTKIVSLKKKVSKEIKKDQLKKENNHGIIDSVDNDRNSTVENIFQEDLPNDKRTSDTDEIAATCTINENVIKEPCVLLTECQFTNKSTSSLAGNVLDSGYNSFNDEKSVSSNLFLPFEEELYIVRTDDQFYNCHSLTKEVLANVERFLSYSPPPLSGLSDLEYEIAKGTALENLLFLPCAEHLRSDKCTCLLSHSAVNSQQNLELNSLKCINYPSEKSCLYDIPNDNISDEPSLCDCDVHKHNQNENLVPNNRVQIHRSPAQNLVGENNHDVDNSDLPVLSTDQDESLLLFEDVNTEFDDVSLSPLNSKSESLPVSDKTAISETPLVSQFLISDELLLDNNSELQDQITRDANSFKSRDQRGVQEEKVKNHEDIFDCSRDLFSVTADLGACSPDSDDEILEHTSDSNRPLDDLYGRYLEIKEISDANYVSNQALIPRDHSKNFTSGTVIIPSNEDMQNPNYVHLPLSAAKNEELLSPGYSQFSLPVQKKVMSTPLSKSNTLNSFSKIRKEILKTPDSSKEKVNLQRFKEALNSTFDYSEFSLEKSKSSGPMYLHKSCHSVEDGQLLTSNESEDDEIFRRKVKRAKGNVLNSPEDQKNSEVDSPLHAVKKRRFPINRSELSSSDESENFPKPCSQLEDFKVCNGNARRGIKVPKRQSHLKHVARKFLDDEAELSEEDAEYVSSDENDESENEQDSSLLDFLNDETQLSQAINDSEMRAIYMKSLRSPMMNNKYKMIHKTHKNINIFSQIPEQDETYLEDSFCVDEEESCKGQSSEEEVCVDFNLITDDCFANSKKYKTRRAVMLKEMMEQNCAHSKKKLSRIILPDDSSEEENNVNDKRESNIAVNPSTVKKNKQQDHCLNSVPSGSSAQSKVRSTPRVNPLAKQSKQTSLNLKDTISEVSDFKPQNHNEVQSTTPPFTTVDSQKDCRKFPVPQKDGSALEDSSTSGASCSKSRPHLAGTHTSLRLPQEGKGTCILVGGHEITSGLEVISSLRAIHGLQVEVCPLNGCDYIVSNRMVVERRSQSEMLNSVNKNKFIEQIQHLQSMFERICVIVEKDREKTGDTSRMFRRTKSYDSLLTTLIGAGIRILFSSCQEETADLLKELSLVEQRKNVGIHVPTVVNSNKSEALQFYLSIPNISYITALNMCHQFSSVKRMANSSLQEISMYAQVTHQKAEEIYRYIHYVFDIQMLPNDLNQDRLKSDI

SEQ ID NO: 26 - protein, 1677 aa, Artificial Sequence
  mutant FANCM protein delta MM3
MSGRQRTLFQTWGSSISRSSGTPGCSSGTERPQSPGSSKAPLPAAAEAQLESDDDVLLVAAYEAERQLCLENGGFCTSAGALWIYPTNCPVRDYQLHISRAALFCNTLVCLPTGLGKTFIAAVVMYNFYRWFPSGKVVFMAPTKPLVTQQIEACYQVMGIPQSHMAEMTGSTQASTRKEIWCSKRVLFLTPQVMVNDLSRGACPAAEIKCLVIDEAHKALGNYAYCQVVRELVKYTNHFRILALSATPGSDIKAVQQVITNLLIGQIELRSEDSPDILTYSHERKVEKLIVPLGEELAAIQKTYIQILESFARSLIQRNVLMRRDIPNLTKYQIILARDQFRKNPSPNIVGIQQGIIEGEFAICISLYHGYELLQQMGMRSLYFFLCGIMDGTKGMTRSKNELGRNEDFMKLYNHLECMFARTRSTSANGISAIQQGDKNKKFVYSHPKLKKLEEVVIEHFKSWNAENTTEKKRDETRVMIFSSFRDSVQEIAEMLSQHQPIIRVMTFVGHASGKSTKGFTQKEQLEVVKQFRDGGYNTLVSTCVGEEGLDIGEVDLIICFDSQKSPIRLVQRMGRTGRKRQGRIVIILSEGREERIYNQSQSNKRSIYKAISSNRQVLHFYQRSPRMVPDGINPKLHKMFITHGVYEPEKPSRNLQRKSSIFSYRDGMRQSSLKKDWFLSEEEFKLWNRLYRLRDSDEIKEITLPQVQFSSLQNEENKPAQESTTGIHQLSLSEWRLWQDHPLPTHQVDHSDRCRHFIGLMQMIEGMRHEEGECSYELEVESYLQMEDVTSTFIAPRNESNNLASDTFITHKKSSFIKNINQGSSSSVIESDEECAEIVKQTHIKPTKIVSLKKKVSKEIKKDQLKKENNHGIIDSVDNDRNSTVENIFQEDLPNDKRTSDTDEIAATCTINENVIKEPCVLLTECQFTNKSTSSLAGNVLDSGYNSFNDEKSVSSNLFLPFEEELYIVRTDDQFYNCHSLTKEVLANVERFLSYSPPPLSGLSDLEYEIAKGTALENLLFLPCAEHLRSDKCTCLLSHSAVNSQQNLELNSLKCINYPSEKSCLYDIPNDNISDEPSLCDCDVHKHNQNENLVPNNRVQIHRSPAQNLVGENNHDVDNSDLPVLSTDQDESLLLFEDVNTEFDDVSLSPLNSKSESLPVSDKTAISETPLVSQFLISDELLLDNNSELQDQITRDANSFKSRDQRGVQEEKVKNHEDIFDCSRDLFSVTFDLGFCSPDSDDEILEHTSDSNRPLDDLYGRYLEIKEISDANYVSNQALIPRDHSKNFTSGTVIIPSNEDMQNPNYVHLPLSAAKNEELLSPGYSQFSLPVQKKVVPSGSSAQSKVRSTPRVNPLAKQSKQTSLNLKDTISEVSDFKPQNHNEVQSTTPPFTTVDSQKDCRKFPVPQKDGSALEDSSTSGASCSKSRPHLAGTHTSLRLPQEGKGTCILVGGHEITSGLEVISSLRAIHGLQVEVCPLNGCDYIVSNRMVVERRSQSEMLNSVNKNKFIEQIQHLQSMFERICVIVEKDREKTGDTSRMFRRTKSYDSLLTTLIGAGIRILFSSCQEETADLLKELSLVEQRKNVGIHVPTVVNSNKSEALQFYLSIPNISYITALNMCHQFSSVKRMANSSLQEISMYAQVTHQKAEEIYRYIHYVFDIQMLPNDLNQDRLKSDI

SEQ ID NO: 27 - protein, 1940 aa, Artificial Sequence
  mutant FANCM protein delta ERCC4
MSGRQRTLFQTWGSSISRSSGTPGCSSGTERPQSPGSSKAPLPAAAEAQLESDDDVLLVAAYEAERQLCLENGGFCTSAGALWIYPTNCPVRDYQLHISRAALFCNTLVCLPTGLGKTFIAAVVMYNFYRWFPSGKVVFMAPTKPLVTQQIEACYQVMGIPQSHMAEMTGSTQASTRKEIWCSKRVLFLTPQVMVNDLSRGACPAAEIKCLVIDEAHKALGNYAYCQVVRELVKYTNHFRILALSATPGSDIKAVQQVITNLLIGQIELRSEDSPDILTYSHERKVEKLIVPLGEELAAIQKTYIQILESFARSLIQRNVLMRRDIPNLTKYQIILARDQFRKNPSPNIVGIQQGIIEGEFAICISLYHGYELLQQMGMRSLYFFLCGIMDGTKGMTRSKNELGRNEDFMKLYNHLECMFARTRSTSANGISAIQQGDKNKKFVYSHPKLKKLEEVVIEHFKSWNAENTTEKKRDETRVMIFSSFRDSVQEIAEMLSQHQPIIRVMTFVGHASGKSTKGFTQKEQLEVVKQFRDGGYNTLVSTCVGEEGLDIGEVDLIICFDSQKSPIRLVQRMGRTGRKRQGRIVIILSEGREERIYNQSQSNKRSIYKAISSNRQVLHFYQRSPRMVPDGINPKLHKMFITHGVYEPEKPSRNLQRKSSIFSYRDGMRQSSLKKDWFLSEEEFKLWNRLYRLRDSDEIKEITLPQVQFSSLQNEENKPAQESTTGIHQLSLSEWRLWQDHPLPTHQVDHSDRCRHFIGLMQMIEGMRHEEGECSYELEVESYLQMEDVTSTFIAPRNESNNLASDTFITHKKSSFIKNINQGSSSSVIESDEECAEIVKQTHIKPTKIVSLKKKVSKEIKKDQLKKENNHGIIDSVDNDRNSTVENIFQEDLPNDKRTSDTDEIAATCTINENVIKEPCVLLTECQFTNKSTSSLAGNVLDSGYNSFNDEKSVSSNLFLPFEEELYIVRTDDQFYNCHSLTKEVLANVERFLSYSPPPLSGLSDLEYEIAKGTALENLLFLPCAEHLRSDKCTCLLSHSAVNSQQNLELNSLKCINYPSEKSCLYDIPNDNISDEPSLCDCDVHKHNQNENLVPNNRVQIHRSPAQNLVGENNHDVDNSDLPVLSTDQDESLLLFEDVNTEFDDVSLSPLNSKSESLPVSDKTAISETPLVSQFLISDELLLDNNSELQDQITRDANSFKSRDQRGVQEEKVKNHEDIFDCSRDLFSVTFDLGFCSPDSDDEILEHTSDSNRPLDDLYGRYLEIKEISDANYVSNQALIPRDHSKNFTSGTVIIPSNEDMQNPNYVHLPLSAAKNEELLSPGYSQFSLPVQKKVMSTPLSKSNTLNSFSKIRKEILKTPDSSKEKVNLQRFKEALNSTFDYSEFSLEKSKSSGPMYLHKSCHSVEDGQLLTSNESEDDEIFRRKVKRAKGNVLNSPEDQKNSEVDSPLHAVKKRRFPINRSELSSSDESENFPKPCSQLEDFKVCNGNARRGIKVPKRQSHLKHVARKFLDDEAELSEEDAEYVSSDENDESENEQDSSLLDFLNDETQLSQAINDSEMRAIYMKSLRSPMMNNKYKMIHKTHKNINIFSQIPEQDETYLEDSFCVDEEESCKGQSSEEEVCVDFNLITDDCFANSKKYKTRRAVMLKEMMEQNCAHSKKKLSRIILPDDSSEEENNVNDKRESNIAVNPSTVKKNKQQDHCLNSVPSGSSAQSKVRSTPRVNPLAKQSKQTSLNLKDTISEVSDFKPQNHNEVQSTTPPFTTVDSQKDCRKFPVPQKDGSALEDSSTSGASCSKSRPHLAGTHTSLRLPQETTLIGAGIRILFSSCQEETADLLKELSLVEQRKNVGIHVPTVVNSNKSEALQFYLSIPNISYITALNMCHQFSSVKRMANSSLQEISMYAQVTHQKAEEIYRYIHYVFDIQMLPNDLNQDRLKSDI

SEQ ID NO: 28 - protein, 1970 aa, Artificial Sequence
  mutant FANCM protein delta HhH
MSGRQRTLFQTWGSSISRSSGTPGCSSGTERPQSPGSSKAPLPAAAEAQLESDDDVLLVAAYEAERQLCLENGGFCTSAGALWIYPTNCPVRDYQLHISRAALFCNTLVCLPTGLGKTFIAAVVMYNFYRWFPSGKVVFMAPTKPLVTQQIEACYQVMGIPQSHMAEMTGSTQASTRKEIWCSKRVLFLTPQVMVNDLSRGACPAAEIKCLVIDEAHKALGNYAYCQVVRELVKYTNHFRILALSATPGSDIKAVQQVITNLLIGQIELRSEDSPDILTYSHERKVEKLIVPLGEELAAIQKTYIQILESFARSLIQRNVLMRRDIPNLTKYQIILARDQFRKNPSPNIVGIQQGIIEGEFAICISLYHGYELLQQMGMRSLYFFLCGIMDGTKGMTRSKNELGRNEDFMKLYNHLECMFARTRSTSANGISAIQQGDKNKKFVYSHPKLKKLEEVVIEHFKSWNAENTTEKKRDETRVMIFSSFRDSVQEIAEMLSQHQPIIRVMTFVGHASGKSTKGFTQKEQLEVVKQFRDGGYNTLVSTCVGEEGLDIGEVDLIICFDSQKSPIRLVQRMGRTGRKRQGRIVIILSEGREERIYNQSQSNKRSIYKAISSNRQVLHFYQRSPRMVPDGINPKLHKMFITHGVYEPEKPSRNLQRKSSIFSYRDGMRQSSLKKDWFLSEEEFKLWNRLYRLRDSDEIKEITLPQVQFSSLQNEENKPAQESTTGIHQLSLSEWRLWQDHPLPTHQVDHSDRCRHFIGLMQMIEGMRHEEGECSYELEVESYLQMEDVTSTFIAPRNESNNLASDTFITHKKSSFIKNINQGSSSSVIESDEECAEIVKQTHIKPTKIVSLKKKVSKEIKKDQLKKENNHGIIDSVDNDRNSTVENIFQEDLPNDKRTSDTDEIAATCTINENVIKEPCVLLTECQFTNKSTSSLAGNVLDSGYNSFNDEKSVSSNLFLPFEEELYIVRTDDQFYNCHSLTKEVLANVERFLSYSPPPLSGLSDLEYEIAKGTALENLLFLPCAEHLRSDKCTCLLSHSAVNSQQNLELNSLKCINYPSEKSCLYDIPNDNISDEPSLCDCDVHKHNQNENLVPNNRVQIHRSPAQNLVGENNHDVDNSDLPVLSTDQDESLLLFEDVNTEFDDVSLSPLNSKSESLPVSDKTAISETPLVSQFLISDELLLDNNSELQDQITRDANSFKSRDQRGVQEEKVKNHEDIFDCSRDLFSVTFDLGFCSPDSDDEILEHTSDSNRPLDDLYGRYLEIKEISDANYVSNQALIPRDHSKNFTSGTVIIPSNEDMQNPNYVHLPLSAAKNEELLSPGYSQFSLPVQKKVMSTPLSKSNTLNSFSKIRKEILKTPDSSKEKVNLQRFKEALNSTFDYSEFSLEKSKSSGPMYLHKSCHSVEDGQLLTSNESEDDEIFRRKVKRAKGNVLNSPEDQKNSEVDSPLHAVKKRRFPINRSELSSSDESENFPKPCSQLEDFKVCNGNARRGIKVPKRQSHLKHVARKFLDDEAELSEEDAEYVSSDENDESENEQDSSLLDFLNDETQLSQAINDSEMRAIYMKSLRSPMMNNKYKMIHKTHKNINIFSQIPEQDETYLEDSFCVDEEESCKGQSSEEEVCVDFNLITDDCFANSKKYKTRRAVMLKEMMEQNCAHSKKKLSRIILPDDSSEEENNVNDKRESNIAVNPSTVKKNKQQDHCLNSVPSGSSAQSKVRSTPRVNPLAKQSKQTSLNLKDTISEVSDFKPQNHNEVQSTTPPFTTVDSQKDCRKFPVPQKDGSALEDSSTSGASCSKSRPHLAGTHTSLRLPQEGKGTCILVGGHEITSGLEVISSLRAIHGLQVEVCPLNGCDYIVSNRMVVERRSQSEMLNSVNKNKFIEQIQHLQSMFERICVIVEKDREKTGDTSRMFRRTKSYDSLLTTLIGAGIRILFSSCQEETADLLKELSLVEQRKNVGIHVPTVVNSNKS

SEQ ID NO: 29 - protein, 34 aa, Artificial Sequence; identical to SEQ ID NO: 5
  wildtype FANCM MM2 peptide

SEQ ID NO: 30 - protein, 34 aa, Artificial Sequence
  mutated FANCM peptide
EDIFDCSRDLFSVTADLGACSPDSDDEILEHTSD

SEQ ID NO: 31 - protein, 2022 aa, Homo sapiens
MSGRQRTLFQTWGSSISRSSGTPGCSSGTERPQSPGSSKAPLPAAAEAQLESDDDVLLVAAYEAERQLCLENGGFCTSAGALWIYPTNCPVRDYQLHISRAALFCNTLVCLPTGLGKTFIAAVVMYNFYRWFPSGKVVFMAPTKPLVTQQIEACYQVMGIPQSHMAEMTGSTQASTRKEIWCSKRVLFLTPQVMVNDLSRGACPAAEIKCLVIDEAHKALGNYAYCQAVQQVITNLLIGQIELRSEDSPDILTYSHERKVEKLIVPLGEELAAIQKTYIQILESFARSLIQRNVLMRRDIPNLTKYQIILARDQFRKNPSPNIVGIQQGIIEGEFAICISLYHGYELLQQMGMRSLYFFLCGIMDGTKGMTRSKNELGRNEDFMKLYNHLECMFARTRSTSANGISAIQQGDKNKKFVYSHPKLKKLEEVVIEHFKSWNAENTTEKKRDETRVMIFSSFRDSVQEIAEMLSQHQPIIRVMTFVGHASGKSTKGFTQKEQLEVVKQFRDGGYNTLVSTCVGEEGLDIGEVDLIICFDSQKSPIRLVQRMGRTGRKRQGRIVIILSEGREERIYNQSQSNKRSIYKAISSNRQVLHFYQRSPRMVPDGINPKLHKMFITHGVYEPEKPSRNLQRKSSIFSYRDGMRQSSLKKDWFLSEEEFKLWNRLYRLRDSDEIKEITLPQVQFSSLQNEENKPAQESTTGIHQLSLSEWRLWQDHPLPTHQVDHSDRCRHFIGLMQMIEGMRHEEGECSYELEVESYLQMEDVTSTFIAPRNESNNLASDTFITHKKSSFIKNINQGSSSSVIESDEECAEIVKQTHIKPTKIVSLKKKVSKEIKKDQLKKENNHGIIDSVDNDRNSTVENIFQEDLPNDKRTSDTDEIAATCTINENVIKEPCVLLTECQFTNKSTSSLAGNVLDSGYNSFNDEKSVSSNLFLPFEEELYIVRTDDQFYNCHSLTKEVLANVERFLSYSPPPLSGLSDLEYEIAKGTALENLLFLPCAEHLRSDKCTCLLSHSAVNSQQNLELNSLKCINYPSEKSCLYDIPNDNISDEPSLCDCDVHKHNQNENLVPNNRVQIHRSPAQNLVGENNHDVDNSDLPVLSTDQDESLLLFEDVNTEFDDVSLSPLNSKSESLPVSDKTAISETPLVSQFLISDELLLDNNSELQDQITRDANSFKSRDQRGVQEEKVKNHEDIFDCSRDLFSVTFDLGFCSPDSDDEILEHTSDSNRPLDDLYGRYLEIKEISDANYVSNQALIPRDHSKNFTSGTVIIPSNEDMQNPNYVHLPLSAAKNEELLSPGYSQFSLPVQKKVMSTPLSKSNTLNSFSKIRKEILKTPDSSKEKVNLQRFKEALNSTFDYSEFSLEKSKSSGPMYLHKSCHSVEDGQLLTSNESEDDEIFRRKVKRAKGNVLNSPEDQKNSEVDSPLHAVKKRRFPINRSELSSSDESENFPKPCSQLEDFKVCNGNARRGIKVPKRQSHLKHVARKFLDDEAELSEEDAEYVSSDENDESENEQDSSLLDFLNDETQLSQAINDSEMRAIYMKSLRSPMMNNKYKMIHKTHKNINIFSQIPEQDETYLEDSFCVDEEESCKGQSSEEEVCVDFNLITDDCFANSKKYKTRRAVMLKEMMEQNCAHSKKKLSRIILPDDSSEEENNVNDKRESNIAVNPSTVKKNKQQDHCLNSVPSGSSAQSKVRSTPRVNPLAKQSKQTSLNLKDTISEVSDFKPQNHNEVQSTTPPFTTVDSQKDCRKFPVPQKDGSALEDSSTSGASCSKSRPHLAGTHTSLRLPQEGKGTCILVGGHEITSGLEVISSLRAIHGLQVEVCPLNGCDYIVSNRMVVERRSQSEMLNSVNKNKFIEQIQHLQSMFERICVIVEKDREKTGDTSRMFRRTKSYDSLLTTLIGAGIRILFSSCQEETADLLKELSLVEQRKNVGIHVPTVVNSNKSEALQFYLSIPNISYITALNMCHQFSSVKRMANSSLQEISMYAQVTHQKAEEIYRYIHYVFDIQMLPNDLNQDRLKSDI

SEQ ID NO: 32 - DNA, 7060 bp, Homo sapiens
tgtgcgaaggaaaccgatggggatcggaaccgtagcggttgagctgctgctgctacggatatctgacagaagccttcggtggttgtcggcctaatgagcggacggcaaagaacgctttttcagacgtggggctcaagtatctcccgatcatctgggactccgggttgcagctccggaactgagcgacctcagagccctggcagctccaaggcgcctttgccagcagcagcggaggctcagctggagtcggacgatgatgtgttgcttgtcgcggcgtacgaggctgagcggcagttgtgtctagagaatggcgggttctgcacctccgcgggcgccctgtggatttaccctaccaattgcccagtgcgggactaccagctgcacatttcccgggctgctctgttttgcaatacgctggtgtgtctgcctaccggactgggaaagacctttattgccgccgtggtcatgtacaatttctaccgctggttcccttcaggaaaggtggtcttcatggccccaacgaaacccttggtgacacagcagatcgaggcttgctaccaggtgatgggtatcccgcaatcccacatggccgaaatgacagggtctacacaagcttccaccaggaaggaaatatggtgcagtaagagagtgctttttcttacacctcaggtcatggtaaatgacctttctagaggagcttgtcccgctgctgaaataaagtgtttagttattgatgaagctcataaagctctcggaaactatgcttattgccaggctgtgcaacaagttattactaacctgctaattgggcagatagagcttcgttctgaagattctccagatattttgacatattctcatgaaagaaaagttgaaaagcttattgttccgcttggtgaagaacttgcagccatccaaaagacctatatccagattttggaatcatttgctcgttctttgattcagaggaatgttttgatgagaagggatatcccaaatctaacaaaatatcagataattctggcaagagatcagtttaggaaaaacccatctccgaatattgtgggaatacaacaaggcataatcgagggagagtttgctatttgtattagtttatatcatggttatgaattattgcagcaaatgggaatgagatcattatatttcttcctttgtggaattatggatggaactaaagggatgacacggtcaaaaaatgaacttggccgaaatgaagacttcatgaaactctataatcatctagagtgtatgtttgcacgtacacgtagtacttcagcaaatggtatttctgctatccaacaaggagataaaaataaaaaatttgtttatagtcatccaaagttaaagaaattagaagaagttgtaattgaacacttcaagtcatggaatgctgaaaacactactgaaaagaaacgtgatgagacccgagttatgatcttctcttcatttcgagatagtgttcaagaaattgcagaaatgctttcacagcatcagccaattattagagtaatgacttttgtcggccatgcctcagggaaaagcacgaagggttttacccagaaggagcaactggaggtagtgaaacagtttcgtgacggtggttacaacacgctggtttctacctgtgtgggtgaagaaggtttggatataggagaagttgatcttataatatgttttgattcccagaagagcccaattcgtcttgtacaacgaatgggtagaactggccgtaaacgtcaaggcaggatagttattatcctttctgaaggacgagaggaacgtatttataatcagagtcagtccaacaaaagaagtatatataaagctatttcaagtaacaggcaggtccttcatttttaccaaagaagtccacgaatggttcctgatggaatcaacccaaaattacacaaaatgttcatcacacatggtgtctatgaaccagagaagccttctcggaacttgcagcgaaagtcatctatcttttcctatagggatggaatgaggcaaagtagcctaaagaaagattggttcttatcagaagaagaatttaaattatggaacagactttatagattaagggacagtgatgaaattaaagagataacattgcctcaagttcagttttcttctttacaaaatgaggaaaacaaaccagctcaagaatcaaccactggaattcatcaactctctctctctgaatggagactgtggcaagatcatcctttgcctacacatcaagttgatcactcagatcgatgccgccattttataggccttatgcaaatgatagagggaatgagacacgaagagggagaatgcagctatgaattggaagttgaatcttatttacaaatggaagatgttacctcaacatttattgctcccaggaatgaatctaataatcttgccagtgacacctttatcactcacaagaaatcgtcatttataaagaacataaatcaaggcagttcatcctcagtgatagaatctgatgaagaatgtgctgaaattgttaaacaaactcatatcaaacctactaaaattgtttctttaaagaaaaaagtgtctaaagaaataaaaaaagatcagcttaaaaaagaaaataatcacggtattatagattctgtagataatgacagaaattccactgttgaaaatatttttcaagaagacctaccaaatgataaaaggacatcagatacagatgaaattgctgccacatgtactattaatgaaaatgttattaaagaaccgtgtgtgttattaacagagtgtcagtttacaaataaatccactagttcacttgctggaaatgttttagattctggttataacagtttcaatgatgaaaaatctgtttcatctaacttatttcttccattcgaagaagagctttatattgttagaacagatgaccaattttataattgtcactcattgacaaaagaggtactagctaatgtagagagatttttatcttattctcctccgcctctcagtggactctcagacttggaatatgaaattgctaagggtactgcacttgagaatttgcttttcttaccctgtgcagagcatttacgaagtgataaatgcacctgtttgctgtcacattcagctgtgaattctcaacagaatttagaattgaattcacttaaatgtataaattatccatctgaaaaaagttgcctttatgatatacctaatgataatatttctgatgagccaagtctctgtgactgtgatgtacataaacataatcaaaatgaaaatttagtacctaacaatcgtgttcaaatacacagaagccctgcacagaatttagttggagagaacaatcatgatgttgataacagtgacctcccagtattgtccactgatcaagatgaaagtttgctgttatttgaagatgttaatacagagttcgacgatgtgagtctttcacccttgaacagtaaaagcgaatctttacctgtgtcagacaaaactgctattagtgaaacgcctctggtctctcagttcttaatttctgatgaacttttgttggacaataattctgaactccaagatcaaatcacccgtgatgctaatagttttaaatctcgtgatcagagaggtgtacaggaagaaaaagtgaagaatcatgaggatatttttgattgctctagggatttattttctgttacctttgatttaggattctgtagtccagattctgatgatgaaatattggaacatacatcagatagcaatagacctctagatgatctatatggaaggtatttggaaattaaggagataagtgatgcaaattatgtttcgaatcaagcactaataccaagagatcatagtaaaaattttactagtggaactgttattatcccatcaaatgaagatatgcagaatccaaattatgtacatttgccactgagtgcagcaaaaaatgaagaattgttatctcctggttattctcagttttctttaccagtgcaaaaaaaagttatgagtacaccactctctaaatcaaacacattgaactcattttctaagataagaaaggaaatacttaagacaccagattctagtaaggaaaaagtaaacctacaaagattcaaagaagcattgaattcaacttttgattattcagaattttctctagaaaagtctaaaagcagtggtccaatgtatctgcataaatcctgtcattctgttgaagatggacaattattaacaagtaacgaaagtgaagatgacgagattttccgaagaaaagttaaaagagcaaaaggaaatgttttaaactctcctgaggatcagaaaaatagtgaagttgattctccacttcatgctgtcaaaaagcgcagatttcctataaacagatcagaattatcatctagtgatgagagtgagaattttcccaaaccatgttcacaattagaagacttcaaggtttgtaacgggaatgccagaagaggcatcaaagtcccaaagagacagagtcacttaaagcatgtagctaggaagtttttagatgatgaagcagaactttctgaagaagatgcagaatatgtttcatcagatgaaaatgatgagtcagaaaatgaacaagattcctcattacttgactttttaaatgatgaaactcaactttcacaggctataaatgattctgaaatgagagctatttacatgaaatctttgcgtagtccaatgatgaacaataagtacaaaatgattcataagacacataaaaacataaacattttctcgcagattcctgaacaagatgaaacctatttagaggatagtttttgtgttgatgaagaggagtcttgcaaaggccaatcaagtgaagaagaagtttgtgttgattttaacttaataactgatgattgctttgcaaatagtaaaaagtataaaactcgacgtgcagtaatgctaaaagaaatgatggaacaaaattgtgcacattcaaaaaagaaattatccagaattattttaccagatgattcaagtgaggaggagaacaatgtaaatgataaaagagaatctaatattgcggttaacccaagcactgttaagaagaacaaacaacaggaccattgtttaaattcagtgccttctggatcttctgcgcagtccaaggtgcgttctactccaagagttaatccattagcaaagcagagcaaacagacatcgctgaatttaaaggatacaatttccgaagtctcagacttcaaacctcagaatcataatgaagtccagtctaccacaccacccttcactactgttgattcacagaaagactgtagaaaatttccagttccacagaaggatggtagtgctttggaggattctagcacttcaggggcatcctgttccaagtcaagaccacatttagctgggacacatacttctcttagacttccgcaggaaggaaaaggaacctgtattcttgtaggtggtcatgaaatcacttctggattagaagtaatttcttccctaagagcaattcatgggttgcaagtagaagtttgtcctcttaatggctgtgattacatcgtgagtaatcgcatggtggtggaaaggaggtctcaatctgagatgttaaatagtgtcaataagaacaagttcattgagcagatccagcacctgcagagtatgtttgaaagaatatgtgtgattgtggaaaaggacagagaaaaaacaggagacacatcaaggatgtttaggagaacaaagagctatgacagcctgctgactaccttaattggcgctggaatccgaattcttttcagttcctgccaagaagaaaccgcagatttgctaaaggaactgtctttagtggaacaaagaaagaatgttggtattcatgttccaacagtggtgaatagtaataaaagtgaggcactccagttttatttaagtattcccaatataagttatataactgcattaaatatgtgtcaccagttttcatctgtgaaaaggatggctaacagctcacttcaagaaatctccatgtatgcacaagtaactcatcagaaggctgaggagatctatagatatattcactatgtatttgacatacaaatgttaccaaatgatcttaaccaagatagactgaaatctgatatataatcaagctgctcaagatggggttttcaaagacctctcacaatattaaatgcacttcaataatcattgctgttttatgtttatttgtaaataagagaatattttatttaaatattttatattgtatacatttttatttatagattatagaaattattaaaaaagaaaaatctgatgttcagtgatcattttgactagattataaaactaatttttcttattaaataaaacaaggtttattaaaagtgttactaaggatagtttaagaaagtaaaagctaagctagagatatactttggaatgtttcccaaaattaaagttgtactgttgtgataaatagtaaagttgacatgtctatgactacagccaacttgtcgattttccctatgtgtagatagtatacttttaagtgtactgattctaaatacatgtacttggtaaggtgtgggtgatgggtgggttgtgagataaatgacccagtaactaggaaagtagaaaacttaactgaatgtttatctgaccaaaggtgtgtcccagttaagtactgtcaaatctattaatatgaactctgatatggtttggctgtgtccccaaccaaaatctcatcttgacttgtaatctgaattataatcccaatatattggggagggacctcctggaacgtgattagctcatgggggcggttcccccatgctgttctagtgatagttctcagaggatctgatggttttataagcttttcctctgttcactctgcagttctcttgcctactgccatgtggaaaaggaaacgtttgcttcccctccaccatgattgtaagttcccgaggcctccccagccatgcaggactgtgagtcaattaaacatcttttccttataaattaaaaaaaaaaaaaaaaa

SEQ ID NO: 33 - DNA, 25 bp, Artificial Sequence
  Synthesized siFa
ggatgtttaggagaacaaagagcta

SEQ ID NO: 34 - DNA, 25 bp, Artificial Sequence
  Synthesized siFb
cccatcaaatgaagatatgcagaat

SEQ ID NO: 35 - protein, 2048 aa, Artificial Sequence
  Synthesized mutant
MSGRQRTLFQTWGSSISRSSGTPGCSSGTERPQSPGSSKAPLPAAAEAQLESDDDVLLVAAYEAERQLCLENGGFCTSAGALWIYPTNCPVRDYQLHISRAALFCNTLVCLPTGLGRTFIAAVVMYNFYRWFPSGKVVFMAPTKPLVTQQIEACYQVMGIPQSHMAEMTGSTQASTRKEIWCSKRVLFLTPQVMVNDLSRGACPAAEIKCLVIDEAHKALGNYAYCQVVRELVKYTNHFRILALSATPGSDIKAVQQVITNLLIGQIELRSEDSPDILTYSHERKVEKLIVPLGEELAAIQKTYIQILESFARSLIQRNVLMRRDIPNLTKYQIILARDQFRKNPSPNIVGIQQGIIEGEFAICISLYHGYELLQQMGMRSLYFFLCGIMDGTKGMTRSKNELGRNEDFMKLYNHLECMFARTRSTSANGISAIQQGDKNKKFVYSHPKLKKLEEVVIEHFKSWNAENTTEKKRDETRVMIFSSFRDSVQEIAEMLSQHQPIIRVMTFVGHASGKSTKGFTQKEQLEVVKQFRDGGYNTLVSTCVGEEGLDIGEVDLIICFDSQKSPIRLVQRMGRTGRKRQGRIVIILSEGREERIYNQSQSNKRSIYKAISSNRQVLHFYQRSPRMVPDGINPKLHKMFITHGVYEPEKPSRNLQRKSSIFSYRDGMRQSSLKKDWFLSEEEFKLWNRLYRLRDSDEIKEITLPQVQFSSLQNEENKPAQESTTGIHQLSLSEWRLWQDHPLPTHQVDHSDRCRHFIGLMQMIEGMRHEEGECSYELEVESYLQMEDVTSTFIAPRNESNNLASDTFITHKKSSFIKNINQGSSSSVIESDEECAEIVKQTHIKPTKIVSLKKKVSKEIKKDQLKKENNHGIIDSVDNDRNSTVENIFQEDLPNDKRTSDTDEIAATCTINENVIKEPCVLLTECQFTNKSTSSLAGNVLDSGYNSFNDEKSVSSNLFLPFEEELYIVRTDDQFYNCHSLTKEVLANVERFLSYSPPPLSGLSDLEYEIAKGTALENLLFLPCAEHLRSDKCTCLLSHSAVNSQQNLELNSLKCINYPSEKSCLYDIPNDNISDEPSLCDCDVHKHNQNENLVPNNRVQIHRSPAQNLVGENNHDVDNSDLPVLSTDQDESLLLFEDVNTEFDDVSLSPLNSKSESLPVSDKTAISETPLVSQFLISDELLLDNNSELQDQITRDANSFKSRDQRGVQEEKVKNHEDIFDCSRDLFSVTADLGACSPDSDDEILEHTSDSNRPLDDLYGRYLEIKEISDANYVSNQALIPRDHSKNFTSGTVIIPSNEDMQNPNYVHLPLSAAKNEELLSPGYSQFSLPVQKKVMSTPLSKSNTLNSFSKIRKEILKTPDSSKEKVNLQRFKEALNSTFDYSEFSLEKSKSSGPMYLHKSCHSVEDGQLLTSNESEDDEIFRRKVKRAKGNVLNSPEDQKNSEVDSPLHAVKKRRFPINRSELSSSDESENFPKPCSQLEDFKVCNGNARRGIKVPKRQSHLKHVARKFLDDEAELSEEDAEYVSSDENDESENEQDSSLLDFLNDETQLSQAINDSEMRAIYMKSLRSPMMNNKYKMIHKTHKNINIFSQIPEQDETYLEDSFCVDEEESCKGQSSEEEVCVDFNLITDDCFANSKKYKTRRAVMLKEMMEQNCAHSKKKLSRIILPDDSSEEENNVNDKRESNIAVNPSTVKKNKQQDHCLNSVPSGSSAQSKVRSTPRVNPLAKQSKQTSLNLKDTISEVSDFKPQNHNEVQSTTPPFTTVDSQKDCRKFPVPQKDGSALEDSSTSGASCSKSRPHLAGTHTSLRLPQEGKGTCILVGGHEITSGLEVISSLRAIHGLQVEVCPLNGCDYIVSNRMVVERRSQSEMLNSVNKNKFIEQIQHLQSMFERICVIVEKDREKTGDTSRMFRRTKSYDSLLTTLIGAGIRILFSSCQEETADLLKELSLVEQRKNVGIHVPTVVNSNKSEALQFYLSIPNISYITALNMCHQFSSVKRMANSSLQEISMYAQVTHQKAEEIYRYIHYVFDIQMLPNDLNQDRLKSDI

SEQ ID NO: 36 - DNA, 25 bp, Artificial Sequence
  Synthesized siBl
gctaggagtctgcgtgcgaggatta

SEQ ID NO: 37 - RNA, 27 nt, Artificial Sequence
  Synthesized siATRXa
gaggaaaccuucaauuguaacaaagua

SEQ ID NO: 38 - RNA, 27 nt, Artificial Sequence
  Synthesized siATRXb
ugcaagcucuaucaguacuacuuagau

SEQ ID NO: 39 - RNA, 27 nt, Artificial Sequence
  synthesized siTRF1
cuuucuuucuuauuaaggucuuguugc

SEQ ID NO: 40 - RNA, 27 nt, Artificial Sequence
  Synthesized siRNaseH1
uugucuaaugccuacauuuaaaggaug

SEQ ID NO: 41 - DNA, 21 bp, Artificial Sequence
  primer
agagtgtctggagcaagttgc

SEQ ID NO: 42 - DNA, 21 bp, Artificial Sequence
  Primer
cgtagtccatgttcacaatcg

SEQ ID NO: 43 - DNA, 23 bp, Artificial Sequence
  Primer
gtggtggccattttttgtctaac

SEQ ID NO: 44 - DNA, 22 bp, Artificial Sequence
  Primer
tgctctagaatgaacggtggaa

SEQ ID NO: 45 - DNA, 20 bp, Artificial Sequence
  Primer
tccctggagaagagctacga

SEQ ID NO: 46 - DNA, 20 bp, Artificial Sequence
  Primer
agcactgtgttggcgtacag

SEQ ID NO: 47 - DNA, 18 bp, Artificial Sequence
  Made in Lab probe
ccctaaccctaaccctaa

SEQ ID NO: 48 - DNA, 30 bp, Artificial Sequence
  Made in Lab oligonucleotide
gtgatccgcccgcctcggcctcccaaagtg

SEQ ID NO: 49 - protein, 12 aa, Homo sapiens
DLFSVTFDLGFC

SEQ ID NO: 50 - protein, 33 aa, Homo sapiens
DIFDCSRDLFSVTFDLGFCSPDSDDEILEHTSD